(19) United States
(12) Patent Application Publication
(10) Pub. No.: US 2010/0010035 A1
(43) Pub. Date: Jan. 14, 2010

(54) Novel Dual Action Receptors Antagonists (Dara) at the Ati and Eta Receptors

(76) Inventors: Ramesh Chandra Gupta, Ahmedabad (IN); Vikrant Vijaykumar Jagtap, Gandhinagar (IN); Appaji Baburao Mandhare, Ahmedabad (IN); Tim Perkins, Molndal (SE); Christer Westerlund, Molndal (SE)

Correspondence Address: JACOBSON HOLMAN PLLC, 400 SEVENTH STREET N.W., SUITE 600, WASHINGTON, DC, 20004, US

(21) Appl. No.: 12/224,617

(22) PCT Filed: Mar. 1, 2007
(86) PCT No.: PCT/SE2007/000199

§ 371 Date: Mar. 12, 2009

Related U.S. Application Data
(60) Provisional application No. 60/778,855, filed on Mar. 3, 2006.

(51) Int. Cl. A61K 31/422; A61K 31/4375; A61K 31/437; A61K 31/4709; A61K 31/4439; C07D 471/04; C07D 413/14; A61P 9/12

(52) U.S. Cl. 514/300; 514/303; 514/312; 514/340; 514/380; 546/119; 546/122; 546/153; 546/272.1; 548/246

(57) ABSTRACT

The present invention relates to new compounds of the formula [Chemical formula should be inserted here. Please see paper copy] wherein R1, R2, R3, and R31 are as specified herein. The invention also relates to a method for preparation thereof, as well as combinations of the new compounds with previously known agents. The invention also relates to the use of the above-mentioned compounds and combinations for the preparation of a medicament for treating hypertension of different kinds, alleviating organ damage of different kinds, treating or preventing diabetic nephropathy, treating endothelin and angiotensin mediated disorders, and treating prostate cancer.

### FIELD OF THE INVENTION

The present invention relates to new compounds and to a method for preparation thereof. These compounds are dual action receptor antagonists (DARA) at the AT1 and ETA receptors. The invention also relates to combinations of the new compounds with previously known agents. The invention also relates to the use of the above-mentioned compounds and combinations for the preparation of a medicament for treating hypertension of different kinds, alleviating organ damage of different kinds, treating or preventing diabetic nephropathy and treating endothelin and angiotensin mediated disorders.

### BACKGROUND OF THE INVENTION AND PRIOR ART

Hypertension is clearly a pervasive condition, with important health and economic implications for both individuals and society. Despite the presence of many classes of antihypertensive agents on the market, more than 40% of treated hypertensive patients do not have their blood pressure well controlled. Given the multi-factorial nature of cardiovascular diseases including hypertension, simultaneous targeting of more than one physiologic mechanism seems a plausible strategy to achieve better effects.

With angiotensin-II (Ang-II) and its receptor AT1 as an established target for the treatment of hypertension and heart failure, there has been increasing interest in the biological effects of other vasoactive peptides. Cardiovascular homeostasis involves a cross talk between the renin-angiotensin and endothelin systems, each system exaggerating the responses of the other. Thus, Ang-II stimulates synthesis of prepro-endothelin mRNA and ET-1 release. ET-1 mediates part of Ang II-induced excessive cellular proliferation inherent in end organ damage. Pre-treatment with an ETA receptor antagonist blunts the increase in blood pressure evoked by an infusion of Ang-II.

The interaction of the renin-angiotensin and endothelin system, two principal vasoactive systems, therefore makes a combined target especially attractive. A mixed endothelin-angiotensin antagonist would not only enhance the antihypertensive effect of AT1 blockade but also attenuate the severity of end-organ damage as shown in several rat models of hypertension. Thus, a sub effective dose of the AT1 receptor antagonist Losartan, resulted in a normalization of blood pressure when combined with an ETA antagonist, and the combination also reduced cardiac hypertrophy and increased survival as compared to treatment with Losartan alone (Bohlender J. et al, Hypertension 2000:35:992-7).

The reasons for pursuing a dual acting receptor antagonist rather than a fixed combination are several. Firstly, from a regulatory point each compound in a fixed combination has to be documented in monotherapy. Thereafter the fixed combination has to be documented clinically in a factorial study and in complementary studies. Also toxicological studies have to be performed with the two drugs in combination. In contrast, a dual acting receptor antagonist will be documented in the routine way for a new compound.

Secondly, to include an ETA antagonist in a fixed combination is not an option since most ETA antagonists have toxicological effects. To avoid the adverse effects inherent in ETA blockade a new dual acting receptor antagonist will be designed to have higher affinity for AT1 than for ETA. However, the affinity for ETA must not be nil. Thus, the new dual acting receptor antagonist has activity for both the AT1 and ETA receptors. In addition, to avoid blockade of the positive actions conveyed via the ETB receptor (vasodilation, natriuresis and ET-1 clearance) and via the AT2 receptors (vasodilation and anti-proliferative effects) the new compounds should preferably selectively target only the AT1 and ETA receptors.

Endothelin antagonists and angiotensin II antagonists, not being dual AT1 and ETA antagonists, for use in the treatment of hypertension are previously known. For instance, WO 98/49162, to Texas Biotechnology Corp., discloses heteroaromatic sulphonamides as endothelin antagonists. Likewise, EP 513 979 A1, to Merck and Co., Inc., discloses angiotensin II antagonists incorporating a substituted thiophene or furan.

Bristol-Myers Squibbs has described several series of dual receptors (Jae et al., Pyrrolidine-3-carboxylic acids as Endothelin antagonist. 5. Highly selective, potent, and orally active ETA antagonist. J. Med. Chem. 2001, 44: 3978-3984; Tellew et al., Discovery of 4′-[(Imidazol-1-yl)methyl]biphenyl-2-sulphonamides as dual endothelin/angiotensin II receptor antagonists. Bio. & Med. Chem. Lett., 2003, 13: 1093-1096, US2002143024, WO00/001389, and WO01/044239).

However, the compounds according to the present invention have not previously been disclosed. Furthermore, it has been shown that the selectivity of compounds of the present invention have an unexpected selectivity for AT1 to ETA, i e ratio between the affinities for AT1 and ETA.

### DESCRIPTION OF THE INVENTION

One object of the present invention is a compound of formula

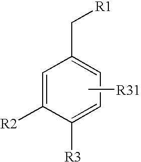

wherein R3 has any of the formulas

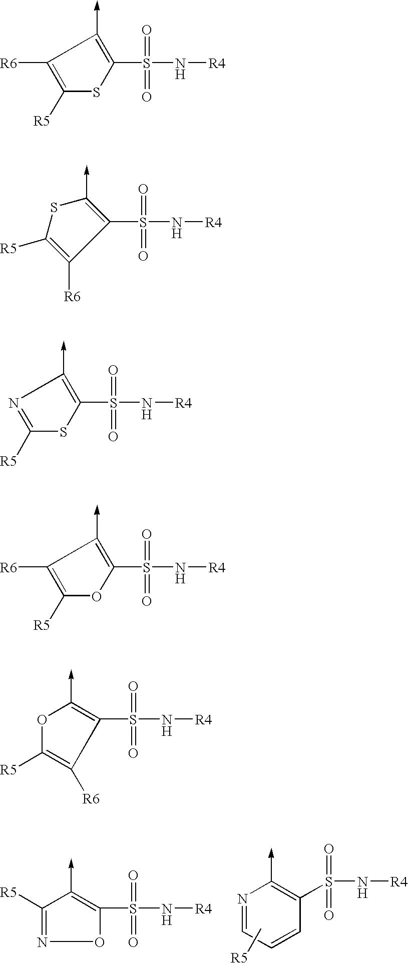

wherein R1 is selected from

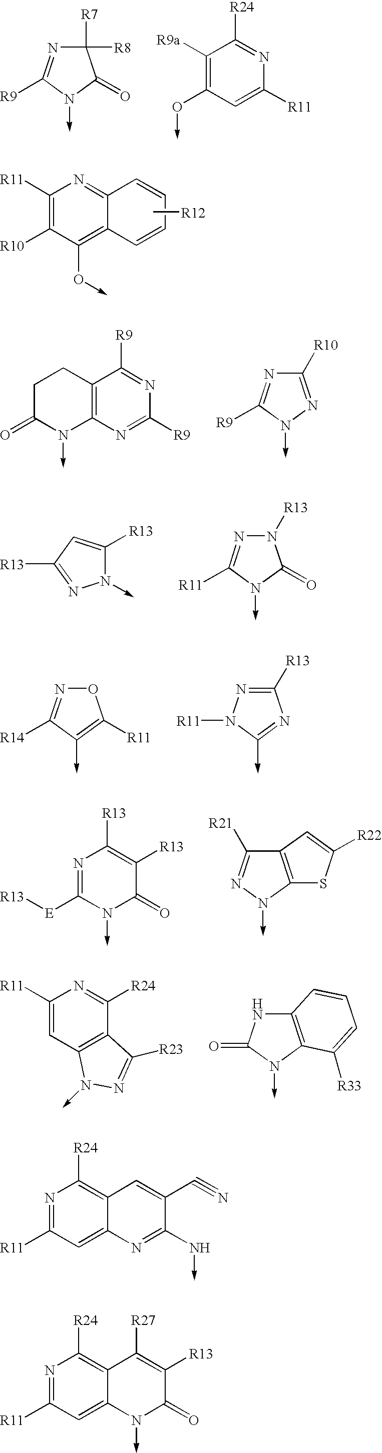

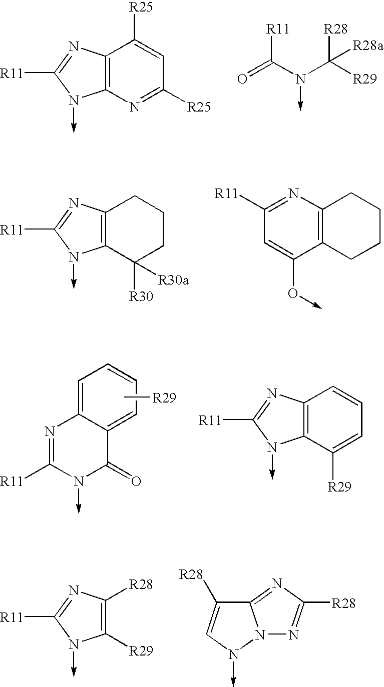

wherein
R2 is each independently hydrogen, halogen, C₁-C₈ alkyl, halo-C₁-C₈ alkyl, C₃-C₈ cycloalkyl, C₂-C₈ alkenyl, C₂-C₈ alkynyl, C₁-C₈ alkoxy-C₁-C₈ alkyl, C₁-C₈ alkoxy, aryloxy, C₁-C₈ alkoxy-C₁-C₈ alkoxy, cyano, hydroxyl, hydroxy-C₁-C₈ alkyl, nitro, —(CH2)_{W}NR18R19 wherein w is 0, 1, 2, or 3 and R18 and R19 are independently hydrogen, C₁-C₈ alkyl, aryl, aryl-C₁-C₈ alkyl, heteroaryl, heteroaryl-C₁-C₈ alkyl or may together form a five or six membered saturated or unsaturated ring structure optionally containing one to two heteroatoms, selected from oxygen, sulphur or nitrogen and may be optionally substituted by C₁-C₈ alkyl, hydroxyl or oxo;
R4 is a five or six membered mono or bicyclic ring system having one to three heteroatoms, selected from O, N and S such as pyridyl, pyrimidyl, pyrazinyl, pyridazinyl, triazinyl, oxazolyl, isoxazolyl, thiazolyl, thiadiazolyl, isothiazolyl, oxadiazolyl, imidazolyl, triazolyl, tetrazolyl and pyridothiazolyl each of which may optionally be substituted, where appropriate by one or more of the following: hydrogen, halogen, cyano, C₁-C₈ alkyl, C₁-C₈ alkoxy, trifluoromethyl, and —COR32;
R5 and R6 are independently hydrogen, halogen, C₁-C₈ alkyl, —COOR13, —CO—NR18R19, cyano and —NR18R19, or R5 and R6 may together form a five or six membered cycloalkyl, aryl ring or heteroaryl ring structure having one to two heteroatoms, selected from O, N and S, which may be further substituted with C₁-C₈ alkyl, C₁-C₈ alkoxy or hydroxy; wherein R18 and R19 are independently selected from hydrogen, C₁-C₈ alkyl, aryl-C₁-C₈ alkyl, heteroaryl-C₁-C₈ alkyl, (C₃-C₈ cycloalkyl)-C₁-C₈ alkyl or may together form a five or six member saturated ring structure optionally containing one to two heteroatoms selected from O, N and S;
R7 and R8 are each independently C₁-C₈ alkyl, hydroxy-C₁-C₈ alkyl, C₃-C₈ cycloalkyl, hydroxy substituted C₃-C₈ cycloalkyl, C₁-C₈ alkoxy-C₁-C₈ alkyl, hydroxy substituted C₁-C₈ alkoxy-C₁-C₈ alkyl, or R7 and R8 together form a cyclobutyl, cyclopentyl, cyclohexyl, tetrahydrofuranyl or tetrahydropyranyl ring, which may be optionally substituted with one or more hydroxyl groups;
R9 is independently C₁-C₈ alkyl, hydroxy-C₁-C₈ alkyl, hydroxy substituted halo-C₁-C₈ alkyl, C₃-C₈ cycloalkyl, (C₃-C₈ cycloalkyl)-C₁-C₈ alkyl, aryl-C₁-C₈ alkyl, C₁-C₈ alkoxy, hydroxy substituted C₁-C₈ alkoxy, C₁-C₈ alkoxy-C₁-C₈ alkyl, hydroxy substituted C₁-C₈ alkoxy-C₁-C₈ alkyl, C₁-C₈ alkylcarbonyl, arylcarbonyl, carboxy, C₁-C₈ alkoxycarbonyl, and heteroaryl-C₁-C₈ alkyl;
R9a is independently C₁-C₈ alkyl, C₁-C₈ alkoxy-C₁-C₈ alkyl, C₁-C₈ alkylcarbonyl, arylcarbonyl, heteroarylcarbonyl, carboxy, C₁-C₈ alkoxy and —COOR13;
R10 is hydrogen, C₁-C₈ alkyl, (C₃-C₈ cycloalkyl)-C₁-C₈ alkyl, or aryl-C₁-C₈ alkyl;
R11 is independently C₁-C₈-alkyl, C₁-C₈ alkoxy, aryl-C₁-C₈ alkyl, heteroaryl-C₁-C₈ alkyl and (C₃-C₈ cycloalkyl)-C₁-C₈ alkyl;
R12 is hydrogen, halogen, C₁-C₈ alkyl, —COOR17, C₁-C₈ alkyl-C₁-C₈ thioalkyl, C₁-C₈ alkoxy or C₁-C₈ alkoxy-C₁-C₈ alkyl, nitro, NHR24;
R13 independently is hydrogen, C₁-C₈ alkyl, aryl and heteroaryl;
R14 is independently hydrogen, C₁-C₈ alkyl, aryl, NHCOR13 and NR18R19, wherein R18, R19 are independently selected from hydrogen, C₁-C₈ alkyl, aryl-C₁-C₈ alkyl, or may together optionally form a five or six membered saturated ring structure optionally containing one to two heteroatoms selected from O, N and S;
E is a single bond, —(CH₂)— or —S—;
R17 is hydrogen, C₁-C₄ alkyl optionally substituted with an aryl;

### R21 is

(a) C₁-C₈ alkyl, halo-C₁-C₈ alkyl, aryl-C₁-C₈ alkyl, or heteroaryl-C₁-C₈ alkyl,
(b) —(CH₂)NR18R19, wherein R18 and R19 are independently hydrogen, C₁-C₈ alkyl, aryl, heteroaryl or may together form a five or six membered saturated or unsaturated ring structure optionally containing having one to two heteroatoms, selected from O, N and S,
(c) aryl or
(d) heteroaryl;

### R22 is

(a) —CO₂R13, —CO₂—C₁-C₈ alkyl, —CO—NR18R19, or
(b) —(CH₂)NR18R19, wherein R18 and R19 are independently hydrogen, C₁-C₈ alkyl, aryl, heteroaryl or may together form a five or six membered saturated or unsaturated ring structure optionally containing having one to two heteroatoms, selected from O, N and S;

### R23 is

(a) hydrogen, C₁-C₈ alkyl, aryl, C₁-C₈ alkoxy, halogen, heteroaryl, heteroaryl-C₁-C₈ alkyl, C₃-C₆ cycloalkyl, (C₃-C₈ cycloalkyl)-C₁-C₈ alkyl, —CH₂COOR13, —CH₂CONHR13, or trifluoromethyl, wherein any aryl and heteroaryl residues are optionally substituted with hydrogen, halogen, C₁-C₈ alkyl, C₁-C₈ alkoxy, cyano, trifluoromethyl, nitro, amino, —NHSO₂—R13, —SO₂NHR13, —COOR13, —CONHR13,
   or
(b) —(CH₂)NR18R19, wherein R18 and R19 are independently hydrogen, C₁-C₈ alkyl, aryl, heteroaryl or may together form a five or six membered saturated or unsaturated ring structure optionally containing one to two heteroatoms, selected from oxygen, sulphur and nitrogen, aryl and heteroaryl optionally substituted with hydrogen, halogen, C₁-C₈ alkyl, C₁-C₈ alkoxy, cyano, trifluoromethyl, nitro, amino, —NHSO₂—R14, —SO₂NHR24, COOH, —COOR17, or —CONHR14;

### R24 is

C₁-C₈ alkyl, C₁-C₈ alkoxy, aryl, heteroaryl, aryl-C₁-C₈ alkyl, heteroaryl-C₁-C₈ alkyl, (C₃-C₈ cycloalkyl)-C₁-C₈ alkyl, and trifluoromethyl,
wherein any aryl and heteroaryl residues are optionally mono- or disubstituted with halogen, C₁-C₈ alkyl, C₁-C₈ alkoxy, cyano, trifluoromethyl, nitro, amino, —NHSO₂—R13, —SO₂NHR13, COOR13, —CONHR13, —(CH₂)NR18R19, wherein R18 and R19 are independently hydrogen, C₁-C₈ alkyl, or may together form a five or six membered saturated or unsaturated ring structure optionally having one to two heteroatoms, selected from O, N and S;
   R25 is independently C₁-C₆ alkyl, (C₃-C₆ cycloalkyl)-C₁-C₈ alkyl;
   R27 is H, aryl, heteroaryl, C₁-C₈ alkyl, C₁-C₈ alkoxy, O-aryl, O-heteroaryl, S-aryl, S-heteroaryl or NR18R19, wherein R18 and R19 are independently selected from H, C₁-C₈ alkyl, heteroaryl-C₁-C₈ alkyl, (C₃-C₈ cycloalkyl)-C₁-C₈ alkyl, or may together form a five or six membered saturated ring structure optionally containing one to two heteroatoms selected from O, N and S, which may be further substituted with C₁-C₈ alkyl, wherein aryl and heteroaryl residues are optionally mono- or disubstituted with halogen, C₁-C₈ alkyl, C₁-C₈ alkoxy, trifluoromethyl;
   R28 and R28a are each independently hydrogen, halogen, C₁-C₈ alkyl, hydroxy-C₁-C₈ alkyl, C₃-C₈ cycloalkyl, (C₃-C₈ cycloalkyl)-C₁-C₈ alkyl, aryl, heteroaryl, aryl-C₁-C₈ alkyl, C₁-C₈ alkyl-C₁-C₈ thioalkyl, C₁-C₈ alkoxy, C₁-C₈ alkoxy-C₁-C₈ alkyl or R28 and R28a together with the carbon atom to which they are bonded form a C₃-C₈ cycloalkyl ring;

### R29 is

(a) —(CH₂)_{W}—COOR17,
(b) —(CH₂)_{W}—(C═O)NR18R19, wherein R18 and R19 are independently selected from H, C₁-C₈ alkyl, aryl, heteroaryl, or R18 and R19 may together form a five or six membered saturated ring structure containing one or two heteroatoms selected from O, N and S, wherein an aryl or heteroaryl residues may be mono- or disubstituted by halogen, C₁-C₈ alkyl, C₁-C₈ alkoxy, and trifluoromethyl
or
(c) —(CH₂)_{W}—CH₂—OH,
   wherein w is 0, 1 or 2;
      R30 and R30a are each independently hydrogen, C₁-C₈ alkoxy or together form a carbonyl;
      R31 is each independently hydrogen, halogen, C₁-C₈ alkyl, C₁-C₈ alkoxy-C₁-C₈ alkyl, cyano, hydroxy, hydroxy-C₁-C₈ alkyl, C₂-C₈ alkynyl and halo-C₁-C₈ alkyl;
      R32 is C₁-C₆ alkyl, C₃-C₆ cycloalkyl, aryl and heteroaryl; and
      R33 is C₁-C₈ alkoxycarbonyl;
      including pharmaceutically acceptable salts, hydrates, solvates, atropisomers, enantiomers, diastereomers, tautomers, polymorphs and prodrug forms thereof.

In another embodiment, the present invention pertains to a compound as above, however only including pharmaceutically acceptable salts thereof.

In another embodiment, the present invention pertains to a compound as above, wherein

R3 has any of the formulas

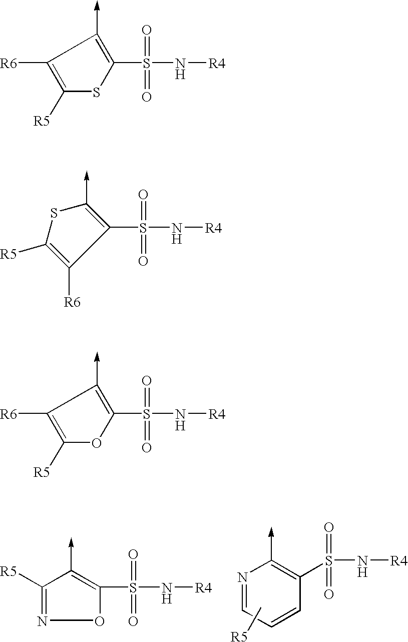

wherein R1 is selected from

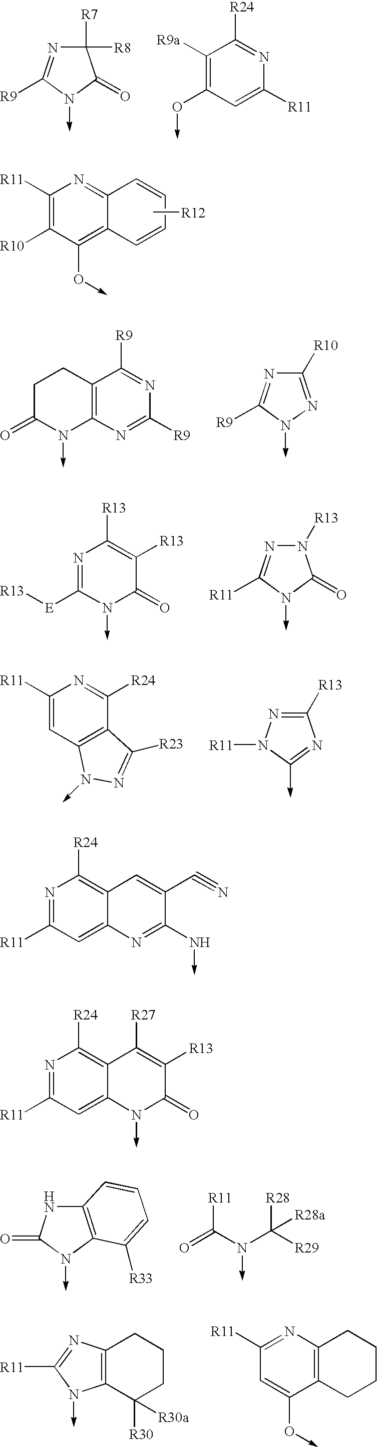

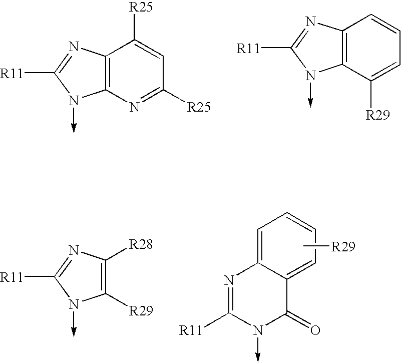

wherein
R2 is each independently hydrogen, halogen, C₁-C₈ alkyl, C₁-C₈ alkoxy-C₁-C₈ alkyl, C₁-C₈ alkoxy, C₁-C₈ alkoxy-C₁-C₈ alkoxy, hydroxyl, hydroxy-C₁-C₈ alkyl, —(CH2)_{W}NR18R19 wherein w is 1 and R18 and R19 form a five or six membered saturated or unsaturated ring structure optionally containing one to two heteroatoms, selected from oxygen, sulphur or nitrogen and may be optionally substituted by C₁-C₈ alkyl or oxo;
R4 is a five or six membered mono or bicyclic ring system having one to three heteroatoms, selected from O, N and S such as pyridyl, pyrimidyl, pyrazinyl, pyridazinyl, isoxazolyl, thiazolyl, thiadiazolyl, tetrazolyl and pyridothiazolyl each of which may optionally be substituted, where appropriate by one or more of the following: hydrogen, halogen, C₁-C₈ alkyl, C₁-C₈ alkoxy, R5 and R6 are independently hydrogen, C₁-C₈ alkyl, or
R5 and R6 may together form a five or six membered cycloalkyl or aryl ring, which may be further substituted with C₁-C₈ alkyl;
R7 and R8 form together cyclobutyl, cyclopentyl, or cyclohexyl;
R9 is C₁-C₈ alkyl;
R9a is independently C₁-C₈ alkyl, C₁-C₈ alkoxy-C₁-C₈ alkyl, C₁-C₈ alkylcarbonyl, arylcarbonyl, heteroarylcarbonyl, carboxy and —COOR13;
R10 is hydrogen, C₁-C₈ alkyl or (C₃-C₈ cycloalkyl)-C₁-C₈ alkyl;
R11 is independently C₁-C₈-alkyl, C₁-C₈ alkoxy, aryl-C₁-C₈ alkyl, heteroaryl-C₁-C₈ alkyl and (C₃-C₈ cycloalkyl)-C₁-C₈ alkyl;
R12 is hydrogen, C₁-C₈ alkoxy or —COOR17;
R13 is hydrogen, C₁-C₈ alkyl, aryl or heteroaryl;
E is a single bond;
R17 is hydrogen;
R23 is hydrogen, C₁-C₈ alkyl, aryl, C₁-C₈ alkoxy, halogen, heteroaryl, heteroaryl-C₁-C₈ alkyl, C₃-C₆ cycloalkyl, or trifluoromethyl, wherein any aryl and heteroaryl residues are optionally substituted with hydrogen, halogen, C₁-C₈ alkyl, C₁-C₈ alkoxy, trifluoromethyl;
R24 is C₁-C₈ alkyl, aryl, heteroaryl, aryl-C₁-C₈ alkyl, heteroaryl-C₁-C₈ alkyl, (C₃-C₈ cycloalkyl)-C₁-C₈ alkyl, and trifluoromethyl, wherein any aryl and heteroaryl residues are optionally mono- or disubstituted with halogen, C₁-C₈ alkyl, C₁-C₈ alkoxy or trifluoromethyl;
R25 is C₁-C₆ alkyl and
R27 is H, aryl, heteroaryl, C₁-C₈ alkyl, C₁-C₈ alkoxy, O-aryl, O-heteroaryl, S-aryl, or NR18R19, wherein R18 and R19 form a five or six membered saturated ring structure optionally containing one to two heteroatoms selected from O, N and S, which may be further substituted with C₁-C₈ alkyl;
R28 and R28a are each independently hydrogen, halogen or C₁-C₈ alkyl;

### R29 is —COOH;

R30 and R30a together form a carbonyl;
R31 is halogen and
R33 is C₁-C₈ alkoxycarbonyl.
Specific examples of the compounds are given in Examples 1-104.

Another object of the present invention is a method for preparation of a compound as above, comprising at least one of the following steps:

a) reaction of a thiophene with a sulphuryl halide to give a thienylsulphuryl halide,
b) reaction of a thienylsulphurylhalide with a primary amine to give a sulphonamide,
c) N-protection of a sulphonamide to give an N-protected sulphonamide,
d) lithiation of a halogenated thiophene to give a lithiated thiophene,
e) coupling of a lithiated thiophene with a halogen substituted alkyl ester of an aromatic carboxylic acid or an aromatic aldehyde to give an arylthienyl ester or aldehyde,
f) reduction of an arylthienyl ester or aldehyde to give an arylthienyl alcohol,
g) conversion of the hydroxy group of an arylthienyl alcohol to an arylthienyl derivative having a leaving group,
h) reaction of an arylthienyl derivative having a leaving group with nucleophile, and
i) deprotection of an N-protected sulphonamide.

Another object of the present invention is a combination comprising a compound as above with at least one of beta blockers, calcium antagonists, diuretics, ACE inhibitors, renin inhibitors, angiotensin II antagonists, vasopeptidase inhibitors, mineralocorticoid receptor antagonists, antihypertensive agents, and antidiabetic agents.

Another object of the present invention is a combination comprising a compound as above with at least one of beta blockers, calcium antagonists, diuretics, ACE inhibitors, renin inhibitors, angiotensin II antagonists, vasopeptidase inhibitors, mineralocorticoid receptor antagonists, antihypertensive agents, antidiabetic agents, fibrinolytic agents, antithrombotic agents, and lipid lowering agents.

Another object is a pharmaceutical composition comprising a compound as above, in admixture with a pharmaceutically adjuvant, diluent or carrier.

In another embodiment, the present invention relates to a pharmaceutical composition comprising a combination as above, in admixture with a pharmaceutically adjuvant, diluent or carrier.

Another object of the present invention is the use of a compound as above for the preparation of a medicament for treating hypertension of different kinds, alleviating organ damage of different kinds, treating or preventing cardiomyopathies of different origins, diabetic vasculopathy and complications thereof, treating endothelin and angiotensin mediated disorders comprising but not limited to vascular inflammatory conditions including atherosclerosis, and treating prostate cancer.

Another object of the present invention is the use of a combination as above for the preparation of a medicament for treating hypertension of different kinds, alleviating organ damage of different kinds, treating or preventing cardiomyopathies of different origins, diabetic vasculopathy and complications thereof, treating endothelin and angiotensin mediated disorders comprising but not limited to vascular inflammatory conditions including atherosclerosis, and treating prostate cancer.

Another object of the present invention is the use of a dual action receptor antagonist at the AT1 and ETA receptors having higher affinity for AT1 than for ETA, for the preparation of a medicament for treating hypertension of different kinds, alleviating organ damage of different kinds, treating or preventing cardiomyopathies of different origins, diabetic vasculopathy and complications thereof, treating endothelin and angiotensin mediated disorders comprising but not limited to vascular inflammatory conditions including atherosclerosis, and treating prostate cancer.

Another object of the present invention is a method for treating hypertension of different kinds, alleviating organ damage of different kinds, treating or preventing cardiomyopathies of different origins, diabetic vasculopathy and complications thereof, treating endothelin and angiotensin mediated disorders comprising but not limited to vascular inflammatory conditions including atherosclerosis, and treating prostate cancer, by administering a compound as above to a mammal in need thereof.

Another object of the present invention is a method for treating hypertension of different kinds, alleviating organ damage of different kinds, treating or preventing cardiomyopathies of different origins, diabetic vasculopathy and complications thereof, treating endothelin and angiotensin mediated disorders comprising but not limited to vascular inflammatory conditions including atherosclerosis, and treating prostate cancer, by administering a combination as above to a mammal in need thereof.

Another object of the present invention is a method for treating hypertension of different kinds, alleviating organ damage of different kinds, treating or preventing cardiomyopathies of different origins, diabetic vasculopathy and complications thereof, treating endothelin and angiotensin mediated disorders comprising but not limited to vascular inflammatory conditions including atherosclerosis, and treating prostate cancer, by administering dual action receptor antagonist at the AT1 and ETA receptors having higher affinity for AT1 than for ETA, to a mammal in need thereof.

### DEFINITIONS

As used herein, the term “C₁-C₈ alkyl” denotes a straight or branched alkyl group having from 1 to 8 carbons. Examples of said C₁-C₈ alkyl include methyl, ethyl, n-propyl, isopropyl, n-butyl, isobutyl, sec-butyl, tert-butyl and straight- and branched-chained pentyl, hexyl, heptyl, and octyl. For parts of the range “C₁-C₈ alkyl”, subranges thereof are contemplated such as C₁-C₇ alkyl, C₁-C₆ alkyl, C₂-C₈ alkyl, C₂-C₇ alkyl, C₂-C₆ alkyl, C₃-C₈ alkyl, C₄-C₆ alkyl, C₅-C₇ alkyl etc.

As used herein, the term “C₁-C₈ alkoxy” denotes a straight or branched alkoxy group having from 1 to 8 carbons. Examples of said C₁-C₈ alkoxy include methoxy, ethoxy, n-propoxy, isopropoxy, n-butoxy, isobutoxy, sec-butoxy, tert-butoxy and straight- and branched-chained pentoxy, hexoxy, heptoxy, and octoxy. For parts of the range “C₁-C₈ alkoxy”, subranges thereof are contemplated such as C₁-C₇ alkoxy, C₁-C₆ alkoxy, C₂-C₈ alkoxy, C₂-C₇ alkoxy, C₂-C₆ alkoxy, C₃-C₅ alkoxy, C₄-C₆ alkoxy, C₅-C₇ alkoxy etc. As used herein, the term “C₂-C₈ alkenyl” denotes a straight or branched alkenyl group having from 2 to 8 carbons. Examples of said C₂-C₈ alkenyl include vinyl, 1-propenyl, 2-propenyl, n-butenyl, isobutenyl, sec-butenyl, and straight- and branched-chained pentenyl, hexenyl, heptenyl, and octenyl. For parts of the range “C₂-C₈ alkenyl”, subranges thereof are contemplated such as C₂-C₇ alkenyl, C₂-C₆ alkenyl, C₃-C₈ alkenyl, C₃-C₇ alkenyl, C₃-C₆ alkenyl, C₃-C₅ alkenyl, C₄-C₆ alkenyl, C₅-C₇ alkenyl etc.

As used herein, the term “C₂-C₈ alkynyl” denotes a straight or branched alkenyl group having from 2 to 8 carbons. Examples of said C₂-C₈ alkynyl include ethynyl, 1-propynyl, 2-propynyl, 1-butynyl, 2-butynyl, 3-butynyl, and straight- and branched-chained pentynyl, hexynyl, heptynyl, and octynyl. For parts of the range “C₂-C₈ alkynyl”, subranges thereof are contemplated such as C₂-C₇ alkynyl, C₂-C₆ alkynyl, C₂-C₈ alkynyl, C₃-C₇ alkynyl, C₃-C₆ alkynyl, C₃-C₅ alkynyl, C₄-C₆ alkynyl, C₅-C₇ alkynyl etc.

As used herein, the term “C₃-C₉ cycloalkyl” denotes a cyclic alkyl group having from 3 to 8 carbons. Examples of said C₃-C₈ cycloalkyl include cyclopropyl, cyclobutyl, cyclopentyl, cyclohexyl, cycloheptyl, and cyclooctyl. For parts of the range “C₃-C₈ cycloalkyl”, subranges thereof are contemplated such as C₃-C₇ cycloalkyl, C₃-C₆ cycloalkyl, C₃-C₅ cycloalkyl, C₄-C₆ cycloalkyl, C₅-C₇ cycloalkyl etc.

As used herein, the term “C₃-C₈ cycloalkoxy” denotes a cyclic alkyl group having from 3 to 8 carbons bonded to an exocyclic oxygen atom. Examples of said C₃-C₈ cycloalkoxy include cyclopropoxy, cyclobutoxy, cyclopentoxy, cyclohexoxy, cycloheptoxy, and cyclooctoxy. For parts of the range “C₃-C₈ cycloalkoxy”, subranges thereof are contemplated such as C₃-C₇ cycloalkoxy, C₃-C₆ cycloalkoxy, C₃-C₅ cycloalkoxy, C₄-C₆ cycloalkoxy, C₅-C₇ cycloalkoxy etc.

As used herein, the term “C₃-C₈ cycloalkenyl” denotes a cyclic alkenyl group having from 3 to 8 carbons. Examples of said C₃-C₈ cycloalkenyl include 1-cyclopropenyl, 2-cyclopropenyl, 1-cyclobutenyl, 1-cyclopentenyl, 1-cyclohexenyl, 1-cycloheptenyl, and 1-cyclooctenyl. For parts of the range “C₃-C₈ cycloalkenyl”, subranges thereof are contemplated such as C₃-C₇ cycloalkenyl, C₃-C₆ cycloalkenyl, C₃-C₅ cycloalkenyl, C₄-C₆ cycloalkenyl, C₅-C₇ cycloalkenyl etc.

As used herein, the term “aryl” denotes mono- or bicyclic aromatic ring systems such as phenyl, naphthyl optionally monosubstituted or disubstituted with groups selected from hydrogen, halogen, C₁-C₈ alkyl, C₁-C₈ alkoxy, cyano, and trifluoromethyl.

As used herein, term “heteroaryl” denotes five or six membered mono- or bicyclic ring systems having one to three heteroatoms selected from O, N and S. Examples of heteroaryl are furyl, thienyl, pyrrolyl, pyridyl, pyrimidyl, pyrazinyl, pyridazinyl, triazinyl, oxazolyl, isoxazolyl, thiazolyl, isothiazolyl, oxadiazolyl, thiadiazolyl, imidazolyl, triazolyl, tetrazolyl and pyridothiazolyl.

As used herein, term “heterocyclyl” denotes five or six membered mono or bicyclic ring saturated or partly saturated systems having one to three heteroatoms selected from O, N and S. Examples of heteroaryl are tetrahydrofuryl, pyrrolidinyl, piperidinyl, piperazinyl, tetrahydrothienyl and imidazolidinyl.

As used herein, the term “halogen” denotes a fluoro, chloro, bromo, or iodo group. The term “perhalo” denotes a group having the highest possible number of halogen atoms bonded thereto.

As used herein, when two or more groups are used in connection with each other, it means that each group is substituted by the immediately preceding group. For instance, C₁-C₈ alkoxycarbonyl means a carbonyl group substituted by a C₁-C₈ alkoxy group. Likewise, heteroaryl-C₁-C₈ alkyl means a C₁-C₈ alkyl group substituted by a heteroaryl group.

As used herein, the term “prevent” or “prevention” is given its ordinary meaning and thus means the avoidance or alleviation of the serious consequences of a disease or a side-effect by early detection.

As used herein, the term “mammal” means a human or an animal such as monkeys, primates, dogs, cats, horses, cows, etc.

As used herein, the single enantiomers, racemic mixtures and unequal mixtures of two enantiomers are within the scope of the invention, where such isomers exist. It should be understood that all the diastereomeric forms possible (pure enantiomers, racemic mixtures and unequal mixtures of two enantiomers), tautomers, and atropisomers are within the scope of the invention.

As used herein, the term “atropisomers” refers to optical isomers that can be separated only because the rotation about single bonds is prevented or greatly slowed down, often referred to in cases of sterically restricted rotation in biaryl systems.

As used herein, the term “polymorphs” pertains to compounds having the same chemical formula, the same salt type and having the same form of hydrate/solvate but having different crystallographic properties.

As used herein, the term “hydrates” pertains to a compound having a number of water molecules bonded to the molecule.

As used herein, the term “solvates” pertains to a compound having a number of solvent molecules bonded to the molecule.

The present invention also encompasses prodrugs of compounds of the invention, i e second compounds which are converted to the first compounds in vivo.

In vivo cleavable esters are just one type of prodrug of the parent molecule. An in vivo hydrolysable (or cleavable) ester of a compound of the present invention that contains a carboxy group is, for example, a pharmaceutically acceptable ester which is hydrolysed in the human or animal body to produce the parent acid. Suitable pharmaceutically acceptable esters for carboxy include C₁-C₈ alkoxymethyl esters, for example, methoxymethyl, C₁-C₈ alkanoloxymethyl ester, for example, pivaloyloxymethyl; phthalidyl esters; C₃-C₈ cycloalkoxycarbonyloxy-C₁-C₈ alkyl esters, for example, 1-cyclohexylcarbonyloxyethyl; 1,3-dioxolen-2-onylmethyl esters, for example, 5-methyl-1,3-dioxolen-2-onylmethyl; and C₁-C₈ alkoxycarbonyloxyethyl esters, for example, 1-methoxycarbonyloxymethyl; and may be formed at any carboxy group in the compounds of the present invention.

As used herein, the term “pharmaceutically acceptable salts” includes acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo or by freeze-drying). Salts may also be prepared by exchanging a counter-ion of a compound of the invention in the form of a salt with another counter-ion using a suitable ion exchange resin.

Suitable acids are non-toxic and include e g, but are not limited to, hydrochloric acid, hydrobromic acid, hydroiodic acid, sulphuric acid, nitric acid, acetic acid, citric acid, asorbic acid, lactic acid, malic acid, and tartaric acid. Suitable bases are non-toxic and include e g, but are not limited to, sodium hydroxide, potassium hydroxide, ammonia, methylamine, dimethylamine, trimethylamine, and triethylamine.

In the context of the present specification, the term “treat” also includes “prophylaxis” unless there are specific indications to the contrary. The term “treat” within the context of the present invention further encompasses to administer an effective amount of a compound of the present invention, to mitigate either a pre-existing disease state, acute or chronic, or a recurring condition. This definition also encompasses prophylactic therapies for prevention of recurring condition and continued therapy for chronic disorders.

The compounds of the present invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the present invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of the present invention, inert, pharmaceutically acceptable carriers can be either solid or liquid. Solid form preparations include powders, tablets, dispersable granules, capsules, cachets, and suppositories.

A solid carrier can be one or more substances, which may also act as diluents, flavouring agents, solubilizers, lubricants, suspending agents, binders, or tablet disintegrating agents; it can also be an encapsulating material.

In powders, the carrier is a finely divided solid, which is in mixture with the finely divided compound of the present invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogenous mixture is then poured into conveniently sized moulds and allowed to cool and solidify.

Suitable carriers are magnesium carbonate, magnesium stearate, talc, lactose, sugar, pectin, dextrin, starch, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavouring agents, stabilizers, and thickening agents as desired. Aqueous solutions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will according to one embodiment of the present invention include 0.05% to 99% weight (percent by weight), according to an alternative embodiment from 0.10 to 50% weight, of the compound of the present invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

The above-mentioned subject-matter for a pharmaceutical composition comprising a compound according to the present invention is applied analogously for a pharmaceutical composition comprising a combination according to the present invention.

In the following, reaction schemes are given to disclose the syntheses of the compounds according to the present invention.

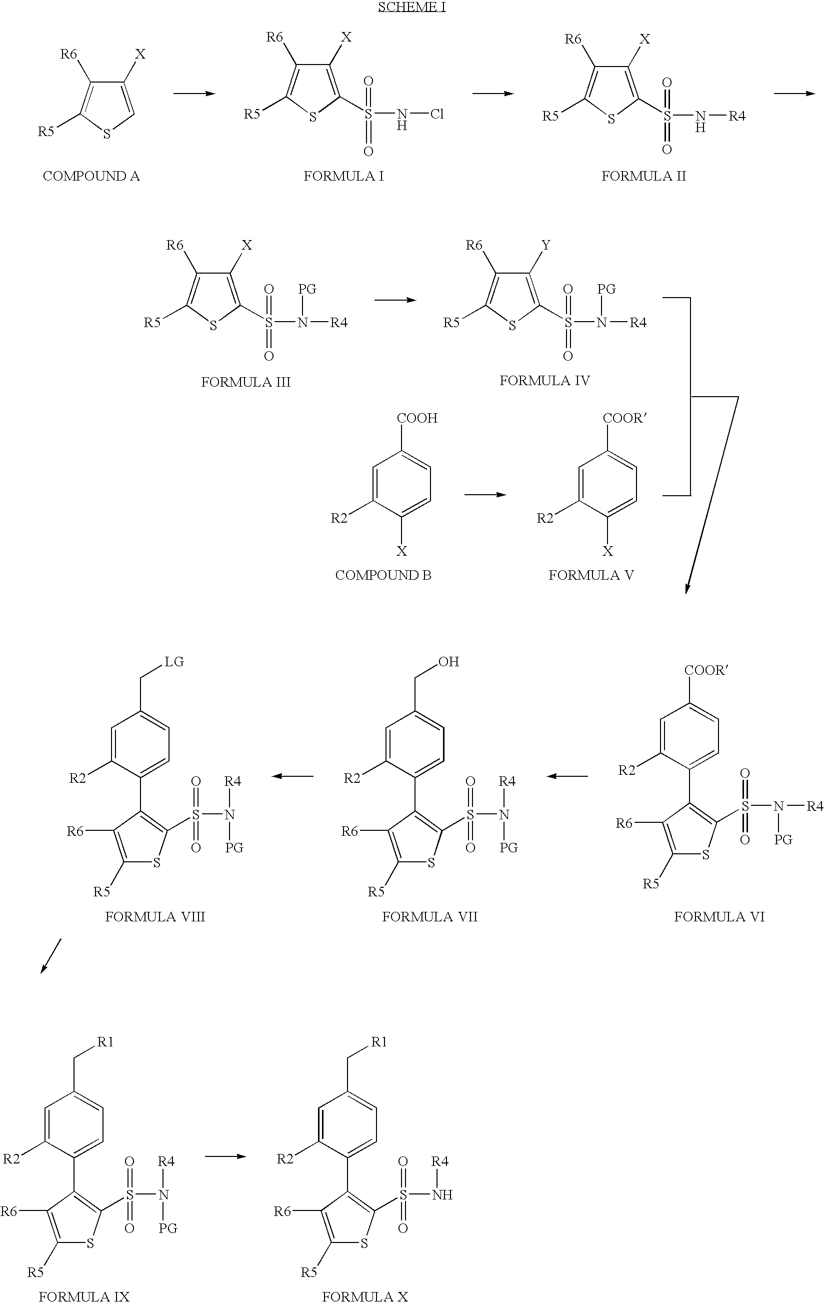

### Description of Scheme I:

Compounds of FORMULA X, wherein R1, R2, R4, R5 and R6 are as previously defined, may be prepared from the deprotection of compound of FORMULA IX wherein PG is a suitable nitrogen protection group. Exemplary conditions for deprotection, and nitrogen protecting groups, may be found in T. W. Greene and P. G. M. Wuts, Protecting Groups in Organic Synthesis, John Wiley and Sons, Inc, New York, 1991, pp. 309-405. Preferred nitrogen protecting groups are the methoxymethyl (MOM), methoxyethoxymethyl (MEM), and 2-(trimethylsilyl)ethoxymethyl (SEM) groups and not limited to for an example a C₁-C₆ alkoxycarbonyl group (such as methoxycarbonyl, ethoxycarbonyl or isobutoxycarbonyl), benzyloxycarbonyl, (in which the benzene ring may be optionally substituted). A protecting group PG may be removed from the FORMULA IX by treatment with one or more deprotecting agents. It will be appreciated that the deprotecting agent or agents will depend on particular protecting group. Suitable deprotecting agents and procedures for their use are well known in the art. For example, an alkoxycarbonyl group may be removed under basic conditions, such as sodium hydroxide or alkoxide (e.g. sodium methoxide) in a suitable solvent such as methanol; a 2-methoxyethoxymethyl group may be removed using acidic conditions, such as hydrochloric acid in a suitable solvent such as ethanol; and a tri C₁-C₄ alkylsilylethoxymethyl group may be removed by using tetrabutylammonium fluoride in tetrahydrofuran, by using trifluoroacetic acid or by using a mixture of hydrochloric acid in a suitable solvent such as ethanol.

Compounds of FORMULA IX may be prepared from compound of FORMULA VIII via displacement of the leaving group (LG) by the conjugate base of a compound R1-H, wherein R1 is as previously defined, using a base in an inert solvent. Exemplary bases include sodium carbonate, potassium carbonate, cesium carbonate, sodium hydride, and potassium hydride or alkyl lithium's. The preferred base is sodium hydride. Exemplary inert solvent include ethers (tetrahydrofuran, 1,4-dioxane, diethyl ether), or N,N-dimethylformamide. The preferred solvent is N,N-dimethylformamide. Exemplary reaction temperatures are between about 0° C. to 120° C., preferably between about 20° C. and 110° C.

Compounds of FORMULA VIII (where LG is a leaving group of type, but not limited to, —OSO₂CH₃, —OSO₂PhCH₃, —OSO₂Ph, —OSO₂CF₃) may be prepared from the reaction of FORMULA VII with for example, but not limited to, ClSO₂CH₃, ClSO₂PhCH₃, ClSO₂Ph, or (CF₃SO₂)₂O in the presence of a base in an inert solvent. Exemplary inert solvent include ethers (tetrahydrofuran, 1,4-dioxane, diethyl ether), or N,N-dimethylformamide.

Compounds of FORMULA VII may be prepared from reduction of a compound of FORMULA VI in an inert solvent using alkali metal hydride such as lithium aluminium hydride.

Compounds of FORMULA VI may be prepared from palladium catalyzed coupling of a compound of FORMULA V with a compound of FORMULA IV, in the presence of suitable base in an inert solvent. Exemplary palladium catalysts include tetrakis (triphenyl phosphine) palladium (0), palladium (II) chloride. The preferred palladium catalyst is tetrakis (triphenyl phosphine) palladium (0). Exemplary bases include tertiary amines, such as, but not limited to, triethylamine, or aqueous potassium, sodium or cesium carbonate. Exemplary solvents include tetrahydrofuran, 1,4-dioxane, acetonitrile, toluene, benzene, or straight chain alcohols, 1,2 dimethoxyethane or a combination thereof. The preferred solvent is a mixture of toluene and ethanol. Exemplary reaction temperature is between about 25° C. to 125° C., Preferably between about 65° C. and 110° C.

Compounds of FORMULA V wherein R′ means lower alkyl groups such as methyl, ethyl, isobutyl and benzyl and X means halogen (chloro, bromo, iodo) may be prepared from COMPOUND B by means known to one skilled in the art.

COMPOUNDs B are either commercially available or available by means known to one skilled in the art.

Compounds of FORMULA IV, where Y is a suitable boron containing substituent may be prepared via lithiation of compounds of FORMULA III where X is hydrogen or a halogen (chloro, bromo, iodo), and reacting the resulting heteroaryllithium with an appropriate borate derivative. The appropriate borate compounds are either commercially available or available by means known to one skilled in the art.

Compounds of FORMULA III may be prepared via the protection of nitrogen in a compound of FORMULA II. Exemplary nitrogen protecting groups and methods of protecting the nitrogen are similar to those for protecting amines, such as those described in T. W. Greene and P. G. M. Wuts, Protecting Groups in Organic Synthesis, John Wiley and Sons, Inc, New York, 1991.

Compounds of FORMULA II may be prepared from the reaction of a compound of FORMULA I with a compound R4-NH₂.

Compounds of FORMULA I are either commercially available or available by means known to one skilled in the art via Compound A.

Compounds A are either commercially available or available by means known to one skilled in the art.

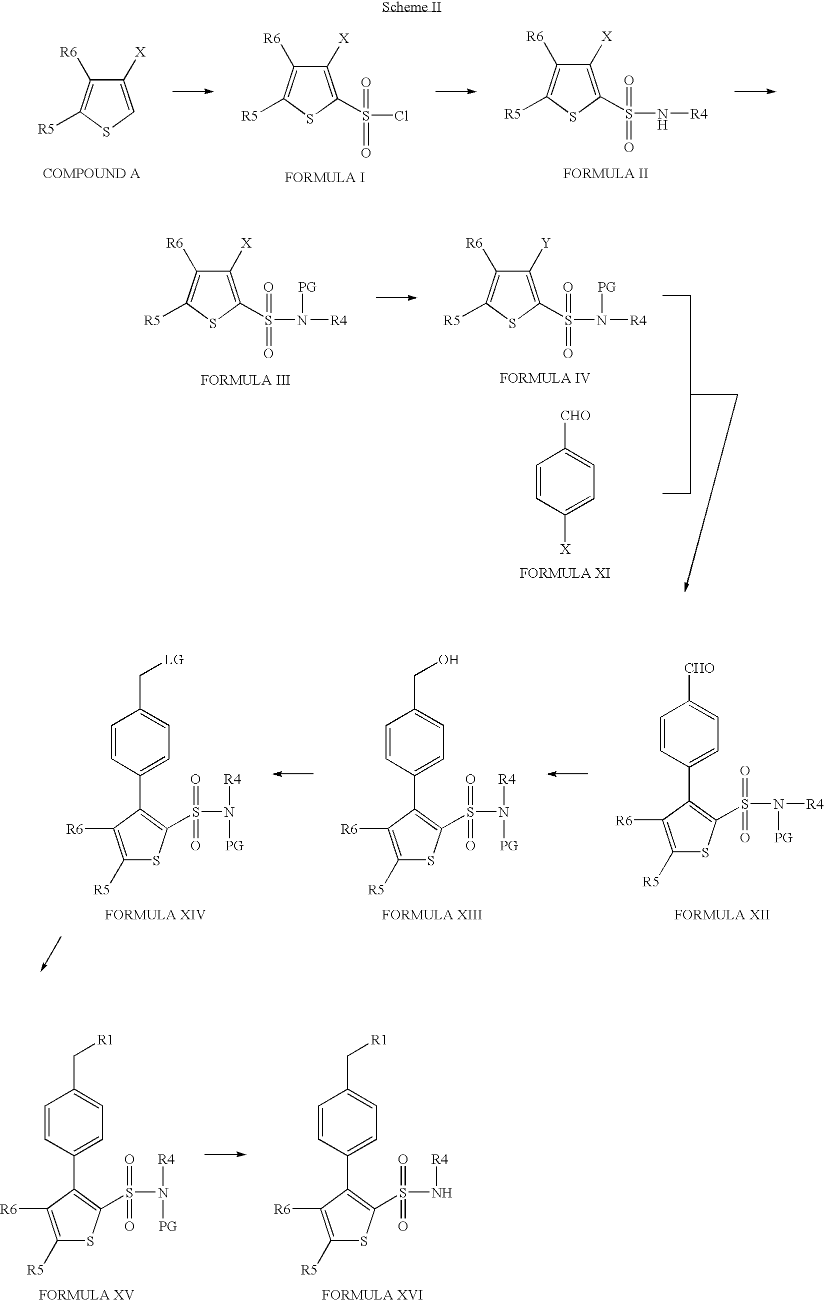

### Description of Scheme II:

Compounds of FORMULA XVI, wherein R1, R4, R5 and R6 are as previously defined, may be prepared from the deprotection of compound of FORMULA XV wherein PG is a suitable nitrogen protection group. A protecting group PG may be removed from the FORMULA XV by treatment with one or more deprotecting agents. It will be appreciated that the deprotecting agent or agents will depend on particular protecting group. Suitable deprotecting agents and procedures for their use are well known in the art.

Compounds of FORMULA XV may be prepared from compound of FORMULA XIV via displacement of the leaving group (LG) by the conjugate base of a compound R1-H, wherein R1 is as previously defined, using a base in an inert solvent. Exemplary bases include sodium carbonate, potassium carbonate, cesium carbonate, sodium hydride, and potassium hydride or alkyl lithium's. The preferred base is sodium hydride. Exemplary inert solvent include ethers (tetrahydrofuran, 1,4-dioxane, diethyl ether), or N,N-dimethylformamide. The preferred solvent is N,N-dimethylformamide. Exemplary reaction temperatures are between about 0° C. to 120° C., preferably between about 20° C. and 110° C.

Compound of FORMULA XIV (where LG is a leaving group of type, but not limited to, —OSO₂CH₃, —OSO₂PhCH₃, —OSO₂Ph, —OSO₂CF₃) may be prepared from the reaction of FORMULA XIII with for example, but not limited to, ClSO₂CH₃, ClSO₂PhCH₃, ClSO₂Ph, or (CF₃SO₂)₂O in the presence of a base in an inert solvent. Exemplary inert solvent includes ethers (tetrahydrofuran, 1,4-dioxane, diethyl ether), or N,N-dimethylformamide.

Compound of FORMULA XIII may be prepared from reduction of a compound of FORMULA XII in an inert solvent by using reducing agents like lithium aluminium hydride, sodium borohydride and sodium cyanoborohydride.

Compounds of FORMULA XII may be prepared from palladium catalyzed coupling of a compound of FORMULA XI with a compound of FORMULA IV, in the presence of suitable base in an inert solvent. Exemplary palladium catalysts include tetrakis (triphenyl phosphine) palladium (0), palladium (II) chloride. The preferred palladium catalyst is tetrakis (triphenyl phosphine) palladium (0). Exemplary bases include tertiary amines, such as, but not limited to, triethylamine, or aqueous potassium, sodium or cesium carbonate. Exemplary solvents include tetrahydrofuran, 1,4-dioxane, acetonitrile, toluene, benzene, or straight chain alcohols, 1,2 dimethoxyethane or a combination thereof. The preferred solvent is a mixture of toluene and ethanol. Exemplary reaction temperature is between about 25° C. to 125° C., preferably between about 65° C. and 110° C.

Compounds of FORMULA XI are either commercially available or available by means known to one skilled in the art.

Compounds of FORMULA IV may be prepared as described in SCHEME I.

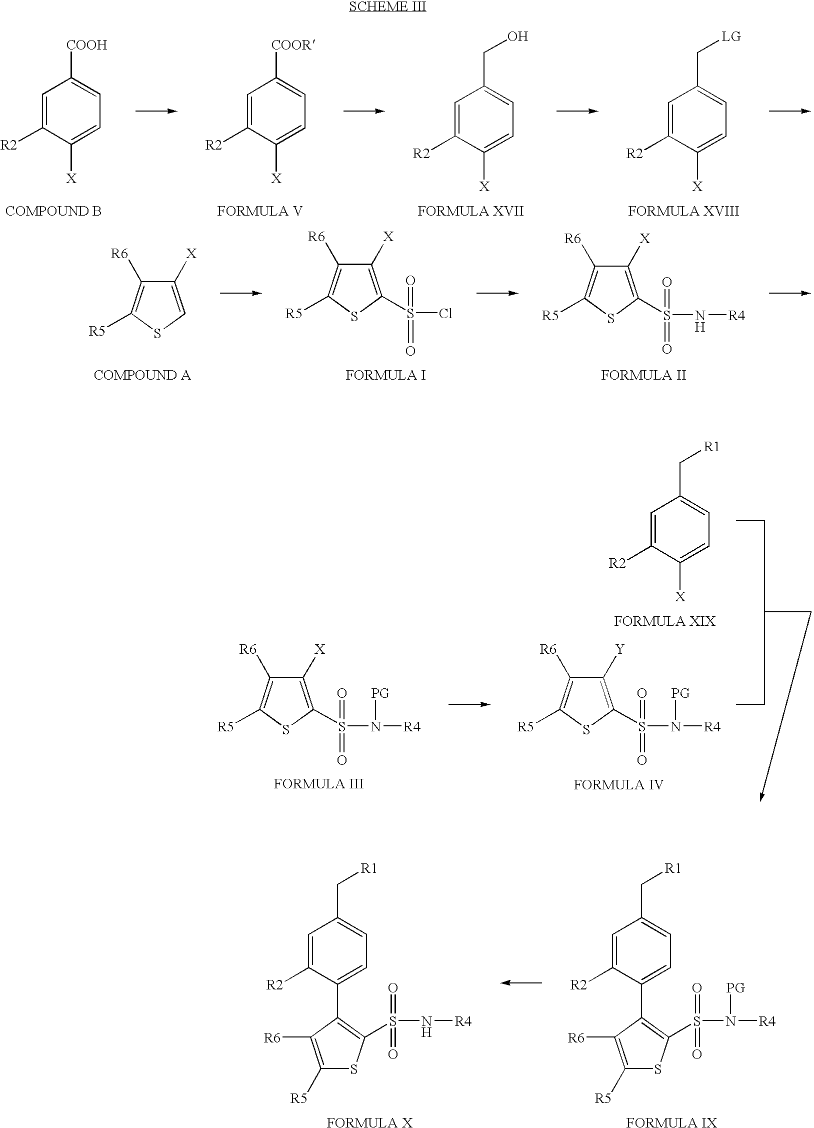

### Description of Scheme III

Compounds of FORMULA X, wherein R1, R2, R4, R5 and R6 are as previously defined, may be prepared from the deprotection of compound of FORMULA IX as described in SCHEME I.

Compounds of FORMULA IX may be prepared from palladium catalyzed coupling of a compound of FORMULA XIX with a compound of FORMULA IV, In the presence of suitable base in an inert solvent. Exemplary palladium catalysts include tetrakis (triphenyl phosphine) palladium (0), palladium (II) chloride. The preferred palladium catalyst is tetrakis (triphenyl phosphine) palladium (0). Exemplary bases include tertiary amines, such as, but not limited to, triethylamine, or aqueous potassium, sodium or cesium carbonate. Exemplary solvents include tetrahydrofuran, 1,4-dioxane, acetonitrile, toluene, benzene, or straight chain alcohols, 1,2 dimethoxyethane or a combination thereof. The preferred solvent is a mixture of toluene and ethanol. Exemplary reaction temperature is between about 25° C. to 125° C., preferably between about 65° C. and 110° C.

Compounds of FORMULA IV may be prepared as described in SCHEME I.

Compounds of FORMULA XIX may be prepared via displacement of the leaving group (LG) of the compound of FORMULA XVIII by the conjugate base of a compound R1-H, wherein R1 is as previously defined, using a base in an inert solvent. Exemplary bases include sodium carbonate, potassium carbonate, cesium carbonate, sodium hydride, and potassium hydride or alkyl lithium's. The preferred base is sodium hydride. Exemplary inert solvent include ethers (tetrahydrofuran, 1,4-dioxane, diethyl ether), or N,N-dimethylformamide. The preferred solvent is N,N-dimethylformamide. Exemplary reaction temperatures are between about 0° C. to 120° C., preferably between about 20° C. and 110° C.

Compounds of FORMULA XVIII (where LG is a leaving group of type, but not limited to, —OSO₂CH₃, —OSO₂PhCH₃, —OSO₂Ph, —OSO₂CF₃) may be prepared from the reaction of FORMULA XVII with for example, but not limited to, ClSO₂CH₃, ClSO₂PhCH₃, ClSO₂Ph, or (CF₃SO₂)₂O in the presence of a base in an inert solvent. Exemplary inert solvent includes ethers (tetrahydrofuran, 1,4-dioxane, diethyl ether), or N,N-dimethylformamide.

Compounds of FORMULA XVII may be prepared from reduction of a compound of FORMULA V in an inert solvent by using reducing agents like lithium aluminium hydride, sodium borohydride and sodium cyanoborohydride

Compounds of FORMULA V may be prepared as described in SCHEME I.

By changing the substitution pattern of FORMULA I the alternative isomer, i.e. FORMULA B, can also be used as starting point in SCHEME I, II OR III, resulting in the isomeric form of FORMULA X, and FORMULA XVI.

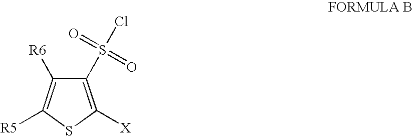

The heterocyclic rings as mentioned in this application, the preparation of which has not been explicitly disclosed, may be prepared analogously to the heterocyclic rings, the preparation of which does have been explicitly disclosed.

In the following, the present invention is illuminated by the following non-limiting Examples.

When used, the expressions “comprise” and “comprising” denote “include” and “including” but not limited to. Thus, other ingredients, carriers and additives may be present.

### EXAMPLES

### Abbreviations

ACE—angiotensin converting enzyme
Ang II—angiotensin II
AT1—angiotensin II receptor 1
AT2—angiotensin II receptor 2
ETA—endothelin receptor A
ETB—endothelin receptor B
LAH—lithium aluminium hydride
rt or RT—room temperature
t—triplet
s—singlet
d—doublet
q—quartet
qvint—quintet
m—multiplet
br—broad
bs—broad singlet
dm—doublet of multiplet
bt—broad triplet
dd—doublet of doublet

### General Experimental Procedures

Mass spectra were recorded on a Thermo Finnigan LC-MS system (LCQ classic). ¹H NMR measurements were recorded on a Bruker Avance DPX 400 MHz. Chemical shifts are given in ppm with TMS as internal standard.

### Example 1

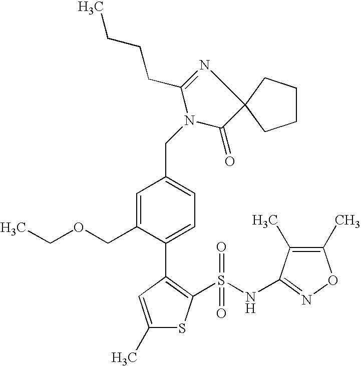

### 3-[4-(2-Butyl-4-oxo-1,3-diaza-spiro[4.4] non-1-en-3-ylmethyl)-2-ethoxymethyl-phenyl]-5-methyl-thiophene-2-sulphonic acid (4,5-dimethyl-isoxazol-3-yl)-amide

### STEP 01: Synthesis of 4-Bromo-3-methyl benzoic acid ethyl ester

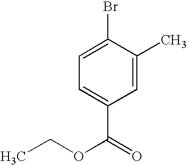

To the cooled (0° C.) solution of 4 bromo-3 methyl benzoic acid (25 gm, 0.116 mol) in (100 ml) ethanol was added (8 ml) of concentrated sulphuric acid with stirring. After the completion of the addition, the reaction mixture was refluxed for 6 hrs. The reaction mixture was cooled and then concentrated under vacuum. To the residue was added (50 ml) of cold water, followed by extraction with diethyl ether (100 ml×2). The organic layer was washed with saturated sodium bicarbonate solution, followed by water and brine solution washings. Finally the ether layer was dried over sodium sulphate and evaporated under vacuum to give 26 gm of 4-bromo-3 methyl benzoic acid ethyl ester.

### STEP 02: Synthesis of ethyl 4-bromo-3-(bromo methyl)benzoate

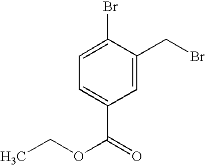

To the solution of 4-bromo-3 methyl benzoic acid ethyl ester (25 gm, 0.102 mol) in (100 ml) carbon tetrachloride at room temperature was added N-bromosuccinimide (20.13 gm, 0.113 mol) and (1.24 gm, 0.005 mol)benzoyl peroxide. Then the reaction mixture was refluxed for 10 hrs. The reaction mixture was cooled and filtered. The filtrate was concentrated under vacuum. The residue thus obtained was purified by triturating it with (100 ml) hexane. Solid thus obtained was filtered and suction dried to give 12 gm of ethyl 4-bromo-3-(bromo methyl)benzoate.

### STEP 03: Synthesis of 4-bromo-3-ethoxymethyl-benzoic acid ethyl ester

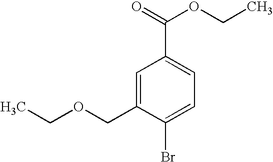

To the cooled (0° C.) solution of ethyl 4-bromo-3-(bromo methyl)benzoate (12 gm, 0.037 mol) in ethanol (25 ml) was added sodium ethoxide (5.0 gm, 0.074 mol) and (4 ml) of N,N-dimethyl formamide. The reaction mixture was stirred for 4 hrs at room temperature. Then the reaction mixture was concentrated under vacuum and residue was diluted with ethyl acetate (100 ml). The ethyl acetate layer was washed with water and brine solution and finally the organic layer was dried over sodium sulphate. The organic layer was evaporated under vacuum to give 10.0 gm of 4-bromo-3-ethoxymethyl-benzoic acid ethyl ester.

### STEP 04: Synthesis of (5-methyl-isoxazol-3-yl) carbamic acid tert-butyl ester

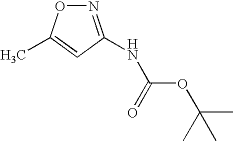

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 05: Synthesis of (4,5-dimethyl-isoxazol-3-yl) carbamic acid tert-butyl ester

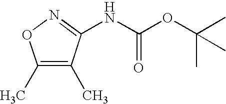

Under the dry nitrogen atmosphere (5-methyl-isoxazol-3-yl) carbamic acid tert-butyl ester (20 gm, 0.10 nmol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 06: Synthesis of 4,5-dimethyl-isoxazol-3-yl-amine

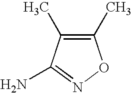

(4,5-dimethyl-isoxazol-3-yl) carbamic acid tert-butyl ester (22 gm, 0.1036 mol) was portion wise added to the trifluoroacetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 07: Synthesis of 5-methyl-thiophene-2-sulphonic acid (4,5-dimethyl-isoxazol-3yl) amide

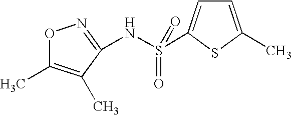

The solution of 5-Methyl thiophene-2-sulphonyl chloride (10.5 gm, 0.053 mol) in (15 ml) methylene chloride was added to the solution of 3-amino-4,5-dimethylisoxazole (4 gm, 0.035 mol) and dimethylaminopyridine (500 mg) in pyridine (20 ml) at 0° C. After the completion of the addition the temperature of the reaction mixture was slowly raised to room temperature and stirred for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 4.0 gm of 5-methyl-thiophene-2-sulphonic acid (4,5-dimethyl-isoxazol-3yl) amide as brown colored solid.

### STEP 08: Synthesis of 5-methyl-thiophene-2-sulphonic acid (4,5 dimethyl-isoxazol-3-yl)-(2-methoxy-ethoxymethyl)-amide

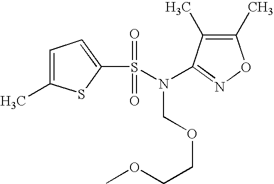

At 0° C., under stirring, sodium hydride (0.800 gm, 0.166 mol, 60% dispersion in mineral oil) was added in to N,N-dimethyl formamide (30 ml), followed by addition of 5-methyl-thiophene-2-sulphonic acid (4,5-dimethyl-isoxazol-3yl) amide (4 gm, 0.0146 mol) to it. After completion of the addition, temperature of the reaction mixture was slowly raised and stirred it at ambient temperature for 30 minutes then recooled the reaction mixture to 0° C., followed by the drop wise addition of methoxyethoxymethyl chloride (2.7 gm, 0.021 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 3.7 gm of 5-methyl-thiophene-2-sulphonic acid (4,5 dimethyl-isoxazol-3-yl)-(2-methoxy-ethoxymethyl)-amide as yellowish oil.

### STEP 09: Synthesis of 3-borono-N-(4,5-dimethyl-3-isoxazolyl)-N-[(2-methoxy-ethoxy) methyl]-5-methyl-thiophene sulphonamide

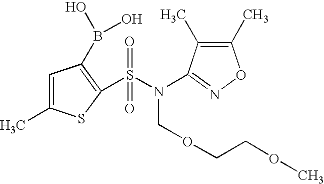

Under the dry nitrogen atmosphere the solution of (3.7 gm, 0.010 mol) 5-methyl-thiophene-2-sulphonic acid (4,5 dimethyl-isoxazol-3-yl)-(2-methoxy-ethoxymethyl)-amide in tetrahydrofuran (30 ml) was cooled to −78° C. To this n-Butyl lithium (11 ml, 0.025 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (3 ml, 0.015 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×2). The combined extract was washed with water and brine solution. Ethyl acetate layer was dried over sodium sulphate and concentrated under vacuum to give 3.4 gm of 3-borono-N-(4,5-dimethyl-3-isoxazolyl)-N-[(2-methoxy-ethoxy)methyl]-5-methyl-thiophene sulphonamide as thick oily mass.

### STEP 10: Synthesis of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester

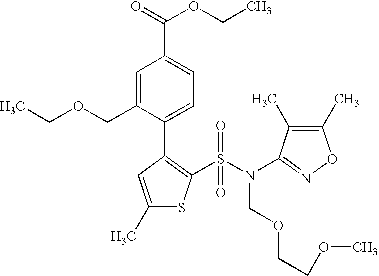

To a stirred solution of 3-borono-N-(4,5-dimethyl-3-isoxazolyl)-N-[(2-methoxy-ethoxy)methyl]-5-methyl-thiophene sulphonamide (5.2 gm, 0.0128 mol) and 4-bromo-3-ethoxymethyl-benzoic acid ethyl ester (3.7 gm, 0.0128 mol) in toluene (50 ml) and ethanol (25 ml) under nitrogen was added 2M aqueous sodium carbonate (4.0 gm in 19 ml water). The reaction mixture was stirred under nitrogen atmosphere for 15 minutes and then tetrakis triphenyl phosphine palladium (0) (0.745 gm, 0.64 mmol) was added into the reaction mixture. The reaction mixture was heated to 85° C. for 6 hrs. The reaction mixture was concentrated and ethyl acetate (25 ml) was added to the residue followed by chilled water and extraction with ethyl acetate (100 ml×2). The combine extracts were washed with water and brine and dried over sodium sulphate and concentrated completely under vacuum. The crude compound was purified by column chromatography on a silica gel column using hexane:ethyl acetate as an eluent to provide 0.300 gm of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester as an oily mass.

### STEP 11: Synthesis of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

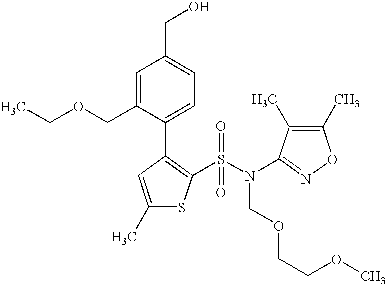

Lithium aluminium hydride (0.100 gm) was added to a stirred solution of tetrahydrofuran (10 ml) at 0° C. under flow of dry nitrogen, followed by addition of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester (0.300 gm) in (15 ml) of tetrahydrofuran. The reaction mixture was stirred at 0° C. for 1 hr and then the temperature was raised to room temperature and the mixture stirred for 4 hrs. The excess lithium aluminium hydride was destroyed by addition of sodium hydroxide solution (1 gm dissolved in 100 ml water) at 0° C. followed by extraction with ethyl acetate (25 ml×2). The organic layer was dried over sodium sulphate and concentrated completely under vacuum to give 0.240 gm of 3-(2-ethoxymethyl-4-hydroxy methyl-phenyl)-5-methyl-thiophene-2-sulphonic acid-(4,5-Dimethyl-isoxazol-3-yl)-2-methoxy-ethoxymethyl) amide

### STEP 12: Synthesis of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester

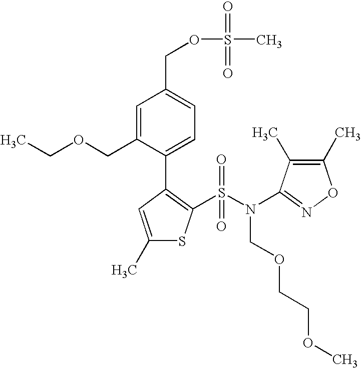

N-Ethyl diisopropyl amine (0.2 ml, 0.0011 mol) was added to a solution of 3-(4-hydroxymethyl-phenyl)-5-methyl-thiophene-2-sulphonic acid (4,5-dimethyl-isoxazol-3-yl)-(2-methoxy-ethoxymethyl)-amide (0.240 gm, 0.00045 mol) in 10 ml of dichloro methane. The reaction mixture was cooled to 0° C., where after slowly methane sulphonyl chloride (0.043 ml, 0.00055 mol) was added into the reaction mixture. The reaction mixture was maintained at room temperature for 3 hrs and was then dumped into ice-cold water followed by extraction with methylene chloride (25 ml×2). The combined extracts were washed with dilute hydrochloric acid followed by water and brine solution and the organic layer was dried over sodium sulphate and concentrated to give 0.220 gm of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester.

### STEP 13: Synthesis of 3-[4-(2-butyl-4-oxo-1,3-diaza-spiro[4.4]non-1-en-3-yl-methyl)-2-ethoxy methyl-phenyl]-5-methyl-thiophene-2-sulphonic acid (4,5-dimethyl-isoxazol-3-yl)-(2-methoxy-ethoxymethyl)-amide

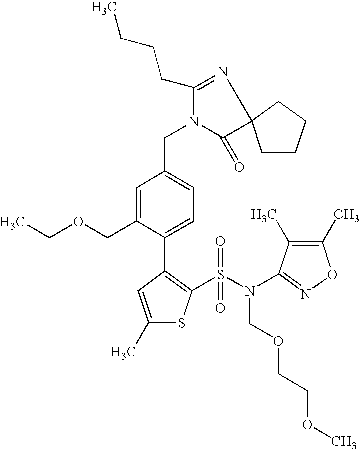

To the stirred solution of 2-butyl-1,3 diaza-spiro[4.4]non-1-en-4-one (0.080 gm, 0.41 mmol) in dimethyl formamide (2 ml) at −15° C. under nitrogen was added portion wise sodium hydride (60% in mineral oil) (0.025 gm, 0.54 mmol). After the addition, the reaction mixture was warmed to ambient temperature and maintained for 30 min. The reaction mixture was cooled to 0° C. and a solution of methane sulphonic acid 4-{2-[(4,5-dimethyl-isoxazol-3-yl)-(2-methoxy-ethoxymethyl)-sulphamoyl]-5-methylthiophene-3-yl}-3-ethoxy methyl-benzyl ester (0.220 gm, 0.36 mmol) in 2 ml dimethyl formamide was added drop wise to the reaction mixture and stirred at room temperature for 24 hrs. The mixture was then diluted with ethyl acetate (20 ml), followed by 5 ml of cold water. The organic layer was washed with water and brine then dried over sodium sulphate and evaporated under vacuum. The residue was purified by column chromatography using silica gel column using hexane/ethyl acetate as an eluent to provide 0.230 gm of 3-[4-(2-butyl-4-oxo-1,3-diaza-spiro[4.4]non-1-en-3-ylmethyl)-2-ethoxymethyl-phenyl]-5-methyl-thiophene-2-sulphonic acid (4,5-dimethyl-isoxazol-3-yl)-(2-methoxy-ethoxymethyl)-amide as a viscous oily mass.

### STEP 14: Synthesis of 1-amino-cyclopentanecarboxylic acid ethyl ester

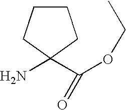

To a stirred solution of 1-amino-cyclopentanecarboxylic acid (25 gm, 0.193 mol) in ethanol (250 ml) at 0° C., 15 ml of concentrated sulphuric acid was added. The reaction mixture was refluxed for 24 hrs and then cooled and evaporated under vacuum. The residue was neutralized with addition of solid sodium bicarbonate followed by addition of 50 ml of cold water. The mixture was extracted with dichloro methane (200 ml×4). The organic layer was washed with water, brine and dried over sodium sulphate and evaporated to give 27 gm of 1-amino-cyclopentane carboxylic acid ethyl ester.

### STEP 15: Synthesis of 2-butyl-1,3-diaza-spiro[4.4] non-1-en-4-one

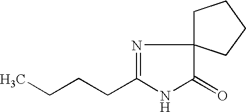

To a stirred solution of 1-amino-cyclopentanecarboxylic acid ethyl ester (7 gm, 0.0445 mol) in 30 ml of toluene added pentanimidic acid ethyl ester (7 gm, 0.054 mol), followed by a catalytic amount of acetic acid (1-2 ml) and the reaction was refluxed for 48 hr. The reaction mixture was cooled and concentrated under vacuum, and the residue was dissolved into ethyl acetate (50 ml) and then washed with water and brine, dried over sodium sulphate and evaporated to give 5 gm 2-butyl-1,3-diaza-spiro[4.4]non-1-en-4-one as a pale yellow oil.

### STEP 16: 3-[4-(2-butyl-4-oxo-1,3-diaza-spiro[4.4]non-1-en-3ylmethyl)-2-ethoxy methyl-phenyl]-5-methyl-thiophene-2-sulphonic acid (4,5-dimethyl-isoxazol-3-yl)-amide

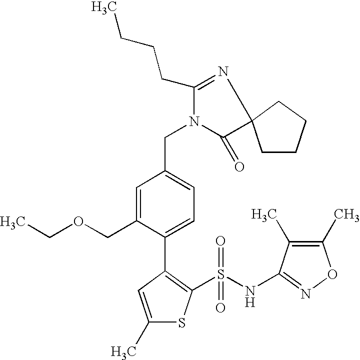

To 3-[4-(2-butyl-4-oxo-1,3-diaza-spiro[4.4]non-1-en-3ylmethyl)-2-ethoxy methyl-phenyl]-5-methyl-thiophene-2-sulphonic acid (4,5-dimethyl-isoxazol-3-yl)-(2-methoxy-ethoxymethyl)-amide (0.230 gm, 0.32 mmol) was added 95% ethanol (5 ml) and 6N aqueous hydrochloric acid (4 ml) at room temperature. The reaction mixture was refluxed for 3 hrs. The reaction mixture was concentrated under vacuum and the pH of the solution was adjusted to 8 using a saturated solution of sodium bicarbonate. The reaction solution was then acidified to pH 5 with acetic acid, and the mixture was extracted with ethyl acetate (25 ml×2). The combined organic extract were washed with water and brine and then dried over sodium sulphate and concentrated under vacuum. The crude product was purified by silica gel flash column chromatography using hexane:ethyl acetate as an eluent to provide 50 mg of 3-[4-(2-butyl-4-oxo-1,3-diaza-spiro[4.4]non-1-en-3ylmethyl)-2-ethoxymethyl-phenyl]-5-methyl-thiophene-2-sulphonic acid (4,5-dimethyl-isoxazol-3-yl)-amide.

Molecular Formula: C₃₁H₄₀N₄O₅S₂

Molecular Weight: 612.80

¹HNMR (DMSOd₆): 0.83 (t, J=7.2 Hz, 3H) 1.06 (t, J=7.2 Hz, 3H) 1.28-1.33 (m, 2H) 1.51-1.54 (m, 2H) 1.56 (s, 3H) 1.66-1.71 (m, 2H) 1.84-1.87 (m, 6H) 2.20 (s, 3H) 2.32-2.38 (m, 2H) 2.48 (s, 3H) 3.23-3.27 (m, 2H) 4.07-4.11 (m, 2H) 4.72-4.74 (m, 2H) 6.74 (s, 1H) 6.97-7.03 (m, 2H) 7.17-7.19 (m 1H) 10.75 (s, 1H)

Mass Spectrum: (m⁺¹) 613

### Example 2

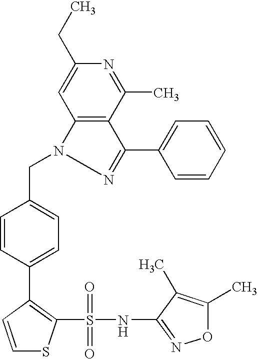

### 3-[4-(6-Ethyl-4-methyl-3-phenyl-pyrazolo[4,3-c]pyridin-1-ylmethyl)-phenyl]-thiophene-2-sulphonic acid (4,5-dimethyl-isoxazol-3-yl) amide

### STEP 01: Synthesis of 1-phenyl-butane-1,3 dione

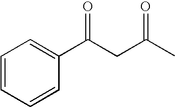

Sodium ethoxide (13.5 gm, 0.198 mol) was added to a stirred solution of dry ethyl acetate (80 ml, 0.72 mol) at −5° C. To this reaction mixture was added acetophenone (20 gm, 0.185 mol) at −5° C. and then the temperature of the reaction mixture was maintained at 0° C. for 12 hrs. The reaction mixture was then acidified with 1N hydrochloric acid and extracted with ethyl acetate (100 ml×2). The combined organic layer was washed with the water and brine. The organic layer was dried over sodium sulphate and evaporated to give 21 gm of yellow colored solid of 1-phenyl-butane-1,3 dione.

### STEP 02: Synthesis of 3-amino-1-phenyl-but-2-en-1-one

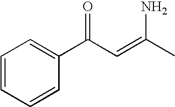

The mixture of 1-phenyl-butane-1,3 dione (20 gm, 0.123 mol) and ammonium acetate (38 gm, 0.49 mol) in dry methanol (200 ml) was stirred and heated to reflux for 24 hrs. The reaction mixture was concentrated under vacuum and to the residue was added chilled water, followed by the extraction with ethyl acetate (100 ml×2). The combined extracts were washed with water and brine. The organic layer was dried over sodium sulphate and evaporated to give 19 gm of yellow colored solid of 3-amino-1-phenyl-but-2-en-1-one.

### STEP 03: Synthesis of 5-(1-hydroxy propylidine)2,2-dimethyl-1,3-dioxane-4,6-dione

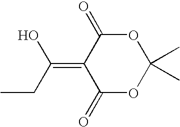

Propionyl chloride (7 ml, 0.0763 mol) was added to a solution of Meldrum's acid (10 gm, 0.069 mol) in pyridine (12 ml, 0.138 mol) and methylene chloride (50 ml) at 0° C. within 30 min. and the temperature of the reaction mixture was allowed to rise to ambient temperature and stirred for 1 hr. The reaction mixture was then acidified using 1N hydrochloric acid and extracted with methylene chloride (50 ml×2). The combined extracts were washed with water and brine. The organic layer was dried over sodium sulphate and concentrated under vacuum to give 10 gm of 5-(1-hydroxy propylidine) 2,2-dimethyl-1,3-dioxane-4,6-dione as a crystalline solid.

### STEP 04: Synthesis of 3-benzoyl-6-ethyl-2-methyl-1H-Pyridin-4-one

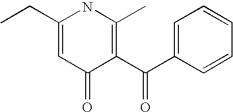

A mixture of 5-(1-hydroxy propylidine) 2,2-dimethyl-1,3-dioxane-4,6-dione (17.39 gm, 0.087 mol) and 3-amino-1-phenyl-but-2-en-1-one (10 gm, 0.062 mol) was stirred and heated at 120° C. for 2 hrs. Then the reaction mixture was purified by column chromatography over silica gel, eluting the desired fraction with 10% methanol and ethyl acetate to give 5.8 gm of 3-Benzoyl-6-ethyl-2-methyl-1H-Pyridin-4-one as yellow colored solid.

### STEP 05: Synthesis of (4-chloro-6-ethyl-2-methyl-pyridin-3-yl) phenyl-methanone

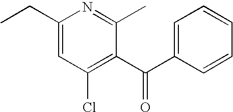

3-Benzoyl-6-ethyl-2-methyl-1H-Pyridin-4-one (2.7 gm, 0.011 mol) was added to phosphorous oxy chloride (8 ml) at 0° C. Then the reaction mixture was stirred and heated to 50° C. and the temperature of the reaction mixture was maintained for 8 hours. The work-up was done by evaporating the phosphorus oxy chloride under vacuum and the residue thus obtained was basified to pH 8 with saturated sodium bicarbonate solution, followed by extraction with methylene dichloride (50 ml×2). The combined organic extracts were washed with water and brine. The organic layer was dried over anhydrous sodium sulphate and concentrated to give 2.6 gm of (4-chloro-6-ethyl-2-methyl-pyridin-3-yl)phenyl-methanone as yellow oil.

### STEP 06: Synthesis of 6-ethyl-4-methyl-3-phenyl-1H-pyrazolo[4,3-c]pyridine

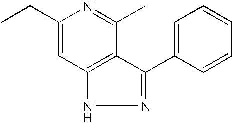

(4-chloro-6-ethyl-2-methyl-pyridin-3-yl)phenyl methanone (2.5 gm 0.0096 mol) was dissolved in ethanol (10 ml) and hydrazine hydrate (2.3 ml, 0.048 mol) was added to the reaction mixture. The reaction mixture was stirred and heated to reflux for 4 hours. Then the reaction mixture was evaporated under vacuum. To the residual mass was added ice water, the solid thus obtained was filtered and suction dried to provide 1.8 gm of 6-ethyl-4-methyl-3-phenyl-1H-pyrazolo[4,3-c]pyridine.

### STEP 07: Synthesis of (5-methyl-isoxazol-3-yl) carbamic acid tert-butyl ester

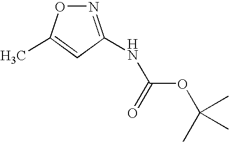

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 08: Synthesis of (4,5-dimethyl-isoxazol-3-yl) carbamic acid tert-butyl ester

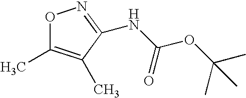

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 09: Synthesis of 4,5-dimethyl-isoxazol-3-yl-amine

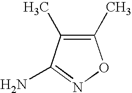

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 10: Synthesis of 3-bromo-thiophene-2-sulphonyl chloride

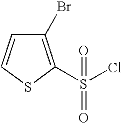

3-Bromothiophene (10 gm, 0.0617 mol) was taken in methylene chloride (60 ml) and cooled this reaction mixture to −78° C. Then chlorosulphonic acid (25 ml, 0.396 mol) was added drop wise to the reaction mixture at −78° C. The temperature of the reaction mixture was slowly raised to 0° C. and maintained for 1 hour. The reaction mixture was slowly poured into the ice cold water, followed by extraction with methylene chloride (100 ml×3). The combined organic extract was washed with water and brine and then dried over anhydrous sodium sulphate and evaporated under vacuum to give a brown color solid. The crude compound was purified by column chromatography on a silica gel column using hexane/ethyl acetate as an eluent to provide 5.4 gm of 3-bromo-thiophene-2-sulphonyl chloride.

### STEP 11: Synthesis of 3-bromo-thiophene-2-sulphonic acid (4,5-dimethyl-isoxazol-3yl) amide

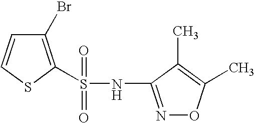

To the solution of 3-amino-4,5-dimethylisoxazole (2 gm, 0.0178 mol) in (25 ml) pyridine and dimethyl amino pyridine (0.230 gm, 0.0019 mol) was added 3-bromo-thiophene-2-sulphonyl chloride (5.0 gm, 0.01912) at 0° C. Then slowly the temperature of reaction mixture was raised to room temperature (28° C.), and then the reaction mixture was stirred for 6 hours. The reaction mixture was then concentrated under vacuum, the residue was acidified using 1N hydrochloric acid to pH 1 followed by extraction with dichloro methane (50 ml×3). The combined organic extract was washed with water and brine. Organic layer was dried over sodium sulphate and concentrated to give 3.5 gm of 3-bromo-thiophene-2-sulphonic acid (4,5-dimethyl-isoxazol-3yl) amide as brown colored solid.

### STEP 12: Synthesis of 3-bromo-thiophene-2-sulphonic acid (4,5 dimethyl-isoxazol-3-yl)-(2-methoxy-ethoxymethyl)-amide

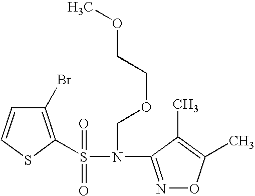

Under the flow of dry nitrogen, sodium hydride (0.600 gm, 0.0125 mol, 60% in mineral oil) was added portion wise to the solution of 3-bromo-thiophene-2-sulphonic acid (4,5-dimethyl-isoxazol-3yl) amide (3.5 gm, 0.0103 mol) in N,N-dimethyl formamide (30 ml) at −15° C. After completion of the addition reaction mixture was stirred at room temperature for 30 min. Then the reaction mixture was recooled to 0° C. To this reaction mixture 2-methoxyethoxy methylchloride (1.55 gm, 0.0124 mol) was added drop wise within 30 min, maintaining the temperature of the reaction mixture at 0° C. The reaction mixture was stirred at 0° C. for 30 min and then at room temperature for 4 hrs. Then the reaction mixture was diluted with ethyl acetate (100 ml) followed by addition of (30 ml) ice cold water. The organic layer was separated, washed with water and brine. Finally the organic layer was dried over sodium sulphate and concentrated under vacuum. The crude compound was purified by column chromatography on a silica gel column using hexane:ethyl acetate as eluent to provide 2.7 gm of 3-bromo-thiophene-2-sulphonic acid (4,5 dimethyl-isoxazol-3-yl)-(2-methoxy-ethoxymethyl)-amide as yellow oil.

### STEP 13: Synthesis of 3-(4-formyl-phenyl)-thiophene-2-sulphonic acid (4,5-dimethyl-isoxazol-3-yl)-(2-methoxy-ethoxymethyl) amide

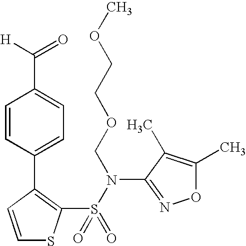

To a stirred solution 2.7 gm (0.0063 mol) of 3-bromo-thiophene-2-sulphonic acid (4,5 dimethyl-isoxazol-3-yl)-(2-methoxy-ethoxymethyl)-amide and 4-formyl boronic acid 1.04 gm (0.006 mol) in toluene (15 ml) and ethanol (12 ml) under nitrogen atmosphere was added 2M aqueous sodium carbonate solution (2 gm in 16 ml water). This reaction mixture was then stirred for 15 minutes, whereupon tetrakis triphenyl phosphine palladium (0) was added into the reaction mixture. The reaction mixture was heated to 85° C. for 6 hrs. The reaction mixture was cooled to room temperature and ethyl acetate (50 ml) was added, followed by evaporation under vacuum. To the residual mass, ethyl acetate (100 ml) was added, followed by chilled water and the mixture was further extracted with ethyl acetate (100 ml×2). The combined extracts were washed with water and brine and dried over sodium sulphate and concentrated under vacuum. The crude compound was purified on a silica gel column using ethyl acetate:hexane as an eluent to provide 1.1 gm of 3-(4-formyl-phenyl)-thiophene-2-sulphonic acid (4,5-dimethyl-isoxazol-3-yl)-(2-methoxy-ethoxymethyl) amide as oil.

### STEP 14: Synthesis of 3-(4-hydroxymethyl-phenyl)-thiophene-2-sulphonic acid (4,5-dimethyl-isoxazol-3-yl)-(2-methoxy-ethoxymethyl)-amide

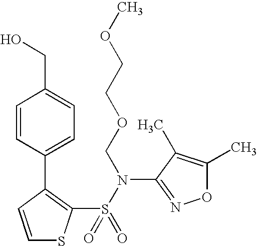

Lithium aluminum hydride (0.100 gm, 0.0029 mol) was added under flow of nitrogen to a stirred solution of tetrahydrofuran at 0° C., followed by addition of 3-(4-formyl-phenyl)-thiophene-2-sulphonic acid (4,5-dimethyl-isoxazol-3-yl)-(2-methoxy-ethoxymethyl) amide (1.1 gm, 0.0024 mol) in (15 ml) tetrahydrofuran. The reaction mixture was stirred at 0° C. for 1 hr and then the temperature was raised to room temperature (28° C.) and stirred for 4 hrs. The reaction was worked up by addition of sodium hydroxide solution (1 gm dissolved in 100 ml water) into the reaction mixture at 0° C. followed by extraction with ethyl acetate (50 ml×2). The combined organic layers were washed with water and brine. The organic layer was dried over sodium sulphate and concentrated under vacuum to give 1.0 gm of 3-(4-hydroxymethyl-phenyl)-thiophene-2-sulphonic acid (4,5-dimethyl-isoxazol-3-yl)-(2-methoxy-ethoxymethyl)-amide as an oil.

### STEP 15: Synthesis of 3-(4-methanesulphonyl methyl-phenyl)-thiophene-2-sulphonic acid (4,5-dimethyl-isoxazol-3-yl)-(2-methoxy-ethoxymethyl)amide

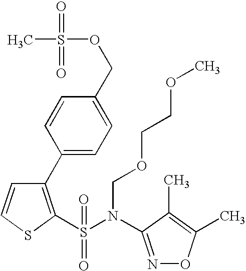

To the 0° C. cooled solution of 3-(4-hydroxymethyl-phenyl)-thiophene-2-sulphonic acid (4,5-dimethyl-isoxazol-3-yl)-(2-methoxy-ethoxymethyl)-amide (1.0 gm, 0.0022 mol) in (60 ml) of dichloro methane, N-Ethyl diisopropyl amine (0.6 ml, 0.0033 mol) was added, followed by slow addition of the solution of methane sulphonyl chloride (0.2 ml, 0.0033 mol) in (10 ml) dichloromethane in to the reaction mixture. The reaction mixture was then warmed and stirred at room temperature for 3 hrs. Workup was done by addition of ice-cold water into the reaction mixture followed by extraction with methylene dichloride (25 ml×2). The combined organic extract was washed with dilute hydrochloric acid followed by washings with water and brine. The organic layer was dried over sodium sulphate and concentrated under vacuum. The crude compound was purified by column chromatography on a silica gel column using hexane/ethyl acetate as an eluent to provide 0.700 gm of 3-(4-methanesulphonyl methyl-phenyl)-thiophene-2-sulphonic acid (4,5-dimethyl-isoxazol-3-yl)-(2-methoxy-ethoxymethyl) amide as a viscous liquid.

### STEP 16: Synthesis of 3-[4-(6-ethyl-4-methyl-3-phenyl-pyrazolo [4,3-c] pyridin-1-yl methyl)-phenyl]-thiophene-2-sulphonic acid (4,5-dimethyl-isoxazol-3-yl)-(2-methoxy-ethoxymethyl)-amide

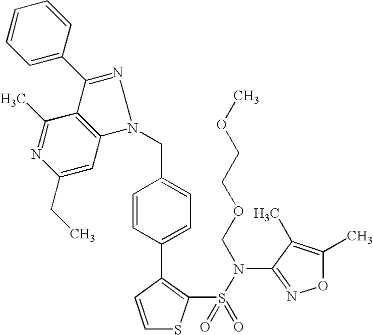

To the stirred solution of 6-ethyl-4-methyl-3-phenyl-1H-pyrazolo[4,3-c]pyridine (0.344 gm, 0.00145 mol) in dimethyl formamide (5 ml) at 0° C. under the flow of dry nitrogen gas, was added portion wise sodium hydride (60% in mineral oil) (0.100 gm, 0.00174 mol). After the addition, the temperature of the reaction mixture was raised to room temperature and maintained for 30 min. The reaction mixture was then re-cooled to 0° C. and a solution of 3-(4-methanesulphonyl methyl-phenyl)-thiophene-2-sulphonic acid (4,5-dimethyl-isoxazol-3-yl)-(2-methoxy-ethoxymethyl) amide (0.700 gm, 0.00145 mol) in dimethyl formamide (5 ml) was added drop wise and the mixture stirred at room temperature for 24 hrs. The mixture was then diluted with ethyl acetate (40 ml) followed by 10 ml of water at 0° C. and extracted with ethyl acetate (50 ml×2) and the combined organic extract was washed with water and brine. Then the organic layer was dried over sodium sulphate and concentrated under vacuum to give 1.0 gm of 3-[4-(6-ethyl-4-methyl-3-phenyl-pyrazolo[4,3-c]pyridin-1-yl methyl)-phenyl]-thiophene-2-sulphonic acid (4,5-dimethyl-isoxazol-3yl-(2-methoxy-ethoxymethyl)-amide as a gum.

### STEP 17: Synthesis of 3-[4-(6-ethyl-4-methyl-3-phenyl-pyrazolo [4,3-c]pyridin-1-ylmethyl)-phenyl]-thiophene-2-sulphonic acid (4,5-dimethyl-isoxazol-3-yl) amide

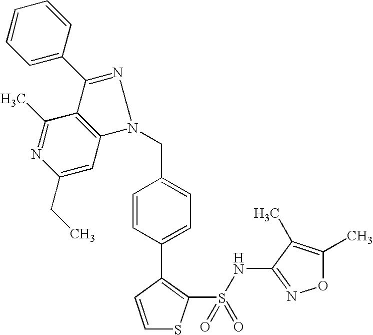

To (1.0 gm, 1.48 mmol) of 3-[4-(6-ethyl-4-methyl-3-phenyl-pyrazolo[4,3-c]pyridin-1-yl methyl)-phenyl]-thiophene-2-sulphonic acid(4,5-dimethyl-isoxazol-3yl-(2-methoxy-ethoxymethyl)-amide was added 95% ethanol (10 ml) and 5 ml of 6N aqueous hydrochloric acid at room temperature. This reaction mixture was then refluxed for 2 hrs. The reaction mixture was cooled and concentrated under vacuum. The residue thus obtained was diluted with water and the pH of the solution was adjusted to pH 5 using sodium bicarbonate solution. The solution was extracted with ethyl acetate (50 ml×2) and the combined organic extract was washed with water and brine then dried over sodium sulphate and concentrated under vacuum. The crude compound was purified on a silica gel column using ethyl acetate:hexane as an eluent to provide 200 mg of amorphous yellowish foam.

Molecular Formula: C₃₁H₂₉N₅O₃S₂

Molecular Weight: 583.72

¹HNMR (DMSOd₆): 1.29 (t, J=7.2 Hz, 3H) 1.44 (s, 3H) 2.08 (s, 3H) 2.47 (s, 3H) 2.80-2.86 (m, 2H) 5.72 (s, 2H) 7.11-7.13 (m, 1H) 7.28-7.31 (m, 2H) 7.40-7.43 (m, 2H) 7.51-7.56 (m, 4H) 7.65-7.68 (m, 2H) 7.90-7.92 (m, 1H) 10.80 (bs, 1H)

Mass Spectrum: (m⁻¹) 582

### Example 3

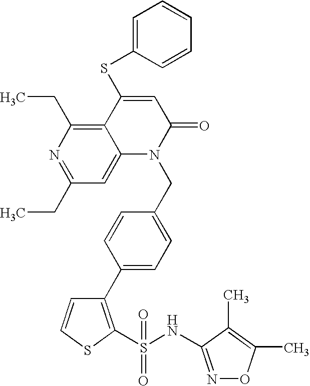

### 3-[4-(5,7-diethyl-2-oxo-4-phenylsulphanyl-2H-[1,6]naphthyridin-1 ylmethyl)-phenyl]-thiophene-2-sulphonic acid(4,5-dimethyl-isoxazol-3-yl)-amide

### STEP 01: Synthesis of methyl 3-amino pentenoate

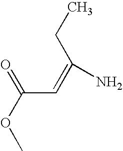

A mixture of methyl propionyl acetate (50 gm, 0.3842 mol) and ammonium acetate (148 gm, 1.921 mol) in dry methanol (500 ml) was stirred and refluxed for 2 days. The reaction mixture was cooled and concentrated under vacuum. The residue thus obtained was basified to pH 8 and extracted with ethyl acetate. The ethyl acetate layer was washed with water, brine and the organic layer was dried over sodium sulphate and concentrated to give 50 gm of methyl 3-amino pentenoate as pale yellow liquid.

### STEP 02: Synthesis of 2,6-Diethyl-4-oxo-1,4-dihydro-pyridine-3-carboxylic acid methyl ester

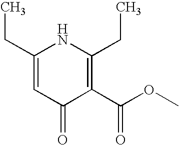

To the mixture of methyl 3-amino pentenoate (50 gm, 0.387 mol) and methyl propionyl acetate (50 gm, 0.384 mol) in o-xylene (200 ml) was added of 40 A molecular sieves (50 gm). Then this reaction mixture was stirred and heated to reflux with a Dean Stark apparatus for 5 days. The reaction mixture was cooled to room temperature and the molecular sieves were filtered off. The filtrate was concentrated and the crude compound was purified by column chromatography on a silica gel column using 50% dichloromethane: methanol as an eluent to provide 20 gm of the desired product as an off white solid

### STEP 03: Synthesis of 2,6-diethyl-4-(toluene-4-sulphonylamino) nicotinic acid methyl ester

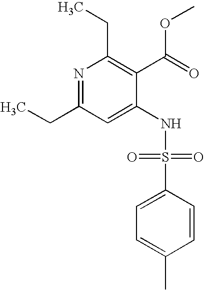

Under the flow of dry nitrogen gas tosyl isocyanate (39 gm, 0.197 mol) was added to a stirred suspension of methyl 2,6-diethyl-4-oxo-1,4-dihydropyridine-3-carboxylate (25 gm, 0.119 mol) in acetonitrile (250 ml). After the initial exotherm had subsided the mixture was refluxed for 2 hours. The reaction mixture was cooled to room temperature and the suspended solid product was collected by filtration to give 20 gm of 2,6-diethyl-4-(toluene-4-sulphonylamino) nicotinic acid methyl ester.

### STEP 04: Synthesis of 4-amino-2,6-diethyl-nicotinic acid methyl ester

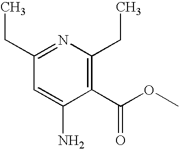

2,6-Diethyl-4-(toluene-4-sulphonylamino)nicotinic acid methyl ester (40 g, 0.110 mol) was added to concentrated sulphuric acid (57 ml, 1.10 mol) at 0° C. and then the reaction mixture was stirred at 50° C. for 1 hour. The reaction mixture was cooled to room temperature and poured onto ice. The pH of this solution was adjusted to 8 by solid sodium carbonate and extracted with dichloro methane (200 ml×2). The combined organic layers were washed with water and brine. The organic layer was dried over sodium sulphate and concentrated to yield 19 gm methyl-4-amino-2,6-diethyl pyridine-3-carboxylate as a white solid

### STEP 05: Synthesis of 5,7-diethyl-4-hydroxy-2-oxo-1,2-dihydro-[1,6]naphthyridine-3-carboxylic acid ethyl ester

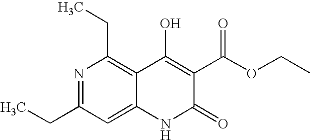

Diethylmalonate (15 ml, 0.093 mol) and methyl-amino-2,6-diethylpyridine-3-carboxylate (19.0 gm, 0.09 mol) were added to a solution of sodium ethoxide (7 gm, 0.10 mol) in ethanol (60 ml) and this reaction mixture was heated at 150° C. and 100 psi pressure for 20 hours in an autoclave. The reaction mixture was allowed to cool and the volatile material was removed by evaporation and the resulting semi solid was triturated with ether to give a white solid, which was collected by filtration and dissolved in water. This solution was then acidified with 1 N hydrochloric acid to give a white solid which was filtered and suction dried to yield 11 gm of ethyl 5,7-diethyl-4-hydroxy-2-oxo-1,2-dihydro-1,6-naphthyridine-3-carboxylate as off white solid.

### STEP 06: Synthesis of 5,7-diethyl-4-hydroxy-1H-[1,6]naphthyridin-2-one

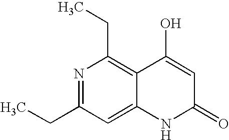

Ethyl 5,7-diethyl-4-hydroxyl-2-oxo-1,2-dihydro-1,6-naphthyridine-3-carboxylate (11 gm) was dissolved in a mixture of water (11 ml), 1,4-dioxane (22 ml) and concentrated hydrochloric acid (11 ml) and the reaction mixture was heated to reflux for 3 hours. The reaction mixture was then cooled and the suspended solid was filtered off, washed with ethanol and ether and suction dried to give 4.3 gm of 5,7-diethyl 4-hydroxy-1,6-naphthyridin-2(1H)-one as an off white solid.

### STEP 07: Synthesis of 4-chloro-5,7-diethyl-1,6-naphthyridin-2(1H)-one

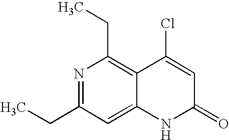

(4.3 gm, 0.019 mol) of 5,7-diethyl-4-hydroxy 1,6-naphthyridin-2(1H)-one was dissolved in (22 ml) phosphorous oxy chloride and the reaction mixture was refluxed for 24 hours. The reaction mixture was concentrated and the residue was dissolved in concentrated hydrochloric acid (16 ml) and 22 ml of water and refluxed for 4 hours. The reaction mixture was diluted with water and basified with solid sodium bicarbonate. The resulting solid was collected by filtration, washed with water and suction dried to give 3.0 gm of 4-chloro-5,7-diethyl-1,6-naphthyridin-2(1H)-one as an orange coloured solid.

### STEP 08: Synthesis of (5-methyl-isoxazol-3-yl) carbamic acid tert-butyl ester

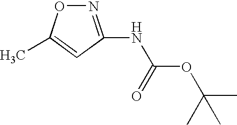

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 09: Synthesis of (4,5-dimethyl-isoxazol-3-yl) carbamic acid tert-butyl ester

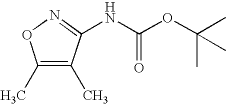

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 10: Synthesis of 4,5-dimethyl-isoxazol-3-yl-amine

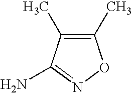

(4,5-dimethyl-isoxazol-3-yl) carbamic acid tert-butyl ester (22 gm, 0.1036 mol) was portion wise added to the trifluoroacetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 11: Synthesis of 3-bromo-thiophene-2-sulphonyl chloride

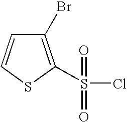

3-Bromothiophene (10 gm, 0.0617 mol) was dissolved in methylene chloride (60 ml) and the reaction mixture was cooled to −78° C. Then chlorosulphonic acid (25 ml, 0.396 mol) was added drop wise at −78° C. The temperature of the reaction mixture was slowly raised to 0° C. and maintained for 1 hour. The reaction mixture was slowly poured into the ice cold water, followed by extraction with methylene chloride (100 ml×3). The combined organic extract was washed with water and brine then dried over anhydrous sodium sulphate, evaporated under vacuum to give a brown color solid. The crude compound was purified on a silica gel column using hexane:ethyl acetate as an eluent to provide 5.4 gm of 3-bromo-thiophene-2-sulphonyl chloride.

### STEP 12: Synthesis of 3-bromo-thiophene-2-sulphonic acid (4,5-dimethyl-isoxazol-3yl) amide

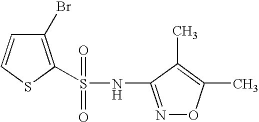

To the solution of 3-amino-4,5-dimethylisoxazole (2 gm, 0.0178 mol) in (25 ml) pyridine and dimethyl amino pyridine (0.230 gm, 0.0019 mol) was added 3-bromo-thiophene-2-sulphonyl chloride (5.0 gm, 0.01912) at 0° C. Then slowly the temperature of reaction mixture was raised to room temperature (28° C.), and then the reaction mixture was stirred for 6 hours. The reaction mixture was then concentrated under vacuum, the residue was acidified using 1N hydrochloric acid to pH 1 followed by extraction with dichloro methane (50 ml×3). The combined organic extract was washed with water and brine. Organic layer was dried over sodium sulphate and concentrated to give 3.5 gm of 3-bromo-thiophene-2-sulphonic acid (4,5-dimethyl-isoxazol-3yl) amide as brown colored solid.

### STEP 13: Synthesis of 3-bromo-thiophene-2-sulphonic acid (4,5 dimethyl-isoxazol-3-yl)-(2-methoxy-ethoxymethyl)-amide

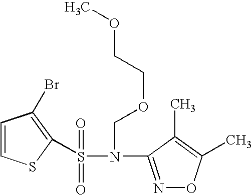

Under the flow of dry nitrogen, sodium hydride (0.600 gm, 0.0125 mol, 60% in mineral oil) was added portion wise to the solution of 3-bromo-thiophene-2-sulphonic acid (4,5-dimethyl-isoxazol-3-yl) amide (3.5 gm, 0.0103 mol) in N,N-dimethyl formamide (30 ml) at −15° C. After completion of the addition reaction mixture was stirred at room temperature for 30 min. Then the reaction mixture was recooled to 0° C. To this reaction mixture 2-methoxyethoxy methylchloride (1.55 gm, 0.0124 mol) was added drop wise within 30 min, maintaining the temperature of the reaction mixture at 0° C. The reaction mixture was stirred at 0° C. for 30 min and then at room temperature for 4 hrs. Then the reaction mixture was diluted with ethyl acetate (100 ml) followed by addition of (30 ml) ice cold water. The organic layer was separated, washed with water and brine. Finally the organic layer was dried over sodium sulphate and concentrated under vacuum. The crude compound was purified by column chromatography on a silica gel column using hexane:ethyl acetate as eluent to provide 2.7 gm of 3-bromo-thiophene-2-sulphonic acid (4,5 dimethyl-isoxazol-3-yl)-(2-methoxy-ethoxymethyl)-amide as yellow oil.

### STEP 14: Synthesis of 3-(4-formyl-phenyl)-thiophene-2-sulphonic acid (4,5-dimethyl-isoxazol-3-yl)-(2-methoxy-ethoxymethyl) amide

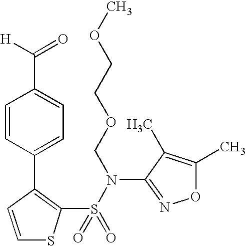

To a stirred solution 2.7 gm (0.0063 mol) of 3-bromo-thiophene-2-sulphonic acid (4,5 dimethyl-isoxazol-3-yl)-(2-methoxy-ethoxymethyl)-amide and 4-formyl boronic acid 1.04 gm (0.006 mol) in toluene (15 ml) and ethanol (12 ml) under nitrogen atmosphere was added 2M aqueous sodium carbonate solution (2 gm in 16 ml water). The reaction mixture was stirred for 15 minutes, and then tetrakis triphenyl phosphine palladium (0) was added. The reaction mixture was then heated to 85° C. for 6 hrs. The reaction mixture was cooled to room temperature and then ethyl acetate (50 ml) was added. The reaction mixture was concentrated under vacuum and to the residual mass ethyl acetate (100 ml) was added, followed by chilled water and further extraction with ethyl acetate (100 ml×2). The combined extract was washed with water and brine and dried over sodium sulphate and concentrated under vacuum. The crude compound was purified on a silica gel column using hexane:ethyl acetate as an eluent to provide 1.1 gm of 3-(4-formyl-phenyl)-thiophene-2-sulphonic acid (4,5-dimethyl-isoxazol-3-yl)-(2-methoxy-ethoxymethyl) amide as oil.

### STEP 15: Synthesis of 3-(4-hydroxymethyl-phenyl)-thiophene-2-sulphonic acid (4,5-dimethyl-isoxazol-3-yl)-(2-methoxy-ethoxymethyl)-amide

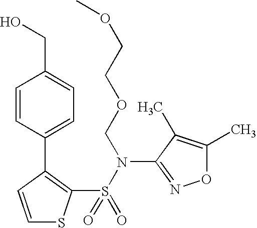

Lithium aluminum hydride (0.100 gm, 0.0029 mol) was added under flow of nitrogen to a stirred solution of tetrahydrofuran (15 ml) at 0° C., followed by addition of 3-(4-formyl-phenyl)-thiophene-2-sulphonic acid (4,5-dimethyl-isoxazol-3-yl)-(2-methoxy-ethoxymethyl) amide (1.1 gm, 0.0024 mol) in (15 ml) tetrahydrofuran. The reaction mixture was stirred at 0° C. for 1 hr and the temperature was then raised to room temperature (28° C.) and stirred for 4 hrs. The reaction was worked up by addition of sodium hydroxide solution (1 gm dissolved in 100 ml water) into the reaction mixture at 0° C. followed by extraction with ethyl acetate (50 ml×2). The combined organic layers were washed with water and brine and the organic layer was dried over sodium sulphate and concentrated under vacuum to give 1.0 gm of 3-(4-hydroxymethyl-phenyl)-thiophene-2-sulphonic acid (4,5-dimethyl-isoxazol-3-yl)-(2-methoxy-ethoxymethyl)-amide as an oil.

### STEP 16: Synthesis of 3-(4-methanesulphonyl methyl-phenyl)-thiophene-2-sulphonic acid (4,5-dimethyl-isoxazol-3-yl)-(2-methoxy-ethoxymethyl)amide

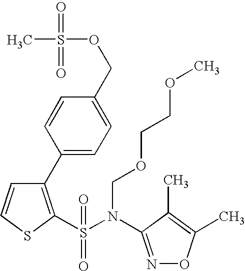

To the 0° C. cooled solution of 3-(4-hydroxymethyl-phenyl)-thiophene-2-sulphonic acid (4,5-dimethyl-isoxazol-3-yl)-(2-methoxy-ethoxymethyl)-amide (1.0 gm, 0.0022 mol) in (60 ml) of dichloro methane, N-Ethyl diisopropyl amine (0.6 ml, 0.0033 mol) was added, followed by slow addition of the solution of methane sulphonyl chloride (0.2 ml, 0.0033 mol) in (10 ml) dichloromethane in to the reaction mixture. The reaction mixture was then warmed and stirred at room temperature for 3 hrs. Workup was done by addition of ice-cold water into the reaction mixture followed by extraction with methylene dichloride (25 ml×2). The combined organic extract was washed with dilute hydrochloric acid followed by washings with water and brine. The organic layer was dried over sodium sulphate and concentrated under vacuum. The crude compound was purified by column chromatography on a silica gel column using hexane/ethyl acetate as an eluent to provide 0.700 gm of 3-(4-methanesulphonyl methyl-phenyl)-thiophene-2-sulphonic acid (4,5-dimethyl-isoxazol-3-yl)-(2-methoxy-ethoxymethyl) amide as a viscous liquid.

### STEP 17: Synthesis of 3-[4-(4-chloro-5,7-diethyl-2-oxo-2H-[1,6]naphthyridin-1ylmethyl)-phenyl]-thiophene-2-sulphonic acid(4,5-dimethyl-isoxazol-3-yl)-(2-methoxy-ethoxymethyl)-amide

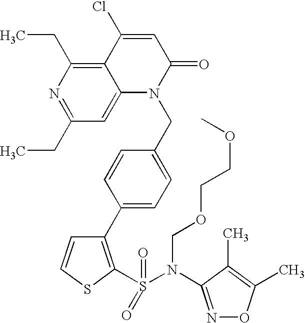

4-chloro-5,7-diethyl-1,6-naphthyridine-2-(1H)-one (1 gm, 4.22 mmol) was charged to a suspension of sodium hydride (0.242 gm, 5.04 mmol, 50%) in (10 ml) N,N dimethyl formamide at 0° C. under nitrogen atmosphere and the mixture was stirred for 30 minutes at room temperature. The reaction mixture was recooled to 0° C. and to this a solution of 3-(4-methanesulphonyl methyl-phenyl)-thiophene-2-sulphonic acid (4,5-dimethyl-isoxazol-3-yl)-(2-methoxy-ethoxymethyl) amide (2.4 gm, 4.65 mmol) in (10 ml) N,N dimethyl formamide was added drop wise. The reaction mixture was then stirred at room temperature for 20 hours. The reaction mixture was diluted with 40 ml ethyl acetate followed by 10 ml cold water, the organic phase was separated and the aqueous layer again extracted with 20 ml of ethyl acetate. The combined organic layer was washed with water and brine solution, dried over sodium sulphate and concentrated. The crude compound was purified by column chromatography on a silica gel column using hexane:ethyl acetate as an eluent to provide 300 mg of 3-[4-(4-chloro-5,7-diethyl-2-oxo-2H-[1,6]naphthyridin-1ylmethyl)-phenyl]-thiophene-2-sulphonic acid(4,5-dimethyl-isoxazol-3-yl)-(2-methoxy-ethoxymethyl)-amide.

### STEP 18: Synthesis of 3-[4-(5,7-diethyl-2-oxo-4-phenylsulphanyl-2H-[1,6]naphthyridin-1ylmethyl)-phenyl]-thiophene-2-sulphonic acid(4,5-dimethyl-isoxazol-3-yl)-(2-methoxy-ethoxymethyl)-amide

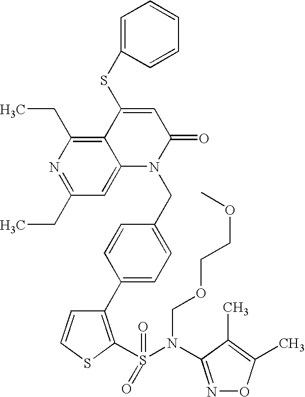

Sodium hydride (25 mg, 0.83 mmol 50%) was added to a solution of thiophenol (0.04 ml, 0.44 mmol) in 2 ml DMF and the mixture was stirred until the effervescence ceased. The solid mass of 3-[4-(4-Chloro-5,7-diethyl-2-oxo-2H-[1,6] naphthyridin-1ylmethyl)-phenyl]-thiophene-2-sulphonic acid (4,5-dimethyl-isoxazol-3-yl)-(2-methoxy-ethoxymethyl)-amide (300 mg, 0.44 mmol, spot-2) was then added portion wise. The reaction mixture was stirred and heated at 50° C. for 2 hours and was then poured into water and stirred for 30 min. mixture was then acidified with dilute hydrochloric acid to pH 1 and extracted with ethyl acetate. The ethyl acetate layer was washed with water and brine solution. Then the organic layer was dried over sodium sulphate and concentrated to give 300 mg of 3-[4-(5,7-diethyl-2-oxo-4-phenyl sulphanyl-2H-[1,6] naphthyridin-1ylmethyl)-phenyl]-thiophene-2-sulphonic acid (4,5-dimethyl-isoxazol-3-yl)-(2-methoxy-ethoxymethyl)-amide as viscous mass.

### STEP 19: Synthesis of 3-[4-(5,7-diethyl-2-oxo-4-phenylsulphanyl-2H-[1,6]naphthyridin-1ylmethyl)-phenyl]-thiophene-2-sulphonic acid(4,5-dimethyl-isoxazol-3-yl)-amide

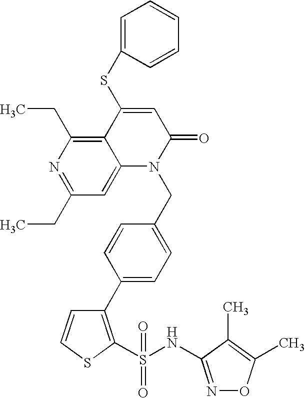

3-[4-(5,7-diethyl-2-oxo-4-phenyl sulphanyl-2H-[1,6]naphthyridin-1 ylmethyl)-phenyl]-thiophene-2-sulphonic acid(4,5-dimethyl-isoxazol-3-yl)-(2-methoxy-ethoxymethyl)-amide (0.3 gm, 0.40 mmol) was taken in ethanol (5 ml) and 6N hydrochloric acid (5 ml) was added to it and the reaction mixture was refluxed for 2 hrs at 100° C. The reaction mixture was concentrated under vacuum and the residue thus obtained was diluted with water and then pH of the solution was adjusted to 5 by saturated sodium bicarbonate solution and extracted with ethyl acetate (25 ml×2). The ethyl acetate layer was washed with water and brine and dried over sodium sulphate and concentrated. The crude compound was purified by column chromatography on a silica gel column using hexane:ethyl acetate as an eluent to provide 70 mg of 3-[4-(5,7-diethyl-2-oxo-4-phenyl sulphanyl-2H-[1,6] naphthyridin-1ylmethyl)-phenyl]-thiophene-2-sulphonic acid (4,5-dimethyl-isoxazol-3-yl)-amide as a white solid.

Molecular Formula: C₃₄H₃₂N₄O₄S₃

Molecular Weight: 656.84

¹HNMR (DMSOd₆): 1.19 (t, J=7.2 Hz, 3H) 1.42 (t, J=7.2 Hz, 3H) 1.46 (s, 3H) 2.11 (s, 3H) 2.76-2.78 (m, 2H) 3.47-3.52 (m, 2H) 5.48 (s, 2H) 5.80 (s, 1H) 7.11 (d, J=4.8 Hz, 1H) 7.21 (d, J=8.4 Hz, 2H) 7.37 (d, J=8 Hz, 2H) 7.65-7.75 (m, 6H) 7.94 (d, J=5.2 Hz, 1H) 10.83 (s, 1H)

Mass Spectrum: (m⁺¹) 657

### Example 4

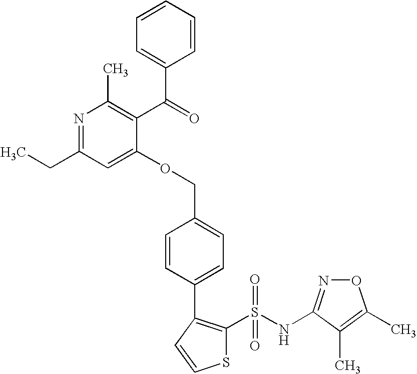

### 3-[4-(3-Benzoyl-6-ethyl-2-methyl-pyridin-4-yloxymethyl)-phenyl]-thiophene-2-sulphonic acid (4,5-dimethyl-isoxazol-3-yl)-amide

### STEP 01: Synthesis of 1-phenyl-butane-1,3 dione

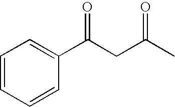

Sodium ethoxide (13.5 gm, 0.198 mol) was added to a stirred solution of dry ethyl acetate (80 ml, 0.72 mol) at −5° C. To this reaction mixture was added acetophenone (20 gm, 0.185 mol) at −5° C. and then the temperature of the reaction mixture was maintained at 0° C. for 12 hrs. The reaction mixture was then acidified with 1N hydrochloric acid and extracted with ethyl acetate (100 ml×2). The combined organic layer was washed with water and brine. The organic layer was then dried over sodium sulphate and evaporated to give 21 gm of a yellow colored solid of 1-phenyl-butane-1,3 dione.

### STEP 02: Synthesis of 3-amino-1-phenyl-but-2-en-1-one

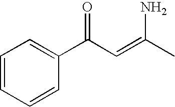

The mixture of 1-phenyl-butane-1,3 dione (20 gm, 0.123 mol) and ammonium acetate (38 gm, 0.49 mol) in dry methanol (200 ml) was stirred and heated to reflux for 24 hrs. The reaction mixture was concentrated under vacuum and to the residue was added chilled water, followed by extraction with ethyl acetate (100 ml×2). The combined extracts were washed with water and brine. The organic layer was dried over sodium sulphate and evaporated to give 19 gm of a yellow colored solid of 3-amino-1-phenyl-but-2-en-1-one.

### STEP 03: Synthesis of 5-(1-hydroxy propylidine)2,2-dimethyl-1,3-dioxane-4,6-dione

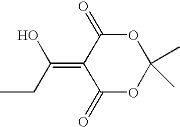

Propionyl chloride (7 ml, 0.0763 mol) was added to a solution of Meldrum's acid (10 gm, 0.069 mol) in pyridine (12 ml, 0.138 mol) and methylene chloride (50 ml) at 0° C. within 30 min. and the temperature of the reaction mixture was allowed to raise to ambient temperature and then stirred for 1 hr. The reaction mixture was then acidified using 1N hydrochloric acid and extracted with methylene chloride (50 ml×2). The combined extracts were washed with water and brine and the organic layer was dried over sodium sulphate and concentrated under vacuum to give 10 gm of 5-(1-hydroxy propylidine) 2,2-dimethyl-1,3-dioxane-4,6-dione as a crystalline solid.

### STEP 04: Synthesis of 3-benzoyl-6-ethyl-2-methyl-1H-pyridin-4-one

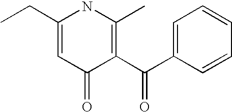

A mixture of 5-(1-hydroxy propylidine) 2,2-dimethyl-1,3-dioxane-4,6-dione (17.39 gm, 0.087 mol) and 3-amino-1-phenyl-but-2-en-1-one (10 gm, 0.062 mol) was stirred and heated at 120° C. for 2 hrs. The reaction mixture was cooled to room temperature and the crude compound was purified on a silica gel column using 10% methanol: ethyl acetate as an eluent to provide 5.8 gm of 3-benzoyl-6-ethyl-2-methyl-1H-pyridin-4-one as a yellow colored solid.

### STEP 05: Synthesis of (5-methyl-isoxazol-3-yl) carbamic acid tert-butyl ester

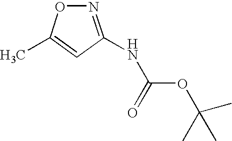

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 06: Synthesis of (4,5-dimethyl-isoxazol-3-yl) carbamic acid tert-butyl ester

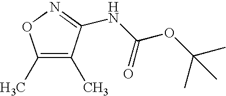

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 07: Synthesis of 4,5-dimethyl-isoxazol-3-yl-amine

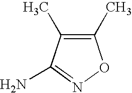

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 08: Synthesis of 3-bromo-thiophene-2-sulphonyl chloride

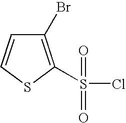

3-Bromothiophene (10 gm, 0.0617 mol) was taken in methylene chloride (60 ml) and cooled this solution was cooled to −78° C. Then chlorosulphonic acid (25 ml, 0.396 mol) was added drop wise to the reaction mixture at −78° C. The temperature of the reaction mixture was slowly raised to 0° C. and maintained for 1 hour. The mixture was then slowly poured into ice cold water, followed by extraction with methylene chloride (100 ml×3). The combined organic extract was washed with water and brine then dried over anhydrous sodium sulphate, and evaporated under vacuum to give brown color solid. The crude compound was purified on a silica gel column using hexane:ethyl acetate as an eluent to provide 5.4 gm of 3-bromo-thiophene-2-sulphonyl chloride.

### STEP 09: Synthesis of 3-bromo-thiophene-2-sulphonic acid (4,5-dimethyl-isoxazol-3yl) amide

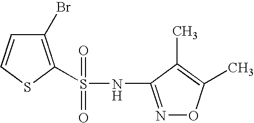

To the solution of 3-amino-4,5-dimethylisoxazole (2 gm, 0.0178 mol) in (25 ml) pyridine and dimethyl amino pyridine (0.230 gm, 0.0019 mol) was added 3-bromo-thiophene-2-sulphonyl chloride (5.0 gm, 0.01912) at 0° C. Then slowly the temperature of reaction mixture was raised to room temperature (28° C.), and then the reaction mixture was stirred for 6 hours. The reaction mixture was then concentrated under vacuum, the residue was acidified using 1N hydrochloric acid to pH 1 followed by extraction with dichloro methane (50 ml×3). The combined organic extract was washed with water and brine. Organic layer was dried over sodium sulphate and concentrated to give 3.5 gm of 3-bromo-thiophene-2-sulphonic acid (4,5-dimethyl-isoxazol-3yl) amide as brown colored solid.

### STEP 10: Synthesis of 3-bromo-thiophene-2-sulphonic acid (4,5 dimethyl-isoxazol-3-yl)-(2-methoxy-ethoxymethyl)-amide

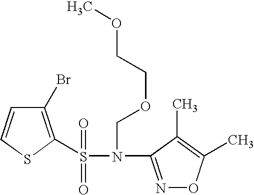

Under the flow of dry nitrogen, sodium hydride (0.600 gm, 0.0125 mol, 60% in mineral oil) was added portion wise to the solution of 3-bromo-thiophene-2-sulphonic acid (4,5-dimethyl-isoxazol-3yl) amide (3.5 gm, 0.0103 mol) in N,N-dimethyl formamide (30 ml) at −15° C. After completion of the addition reaction mixture was stirred at room temperature for 30 min. Then the reaction mixture was recooled to 0° C. To this reaction mixture 2-methoxyethoxy methylchloride (1.55 gm, 0.0124 mol) was added drop wise within 30 min, maintaining the temperature of the reaction mixture at 0° C. The reaction mixture was stirred at 0° C. for 30 min and then at room temperature for 4 hrs. Then the reaction mixture was diluted with ethyl acetate (100 ml) followed by addition of (30 ml) ice cold water. The organic layer was separated, washed with water and brine. Finally the organic layer was dried over sodium sulphate and concentrated under vacuum. The crude compound was purified by column chromatography on a silica gel column using hexane:ethyl acetate as eluent to provide 2.7 gm of 3-bromo-thiophene-2-sulphonic acid (4,5 dimethyl-isoxazol-3-yl)-(2-methoxy-ethoxymethyl)-amide as yellow oil.

### STEP 11: Synthesis of 3-(4-formyl-phenyl)-thiophene-2-sulphonic acid (4,5-dimethyl-isoxazol-3-yl)-(2-methoxy-ethoxymethyl) amide

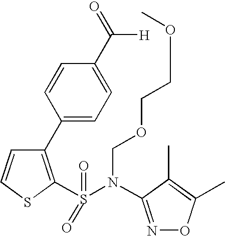

To a stirred solution (2.7 gm, 0.0063 mol) of 3-bromo-thiophene-2-sulphonic acid (4,5 dimethyl-isoxazol-3-yl)-(2-methoxy-ethoxymethyl)-amide and 4-formyl boronic acid (1.04 gm, 0.006 mol) in toluene (15 ml) and ethanol (12 ml) under nitrogen atmosphere was added 2M aqueous sodium carbonate solution (2 gm in 16 ml water). This reaction mixture was then stirred for 15 minutes, whereupon tetrakis triphenyl phosphine palladium (0) (0.40 gm, 0.00034 mol) was added into the reaction mixture. The mixture was heated to 85° C. for 6 hrs and was then cooled to room temperature hereafter ethyl acetate (50 ml) was added. This reaction mixture was concentrated under vacuum and to the residual mass; ethyl acetate (100 ml) was added, followed by chilled water and further extraction with ethyl acetate (100 ml×2). The combined extract was washed with water and brine and dried over sodium sulphate and concentrated under vacuum. The crude compound was purified on a silica gel column using hexane:ethyl acetate as an eluent to provide 1.1 gm of 3-(4-formyl-phenyl)-thiophene-2-sulphonic acid (4,5-dimethyl-isoxazol-3-yl)-(2-methoxy-ethoxymethyl) amide as an oil.

### STEP 12: Synthesis of 3-(4-hydroxymethyl-phenyl)-thiophene-2-sulphonic acid (4,5-dimethyl-isoxazol-3-yl)-(2-methoxy-ethoxymethyl)-amide

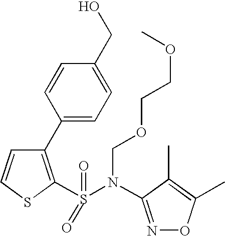

Lithium aluminum hydride (0.100 gm, 0.0029 mol) was added under the flow of nitrogen and stirring to tetrahydrofuran (25 ml) at 0° C., followed by addition of 3-(4-formyl-phenyl)-thiophene-2-sulphonic acid (4,5-dimethyl-isoxazol-3-yl)-(2-methoxy-ethoxymethyl) amide (1.1 gm, 0.0024 mol) in (5 ml) tetrahydrofuran. The reaction mixture was stirred at 0° C. for 1 hr and the temperature was then raised to room temperature (28° C.) and the mixture stirred for 4 hrs. The reaction was worked up by addition of sodium hydroxide solution (1 gm dissolved in 100 ml water) at 0° C. followed by extraction with ethyl acetate (50 ml×2). The combined organic layers were washed with water and brine solution and then organic layer was dried over sodium sulphate and concentrated under vacuum to give 1.0 gm of 3-(4-hydroxymethyl-phenyl)-thiophene-2-sulphonic acid (4,5-dimethyl-isoxazol-3-yl)-(2-methoxy-ethoxymethyl)-amide as an oil.

### STEP 13: Synthesis of 3-(4-methanesulphonyl methyl-phenyl)-thiophene-2-sulphonic acid (4,5-dimethyl-isoxazol-3-yl)-(2-methoxy-ethoxymethyl)amide

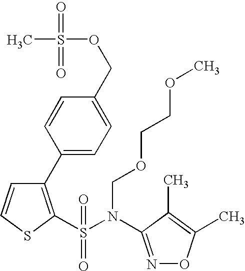

To the 0° C. cooled solution of 3-(4-hydroxymethyl-phenyl)-thiophene-2-sulphonic acid (4,5-dimethyl-isoxazol-3-yl)-(2-methoxy-ethoxymethyl)-amide (1.0 gm, 0.0022 mol) in (60 ml) of dichloro methane, N-Ethyl diisopropyl amine (0.6 ml, 0.0033 mol) was added, followed by slow addition of the solution of methane sulphonyl chloride (0.2 ml, 0.0033 mol) in (10 ml) dichloromethane in to the reaction mixture. The reaction mixture was then warmed and stirred at room temperature for 3 hrs. Workup was done by addition of ice-cold water into the reaction mixture followed by extraction with methylene dichloride (25 ml×2). The combined organic extract was washed with dilute hydrochloric acid followed by washings with water and brine. The organic layer was dried over sodium sulphate and concentrated under vacuum. The crude compound was purified by column chromatography on a silica gel column using hexane/ethyl acetate as an eluent to provide 0.700 gm of 3-(4-methanesulphonyl methyl-phenyl)-thiophene-2-sulphonic acid (4,5-dimethyl-isoxazol-3-yl)-(2-methoxy-ethoxymethyl) amide as a viscous liquid.

### STEP 14: Synthesis of 3-[4-(3-benzoyl-6-ethyl-2-methyl-pyridin-4-yloxymethyl)-phenyl]-thiophene-2-sulphonic acid (4,5-dimethyl-isoxazol-3-yl)-(2-methoxy-ethoxymethyl)-amide

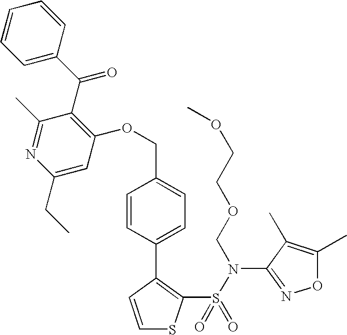

To the stirred solution of 3-benzoyl-6-ethyl-2-methyl-1H-pyridin-4-one (0.294 gm, 0.0012 mol) in N,N-dimethyl formamide (5 ml) at 0° C. under a flow of dry nitrogen gas sodium hydride (60% in mineral oil) (0.070 gm, 0.0014 mol) was added portion wise. After the addition, the temperature of the reaction mixture was raised to room temperature and maintained for 30 min. The mixture was re-cooled to 0° C. and a solution of 3-(4-methanesulphonyl methyl-phenyl)-thiophene-2-sulphonic acid (4,5-dimethyl-isoxazol-3-yl)-(2-methoxy-ethoxymethyl) amide (0.6 gm, 0.0011 mol) in (5 ml) dimethyl formamide was added drop wise and the mixture stirred at room temperature for 24 hrs. The mixture was then cooled and diluted with ethyl acetate (40 ml) followed by addition of water (10 ml) at 0° C. and further extracted with ethyl acetate (50 ml×2) The combined organic extract was washed with water and brine and dried over sodium sulphate and concentrated under vacuum to give 0.5 gm of 3-[4-(3-Benzoyl-6-ethyl-2-methyl-pyridin-4-yloxymethyl)-phenyl]-thiophene-2-sulphonic acid (4,5-dimethyl-isoxazol-3-yl)-(2-methoxy-ethoxymethyl)-amide as a gum.

### STEP 15: Synthesis of 3-[4-(3-benzoyl-6-ethyl-2-methyl-pyridin-4-yloxymethyl)-phenyl]-thiophene-2-sulphonic acid (4,5-dimethyl-isoxazol-3-yl)-amide

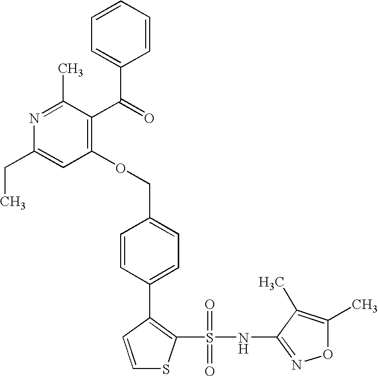

To (0.5 gm, 0.73 mmol) of 3-[4-(3-Benzoyl-6-ethyl-2-methyl-pyridin-4-yloxymethyl)-phenyl]-thiophene-2-sulphonic acid (4,5-dimethyl-isoxazol-3-yl)-(2-methoxy-ethoxymethyl)-amide was added 95% ethanol (5 ml) and 5 ml of 6N aqueous hydrochloric acid at room temperature. The reaction mixture was refluxed for 3 hrs and then concentrated under vacuum. The residue thus obtained was diluted with water and the pH of the solution was adjusted to 5 using sodium bicarbonate solution. Then the mixture was extracted with ethyl acetate (50 ml×2). The combined organic extracts were washed with water and brine and then dried over sodium sulphate and concentrated under vacuum. The crude compound was purified using flash chromatography on a silica gel column using hexane/ethyl acetate as an eluent to provide 70 mg of amorphous yellowish foam of 3-[4-(3-benzoyl-6-ethyl-2-methyl-pyridin-4-yloxymethyl)-phenyl]-thiophene-2-sulphonic acid (4,5-dimethyl-isoxazol-3-yl)-amide.

Molecular Formula: C₃₁H₂₉N₃O₅S₂

Molecular Weight: 587.71

¹HNMR (DMSOd₆): 1.26 (t, J=7.2 Hz, 3H) 1.48 (s, 3H) 2.12 (s, 3H) 2.22 (s, 3H) 2.74-2.81 (m, 2H) 5.19 (s, 2H) 6.98-7.01 (m, 1H) 7.11-7.13 (m, 3H) 7.27-7.29 (m, 2H) 7.55-7.57 (m, 2H) 7.69-7.77 (m, 3H) 7.94 (m, 1H) 10.85 (s, 1H)

Mass Spectrum: (m⁺¹) 588

### Example 5

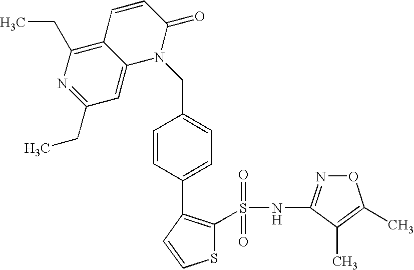

### 3-[4-(5,7-diethyl-2-oxo-2H-[1,6] naphthyridin-1-ylmethyl)-phenyl]-thiophene-2-sulphonic acid (4,5-dimethyl-isoxazol-3-yl)-amide

### STEP 01: Synthesis of (5-methyl-isoxazol-3-yl) carbamic acid tert-butyl ester

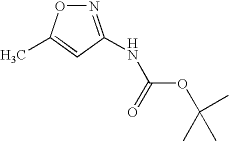

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 02: Synthesis of (4,5-dimethyl-isoxazol-3-yl) carbamic acid tert-butyl ester

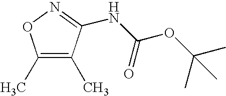

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 03: Synthesis of 4,5-dimethyl-isoxazol-3-yl-amine

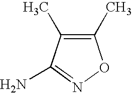

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 04: Synthesis of 3-bromo-thiophene-2-sulphonyl chloride

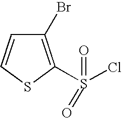

3-Bromothiophene (10 gm, 0.0617 mol) was dissolved in methylene chloride (60 ml) and the solution cooled to −78° C. Then chlorosulphonic acid (25 ml, 0.396 mol) was added drop wise to the reaction mixture at −78° C. The temperature of the reaction mixture was slowly raised to 0° C. and maintained for 1 hour. The reaction mixture was slowly poured into ice cold water, followed by extraction with methylene chloride (100 ml×3). The combined organic extract was washed with water and brine and then dried over anhydrous sodium sulphate, evaporated under vacuum to give a brown colored solid. The crude compound was purified on a silica gel column using hexane:ethyl acetate as an eluent to provide 5.4 gm of 3-bromo-thiophene-2-sulphonyl chloride.

### STEP 05: Synthesis of 3-bromo-thiophene-2-sulphonic acid (4,5-dimethyl-isoxazol-3yl) amide

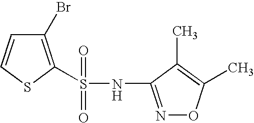

To the solution of 3-amino-4,5-dimethylisoxazole (2 gm, 0.0178 mol) in (25 ml) pyridine and dimethyl amino pyridine (0.230 gm, 0.0019 mol) was added 3-bromo-thiophene-2-sulphonyl chloride (5.0 gm, 0.01912) at 0° C. Then slowly the temperature of reaction mixture was raised to room temperature (28° C.), and then the reaction mixture was stirred for 6 hours. The reaction mixture was then concentrated under vacuum, the residue was acidified using 1N hydrochloric acid to pH 1 followed by extraction with dichloro methane (50 ml×3). The combined organic extract was washed with water and brine. Organic layer was dried over sodium sulphate and concentrated to give 3.5 gm of 3-bromo-thiophene-2-sulphonic acid (4,5-dimethyl-isoxazol-3yl) amide as brown colored solid.

### STEP 06: Synthesis of 3-bromo-thiophene-2-sulphonic acid (4,5 dimethyl-isoxazol-3-yl)-(2-methoxy-ethoxymethyl)-amide

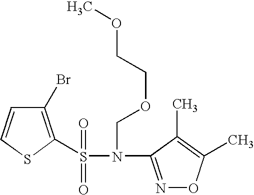

Under the flow of dry nitrogen, sodium hydride (0.600 gm, 0.0125 mol, 60% in mineral oil) was added portion wise to the solution of 3-bromo-thiophene-2-sulphonic acid (4,5-dimethyl-isoxazol-3yl) amide (3.5 gm, 0.0103 mol) in N,N-dimethyl formamide (30 ml) at −15° C. After completion of the addition reaction mixture was stirred at room temperature for 30 min. Then the reaction mixture was recooled to 0° C. To this reaction mixture 2-methoxyethoxy methylchloride (1.55 gm, 0.0124 mol) was added drop wise within 30 min, maintaining the temperature of the reaction mixture at 0° C. The reaction mixture was stirred at 0° C. for 30 min and then at room temperature for 4 hrs. Then the reaction mixture was diluted with ethyl acetate (100 ml) followed by addition of (30 ml) ice cold water. The organic layer was separated, washed with water and brine. Finally the organic layer was dried over sodium sulphate and concentrated under vacuum. The crude compound was purified by column chromatography on a silica gel column using hexane:ethyl acetate as eluent to provide 2.7 gm of 3-bromo-thiophene-2-sulphonic acid (4,5 dimethyl-isoxazol-3-yl)-(2-methoxy-ethoxymethyl)-amide as yellow oil.

### STEP 07: Synthesis of 3-(4-formyl-phenyl)-thiophene-2-sulphonic acid (4,5-dimethyl-isoxazol-3-yl)-(2-methoxy-ethoxymethyl) amide

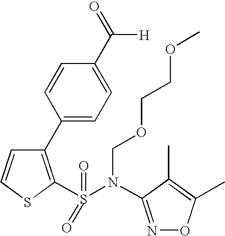

To a stirred solution of 2.7 gm (0.0063 mol) of 3-bromo-thiophene-2-sulphonic acid (4,5 dimethyl-isoxazol-3-yl)-(2-methoxy-ethoxymethyl)-amide and 4-formyl boronic acid 1.04 gm (0.006 mol) in toluene (15 ml) and ethanol (12 ml) under nitrogen atmosphere was added a 2M aqueous sodium carbonate solution (2 gm in 16 ml water). The reaction mixture was stirred for 15 minutes and then tetrakis triphenyl phosphine palladium (0) (0.40 gm, 0.00034 mol) was added. The reaction mixture was heated to 85° C. for 6 hrs and was then cooled to room temperature and to it ethyl acetate (50 ml) was added. The reaction mixture was then concentrated under vacuum. To the residual material ethyl acetate (100 ml) was added, followed by chilled water and further extraction with ethyl acetate (100 ml×2). The combined extract was washed with water and brine and dried over sodium sulphate and concentrated under vacuum. The crude compound was purified on a silica gel column using 1:2 hexane/ethyl acetate as an eluent to provide 1.1 gm of 3-(4-formyl-phenyl)-thiophene-2-sulphonic acid (4,5-dimethyl-isoxazol-3-yl)-(2-methoxy-ethoxymethyl) amide as an oil.

### STEP 08: Synthesis of 3-(4-hydroxymethyl-phenyl)-thiophene-2-sulphonic acid (4,5-dimethyl-isoxazol-3-yl)-(2-methoxy-ethoxymethyl)-amide

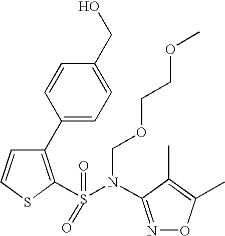

Lithium aluminum hydride (0.100 gm, 0.0029 mol) was added under flow of nitrogen to a stirred solution of tetrahydrofuran at 0° C., followed by addition of 3-(4-formyl-phenyl)-thiophene-2-sulphonic acid (4,5-dimethyl-isoxazol-3-yl)-(2-methoxy-ethoxymethyl) amide (1.1 gm, 0.0024 mol) in tetrahydrofuran (15 ml). The reaction mixture was stirred at 0° C. for 1 hr and the temperature then raised to room temperature (28° C.) and stirred for 4 hrs. The reaction was worked up by addition of sodium hydroxide solution (1 gm dissolved in 100 ml water) at 0° C. followed by extraction with ethyl acetate (50 ml×2). The combined organic layers were washed with water and brine and dried over sodium sulphate and concentrated under vacuum to give 1.0 gm of 3-(4-hydroxymethyl-phenyl)-thiophene-2-sulphonic acid (4,5-dimethyl-isoxazol-3-yl)-(2-methoxy-ethoxymethyl)-amide as an oil.

### STEP 09: Synthesis of 3-(4-methanesulphonyl methyl-phenyl)-thiophene-2-sulphonic acid (4,5-dimethyl-isoxazol-3-yl)-(2-methoxy-ethoxymethyl)amide

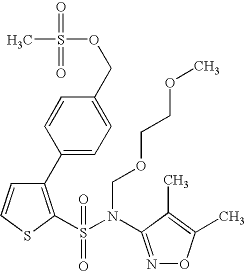

To the 0° C. cooled solution of 3-(4-hydroxymethyl-phenyl)-thiophene-2-sulphonic acid (4,5-dimethyl-isoxazol-3-yl)-(2-methoxy-ethoxymethyl)-amide (1.0 gm, 0.0022 mol) in (60 ml) of dichloro methane, N-Ethyl diisopropyl amine (0.6 ml, 0.0033 mol) was added, followed by slow addition of the solution of methane sulphonyl chloride (0.2 ml, 0.0033 mol) in (10 ml) dichloromethane in to the reaction mixture. The reaction mixture was then warmed and stirred at room temperature for 3 hrs. Workup was done by addition of ice-cold water into the reaction mixture followed by extraction with methylene dichloride (25 ml×2). The combined organic extract was washed with dilute hydrochloric acid followed by washings with water and brine. The organic layer was dried over sodium sulphate and concentrated under vacuum. The crude compound was purified by column chromatography on a silica gel column using hexane/ethyl acetate as an eluent to provide 0.700 gm of 3-(4-methanesulphonyl methyl-phenyl)-thiophene-2-sulphonic acid (4,5-dimethyl-isoxazol-3-yl)-(2-methoxy-ethoxymethyl) amide as a viscous liquid.

### STEP 10: Synthesis of 3-[4-(5,7-diethyl-2-oxo-2H-[1,6]-1-ylmethyl)-phenyl]-thiophene-2-sulphonic acid(4,5-dimethyl-isoxazol-3-yl)ethoxymethyl-amide

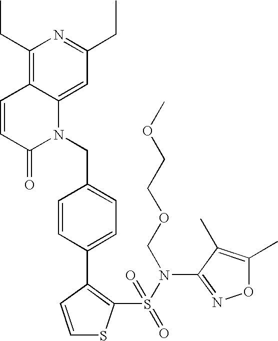

To the stirred solution of 5,7-diethyl-1H-[1,6] naphthyridin-2-one (0.4 gm, 0.0020 mol) in N,N-dimethyl formamide (5 ml) at 0° C. under the flow of dry nitrogen gas was added portion wise sodium hydride (60% in mineral oil) (0.115 gm, 0.0025 mol). After the addition, the temperature of the reaction mixture was raised to room temperature and maintained for 30 min. The reaction mixture was re-cooled to 0° C. and a solution of 3-(4-methanesulphonyl methyl-phenyl)-thiophene-2-sulphonic acid (4,5-dimethyl-isoxazol-3-yl)-(2-methoxy-ethoxymethyl) amide (1.0 gm, 0.0020 mol) in N,N dimethyl formamide (5 ml) was added drop wise and mixture was stirred at room temperature for 24 hrs. The reaction mixture was then diluted with ethyl acetate (40 ml) followed by of water (10 ml) at 0° C. and then extracted with ethyl acetate (50 ml×2). The combined extract was washed with water and brine and dried over sodium sulphate and concentrated under vacuum to give the crude compound. The crude compound was purified on a silica gel column using hexane:ethyl acetate as an eluent to provide 460 mg of 3-[4-(5,7-Diethyl-2-oxo-2H-[1,6]naptharidin-1-yl)-phenyl]-thiophene-2-sulphonic acid(4,5-dimethyl-isoxazol-3-yl)ethoxymethyl-amide as a gum.

### STEP 11: Synthesis of 3-[4-(5,7-diethyl-2-oxo-2H-[1,6] naphthyridin-1-ylmethyl)-phenyl]-thiophene-2-sulphonic acid(4,5-dimethyl-isoxazol-3-yl)-amide

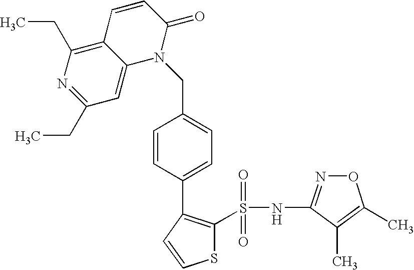

To (0.460 gm, 0.75 mmol) of 3-[4-(5,7-diethyl-2-oxo-2H-[1,6] naphthyridin-1-yl)-phenyl]-thiophene-2-sulphonic acid(4,5-dimethyl-isoxazol-3-yl)ethoxymethyl-amide was added 95% ethanol (5 ml) and 5 ml of 6N aqueous hydrochloric acid at room temperature. The reaction mixture was refluxed for 3 hrs and then concentrated under vacuum. The residue thus obtained was diluted with water and the pH of the solution was adjusted to 5 using sodium bicarbonate solution and the mixture was extracted with ethyl acetate (50 ml×2). The combined organic extracts were washed with water and brine then dried over sodium sulphate and concentrated under vacuum. The crude compound was purified using flash chromatography on a silica gel column using 1:1 hexane/ethyl acetate as an eluent to provide 200 mg of a yellowish foam of 3-[4-(5,7-diethyl-2-oxo-2H-[1,6] naphthyridin-1-ylmethyl)-phenyl]-thiophene-2-sulphonic acid(4,5-dimethyl-isoxazol-3-yl)-amide.

Molecular Formula: C₂₈H₂₈N₄O₄S₂

Molecular Weight: 548.67

¹HNMR (DMSOd₆): 1.17 (t, J=7.2 Hz, 3H) 1.25 (t, J=7.2 Hz, 3H) 1.45 (s, 3H) 2.10 (s, 3H) 2.70-2.76 (m, 2H) 3.04-3.11 (m 2H) 5.53 (s, 2H) 6.72 (d, J=10 Hz, 1H) 7.12 (d, J=5.2 Hz, 1H) 7.16 (s, 1H) 7.23 (d, J=8 Hz 2H) 7.39 (d, J=8 Hz, 2H) 7.94 (d, J=4.8 Hz, 1H) 8.24 (d, J=10 Hz, 1H) 10.83 (s, 1H)

Mass Spectrum: (m⁻¹) 547

### Example 6

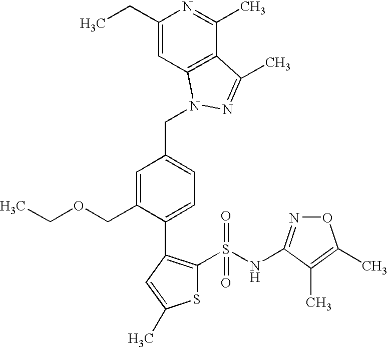

### 3-[2-Ethoxymethyl-4-(6-ethyl-3,4-dimethyl-pyrazolo[4,3-c]pyridine-1-ylmethyl)-phenyl]-5-methyl-thiophene-2-sulphonic acid(4,5-dimethyl-isoxazol-3-yl)-amide

### STEP 01: Synthesis of 5-(1-hydroxy propylidine)2,2-dimethyl-1,3-dioxane-4,6-dione

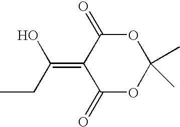

Propionyl chloride (35 ml, 0.381 mol) was added within 30 min to a solution of Meldrum's acid (50 gm, 0.345 mol) in pyridine (60 ml, 0.690 mol) and methylene chloride (200 ml) kept at 0° C., the temperature of the reaction mixture was allowed to raise to ambient temperature and stirred for 1 hr. The mixture was then acidified using 1N hydrochloric acid and extracted with methylene chloride (200 ml×2). The combined extracts were washed with water and brine and then dried over sodium sulphate and concentrated under vacuum to give 50 gm of 5-(1-hydroxy propylidine) 2,2-dimethyl-1,3-dioxane-4,6-dione as a crystalline solid.

### STEP 02: Synthesis of 3-acetyl-6-ethyl-2-methyl-1H-pyridin-4-one

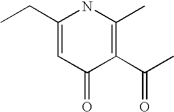

A mixture of 5-(1-hydroxy propylidine) 2,2-dimethyl-1,3-dioxane-4,6-dione (37.8 gm, 0.189 mol) and 4-amino-pent-3-en-2-one (12.5 gm, 0.126 mol) was heated to reflux at 120° C. for 2 hrs. The reaction mixture was cooled and the crude compound was purified on a silica gel column using 10% methanol: ethyl acetate as an eluent to provide 6 gm of 3-acetyl-6-ethyl-2-methyl-1H-pyridin-4-one as a yellow colored solid.

### STEP 03: Synthesis of 1-(4-chloro-6-ethyl-2-methyl-pyridin-3-yl)ethanone

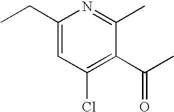

3-Acetyl-6-ethyl-2-methyl-1H-pyridin-4-one (5 gm, 0.027 mol) was added to 10 ml phosphorous oxychloride at 0° C. The reaction mixture was heated and stirred to 50° C. and maintained at this temperature for 8 hours. The reaction mixture was evaporated under vacuum and the residue thus obtained was basified to pH 8 with saturated sodium bicarbonate solution, followed by extraction with methylene dichloride (50 ml×2). The combined organic extracts were washed with water and brine and dried over anhydrous sodium sulphate and concentrated to give 3.2 gm of 1-(4-chloro-6-ethyl-2-methyl-pyridin-3-yl)ethanone as a yellow oily liquid.

### STEP 04: Synthesis of 6-ethyl-3,4 dimethyl-1H-pyrazolo [4,3-c] pyridine

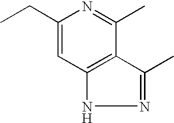

1-(4-Chloro-6-ethyl-2-methyl-pyridin-3-yl)ethanone (1.7 gm, 0.0086 mol) was dissolved in ethanol (20 ml) and hydrazine hydrate (2.15 ml, 0.038 mol) was added to the solution. The reaction mixture was slowly heated to reflux, and maintained at reflux for 4 hours. The reaction mixture was evaporated under vacuum and the residue dumped onto ice. The solid thus obtained was filtered off and suction dried to provide 1 gm of 6-ethyl-3,4 dimethyl-1H-pyrazolo[4,3-c]pyridine.

### STEP 05: Synthesis of (5-methyl-isoxazol-3-yl) carbamic acid tert-butyl ester

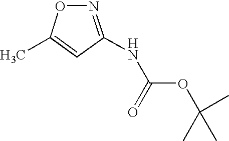

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 06: Synthesis of (4,5-dimethyl-isoxazol-3-yl) carbamic acid tert-butyl ester

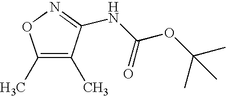

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 07: Synthesis of 4,5-dimethyl-isoxazol-3-yl-amine

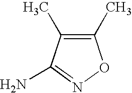

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 08: Synthesis of 5-methyl thiophene-2-sulphonyl chloride

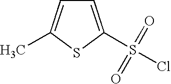

2-Methyl thiophene (50 gm, 0.51 mol) in chloroform was added to a solution of chloro sulphonic acid (105 ml, 1.53 mol) in chloroform (100 ml) at −5° C. to 0° C. The reaction mixture was maintained at 0° C. for 3 hrs and the crude reaction material was slowly dumped into ice cold water, followed by extraction with chloroform (100 ml×2). The combined extract was washed with water and brine and dried over anhydrous sodium sulphate, and evaporated under vacuum to give 16 gm of 5-methyl thiophene-2-sulphonyl chloride as brown colored liquid.

### STEP 09: Synthesis of 5-methyl-thiophene-2-sulphonic acid (4,5-dimethyl-isoxazol-3yl) amide

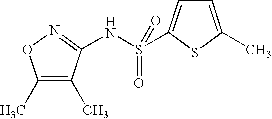

The solution of 5-Methyl thiophene-2-sulphonyl chloride (10.5 gm, 0.053 mol) in (15 ml) methylene chloride was added to the solution of 3-amino-4,5-dimethylisoxazole (4 gm, 0.035 mol) and dimethylaminopyridine (500 mg) in pyridine (20 ml) at 0° C. After the completion of the addition the temperature of the reaction mixture was slowly raised to room temperature and stirred for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 4.0 gm of 5-methyl-thiophene-2-sulphonic acid (4,5-dimethyl-isoxazol-3yl) amide as brown colored solid.

### STEP 10: Synthesis of 5-methyl-thiophene-2-sulphonic acid (4,5 dimethyl-isoxazol-3-yl)-(2-methoxy-ethoxymethyl)-amide

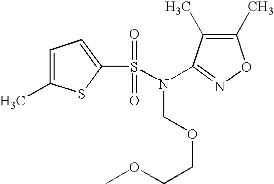

Under the flow of dry nitrogen and stirring, sodium hydride (3.4 gm, 0.07 mol, 60% dispersion in mineral oil) was added in to N,N-dimethyl formamide (40 ml) at 0° C., followed by addition of 5-methyl-thiophene-2-sulphonic acid (4,5-dimethyl-isoxazol-3yl) amide (16.5 gm, 0.060 mol) to it. After completion of the addition, temperature of the reaction mixture was slowly raised and stirred it at ambient temperature for 30 minutes then recooled the reaction mixture to 0° C., followed by the drop wise addition of methoxy ethoxy methyl chloride (8.03 gm, 0.064 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 18.2 gm of 5-methyl-thiophene-2-sulphonic acid (4,5-dimethyl-isoxazol-3yl) amide as yellowish oil.

### STEP 11: Synthesis of 3-borono-N-(4,5-dimethyl-3-isoxazolyl)-N-[(2-methoxy-ethoxy)methyl]-5-methyl-thiophene sulphonamide

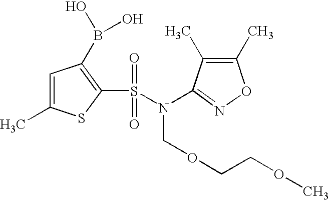

Under the dry nitrogen atmosphere the solution of (14 gm, 0.038 mol) 5-methyl-thiophene-2-sulphonic acid (4,5 dimethyl-isoxazol-3-yl)-(2-methoxy-ethoxymethyl)-amide in tetrahydrofuran (80 ml) was cooled to −78° C. To this n-Butyl lithium (60 ml, 0.097 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (15 ml, 0.062 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×3). The combined extract was washed with water and brine solution. The organic layer was dried over sodium sulphate and concentrated under vacuum to give 15 gm of 3-borono-N-(4,5-dimethyl-3-isoxazolyl)-N-[(2-methoxy-ethoxy)methyl]-5-methyl-thiophene sulphonamide as thick oily mass.

### STEP 12: Synthesis of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester

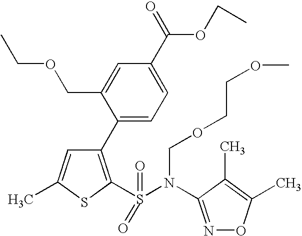

To a stirred solution of 3-borono-N-(4,5-dimethyl-3-isoxazolyl)-N-[(2-methoxy-ethoxy) methyl]-5-methyl-thiophene sulphonamide (15 gm, 0.037 mol) and 4-bromo-3-ethoxymethyl-benzoic acid ethyl ester (11 gm, 0.038 mol) in toluene (120 ml) and ethanol (60 ml) under nitrogen, a solution of 2M aqueous sodium carbonate (4.0 gm in 19 ml water) was added. The reaction mixture was stirred under nitrogen atmosphere for 15 minutes and then tetrakis triphenyl phosphine palladium (0) (2.15 gm, 0.0018 mol) was added. The mixture was heated to 85° C. for 6 hrs. The reaction mixture was concentrated and ethyl acetate (25 ml) was added to the residue followed by chilled water and extraction with ethyl acetate (100 ml×2). The combined extracts were washed with water and brine and dried over sodium sulphate and concentrated completely under vacuum. The crude compound was purified by column chromatography on a silica gel column using hexane:ethyl acetate as eluent to provide 8 gm of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester as an oily mass.

### STEP 13: Synthesis of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

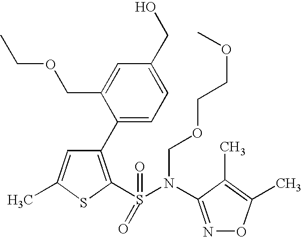

Lithium aluminium hydride (1.4 gm, 0.037 mol) was added to a stirred solution of tetrahydrofuran at 0° C. under flow of nitrogen, followed by addition of (4-{2-[(4,5-dimethyl-isoxazol-3-yl)-(2-methoxy-ethoxymethyl)-sulphamoyl]-5-methyl-thiophene-3-yl}-3-ethoxymethyl-phenyl)-acetic acid ethyl ester (8 gm, 0.014 mol) in 35 ml of tetrahydrofuran. The reaction mixture was stirred at 0° C. for 1 hr and then the temperature was raised to room temperature and the mixture stirred for 4 hrs. The excess lithium aluminium hydride was destroyed by addition of a sodium hydroxide solution (1 gm dissolved in 100 ml water) at 0° C. followed by extraction with ethyl acetate (25 ml×2). The organic layer was dried over sodium sulphate and concentrated completely under vacuum to give 4.7 gm of 3-(2-ethoxymethyl-4-hydroxy methyl-phenyl)-5-methyl-thiophene-2-sulphonic acid-(4,5-Dimethyl-isoxazol-3-yl)-2-methoxy-ethoxymethyl) amide

### STEP 14: Synthesis of methane sulphonic acid 4-{2-[(4,5-dimethyl-isoxazol-3yl)-(2-methoxy-ethoxy methyl)-sulphamoyl]-5-methylthiophene-3-yl}-3-ethoxy methyl-benzyl ester

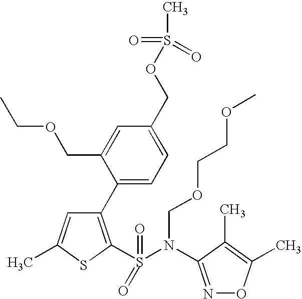

N-Ethyl diisopropyl amine (2.13 ml, 0.012 mol) was added to a solution of 3-(4-hydroxymethyl-phenyl)-5-methyl-thiophene-2-sulphonic acid (4,5-dimethyl-isoxazol-3-yl)-(2-methoxy-ethoxymethyl)-amide (3.2 gm, 0.0060 mol) in 10 ml of dichloro methane. The reaction mixture was cooled to 0° C., hereafter methane sulphonyl chloride (0.6 ml, 0.0073 mol) was slowly added into the reaction mixture. The mixture was maintained at room temperature for 3 hrs and was then dumped into ice-cold water followed by extraction with methylene chloride (50 ml×2). The combined extracts were washed with dilute hydrochloric acid followed by water and brine solution and the organic layer was dried over sodium sulphate and concentrated to give 3.3 gm of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester.

### STEP 15: Synthesis of 3-[2-ethoxymethyl-4-(6-ethyl-3,4-dimethyl-pyrazolo[4,3-c]pyridine-1-yl methyl)-phenyl]-5-methyl-thiophene-2sulphonic acid(4,5-dimethyl-isoxazol-3-yl)-(2-methoxy-ethoxymethyl)-amide

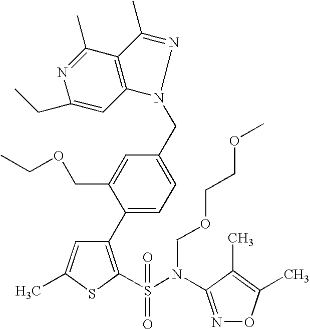

To the stirred solution of 6-ethyl-3,4-dimethyl-pyrazolo[4,3-c]pyridine (0.380 gm, 0.0021 mol) in N,N-dimethyl formamide (5 ml) at −15° C. under nitrogen was added portion wise sodium hydride (60% in mineral oil) (0.125 gm, 0.0026 mol). After the addition was over, the reaction mixture was warmed to ambient temperature and maintained for 30 min. The reaction mixture was then re-cooled to 0° C. and a solution of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester (1.25 gm, 0.002 mmol) in (5 ml) N,N-dimethyl formamide was added drop wise to the and the reaction mixture was then stirred at room temperature for 24 hrs. The reaction mixture was then diluted with ethyl acetate (40 ml), followed by 10 ml of cold water. The organic layer was separated and then washed with water and brine and finally dried over sodium sulphate and evaporated under vacuum. The crude compound was purified by column chromatography on a silica gel column using hexane:ethyl acetate as an eluent to provide 1.0 gm of 3-[2-ethoxymethyl-4-(6-ethyl-3,4-dimethyl-pyrazolo[4,3-c]pyridine-1-yl methyl)-phenyl]-5-methyl-thiophene-2sulphonic acid(4,5-dimethyl-isoxazol-3-yl)-(2-methoxy-ethoxymethyl)-amide as a viscous oily mass.

### STEP 16: Synthesis of 3-[2-ethoxymethyl-4-(6-ethyl-3,4-dimethyl-pyrazolo[4,3-c]pyridine-1-yl methyl)-phenyl]-5-methyl-thiophene-2-sulphonic acid(4,5-dimethyl-isoxazol-3-yl)-amide

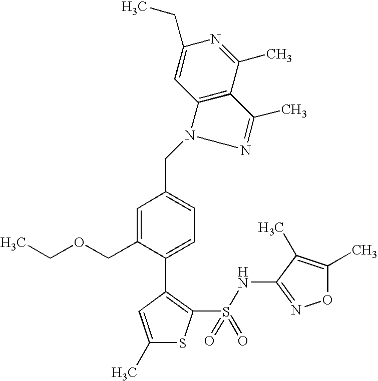

To 3-[2-Ethoxymethyl-4-(6-ethyl-3,4-dimethyl-pyrazolo[4,3-c]pyridine-1-yl methyl)-phenyl]-5-methyl-thiophene-2-sulphonic acid(4,5-dimethyl-isoxazol-3-yl)-(2-methoxy-ethoxymethyl)-amide (1.0 gm, 1.46 mmol) was added 95% ethanol (7 ml) and 6N aqueous hydrochloric acid (7 ml) at room temperature. The reaction mixture was refluxed for 3 hrs and then concentrated under vacuum. The residue thus obtained was diluted with water and the pH of the solution was adjusted to 8 using a saturated solution of sodium bicarbonate. This solution was then acidified to pH5 with acetic acid, and extracted with ethyl acetate (25 ml×2). The combined organic layers were washed with water and brine then dried over sodium sulphate and concentrated under vacuum. The crude compound was purified by column chromatography on a silica gel column using hexane:ethyl acetate as an eluent to provide 200 mg of 3-[2-Ethoxymethyl-4-(6-ethyl-3,4-dimethyl-pyrazolo[4,3-c]pyridine-1-yl methyl)-phenyl]-5-methyl-thiophene-2-sulphonic acid(4,5-dimethyl-isoxazol-3-yl)-amide.

Molecular Formula: C₃₀H₃₅N₅O₄S₂

Molecular Weight: 593.76

¹HNMR (DMSOd₆): 1.02 (t, J=6.8 Hz, 3H) 1.26 (t, J=6.8 Hz, 3H) 1.49 (s, 3H) 2.14 (s, 3H) 2.46 (s, 3H) 2.63 (s, 3H) 2.76 (m, 5H) 3.24-3.28 (m, 2H) 4.05 (s, 2H) 5.55 (s, 2H) 6.68 (s, 1H) 6.92 (d, J=7.6 Hz, 1H) 6.99 (d, J=7.6 Hz, 1H) 7.28 (s, 1H) 7.38 (s, 1H) 10.85 (s, 1H)

Mass Spectrum: (m⁺¹) 594

### Example 7

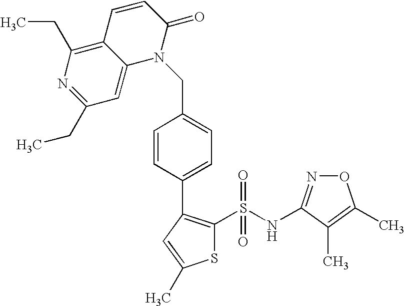

### 3-[4-(5,7-diethyl-2-oxo-2H-[1,6] naphthyridin-1-yl-methyl)-phenyl]-5-methyl-thiophene-2-sulphonic acid-(4,5 dimethyl-isoxazol-3-yl)-amide

### STEP 01: Synthesis of (5-methyl-isoxazol-3-yl) carbamic acid tert-butyl ester

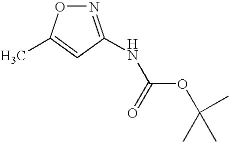

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 02: Synthesis of (4,5-dimethyl-isoxazol-3-yl) carbamic acid tert-butyl ester

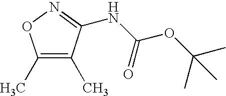

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 03: Synthesis of 4,5-dimethyl-isoxazol-3-yl-amine

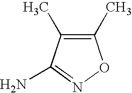

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 04: Synthesis of 5-methyl thiophene-2-sulphonyl chloride

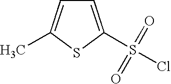

A solution of 2-methyl thiophene (50 gm, 0.51 mol) in chloroform was added to a solution of chloro sulphonic acid (105 ml, 1.53 mol) in chloroform at −5° C. to 0° C. The temperature of the reaction mixture was maintained at 0° C. for 3 hrs. The crude reaction mass was slowly dumped into ice cold water, followed by extraction with chloroform (100 ml×2). The combined extract was washed with water and brine and dried over anhydrous sodium sulphate, and evaporated under vacuum to give 16 gm of 5-Methyl thiophene-2-sulphonyl chloride as brown colored liquid.

### STEP 05: Synthesis of 5-methyl-thiophene-2-sulphonic acid (4,5-dimethyl-isoxazol-3yl) amide

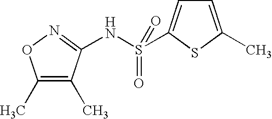

The solution of 5-Methyl thiophene-2-sulphonyl chloride (10.5 gm, 0.053 mol) in (15 ml) methylene chloride was added to the solution of 3-amino-4,5-dimethylisoxazole (4 gm, 0.035 mol) and dimethylaminopyridine (500 mg) in pyridine (20 ml) at 0° C. After the completion of the addition the temperature of the reaction mixture was slowly raised to room temperature and stirred for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 4.0 gm of 5-methyl-thiophene-2-sulphonic acid (4,5-dimethyl-isoxazol-3yl) amide as brown colored solid.

### STEP 06: Synthesis of 5-methyl-thiophene-2-sulphonic acid (4,5 dimethyl-isoxazol-3-yl)-(2-methoxy-ethoxymethyl)-amide

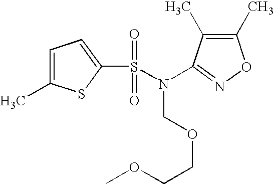

At 0° C., under stirring, sodium hydride (0.800 gm, 0.166 mol, 60% dispersion in mineral oil) was added in to N,N-dimethyl formamide (30 ml), followed by addition of 5-methyl-thiophene-2-sulphonic acid (4,5-dimethyl-isoxazol-3yl) amide (4 gm, 0.0146 mol) to it. After completion of the addition, temperature of the reaction mixture was slowly raised and stirred it at ambient temperature for 30 minutes then recooled the reaction mixture to 0° C., followed by the drop wise addition of methoxy ethoxy methyl chloride (2.7 gm, 0.021 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 3.7 gm of 5-Methyl-thiophene-2-sulphonic acid (4,5-dimethyl-isoxazol-3yl) amide as yellowish oil.

### STEP 07: Synthesis of 3-borono-N-(4,5-dimethyl-3-isoxazolyl)-N-[(2-methoxy-ethoxy)methyl]-5-methyl-thiophene sulphonamide

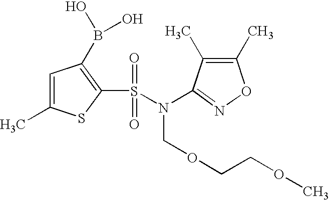

Under the dry nitrogen atmosphere the solution of (14 gm, 0.038 mol) 5-methyl-thiophene-2-sulphonic acid (4,5 dimethyl-isoxazol-3-yl)-(2-methoxy-ethoxymethyl)-amide in tetrahydrofuran (80 ml) was cooled to −78° C. To this n-Butyl lithium (60 ml, 0.097 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (15 ml, 0.062 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×3). The combined extract was washed with water and brine solution. The organic layer was dried over sodium sulphate and concentrated under vacuum to give 15 gm of 3-borono-N-(4,5-dimethyl-3-isoxazolyl)-N-[(2-methoxy-ethoxy)methyl]-5-methyl-thiophene sulphonamide as thick oily mass.

### STEP 08: Synthesis of 3-(4-formyl-phenyl)-5-methyl-thiophene-2-sulphonic acid (4,5-dimethyl-isoxazol-3-yl)-(2-methoxy-ethoxymethyl)amide

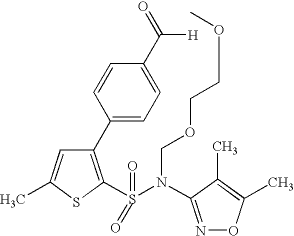

To the stirred solution of 3-borono-N-(4,5-dimethyl-3-isoxazolyl)-N-[(2-methoxy-ethoxy) methyl]-5-methyl-thiophene sulphonamide (3.4 gm, 0.008 mol) and 4-bromo benzaldehyde (1.5 gm, 0.0081 mol) in toluene (20 ml) and ethanol (10 ml) under nitrogen atmosphere, was added a solution of 2M aqueous sodium carbonate (2.36 gm in 11 ml water). The reaction mixture was stirred under nitrogen atmosphere for 15 minutes, and then tetrakis triphenyl phosphine palladium (0) (0.430 gm, 0.00037 mol) was added and the reaction mixture was heated to 85° C. for 6 hrs. The mixture was cooled, and ethyl acetate (25 ml) was added followed by stirring at room temperature for 10 min. The reaction mixture was concentrated and the residue thus obtained was dissolved in ethyl acetate (100 ml) followed by washings with water and brine. The organic layer was dried over sodium sulphate and concentrated under vacuum. The crude compound was purified by a silica gel column using hexane:ethyl acetate as an eluent to provide 1.8 gm of 3-(4-formyl-phenyl)-5-methyl-thiophene-2-sulphonic acid (4,5-dimethyl-isoxazol-3-yl)-(2-methoxy-ethoxymethyl)amide as an oily mass.

### STEP 09: Synthesis of 3-(4-hydroxymethyl-phenyl)-5-methyl-thiophene-2-sulphonic acid (4,5-dimethyl-isoxazol-3-yl)-(2-methoxy-ethoxymethyl)-amide

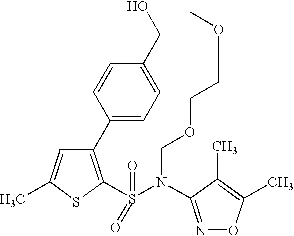

Lithium aluminium hydride (0.300 gm, 0.0088 mol) was added to tetrahydrofuran at 0° C. under dry nitrogen atmosphere, followed by addition of 3-(4-formyl-phenyl)-thiophene-2-sulphonic acid (4,5-dimethyl-isoxazol-3-yl)-(2-methoxy-ethoxymethyl) amide (1.8 gm, 0.0039 mol) in (15 ml) tetrahydrofuran. The reaction mixture was stirred at 0° C. for 1 hr and then the temperature was raised to room temperature and stirred for 4 hrs. The excess lithium aluminium hydride was destroyed by addition of sodium hydroxide solution (1 gm dissolved in 100 ml water) at 0° C. followed by extraction with ethyl acetate (50 ml×2). The organic layer was dried over sodium sulphate and concentrated under vacuum to give 1.5 gm of 3-(4-hydroxymethyl-phenyl)-5-methyl-thiophene-2-sulphonic acid (4,5-dimethyl-isoxazol-3-yl)-(2-methoxy-ethoxymethyl)-amide as an oily liquid.

### STEP 10: Synthesis of 3-(4-methanesulphonyl methyl-phenyl)-5-methyl-thiophene-2-sulphonic acid (4,5-dimethyl-isoxazol-3-yl)-(2-methoxy-ethoxy methyl)amide

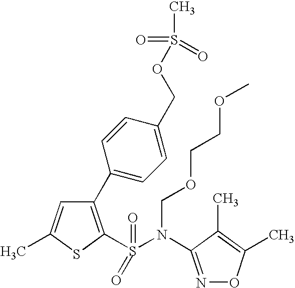

To a solution of 3-(4-hydroxymethyl-phenyl)-5-methyl-thiophene-2-sulphonic acid (4,5-dimethyl-isoxazol-3-yl)-(2-methoxy-ethoxymethyl)-amide (1.5 gm, 0.0032 mol) in 20 ml of dichloro methane was added N-ethyl diisopropyl amine (1.2 ml, 0.0034 mol). The reaction mixture was cooled to 0° C., and then methane sulphonyl chloride (0.3 ml, 0.0038 mol) was added slowly into the reaction mixture. After the completion of the addition, the temperature of the reaction mixture was slowly raised to room temperature and stirred for 3 hrs. Then the mixture was poured into ice-cold water followed by extraction with methylene chloride (25 ml×2). The combined extracts were washed with dilute hydrochloric acid followed by water and brine solution. The organic layer was dried over sodium sulphate and concentrated under vacuum. The crude compound was purified by a silica gel column using hexane/ethyl acetate as an eluent to provide 0.800 gm of 3-(4-methanesulphonyl methyl-phenyl)-5-methyl-thiophene-2-sulphonic acid (4,5-dimethyl-isoxazol-3-yl)-(2-methoxy-ethoxymethyl) amide as a viscous liquid.

### STEP 11: Synthesis of 3-[4-(5,7-diethyl-2-oxo-2H-[1,6] naphthyridin-1ylmethyl)-phenyl]-5-methyl-thiophene-2-sulphonic acid(4,5 dimethyl-isoxazol-3-yl)-(2-methoxy-ethoxymethyl)-amide

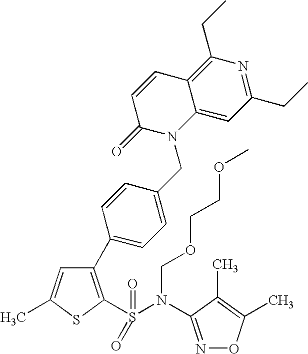

To the stirred solution of 5,7-diethyl-1H-[1,6] naphthyridin-2-one (0.467 gm, 2.3 mmol) in N,N-dimethyl formamide (3 ml) at −15° C. under nitrogen atmosphere was added portion wise sodium hydride (60% in mineral oil) (0.170 gm, 3.5 m mol). After the completion of addition, the temperature of the reaction mixture was slowly raised to room temperature and stirred for 30 min. Then the reaction mixture was re-cooled to 0° C. and a solution of 3-(4-methanesulphonyl methyl-phenyl)-5-methyl-thiophene-2-sulphonic acid (4,5-dimethyl-isoxazol-3-yl)-(2-methoxy-ethoxymethyl) amide (1.2 gm, 2.2 mmol) in (10 ml) N,N-dimethyl formamide was added drop wise the reaction mixture was stirred at room temperature for 24 hrs. >The reaction mixture was then diluted with ethyl acetate (40 ml) and water (20 ml). The organic layer was separated, washed with water and brine and finally dried over sodium sulphate and evaporated under vacuum. The crude compound was purified by a silica gel column using 1:4 hexane/ethyl acetate as an eluent to provide 0.500 gm of 3-[4-(5,7-Diethyl-2-oxo-2H-[1,6] naphthyridin-1 ylmethyl)-phenyl]-5-methyl-thiophene-2-sulphonic acid(4,5 dimethyl-isoxazol-3-yl)-(2-methoxy-ethoxymethyl)-amide as an oily liquid.

### STEP 12: Synthesis of 3-[4-(5,7-diethyl-2-oxo-2H-[1,6] naphthyridin-1ylmethyl)-phenyl]-5-methyl-thiophene-2-sulphonic acid(4,5 dimethyl-isoxazol-3-yl)-amide

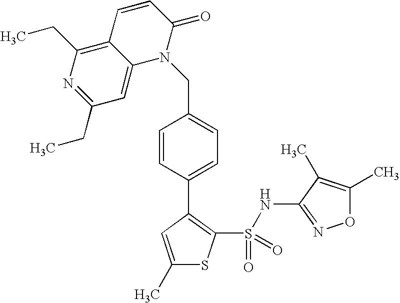

To a solution of 95% ethanol (6 ml) and 6N aqueous hydrochloric acid (6 ml) at room temperature was added (0.500 gm, 0.76 mmol) of 3-[4-(5,7-Diethyl-2-oxo-2H-[1,6] naphthyridin-1ylmethyl)-phenyl]-5-methyl-thiophene-2-sulphonic acid(4,5 dimethyl-isoxazol-3-yl)-(2-methoxy-ethoxymethyl)-amide. The reaction mixture was then refluxed for 3 hrs and concentrated under vacuum. The residue thus obtained was diluted with water and the pH of the solution was adjusted to 5 with sodium bicarbonate solution and extracted with ethyl acetate (30 ml×2). The combined organic extract was washed with water and brine and dried over sodium sulphate, and concentrated to give 400 mg of crude product, which was purified by a silica gel column using 1:4 hexane/ethyl acetate as an eluent to provide 340 mg of 3-[4-(5,7-diethyl-2-oxo-2H-[1,6]naphthyridin-1ylmethyl)-phenyl]-5-methyl-thiophene-2-sulphonic acid(4,5 dimethyl-isoxazol-3-yl)-amide as a off white solid.

Molecular Formula: C₂₉H₃₀N₄O₄S₂

Molecular Weight: 562.70

¹HNMR (DMSOd₆): 1.18 (t, J=7.2 Hz, 3H) 1.26 (t, J=7.2 Hz, 3H) 1.46 (s, 3H) 2.11 (s, 3H) 2.47 (s, 3H) 2.76-2.80 (m, 2H) 3.12-3.17 (m, 2H) 5.54 (s, 2H) 6.79-6.81 (m, 1H) 6.86 (s, 1H) 7.20-7.26 (m, 3H) 7.36 (d, J=8.4 Hz, 2H) 8.28-8.31 (m, 1H) 10.74 (s, 1H)

Mass Spectrum: (m⁻¹) 561

### Example 8

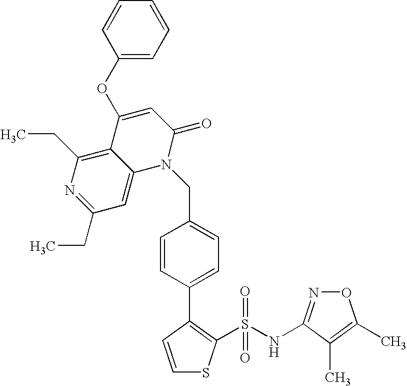

### 3-[4-(5,7-diethyl-2-oxo-4-phenoxy-2H-[1,6]naphthyridin-1-ylmethyl)-phenyl]-thiophene-2-sulphonic acid(4,5-dimethyl-isoxazol-3-yl)-amide

### STEP 01: Synthesis of methyl 3-amino pentenoate

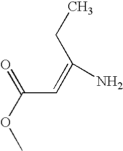

A mixture of methyl propionyl acetate (50 gm, 0.3842 mol) and ammonium acetate (148 gm, 1.921 mol) in dry methanol (500 ml) was stirred and refluxed for 2 days. The reaction mixture was cooled and concentrated under vacuum. The residue thus obtained was basified to pH 8 and extracted with ethyl acetate. The ethyl acetate layer was washed with water, brine and the organic layer was dried over sodium sulphate and concentrated to give 50 gm of methyl 3-amino pentenoate as a pale yellow liquid.

### STEP 02: Synthesis of 2,6-diethyl-4-oxo-1,4-dihydro-pyridine-3-carboxylic acid methyl ester

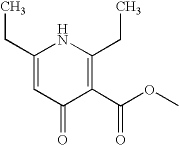

To a mixture of methyl 3-amino pentenoate (50 gm, 0.387 mol) and methyl propionyl acetate (50 gm, 0.384 mol) in o-xylene (200 ml) was added (50 gm) of 40 A molecular sieves. The reaction mixture was stirred and heated to reflux with a Dean Stark apparatus for 5 days. The mixture was cooled to room temperature and molecular sieves were filtered off from the reaction mixture. The filtrate was concentrated and the crude compound was purified on a silica gel column using 50% dichloromethane: methanol as an eluent to provide 20 gm of desired product as an off white solid

### STEP 03: Synthesis of 2,6-diethyl-4-(toluene-4-sulphonylamino) nicotinic acid methyl ester

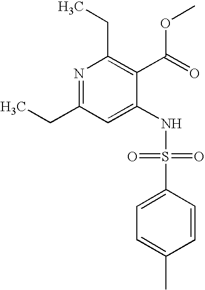

Under flow of dry nitrogen gas tosyl isocyanate (39 gm, 0.197 mol) was added to a stirred suspension of methyl 2,6-diethyl-4-oxo-1,4-dihydropyridine-3-carboxylate (25 gm, 0.119 mol) in acetonitrile (250 ml). After the initial exotherm had subsided the mixture was refluxed for 2 hours. The reaction mixture was cooled to room temperature and the suspended solid product was collected by filtration to give 20 gm of 2,6-diethyl-4-(toluene-4-sulphonylamino) nicotinic acid methyl ester.

### STEP 04: Synthesis of 4-amino-2,6-diethyl-nicotinic acid methyl ester

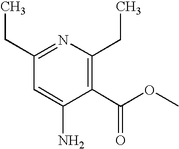

2,6-Diethyl-4-(toluene-4-sulphonylamino)nicotinic acid methyl ester (40 g, 0.110 mol) was added to concentrated sulphuric acid (57 ml, 1.10 mol) at 0° C. and then the reaction mixture was stirred at 50° C. for 1 hour. The reaction mixture was cooled to room temperature and poured onto ice. The pH of this solution was adjusted to 8 by addition of solid sodium carbonate and the solution was then extracted with dichloro methane (200 ml×2). The combined organic layers were washed with water and brine and dried over sodium sulphate and concentrated to yield 19 gm methyl-4-amino-2,6-diethyl pyridine-3-carboxylate as a white solid

### STEP 05: Synthesis of 5,7-diethyl-4-hydroxy-2-oxo-1,2-dihydro-[1,6]naphthyridine-3-carboxylic acid ethyl ester

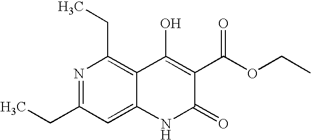

Diethylmalonate (15 ml, 0.093 mol) and methyl-4-amino-2,6-diethylpyridine-3-carboxylate (19.0 gm, 0.09 mol) were added to a solution of sodium ethoxide (7 gm, 0.10 mol) in ethanol (60 ml) and this reaction mixture was heated at 150° C. and 100 psi pressure for 20 hours in an autoclave. The mixture was allowed to cool and the volatile material was removed by evaporation and the resulting semi solid was triturated with ether to give a white solid which was collected by filtration The solid was dissolved in water and the solution was then acidified with 1.5 N hydrochloric acid to give a white solid which was filtered and suction dried to yield 11 gm of ethyl 5,7-diethyl-4-hydroxy-2-oxo-1,2-dihydro-1,6-naphthyridine-3-carboxylate as an off white solid.

### STEP 06: Synthesis of 5,7-diethyl-4-hydroxy-1H-[1,6]naphthyridin-2-one

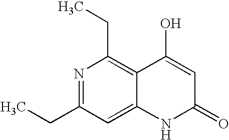

Ethyl 5,7-diethyl-4-hydroxyl-2-oxo-1,2-dihydro-1,6-naphthyridine-3-carboxylate (11 gm) was dissolved in a mixture of water (11 ml), 1,4-dioxane (22 ml) and concentrated hydrochloric acid (11 ml) and the reaction mixture was heated to reflux for 3 hours. The reaction mixture was then cooled and the suspended solid was filtered off, washed with ethanol and ether and suction dried to give 4.3 gm of 5,7-diethyl-4-hydroxy-1,6-naphthyridin-2(1H)-one as an off white solid.

### STEP 07: Synthesis of 4-chloro-5,7-diethyl-1,6-naphthyridin-2(1H)-one

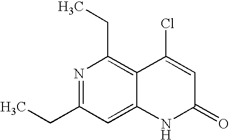

(4.3 gm, 0.019 mol) of 5,7-diethyl-4-hydroxy 1,6-naphthyridin-2(1H)-one was dissolved in 22 ml phosphorous oxy chloride and the reaction mixture was refluxed for 24 hours. The reaction mixture was concentrated and the residue was dissolved in concentrated hydrochloric acid (16 ml) and 22 ml of water and refluxed for 4 hours. The mixture was diluted with water and basified with solid sodium bicarbonate and the resulting solid was collected by filtration, washed with water and suction dried to give 3.0 gm of 4-chloro-5,7-diethyl-1,6-naphthyridin-2(1H)-one as an orange coloured solid.

### STEP 08: Synthesis of (5-methyl-isoxazol-3-yl) carbamic acid tert-butyl ester

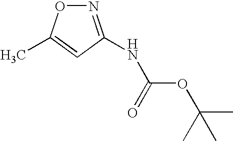

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 09: Synthesis of (4,5-dimethyl-isoxazol-3-yl) carbamic acid tert-butyl ester

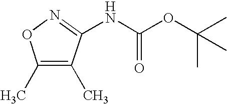

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 10: Synthesis of 4,5-dimethyl-isoxazol-3-yl-amine

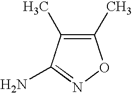

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 11: Synthesis of 3-bromo-thiophene-2-sulphonyl chloride

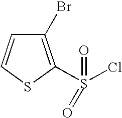

3-Bromothiophene (10 gm, 0.0617 mol) was dissolved in methylene chloride (60 ml) and the reaction mixture was cooled to −78° C. Then chlorosulphonic acid (25 ml, 0.396 mol) was added drop wise at −78° C. The temperature of the reaction mixture was slowly raised to 0° C. and maintained for 1 hour. The reaction mixture was slowly poured into ice cold water, followed by extraction with methylene chloride (100 ml×3). The combined organic extract was washed with water and brine and then dried over anhydrous sodium sulphate, evaporated under vacuum to give a brown color solid. The crude compound was purified by column chromatography on a silica gel column using hexane:ethyl acetate as an eluent to provide 5.4 gm of 3-bromo-thiophene-2-sulphonyl chloride.

### STEP 12: Synthesis of 3-bromo-thiophene-2-sulphonic acid (4,5-dimethyl-isoxazol-3yl) amide

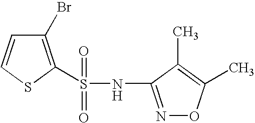

To the solution of 3-amino-4,5-dimethylisoxazole (2 gm, 0.0178 mol) in (25 ml) pyridine and dimethyl amino pyridine (0.230 gm, 0.0019 mol) was added 3-bromo-thiophene-2-sulphonyl chloride (5.0 gm, 0.01912) at 0° C. Then slowly the temperature of reaction mixture was raised to room temperature (28° C.), and then the reaction mixture was stirred for 6 hours. The reaction mixture was then concentrated under vacuum, the residue was acidified using 1N hydrochloric acid to pH 1 followed by extraction with dichloro methane (50 ml×3). The combined organic extract was washed with water and brine. Organic layer was dried over sodium sulphate and concentrated to give 3.5 gm of 3-bromo-thiophene-2-sulphonic acid (4,5-dimethyl-isoxazol-3yl) amide as brown colored solid.

### STEP 13: Synthesis of 3-bromo-thiophene-2-sulphonic acid (4,5 dimethyl-isoxazol-3-yl)-(2-methoxy-ethoxymethyl)-amide

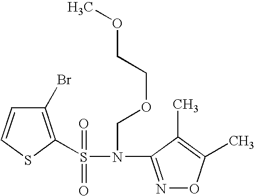

Under the flow of dry nitrogen, sodium hydride (0.600 gm, 0.0125 mol, 60% in mineral oil) was added portion wise to the solution of 3-bromo-thiophene-2-sulphonic acid (4,5-dimethyl-isoxazol-3yl) amide (3.5 gm, 0.0103 mol) in N,N-dimethyl formamide (30 ml) at −15° C. After completion of the addition reaction mixture was stirred at room temperature for 30 min. Then the reaction mixture was recooled to 0° C. To this reaction mixture 2-methoxyethoxy methylchloride (1.55 gm, 0.0124 mol) was added drop wise within 30 min, maintaining the temperature of the reaction mixture at 0° C. The reaction mixture was stirred at 0° C. for 30 min and then at room temperature for 4 hrs. Then the reaction mixture was diluted with ethyl acetate (100 ml) followed by addition of (30 ml) ice cold water. The organic layer was separated, washed with water and brine. Finally the organic layer was dried over sodium sulphate and concentrated under vacuum. The crude compound was purified by column chromatography on a silica gel column using hexane:ethyl acetate as eluent to provide 2.7 gm of 3-bromo-thiophene-2-sulphonic acid (4,5 dimethyl-isoxazol-3-yl)-(2-methoxy-ethoxymethyl)-amide as yellow oil.

### STEP 14: Synthesis of 3-(4-formyl-phenyl)-thiophene-2-sulphonic acid (4,5-dimethyl-isoxazol-3-yl)-(2-methoxy-ethoxymethyl) amide

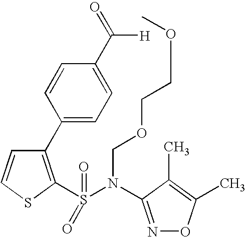

To a stirred solution 2.7 gm (0.0063 mol) of 3-bromo-thiophene-2-sulphonic acid (4,5 dimethyl-isoxazol-3-yl)-(2-methoxy-ethoxymethyl)-amide and 4-formyl boronic acid

1.04 gm (0.006 mol) in toluene (15 ml) and ethanol (12 ml) under nitrogen atmosphere was added an 2M aqueous sodium carbonate solution (2 gm in 16 ml water). This reaction mixture was stirred for 15 minutes, then tetrakis triphenyl phosphine palladium (0) (0.40 gm, 0.00034 mol) was added. The reaction mixture was heated to 85° C. for 6 hrs and then cooled to room temperature where upon ethyl acetate (50 ml) was added. The mixture was concentrated under vacuum and to the residual mass ethyl acetate (100 ml) was added, followed by chilled water and further extraction with ethyl acetate (100 ml×2). The combined extract was washed with water and brine and dried over sodium sulphate and concentrated under vacuum. The crude compound was purified on a silica gel column using hexane:ethyl acetate as an eluent to provide 1.1 gm of 3-(4-formyl-phenyl)-thiophene-2-sulphonic acid (4,5-dimethyl-isoxazol-3-yl)-(2-methoxy-ethoxymethyl) amide as an oil.

### STEP 15: Synthesis of 3-(4-hydroxymethyl-phenyl)-thiophene-2-sulphonic acid (4,5-dimethyl-isoxazol-3-yl)-(2-methoxy-ethoxymethyl)-amide

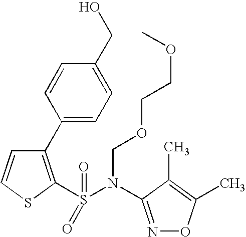

Lithium aluminum hydride (0.100 gm, 0.0029 mol) was added under flow of nitrogen to a stirred solution of tetrahydrofuran (15 ml) at 0° C., followed by addition of 3-(4-formyl-phenyl)-thiophene-2-sulphonic acid (4,5-dimethyl-isoxazol-3-yl)-(2-methoxy-ethoxymethyl) amide (1.1 gm, 0.0024 mol) in (15 ml) tetrahydrofuran. The reaction mixture was stirred at 0° C. for 1 hr and the temperature was then raised to room temperature (28° C.) and stirred for 4 hrs. The reaction was worked up by addition of sodium hydroxide solution (1 gm dissolved in 100 ml water) at 0° C. followed by extraction with ethyl acetate (50 ml×2). The combined organic layers were washed with water and brine and dried over sodium sulphate and concentrated under vacuum to give 1.0 gm of 3-(4-hydroxymethyl-phenyl)-thiophene-2-sulphonic acid (4,5-dimethyl-isoxazol-3-yl)-(2-methoxy-ethoxymethyl)-amide as an oil.

### STEP 16: Synthesis of 3-(4-methanesulphonyl methyl-phenyl)-thiophene-2-sulphonic acid (4,5-dimethyl-isoxazol-3-yl)-(2-methoxy-ethoxymethyl)amide

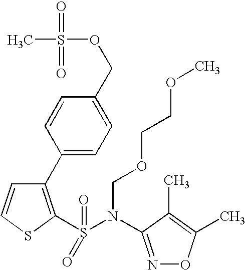

To the 0° C. cooled solution of 3-(4-hydroxymethyl-phenyl)-thiophene-2-sulphonic acid (4,5-dimethyl-isoxazol-3-yl)-(2-methoxy-ethoxymethyl)-amide (1.0 gm, 0.0022 mol) in (60 ml) of dichloro methane, N-Ethyl diisopropyl amine (0.6 ml, 0.0033 mol) was added, followed by slow addition of the solution of methane sulphonyl chloride (0.2 ml, 0.0033 mol) in (10 ml) dichloromethane in to the reaction mixture. The reaction mixture was then warmed and stirred at room temperature for 3 hrs. Workup was done by addition of ice-cold water into the reaction mixture followed by extraction with methylene dichloride (25 ml×2). The combined organic extract was washed with dilute hydrochloric acid followed by washings with water and brine. The organic layer was dried over sodium sulphate and concentrated under vacuum. The crude compound was purified by column chromatography on a silica gel column using hexane/ethyl acetate as an eluent to provide 0.700 gm of 3-(4-methanesulphonyl methyl-phenyl)-thiophene-2-sulphonic acid (4,5-dimethyl-isoxazol-3-yl)-(2-methoxy-ethoxymethyl) amide as a viscous liquid.

### STEP 17: Synthesis of 3-[4-(4-chloro-5,7-diethyl-2-oxo-2H-[1,6]naphthyridin-1ylmethyl)-phenyl]-thiophene-2-sulphonic acid(4,5-dimethyl-isoxazol-3-yl)-(2-methoxy-ethoxymethyl)-amide

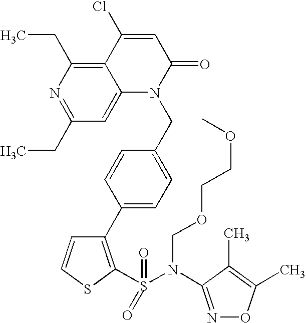

4-chloro-5,7-diethyl-1,6-naphthyridine-2-(1H)-one (0.16 gm, 0.6 mmol) was charged to a suspension of sodium hydride (0.053 gm, 1.0 mmol, 50%) in (5 ml) N,N dimethyl formamide at 0° C. under nitrogen atmosphere and the mixture was stirred for 30 minutes at room temperature. The reaction mixture was re-cooled to 0° C. and to this a solution of 3-(4-methanesulphonyl methyl-phenyl)-thiophene-2-sulphonic acid (4,5-dimethyl-isoxazol-3-yl)-(2-methoxy-ethoxymethyl) amide (0.367 gm, 0.7 mmol) in (5 ml) N,N dimethyl formamide was added drop wise. The reaction mixture was then stirred at room temperature for 20 hours and was then diluted with 40 ml ethyl acetate followed by 10 ml cold water. The organic phase was separated and the aqueous layer again extracted with 20 ml of ethyl acetate. The combined organic layer was washed with water and brine, dried over sodium sulphate and concentrated to give crude 0.350 gm of 3-[4-(4-Chloro-5,7-diethyl-2-oxo-2H-[1,6]naphthyridin-1ylmethyl)-phenyl]-thiophene-2-sulphonic acid(4,5-dimethyl-isoxazol-3-yl)-(2-methoxy-ethoxymethyl)-amide as an brown oil.

### STEP 18: 3-[4-(5,7-diethyl-2-oxo-4-phenoxy-2H-[1,6]naphthyridin-1ylmethyl)-phenyl]-thiophene-2-sulphonic acid(4,5-dimethyl-isoxazol-3-yl)-(2-methoxy-ethoxymethyl)-amide

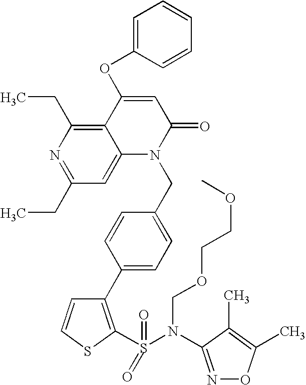

To a solution of phenol (56 mg, 0.59 mmol) in (3 ml) N,N-dimethyl formamide, at 0° C. was added portion wise sodium hydride (31 mg, 0.64 mmol 50%) and the reaction mixture was stirred until the effervescence ceased. Then a solution of 3-[4-(4-chloro-5,7-diethyl-2-oxo-2H-[1,6]naphthyridin-1 ylmethyl)-phenyl]-thiophene-2-sulphonic acid(4,5-dimethyl-isoxazol-3-yl)-(2-methoxy-ethoxymethyl)-amide (350 mg, 0.50 mmol,) in (3 ml) of N,N-dimethyl formamide was added drop wise. After the completion of addition, the temperature of the reaction mixture was slowly raised to room temperature and stirred at room temperature for 14 hrs. The reaction mixture was diluted with ethyl acetate and stirred for 10 min and then acidified with dilute hydrochloric acid to pH 5 and extracted with ethyl acetate. The organic layer was separated and washed with water and brine and dried over sodium sulphate and concentrated under vacuum to give crude 300 mg of 3-[4-(5,7-diethyl-2-oxo-4-phenoxy-2H-[1,6]naphthyridin-1ylmethyl)-phenyl]-thiophene-2-sulphonic acid(4,5-dimethyl-isoxazol-3-yl)-(2-methoxy-ethoxymethyl)-amide as a viscous mass.

### STEP 19: 3-[4-(5,7-diethyl-2-oxo-4-phenoxy-2H-[1,6]naphthyridin-1ylmethyl)-phenyl]-thiophene-2-sulphonic acid(4,5-dimethyl-isoxazol-3-yl)-amide

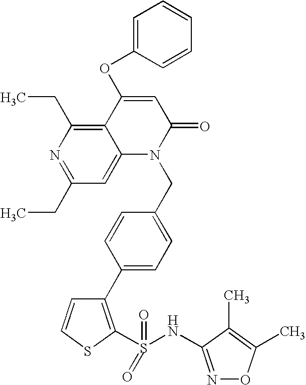

(0.30 gm, 0.41 mmol) of 3-[4-(5,7-diethyl-2-oxo-4-phenyl sulphanyl-2H-[1,6]naphthyridin-1 ylmethyl)-phenyl]-thiophene-2-sulphonic acid(4,5-dimethyl-isoxazol-3-yl)-(2-methoxy-ethoxymethyl)-amide was dissolved in (5 ml) ethanol hereafter (5 ml) 6N hydrochloric acid was added to it and the mixture refluxed for 3 hrs. The reaction mixture was then concentrated under vacuum and the residue thus obtained was diluted with water and the pH of this solution was adjusted to 5 by saturated sodium bicarbonate solution and the mixture was then extracted with ethyl acetate (25 ml×2). The ethyl acetate layer was washed with water and brine and dried over sodium sulphate and concentrated under vacuum. The crude compound was purified by column chromatography on a silica gel column using hexane/ethyl acetate as an eluent to provide 20 mg of 3-[4-(5,7-diethyl-2-oxo-4-phenoxy-2H-[1,6]naphthyridin-1ylmethyl)-phenyl]-thiophene-2-sulphonic acid(4,5-dimethyl-isoxazol-3-yl)-amide as an off white solid.

Molecular Formula: C₃₄H₃₂N₄O₅S₂

Molecular Weight: 640.77

¹HNMR (DMSOd₆): 1.19 (t, J=7.2 Hz, 3H) 1.30 (t, J=7.2 Hz, 3H) 1.47 (s, 3H) 2.12 (s, 3H) 2.70-2.76 (m, 2H) 3.12-3.17 (m, 2H) 5.50 (s, 2H) 7.14-7.97 (m, 13H) 10.83 (s, 1H)

Mass Spectrum: (m⁺¹) 641

### Example 9

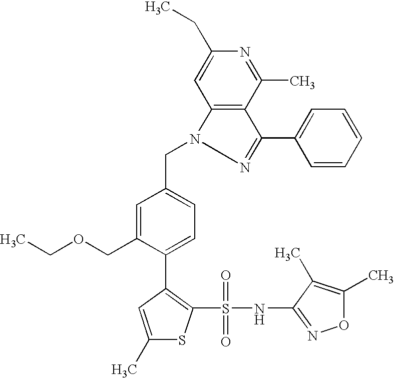

### 3-[2-ethoxymethyl-4-(6-ethyl-4-methyl-3-phenyl-pyrazolo[4,3-c]pyridine-1-ylmethyl)-phenyl]-5-methyl-thiophene-2-sulphonic acid-(4,5-dimethyl-isoxazol-3-yl)-(2-methoxy-ethoxymethyl)-amide

### STEP 01: Synthesis of 1-phenyl-butane-1,3 dione

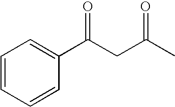

Sodium ethoxide (13.5 gm, 0.198 mol) was added to a stirred solution of dry ethyl acetate (80 ml, 0.72 mol) at −5° C. To this reaction mixture was added acetophenone (20 gm, 0.185 mol) at −5° C. and then the temperature of the reaction mixture was maintained at 0° C. for 12 hrs. The reaction mixture was then acidified with 1N hydrochloric acid and extracted with ethyl acetate (100 ml×2). The combined organic layer was washed with the water and brine. The organic layer was then dried over sodium sulphate and evaporated to give 21 gm of a yellow colored solid of 1-phenyl-butane-1,3 dione.

### STEP 02: Synthesis of 3-amino-1-phenyl-but-2-en-1-one

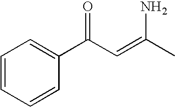

The mixture of 1-phenyl-butane-1,3 dione (20 gm, 0.123 mol) and ammonium acetate (38 gm, 0.49 mol) in dry methanol (200 ml) was stirred and heated at reflux for 24 hrs. The reaction mixture was concentrated under vacuum and to the residue was added chilled water, followed by extraction with ethyl acetate (100 ml×2). The combined extracts were washed with water and brine. Then the organic layer was dried over sodium sulphate and evaporated to give 19 gm of a yellow colored solid of 3-amino-1-phenyl-but-2-en-1-one.

### STEP 03: Synthesis of 5-(1-hydroxy propylidine)2,2-dimethyl-1,3-dioxane-4,6-dione

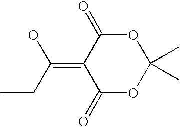

Propionyl chloride (7 ml, 0.0763 mol) was added within 30 min to a solution of Meldrum's acid (10 gm, 0.069 mol) in pyridine (12 ml, 0.138 mol) and methylene chloride (50 ml) at 0° C., and the temperature of the reaction mixture was then allowed to rise to ambient temperature and stirred for 1 hr. The reaction mixture was then acidified using 1N hydrochloric acid and extracted with methylene chloride (50 ml×2). The combined extracts were washed with water and brine. The organic layer was dried over sodium sulphate and concentrated under vacuum to give 10 gm of 5-(1-hydroxy propylidine) 2,2-dimethyl-1,3-dioxane-4,6-dione as a crystalline solid.

### STEP 04: Synthesis of 3-benzoyl-6-ethyl-2-methyl-1H-pyridin-4-one

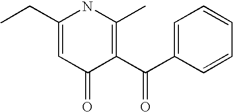

A mixture of 5-(1-hydroxy propylidine) 2,2-dimethyl-1,3-dioxane-4,6-dione (17.39 gm, 0.087 mol) and 3-amino-1-phenyl-but-2-en-1-one (10 gm, 0.062 mol) was stirred and heated at 120° C. for 2 hrs. The reaction mixture was purified by column chromatography over silica gel, eluting the desired fraction with 10% methanol and ethyl acetate to give 5.8 gm of 3-benzoyl-6-ethyl-2-methyl-1H-pyridin-4-one as a yellow colored solid.

### STEP 05: Synthesis of (4-chloro-6-ethyl-2-methyl-pyridin-3-yl)phenyl-methanone

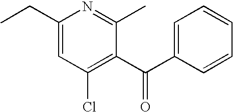

3-Benzoyl-6-ethyl-2-methyl-1H-pyridin-4-one (2.7 gm, 0.011 mol) was added to phosphorous oxy chloride (8 ml) at 0° C. The reaction mixture was stirred and heated to 50° C. and then the temperature was maintained for 8 hours. The work-up was done by evaporating the phosphorus oxy chloride under vacuum and the residue thus obtained was basified to pH 8 with saturated sodium bicarbonate solution, followed by extraction with methylene dichloride (50 ml×2). The combined organic extracts were washed with water and brine. The organic layer was dried over anhydrous sodium sulphate and concentrated to give 2.6 gm of (4-chloro-6-ethyl-2-methyl-pyridin-3-yl)phenyl-methanone as yellow oil.

### STEP 06: Synthesis of 6-ethyl-4-methyl-3-phenyl-1H-pyrazolo[4,3-c]pyridine

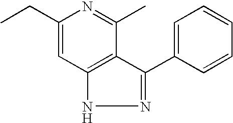

(4-Chloro-6-ethyl-2-methyl-pyridin-3-yl)phenyl-methanone (2.5 gm 0.0096 mol) was dissolved in ethanol (10 ml) and hydrazine hydrate (2.3 ml, 0.048 mol) was added to the reaction mixture. The mixture was stirred and heated to reflux for 4 hours. Then the reaction mixture was evaporated under vacuum. To the residue was added ice water, and the solid thus obtained was filtered off and suction dried to provide 1.8 gm of 6-ethyl-4-methyl-3-phenyl-1H-pyrazolo[4,3-c]pyridine.

### STEP 07: Synthesis of (5-methyl-isoxazol-3-yl) carbamic acid tert-butyl ester

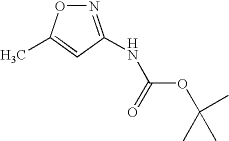

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 08: Synthesis of (4,5-dimethyl-isoxazol-3-yl) carbamic acid tert-butyl ester

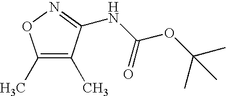

Under the dry nitrogen atmosphere (5-methyl-isoxazol-3-yl) carbamic acid tert-butyl ester

(20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 09: Synthesis of 4,5-dimethyl-isoxazol-3-yl-amine

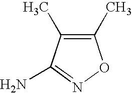

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 10: Synthesis of 5-methyl thiophene-2-sulphonyl chloride

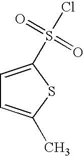

A solution of 2-methyl thiophene (50 gm, 0.51 mol) in chloroform was added to the solution of chloro sulphonic acid (105 ml, 1.53 mol) in chloroform at −5° C. to 0° C. The reaction temperature was then maintained at 0° C. for 3 hrs. The crude reaction mass was slowly dumped into the ice cold water, followed by extraction with chloroform (100 ml×2). The combined extract was washed with water and brine, dried over anhydrous sodium sulphate and evaporated under vacuum to give 16 gm of 5-methyl thiophene-2-sulphonyl chloride as a brown colored liquid.

### STEP 11: Synthesis of 5-methyl-thiophene-2-sulphonic acid (4,5-dimethyl-isoxazol-3yl) amide

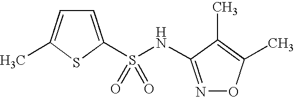

The solution of 5-Methyl thiophene-2-sulphonyl chloride (16.0 gm, 0.081 mol) in (25 ml) methylene chloride was added to the solution of 3-amino-4,5-dimethylisoxazole (6.2 gm, 0.055 mol) and dimethylaminopyridine (500 mg) in pyridine (40 ml) at 0° C. After the completion of the addition the temperature of the reaction mixture was slowly raised to room temperature and stirred it for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 18 gm of 5-methyl-thiophene-2-sulphonic acid (4,5-dimethyl-isoxazol-3yl) amide as brown colored solid.

### STEP 12: Synthesis of 5-methyl-thiophene-2-sulphonic acid (4,5 dimethyl-isoxazol-3-yl)-(2-methoxy-ethoxymethyl)-amide

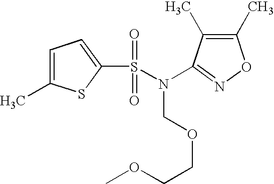

Under the flow of dry nitrogen and stirring, sodium hydride (3.4 gm, 0.07 mol, 60% dispersion in mineral oil) was added in to N,N-dimethyl formamide (40 ml) at 0° C., followed by addition of 5-methyl-thiophene-2-sulphonic acid (4,5-dimethyl-isoxazol-3yl) amide (16.5 gm, 0.060 mol) to it. After completion of the addition, temperature of the reaction mixture was slowly raised and stirred it at ambient temperature for 30 minutes then recooled the reaction mixture to 0° C., followed by the drop wise addition of methoxy ethoxy methyl chloride (8.03 gm, 0.064 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 18.2 gm of 5-methyl-thiophene-2-sulphonic acid (4,5-dimethyl-isoxazol-3yl) amide as yellowish oil.

### STEP 13: Synthesis of 3-borono-N-(4,5-dimethyl-3-isoxazolyl)-N-[(2-methoxy-ethoxy)methyl]-5-methyl-thiophene sulphonamide

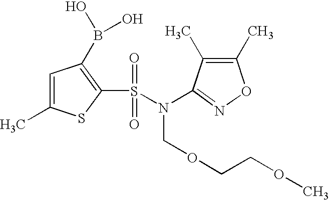

Under the dry nitrogen atmosphere the solution of (14 gm, 0.038 mol) 5-methyl-thiophene-2-sulphonic acid (4,5 dimethyl-isoxazol-3-yl)-(2-methoxy-ethoxymethyl)-amide in tetrahydrofuran (80 ml) was cooled to −78° C. To this n-Butyl lithium (60 ml, 0.097 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (15 ml, 0.062 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×3). The combined extract was washed with water and brine solution. The organic layer was dried over sodium sulphate and concentrated under vacuum to give 15 gm of 3-borono-N-(4,5-dimethyl-3-isoxazolyl)-N-[(2-methoxy-ethoxy)methyl]-5-methyl-thiophene sulphonamide as thick oily mass.

### STEP 14: Synthesis of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester

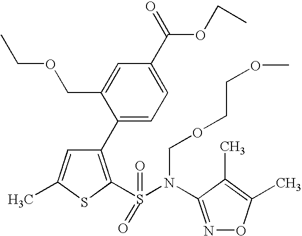

To a stirred solution of 3-borono-N-(4,5-dimethyl-3-isoxazolyl)-N-[(2-methoxy-ethoxy) methyl]-5-methyl-thiophene sulphonamide (15 gm, 0.037 mol) and 4-bromo-3-ethoxymethyl-benzoic acid ethyl ester (11 gm, 0.038 mol) in toluene (120 ml) and ethanol (60 ml) under nitrogen was added 2M aqueous sodium carbonate (4.0 gm in 19 ml water). The reaction mixture was stirred under nitrogen atmosphere for 15 minutes and then tetrakis triphenyl phosphine palladium (0) (2.15 gm, 0.0018 mol) was added. The reaction mixture was heated to 85° C. for 6 hrs, and was then concentrated and ethyl acetate (25 ml) was added to the residue followed by chilled water followed by extraction with ethyl acetate (100 ml×2). The combined extracts were washed with water and brine and dried over sodium sulphate and concentrated completely under vacuum. The crude compound was purified by column chromatography on a silica gel column using 4:1 hexane/ethyl acetate as eluent to provide 8 gm of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester as an oily mass.

### STEP 15: Synthesis of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

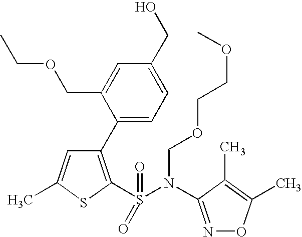

Lithium aluminium hydride (1.4 gm, 0.037 mol) was added to a stirred solution of tetrahydrofuran (25 ml) at 0° C. under a flow of nitrogen, followed by addition of (4-{2-[(4,5-dimethyl-isoxazol-3-yl)-(2-methoxy-ethoxymethyl)-sulphamoyl]-5-methyl-thiophene-3-yl}-3-ethoxymethyl-phenyl)-acetic acid ethyl ester (8 gm, 0.014 mol) in 35 ml of tetrahydrofuran. The reaction mixture was stirred at 0° C. for 1 hr and then the temperature was raised to room temperature and the mixture stirred for 4 hrs. The excess lithium aluminium hydride was destroyed by addition of sodium hydroxide solution (1 gm dissolved in 100 ml water) at 0° C. followed by extraction with ethyl acetate (25 ml×2). The organic layer was dried over sodium sulphate and concentrated completely under vacuum to give 4.7 gm of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

### STEP 16: Synthesis of methane sulphonic acid 4-{2-[(4,5-dimethyl-isoxazol-3yl)-(2-methoxy-ethoxy methyl)-sulphamoyl]-5-methylthiophene-3-yl}-3-ethoxy methyl-benzyl ester

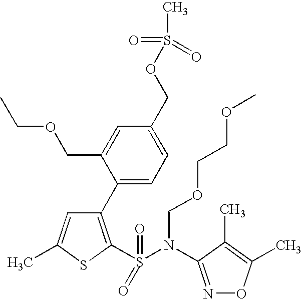

N-Ethyl diisopropyl amine (2.13 ml, 0.012 mol) was added to a solution of 3-(4-hydroxymethyl-phenyl)-5-methyl-thiophene-2-sulphonic acid (4,5-dimethyl-isoxazol-3-yl)-(2-methoxy-ethoxymethyl)-amide (3.2 gm, 0.0060 mol) in 10 ml of dichloro methane. The reaction mixture was cooled to 0° C., hereafter slowly methane sulphonyl chloride (0.6 ml, 0.0073 mol) was added into the reaction mixture. The mixture was maintained at room temperature for 3 hrs and was then dumped into ice-cold water followed by extraction with methylene chloride (50 ml×2). The combined extracts were washed with dilute hydrochloric acid followed by water and brine solution and the organic layer was dried over sodium sulphate and concentrated to give 3.3 gm of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester.

### STEP 17: Synthesis of 3-[2-ethoxymethyl-4-(6-ethyl-3,4-dimethyl-pyrazolo[4,3-c]pyridine-1-yl methyl)-phenyl]-5-methyl-thiophene-2sulphonic acid(4,5-dimethyl-isoxazol-3-yl)-(2-methoxy-ethoxymethyl)-amide

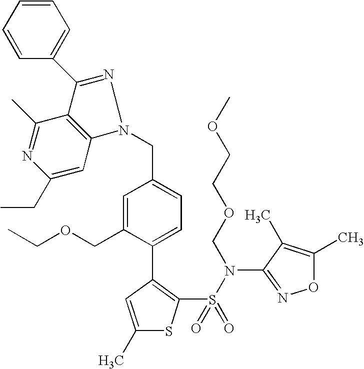

To a stirred solution of 6-ethyl-4-methyl-3-phenyl-1H pyrazolo[4,3-c]pyridine (0.454 gm, 0.0019 mol) in N,N-dimethyl formamide (6 ml) at −15° C. under flow of dry nitrogen, sodium hydride (60% in mineral oil) (0.110 gm, 0.0023 mol) was added portion wise. After the completion of the addition, the reaction mixture was warmed to ambient temperature and maintained for 30 min. The reaction mixture was re-cooled to 0° C. and the solution of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester (1.1 gm, 0.0018 mmol) in (6) ml N,N-dimethyl formamide was added drop wise to the reaction mixture. After the completion of the addition, the temperature of the reaction mixture was slowly raised to ambient temperature and was then stirred at room temperature for 24 hrs. The mixture was then diluted with ethyl acetate (40 ml), followed by addition of (10 ml) of cold water. The organic layer was washed with water and brine solution. Finally the organic layer was dried over sodium sulphate and evaporated under vacuum. The crude compound was purified on a silica gel column using 1:4 hexane/ethyl acetate as an eluent to provide 0.90 gm of 3-[2-ethoxymethyl-4-(6-ethyl-3,4-dimethyl-pyrazolo[4,3-c]pyridine-1-ylmethyl)-phenyl]-5-methyl-thiophene-2sulphonic acid(4,5-dimethyl-isoxazol-3-yl)-(2-methoxy-ethoxymethyl)-amide as a viscous oily mass.

### STEP 18: Synthesis of 3-[2-ethoxymethyl-4-(6-ethyl-4-methyl-3-phenyl-pyrazolo[4,3-c]pyridine-1-yl methyl)-phenyl]-5-methyl-thiophene-2-sulphonic acid(4,5 dimethyl-isoxazol-3-yl)-(2-methoxy-ethoxymethyl)-amide

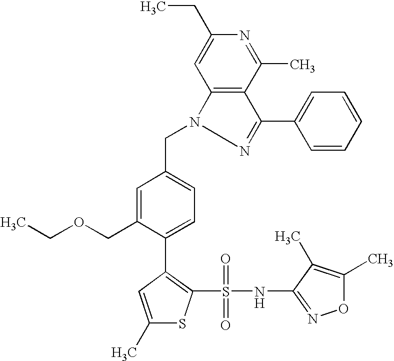

95% ethanol (10 ml) and 6N aqueous hydrochloric acid (10 ml) was added to (0.90 gm, 1.20 mmol) of 3-[2-ethoxymethyl-4-(6-ethyl-3,4-dimethyl-pyrazolo[4,3-c]pyridine-1-yl methyl)-phenyl]-5-methyl-thiophene-2sulphonic acid(4,5-dimethyl-isoxazol-3-yl)-(2-methoxy-ethoxymethyl)-amide at room temperature under stirring. The reaction mixture was refluxed for 3 hrs and then concentrated under vacuum. The residue thus obtained was diluted with water and the pH of the solution was adjusted to 8 using a saturated solution of sodium bicarbonate. The reaction solution was then acidified to pH 5 with acetic acid, and was then extracted with ethyl acetate (25 ml×2). The combined organic extract were washed with water and brine, and finally dried over sodium sulphate and concentrated under vacuum. The crude compound was purified on a silica gel column using 1:2 hexane/ethyl acetate as an eluent to provide 100 mg of 3-[2-ethoxymethyl-4-(6-ethyl-4-methyl-3-phenyl-pyrazolo[4,3-c]pyridine-1-yl methyl)-phenyl]-5-methyl-thiophene-2-sulphonic acid(4,5-dimethyl-isoxazol-3-yl)-(2-methoxy-ethoxymethyl)-amide.

Molecular Formula: C₃₅H₃₇N₅O₄S₂

Molecular Weight: 655.83

¹HNMR (DMSOd₆): 1.01 (t, J=6.8 Hz, 3H) 1.29 (t, J=7.6 Hz, 3H) 1.47 (s, 3H) 2.11 (s, 3H) 2.47 (s, 3H) 2.63 (s, 3H) 2.79-2.86 (m, 2H) 3.23-3.29 (m, 2H) 4.06 (s, 2H) 5.71 (s, 2H) 6.70 (s, 1H) 6.94-6.97 (m, 1H) 7.11-7.13 (m, 1H) 7.37 (s, 1H) 7.50-7.56 (m, 4H) 7.64-7.67 (m, 2H) 10.71 (s, 1H)

Mass Spectrum: (m⁺¹) 656

### Example 10

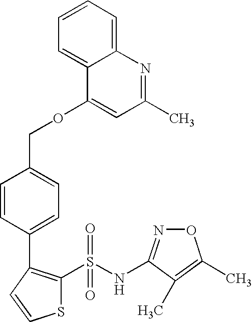

### 3-[4-(2-methyl-quinolin-4-yloxymethyl)-phenyl]-thiophene-2-sulphonic acid (4,5-dimethyl-isoxazol-3-yl)-amide

### STEP 01: Synthesis of (5-methyl-isoxazol-3-yl) carbamic acid tert-butyl ester

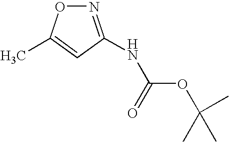

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 02: Synthesis of (4,5-dimethyl-isoxazol-3-yl) carbamic acid tert-butyl ester

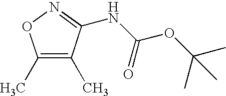

Under the dry nitrogen atmosphere (5-methyl-isoxazol-3-yl) carbamic acid tert-butyl ester

(20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 03: Synthesis of 4,5-dimethyl-isoxazol-3-yl-amine

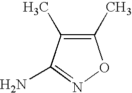

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 04: Synthesis of 3-bromo-thiophene-2-sulphonyl chloride

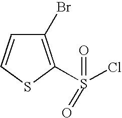

3-Bromothiophene (10 gm, 0.0617 mol) was dissolved in methylene chloride (60 ml) and this reaction mixture was cooled to −78° C. Then chlorosulphonic acid (25 ml, 0.396 mol) was added drop wise to the reaction mixture at −78° C. Slowly The temperature was then slowly raised to 0° C. and maintained at that temperature for 1 hour. The reaction mixture was slowly poured in to ice cold water, followed by extraction with methylene chloride (100 ml×3). The combined organic extract was washed with water and brine then dried over anhydrous sodium sulphate, evaporated under vacuum to give a brown colored solid. The crude compound was purified on a silica gel column using 4:1 hexane/ethyl acetate as an eluent to provide 5.4 gm of 3-bromo-thiophene-2-sulphonyl chloride.

### STEP 05: Synthesis of 3-bromo-thiophene-2-sulphonic acid (4,5-dimethyl-isoxazol-3yl) amide

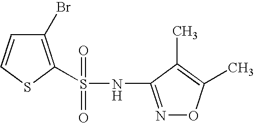

To the solution of 3-amino-4,5-dimethylisoxazole (2 gm, 0.0178 mol) in (25 ml) pyridine and dimethyl amino pyridine (0.230 gm, 0.0019 mol) was added 3-bromo-thiophene-2-sulphonyl chloride (5.0 gm, 0.01912) at 0° C. Then slowly the temperature of reaction mixture was raised to room temperature (28° C.), and then the reaction mixture was stirred for 6 hours. The reaction mixture was then concentrated under vacuum, the residue was acidified using 1N hydrochloric acid to pH 1 followed by extraction with dichloro methane (50 ml×3). The combined organic extract was washed with water and brine. Organic layer was dried over sodium sulphate and concentrated to give 3.5 gm of 3-bromo-thiophene-2-sulphonic acid (4,5-dimethyl-isoxazol-3yl) amide as brown colored solid.

### STEP 06: Synthesis of 3-bromo-thiophene-2-sulphonic acid (4,5 dimethyl-isoxazol-3-yl)-(2-ethoxymethyl)-amide

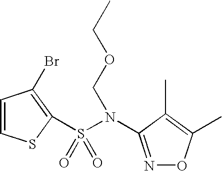

To the solution of 3-bromo-thiophene-2-sulphonic acid (4,5-dimethyl-isoxazol-3yl) amide (18.6 gm, 0.055 mol) in N,N-dimethyl formamide (60 ml) at −15° C., sodium hydride (60% in mineral oil) (3.17 gm, 0.066 mol) was added portion wise. After stirring at room temperature for 30 min, the reaction mixture was cooled to 0° C. using an ice-salt bath. To this reaction mixture, chloromethoxy ethane (7.82 gm, 0.082 mol) was added drop wise during 30 min, maintaining the temperature of the reaction mixture at 0° C. The reaction mixture was stirred with an ice-salt bath for 30 min and then at room temperature for 4 hrs. The reaction mixture was then diluted with ethyl acetate (100 ml) followed by 30 ml of ice cold water and the organic layer was separated, washed with water and brine and finally dried over sodium sulphate and concentrated under vacuum. The crude compound was purified on a silica gel column using 4:1 hexane/ethyl acetate as an eluent to provide 8.2 gm of 3-bromo-thiophene-2-sulphonic acid (4,5 dimethyl-isoxazol-3-yl)-(2-methoxy-ethoxymethyl)-amide as yellow oil.

### STEP 07: Synthesis of 3-(4-formyl-phenyl)-thiophene-2-sulphonic acid (4,5-dimethyl-isoxazol-3-yl)-ethoxymethyl-amide

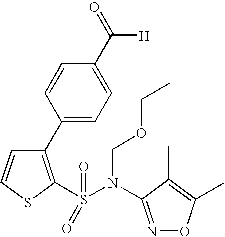

To a stirred solution of (7.8 gm, 0.0195 mol) 3-bromo-thiophene-2-sulphonic acid (4,5 dimethyl-isoxazol-3-yl)-ethoxymethyl-amide and 4-formyl boronic acid (2.96 gm, 0.0198 mol) in toluene (60 ml) and ethanol (30 ml) under nitrogen atmosphere was added 2M aqueous sodium carbonate (6.27 gm in 30 ml water) and the stirring of the mixture was continued for 15 minutes. Then tetrakis triphenyl phosphine palladium (0) (1.14 gm, 0.99 mmol) was added to the reaction mixture, which was then heated to 85° C. for 6 hrs. The reaction mixture was cooled to room temperature and 50 ml ethyl acetate was added. The reaction mixture was concentrated under vacuum and to the residue thus obtained was added ethyl acetate (100 ml) followed by chilled water. The layers were separated and the aqueous layer was further extracted with ethyl acetate (100 ml). The combined extract was washed with water and brine and dried over sodium sulphate and concentrated under vacuum. The crude compound was purified on a silica gel column using 4:1 hexane/ethyl acetate as an eluent to provide 10.2 gm of 3-(4-formyl-phenyl)-thiophene-2-sulphonic acid (4,5-dimethyl-isoxazol-3-yl)-ethoxymethyl-amide as an oily mass.

### STEP 08: Synthesis of 3-(4-hydroxymethyl-phenyl)-thiophene-2-sulphonic acid (4,5-dimethyl-isoxazol-3-yl)-ethoxymethyl-amide

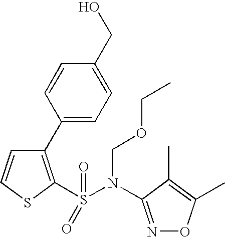

Under the flow of dry nitrogen lithium aluminium hydride (1.4 gm, 0.036 mol) was added to a stirred solution of tetrahydrofuran (20 ml) at 0° C., followed by addition of 3-(4-formyl-phenyl)-thiophene-2-sulphonic acid (4,5-dimethyl-isoxazol-3-yl)-ethoxymethyl-amide (10.0 gm, 0.024 mol) in 50 ml tetrahydrofuran. The reaction mixture was stirred at 0° C. for 1 hr and then the temperature was raised to room temperature and stirring continued for 4 hrs. The reaction mixture was then cooled to 0° C. and to it was added a sodium hydroxide solution 50 ml, (1 gm dissolved in 100 ml water) followed by extraction with ethyl acetate (50 ml×2). The organic layer was separated and washed with water and brine solution and was then dried over sodium sulphate and concentrated under vacuum to give 6.0 gm of 3-(4-hydroxymethyl-phenyl)-thiophene-2-sulphonic acid (4,5-dimethyl-isoxazol-3-yl)-ethoxymethyl-amide as an oily liquid.

### STEP 09: Synthesis of 3-(4-methanesulphonyl methyl-phenyl)-thiophene-2-sulphonic acid (4,5-dimethyl-isoxazol-3-yl)-ethoxymethyl-amide

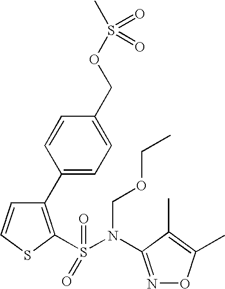

N-Ethyl diisopropyl amine (3.7 ml, 0.02 mol) was added to a solution of 3-(4-hydroxymethyl-phenyl)-thiophene-2-sulphonic acid (4,5-dimethyl-isoxazol-3-yl)-ethoxymethyl-amide, (6.0 gm, 0.0142 mol) in 60 ml of dichloro methane. The reaction mixture was cooled to 0° C. and then slowly a solution of methane sulphonyl chloride (1.32 ml, 0.0161 mol) in (10 ml) dichloromethane was added. After the addition, the temperature of the reaction mixture was maintained at room temperature for 3 hrs. Then ice-cold water was added, followed by extraction with methylene dichloride (25 ml×2). The combined organic extract was washed with dilute hydrochloric acid followed by water and brine solution. Finally the organic layer was dried over sodium sulphate and concentrated under vacuum. The crude compound was purified on a silica gel column using hexane/ethyl acetate as an eluent to provide 6.0 gm of 3-(4-methanesulphonyl methyl-phenyl)-thiophene-2-sulphonic acid (4,5-dimethyl-isoxazol-3-yl)-ethoxymethyl-amide as a viscous liquid.

### STEP 10: Synthesis of 3-[4-(2-methyl-quinolin-4-yloxymethyl)-phenyl]-thiophene-2-sulphonic acid (4,5-dimethyl-isoxazol-3-yl)ethoxymethyl-amide

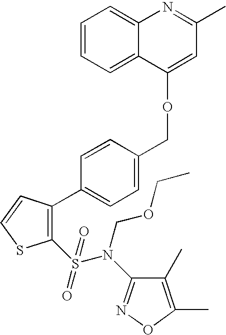

To a stirred solution of 2-methyl-1H-quinolin-4-one (0.16 gm, 1.0 mmol) in N,N-dimethyl formamide (5 ml) at 0° C. under the flow of dry nitrogen gas sodium hydride (60% in mineral oil) (72.0 mg, 1.5 mmol) was added portion wise. After completion of the addition, the temperature of the reaction mixture was raised to room temperature and maintained for 30 min. The reaction mixture was re-cooled to 0° C. and a solution of 3-(4-methanesulphonyl methyl-phenyl)-thiophene-2-sulphonic acid (4,5-dimethyl-isoxazol-3-yl)-ethoxymethyl-amide (0.5 gm, 1.0 mmol) in 5 ml N,N-dimethyl formamide was added drop wise. The reaction mixture was stirred at room temperature for 24 hrs and was then cooled to 0° C., hereafter ethyl acetate (40 ml) followed by 10 ml of water was added. The organic layers were separated; the aqueous layer extracted with ethyl acetate (50 ml×2) and the combined extract was washed with water and brine. Finally the organic layer was dried over sodium sulphate and concentrated under vacuum to give crude compound. The crude compound was purified on a silica gel column using 1:1 hexane/ethyl acetate as an eluent to provide 510 mg of 3-[4-(2-methyl-quinolin-4-yloxymethyl)-phenyl]-thiophene-2-sulphonic acid(4,5-dimethyl-isoxazol-3-yl)ethoxymethyl-amide as a gummy liquid.

### STEP 11: Synthesis of 3-[4-(2-methyl-quinolin-4-yloxymethyl)-phenyl]-thiophene-2-sulphonic acid(4,5-dimethyl-isoxazol-3-yl)-amide

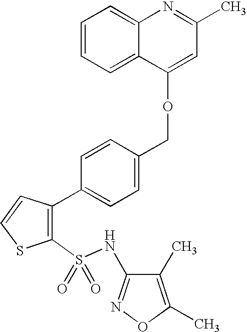

To (0.51 gm, 0.90 mmol) of 3-[4-(2-methyl-quinolin-4-yloxymethyl)-phenyl]-thiophene-2-sulphonic acid (4,5-dimethyl-isoxazol-3-yl)ethoxymethyl-amide was added ethanol (6 ml) and 6 ml of 6N aqueous hydrochloric acid at room temperature. The reaction mixture was refluxed for 3 hrs, and then concentrated under vacuum and the residue obtained was diluted with water. The pH of this solution was adjusted to 5 using sodium bicarbonate solution and extracted with ethyl acetate (50 ml×2). The combined organic extract was washed with water and brine solution. Finally the organic layer was dried over sodium sulphate and concentrated under vacuum. The crude compound was purified on a silica gel column using 1:1 hexane/ethyl acetate as an eluent to provide 90 mg of yellowish solid of 3-[4-(2-methyl-quinolin-4-yloxymethyl)-phenyl]-thiophene-2-sulphonic acid(4,5-dimethyl-isoxazol-3-yl)-amide.

Molecular Formula: C₂₆H₂₃N₃O₄S₂

Molecular Weight: 505.61

¹HNMR (DMSOd₆): 1.54 (s, 3H) 2.12 (s, 3H) 2.63 (s, 3H) 5.41 (s, 2H) 7.12-7.18 (m, 2H) 7.48-7.53 (m, 1H) 7.55-7.58 (m, 2H) 7.59-7.61 (m, 2H) 7.68-7.73 (m, 1H) 7.85-7.88 (m, 2H) 8.13-8.16 (m, 1H) 10.93 (s, 1H)

Mass Spectrum: (m⁻¹) 504

### Example 11

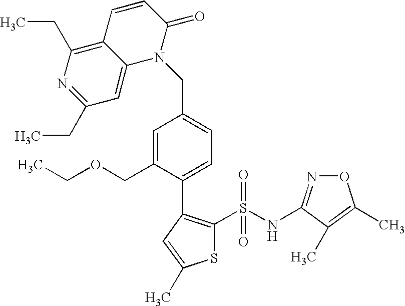

### 3-[4-(5,7-Diethyl-2-oxo-2H-[1,6]naphthyridin-1-ylmethyl)-2-ethoxymethyl-phenyl]-5-methyl-thiophene-2-sulphonic acid-(4,5-dimethyl-isoxazol-3-yl)-amide

### STEP 01: Synthesis of (5-methyl-isoxazol-3-yl) carbamic acid tert-butyl ester

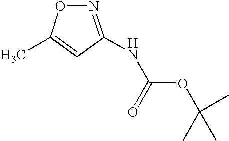

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 02: Synthesis of (4,5-dimethyl-isoxazol-3-yl) carbamic acid tert-butyl ester

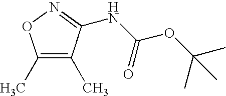

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 03: Synthesis of 4,5-dimethyl-isoxazol-3-yl-amine

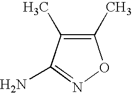

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 04: Synthesis of 5-methyl thiophene-2-sulphonyl chloride

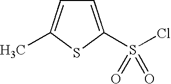

A solution of 2-methyl thiophene (50 gm, 0.51 mol) in chloroform (100 ml) was added to a solution of chloro sulphonic acid (105 ml, 1.53 mol) in chloroform at −5° C. to 0° C. The reaction mixture was maintained at 0° C. for 3 hrs. The crude reaction mass was slowly dumped into ice cold water, followed by extraction with chloroform (100 ml×2). The combined extract was washed with water and brine. Finally the organic layer was dried over anhydrous sodium sulphate, evaporated under vacuum to give 16 gm of 5-methyl thiophene-2-sulphonyl chloride as a brown colored liquid.

### STEP 05: Synthesis of 5-methyl-thiophene-2-sulphonic acid (4,5-dimethyl-isoxazol-3yl) amide

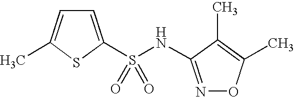

The solution of 5-Methyl thiophene-2-sulphonyl chloride (16.0 gm, 0.081 mol) in (25 ml) methylene chloride was added to the solution of 3-amino-4,5-dimethylisoxazole (6.2 gm, 0.055 mol) and dimethylaminopyridine (500 mg) in pyridine (40 ml) at 0° C. After the completion of the addition the temperature of the reaction mixture was slowly raised to room temperature and stirred it for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 18 gm of 5-methyl-thiophene-2-sulphonic acid (4,5-dimethyl-isoxazol-3yl) amide as brown colored solid.

### STEP 06: Synthesis of 5-methyl-thiophene-2-sulphonic acid (4,5 dimethyl-isoxazol-3-yl)-(2-methoxy-ethoxymethyl)-amide

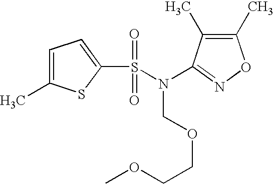

Under the flow of dry nitrogen and stirring, sodium hydride (3.4 gm, 0.07 mol, 60% dispersion in mineral oil) was added in to N,N-dimethyl formamide (40 ml) at 0° C., followed by addition of 5-methyl-thiophene-2-sulphonic acid (4,5-dimethyl-isoxazol-3yl) amide (16.5 gm, 0.060 mol) to it. After completion of the addition, temperature of the reaction mixture was slowly raised and stirred it at ambient temperature for 30 minutes then recooled the reaction mixture to 0° C., followed by the drop wise addition of methoxy ethoxy methyl chloride (8.03 gm, 0.064 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 18.2 gm of 5-methyl-thiophene-2-sulphonic acid (4,5-dimethyl-isoxazol-3yl) amide as yellowish oil.

### STEP 07: Synthesis of 3-borono-N-(4,5-dimethyl-3-isoxazolyl)-N-[(2-methoxy-ethoxy)methyl]-5-methyl-thiophene sulphonamide

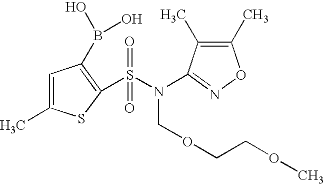

Under the dry nitrogen atmosphere the solution of (14 gm, 0.038 mol) 5-methyl-thiophene-2-sulphonic acid (4,5 dimethyl-isoxazol-3-yl)-(2-methoxy-ethoxymethyl)-amide in tetrahydrofuran (80 ml) was cooled to −78° C. To this n-Butyl lithium (60 ml, 0.097 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (15 ml, 0.062 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×3). The combined extract was washed with water and brine solution. The organic layer was dried over sodium sulphate and concentrated under vacuum to give 15 gm of 3-borono-N-(4,5-dimethyl-3-isoxazolyl)-N-[(2-methoxy-ethoxy)methyl]-5-methyl-thiophene sulphonamide as thick oily mass.

### STEP 08: Synthesis of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester

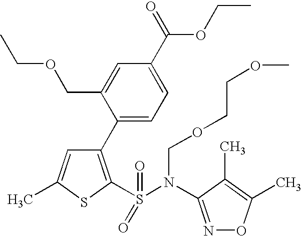

To a stirred solution of 4-bromo-3-ethoxymethyl-benzoic acid ethyl ester (4 gm, 0.0139 mol) in dimethoxy ethane (50 ml) under flow of dry nitrogen was added Bis (triphenylphosphine)palladium(II)chloride (1 gm, 0.00142 mol) followed by a 2M aqueous sodium carbonate solution (4.3 gm in 20 ml water). The reaction mixture was stirred at room temperature for 10 min and then heated at 60° C. To this a solution of 3-borono-N-(4,5-dimethyl-3-isoxazolyl)-N-[(2-methoxy-ethoxy)methyl]-5-methyl-thiophene sulphonamide (5.5 gm, 0.0136 mol in 25 ml dimethoxy ethane) was added drop wise within 45 min, and the reaction was refluxed for 60 min. After 60 min the same procedure was repeated with further addition of 3-borono-N-(4,5-dimethyl-3-isoxazolyl)-N-[(2-methoxy-ethoxy)methyl]-5-methyl-thiophene sulphonamide (5.5 gm, 0.0136 mol in 25 ml dimethoxy ethane) within 45 min, and finally the reaction mixture was refluxed for 4 hrs and stirred at room temperature for 12 hrs. The reaction mixture was cooled to room temperature and ethyl acetate (100 ml) was added followed by addition of water. The organic layers were separated, and the aqueous layer further extracted with ethyl acetate (50 ml×2). The combined organic extracts were washed with water and brine. Finally the organic layer was dried over sodium sulphate and concentrated under vacuum. The crude compound was purified on a silica gel column using 4:1 hexane/ethyl acetate as an eluent to provide 7 gm of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester as pale yellow oily mass.

### STEP 09: Synthesis of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

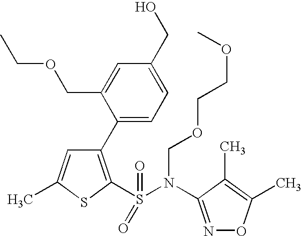

Lithium aluminium hydride (1.4 gm, 0.037 mol) was added to a stirred solution of tetrahydrofuran (20 ml) at 0° C. under flow of nitrogen, followed by addition of (4-{2-[(4,5-dimethyl-isoxazol-3-yl)-(2-methoxy-ethoxymethyl)-sulphamoyl]-5-methyl-thiophene-3-yl}-3-ethoxymethyl-phenyl)-acetic acid ethyl ester (8 gm, 0.014 mol) in 35 ml of tetrahydrofuran. The reaction mixture was stirred at 0° C. for 1 hr and then the temperature was raised to room temperature and the mixture stirred for 4 hrs. The excess lithium aluminium hydride was destroyed by addition of sodium hydroxide solution (1 gm dissolved in 100 ml water) at 0° C. followed by extraction with ethyl acetate (25 ml×2). The organic layer was dried over sodium sulphate and concentrated under vacuum to give 4.5 gm of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

### STEP 10: Synthesis of methane sulphonic acid 4-{2-[(4,5-dimethyl-isoxazol-3yl)-(2-methoxy-ethoxy methyl)-sulphamoyl]-5-methylthiophene-3-yl}-3-ethoxy methyl-benzyl ester

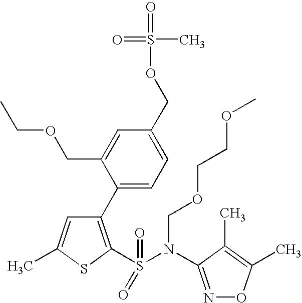

N-Ethyl diisopropyl amine (3.35 ml, 0.0193 mol) was added to a solution of 3-(4-hydroxymethyl-phenyl)-5-methyl-thiophene-2-sulphonic acid (4,5-dimethyl-isoxazol-3-yl)-(2-methoxy-ethoxymethyl)-amide (6.7 gm, 0.0127 mol) in 50 ml of dichloro methane. The reaction mixture was cooled to 0° C., where after methane sulphonyl chloride (1.8 gm, 0.0157 mol) was added slowly into the reaction mixture. The mixture was maintained at room temperature for 3 hrs and was then dumped into ice-cold water followed by extraction with methylene chloride (50 ml×2). The combined extracts were washed with dilute hydrochloric acid followed by water and brine solution and the organic layer was dried over sodium sulphate and concentrated to give 7.2 gm of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester as brown oil.

### STEP 11: Synthesis of 3-[4-(5,7-diethyl-2-oxo-2H-[1,6]naphthyridin-1ylmethyl)-2-ethoxymethyl-phenyl]-5-methyl-thiophene-2-sulphonic acid(4,5-dimethyl-isoxazol-3-yl)-(2-methoxy-ethoxymethyl)-amide

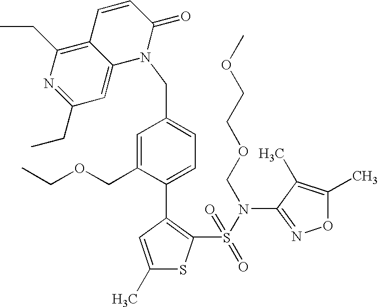

To the stirred solution of 5,7-diethyl-1H-[1,6] naphthyridin-2-one (0.7 gm, 0.00346 mol) in N,N-dimethyl formamide (6 ml) at −15° C. under flow of dry nitrogen, sodium hydride (60% in mineral oil) (0.166 gm, 0.00346 mol) was added portion wise. After the completion of the addition, the reaction mixture was warmed to ambient temperature and maintained for 30 min. Then the reaction mixture was re-cooled to 0° C. and a solution of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester (1.9 gm, 0.00315 mmol) in 6 ml N,N-dimethyl formamide was added drop wise at 0° C. The temperature of the reaction mixture was slowly raised to room temperature and was stirred for 24 hrs. The reaction mixture was then diluted with ethyl acetate (40 ml), followed by addition of 10 ml of cold water. The organic layer was separated and washed with water and brine solution. Finally the organic layer was dried over sodium sulphate and evaporated under vacuum. The crude compound was purified on a silica gel column using 1:1 hexane/ethyl acetate as an eluent to provide 1.2 gm of 3-[4-(5,7-diethyl-2-oxo-2H-[1,6]naphthyridin-1ylmethyl)-2-ethoxymethyl-phenyl]-5-methyl-thiophene-2-sulphonic acid(4,5-dimethyl-isoxazol-3-yl)-(2-methoxy-ethoxymethyl)-amide as a viscous oily mass.

### STEP 12: Synthesis of 3-[4-(5,7-diethyl-2-oxo-2H-[1,6]naphthyridin-1ylmethyl)-2-ethoxymethyl-phenyl]-5-methyl-thiophene-2-sulphonic acid(4,5-dimethyl-isoxazol-3-yl)-amide

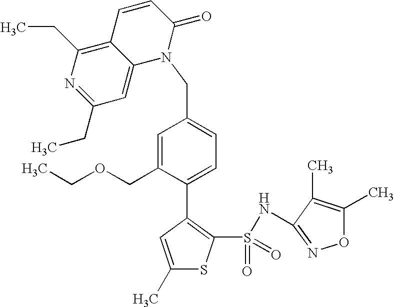

To (1.2 gm, 1.69 mmol) of 3-[4-(5,7-diethyl-2-oxo-2H-[1,6]naphthyridin-1 ylmethyl)-2-ethoxymethyl-phenyl]-5-methyl-thiophene-2-sulphonic acid(4,5-dimethyl-isoxazol-3-yl)-(2-methoxy-ethoxymethyl)-amide was added ethanol (10 ml) and 6N aqueous hydrochloric acid (8 ml) at room temperature under stirring. The reaction mixture was heated to 90° C. for 3 hrs and then concentrated under vacuum. The residue thus obtained was diluted with water and the pH of the solution was adjusted to 8 using a saturated solution of sodium bicarbonate. This solution was then extracted with ethyl acetate (35 ml×2) and the combined organic extract was washed with water and brine solution. Finally the organic layer was dried over sodium sulphate and concentrated under vacuum. The crude compound was purified by column chromatography on a silica gel column using hexane:ethyl acetate as an eluent to provide 700 mg of 3-[4-(5,7-diethyl-2-oxo-2H-[1,6]naphthyridin-1 ylmethyl)-2-ethoxymethyl-phenyl]-5-methyl-thiophene-2-sulphonic acid(4,5-dimethyl-isoxazol-3-yl)-amide.

Molecular Formula: C₃₂H₃₆N₄O₅S₂

Molecular Weight: 620.78

¹HNMR (DMSOd₆): 0.98 (t, J=6.8 Hz, 3H) 1.19 (t, J=6.8 Hz, 3H) 1.24 (t, J=7.6 Hz, 3H) 1.47 (s, 3H) 2.11 (s, 3H) 2.47 (s, 3H) 2.69-2.75 (m, 2H) 3.04-3.10 (m, 2H) 3.19-3.25 (m, 2H) 4.05 (s, 2H) 5.52 (s, 2H) 6.70-6.75 (m, 2H) 6.89-6.92 (m, 1H) 7.03-7.06 (m, 1H) 7.16 (s, 1H) 7.32 (s, 1H) 8.23-8.26 (m, 1H) 10.65 (s, 1H)

Mass Spectrum: (m⁻¹) 619

### Example 12

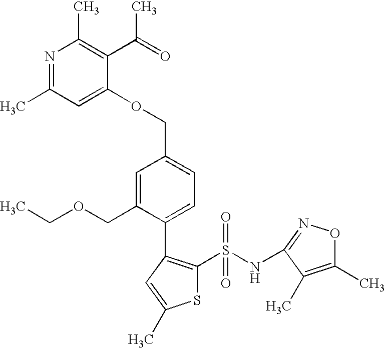

### 3-[4-(3-Acetyl-2,6-dimethyl-pyridine-4-yloxymethyl)-2-ethoxymethyl-phenyl]-5-methyl-thiophene-2-sulphonic acid(4,5-dimethyl-isoxazol-3-yl)-amide

### STEP 01: Synthesis of (5-methyl-isoxazol-3-yl) carbamic acid tert-butyl ester

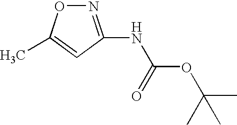

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 02: Synthesis of (4,5-dimethyl-isoxazol-3-yl) carbamic acid tert-butyl ester

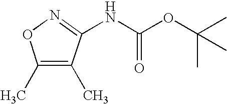

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 03: Synthesis of 4,5-dimethyl-isoxazol-3-yl-amine

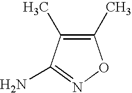

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 04: Synthesis of 5-methyl thiophene-2-sulphonyl chloride

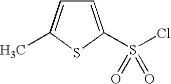

A solution of 2-methyl thiophene (50 gm, 0.51 mol) in chloroform was added to a solution of chloro sulphonic acid (105 ml, 1.53 mol) in chloroform at −5° C. to 0° C. The reaction mixture was maintained at 0° C. for 3 hrs and the crude reaction mass was then slowly dumped into the ice cold water, followed by extraction with chloroform (100 ml×2). The combined extract was washed with water and brine. Finally the organic layer was dried over anhydrous sodium sulphate, evaporated under vacuum to give 16 gm of 5-methyl thiophene-2-sulphonyl chloride as a brown colored liquid.

### STEP 05: Synthesis of 5-methyl-thiophene-2-sulphonic acid (4,5-dimethyl-isoxazol-3yl) amide

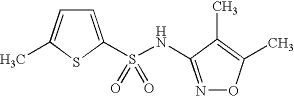

The solution of 5-Methyl thiophene-2-sulphonyl chloride (16.0 gm, 0.081 mol) in (25 ml) methylene chloride was added to the solution of 3-amino-4,5-dimethylisoxazole (6.2 gm, 0.055 mol) and dimethylaminopyridine (500 mg) in pyridine (40 ml) at 0° C. After the completion of the addition the temperature of the reaction mixture was slowly raised to room temperature and stirred it for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 18 gm of 5-methyl-thiophene-2-sulphonic acid (4,5-dimethyl-isoxazol-3yl) amide as brown colored solid.

### STEP 06: Synthesis of 5-methyl-thiophene-2-sulphonic acid (4,5 dimethyl-isoxazol-3-yl)-(2-methoxy-ethoxymethyl)-amide

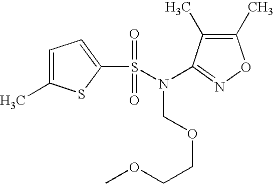

Under the flow of dry nitrogen and stirring, sodium hydride (3.4 gm, 0.07 mol, 60% dispersion in mineral oil) was added in to N,N-dimethyl formamide (40 ml) at 0° C., followed by addition of 5-methyl-thiophene-2-sulphonic acid (4,5-dimethyl-isoxazol-3yl) amide (16.5 gm, 0.060 mol) to it. After completion of the addition, temperature of the reaction mixture was slowly raised and stirred it at ambient temperature for 30 minutes then recooled the reaction mixture to 0° C., followed by the drop wise addition of methoxy ethoxy methyl chloride (8.03 gm, 0.064 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 18.2 gm of 5-methyl-thiophene-2-sulphonic acid (4,5-dimethyl-isoxazol-3yl) amide as yellowish oil.

### STEP 07: Synthesis of 3-borono-N-(4,5-dimethyl-3-isoxazolyl)-N-[(2-methoxy-ethoxy)methyl]-5-methyl-thiophene sulphonamide

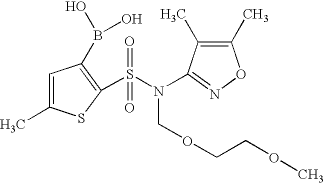

Under the dry nitrogen atmosphere the solution of (14 gm, 0.038 mol) 5-methyl-thiophene-2-sulphonic acid (4,5 dimethyl-isoxazol-3-yl)-(2-methoxy-ethoxymethyl)-amide in tetrahydrofuran (80 ml) was cooled to −78° C. To this n-Butyl lithium (60 ml, 0.097 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (15 ml, 0.062 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×3). The combined extract was washed with water and brine solution. The organic layer was dried over sodium sulphate and concentrated under vacuum to give 15 gm of 3-borono-N-(4,5-dimethyl-3-isoxazolyl)-N-[(2-methoxy-ethoxy)methyl]-5-methyl-thiophene sulphonamide as thick oily mass.

### STEP 08: Synthesis of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester

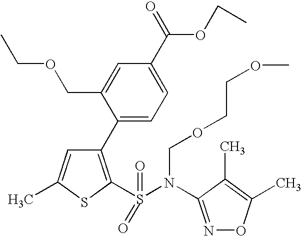

To a stirred solution of 4-bromo-3-ethoxymethyl-benzoic acid ethyl ester (4 gm, 0.0139 mol) in dimethoxy ethane (50 ml) under flow of dry nitrogen, bis(triphenylphosphine)palladium(II)chloride (1 gm, 0.00142 mol) was added followed by a 2M aqueous sodium carbonate solution (4.3 gm in 20 ml water). The reaction mixture was stirred at room temperature for 10 min and then heated at 60° C. To this mixture a solution of 3-borono-N-(4,5-dimethyl-3-isoxazolyl)-N-[(2-methoxy-ethoxy)methyl]-5-methyl-thiophene sulphonamide (5.5 gm, 0.0136 mol in 25 ml dimethoxy ethane) was added within 45 min and the reaction was then refluxed for 60 min. After 60 min the same procedure was repeated with further addition of 3-borono-N-(4,5-dimethyl-3-isoxazolyl)-N-[(2-methoxy-ethoxy)methyl]-5-methyl-thiophene sulphonamide (5.5 gm, 0.0136 mol in 25 ml dimethoxy ethane) within 45 min. Finally the reaction mixture was refluxed for 4 hrs and stirred at room temperature for 12 hrs. The reaction mixture was cooled to room temperature and ethyl acetate (100 ml) was added in it followed by addition of water. Then the organic layers were separated, and the aqueous layer further extracted with ethyl acetate (50 ml×2). The combined organic extract was washed with water and brine. Finally the organic layer was dried over sodium sulphate and concentrated under vacuum. The crude compound was purified on a silica gel column using 4:1 hexane/ethyl acetate as an eluent to provide 7 gm of (4-{2-[(4,5-Dimethyl-isoxazol-3-yl)-(2-methoxy-ethoxymethyl)-sulphamoyl]-5-methyl-thiophene-3-yl}-3-ethoxymethyl-benzoic acid ethyl ester as a pale yellow oily mass.

### STEP 09: Synthesis of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

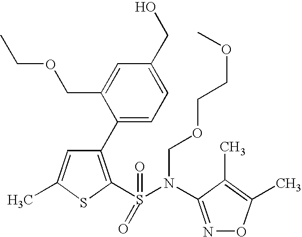

Lithium aluminium hydride (1.4 gm, 0.037 mol) was added to a stirred solution of tetrahydrofuran (20 ml) at 0° C. under flow of nitrogen, followed by addition of (4-{2-[(4,5-dimethyl-isoxazol-3-yl)-(2-methoxy-ethoxymethyl)-sulphamoyl]-5-methyl-thiophene-3-yl}-3-ethoxymethyl-phenyl)-acetic acid ethyl ester (8 gm, 0.014 mol) in 35 ml of tetrahydrofuran. The reaction mixture was stirred at 0° C. for 1 hr and then the temperature was raised to room temperature and the mixture stirred for 4 hrs. The excess lithium aluminium hydride was destroyed by addition of a sodium hydroxide solution (1 gm dissolved in 100 ml water) at 0° C. followed by extraction with ethyl acetate (25 ml×2). The organic layer was dried over sodium sulphate and concentrated under vacuum to give 4.5 gm of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

### STEP 10: Synthesis of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester

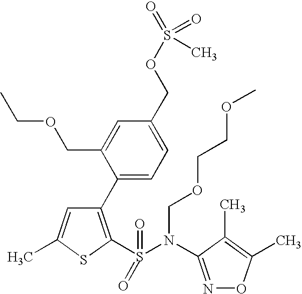

N-Ethyl diisopropyl amine (3.35 ml, 0.0193 mol) was added to a solution of 3-(4-hydroxymethyl-phenyl)-5-methyl-thiophene-2-sulphonic acid (4,5-dimethyl-isoxazol-3-yl)-(2-methoxy-ethoxymethyl)-amide (6.7 gm, 0.0127 mol) in 50 ml of dichloro methane. The reaction mixture was cooled to 0° C., where after methane sulphonyl chloride (1.8 gm, 0.0157 mol) was added slowly. The reaction mixture was maintained at room temperature for 3 hrs and was then dumped into ice-cold water followed by extraction with methylene chloride (50 ml×2). The combined extracts were washed with dilute hydrochloric acid followed by water and brine solution and the organic layer was dried over sodium sulphate and concentrated to give 7.2 gm of methane sulphonic acid 4-{2-[(4,5-dimethyl-isoxazol-3yl)-(2-methoxy-ethoxy methyl)-sulphamoyl]-5-methylthiophene-3-yl}-3-ethoxy methyl-benzyl ester as brown oil.

### STEP 11: Synthesis of 3-acetyl-2,6-dimethyl-1H-pyridin-4-one

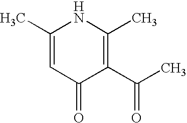

A mixture of 12 gm (0.12 mol) 4-amino-3-pentene-2-one and 25 gm (0.176 mol) of 2,2,6-tritrimethyl-1,3-dioxene-4-one was stirred and heated to 120° C. for 3 hrs in an oil bath. The reaction mixture was cooled to room temperature and the solid which separated out was filtered off under vacuum and washed with ether and suction dried to give 5 gm of a white crystalline solid of 3-acetyl-2,6-dimethyl-1H-pyridin-4-one.

### STEP 12: Synthesis of 3-[4-(3-acetyl-2,6-dimethyl-pyridine-4-yloxymethyl)-2-ethoxymethyl-phenyl]-5-methyl-thiophene-2-sulphonic acid(4,5-dimethyl-isoxazol-3-yl)-(2-methoxy-ethoxymethyl)-amide

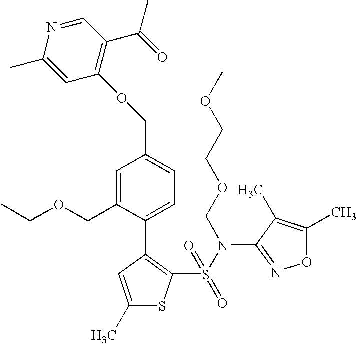

To a stirred solution of 3-acetyl-2,6-dimethyl-1H-pyridin-4-one (0.451 gm, 0.0027 mol) in N,N-dimethyl formamide (6 ml) at −15° C. under flow of dry nitrogen, sodium hydride (60% in mineral oil) (0.132 gm, 0.00275 mol) was added drop wise. After the completion of the addition, the temperature of the reaction mixture was slowly raised to room temperature and maintained for 30 min. The reaction mixture was re-cooled to 0° C. and a solution of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester (1.5 gm, 0.00248 mol) in 9 ml N,N-dimethyl formamide was added drop wise. After the completion of the addition, the temperature of the reaction mixture was slowly raised to room temperature and stirred at room temperature for 24 hrs. The reaction mixture was then diluted with ethyl acetate (40 ml), followed by (10 ml) of cold water. The organic layer was separated and then washed with water and brine solution. Finally the organic layer was dried over sodium sulphate and evaporated under vacuum. The crude compound was purified on a silica gel column using 1:1 hexane/ethyl acetate as an eluent to provide 800 mg of 3-[4-(3-acetyl-2,6-dimethyl-pyridine-4-yloxymethyl)-2-ethoxymethyl-phenyl]-5-methyl-thiophene-2-sulphonic acid(4,5-dimethyl-isoxazol-3-yl)-(2-methoxy-ethoxymethyl)-amide as a viscous oily mass.

### STEP 13: Synthesis of 3-[4-(3-acetyl-2,6-dimethyl-pyridine-4-yloxymethyl)-2-ethoxymethyl-phenyl]-5-methyl-thiophene-2-sulphonic acid (4,5-dimethyl-isoxazol-3-yl)-amide

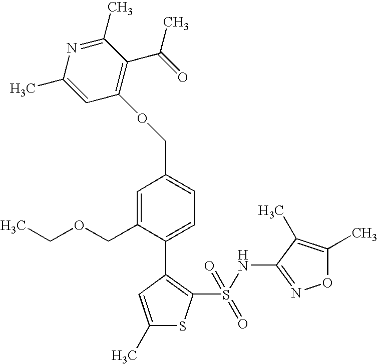

To 3-[4-(3-Acetyl-2,6-dimethyl-pyridine-4-yloxymethyl)-2-ethoxymethyl-phenyl]-5-methyl-thiophene-2-sulphonic acid(4,5-dimethyl-isoxazol-3-yl)-(2-methoxy-ethoxymethyl)-amide (0.80 gm, 1.19 mmol) was added ethanol (10 ml) and 6N aqueous hydrochloric acid (8 ml) at room temperature. The reaction mixture was heated to 90° C. for 3 hrs and then concentrated under vacuum and the residue thus obtained was diluted with water. The pH of the solution was adjusted to 8 using a saturated solution of sodium bicarbonate. This solution was extracted with ethyl acetate (25 ml×2) and the combined organic extracts were washed with water and brine solution. Finally the organic layer was dried over sodium sulphate and concentrated under vacuum. The crude compound was purified by column chromatography on a silica gel column using hexane:ethyl acetate as an eluent to provide 200 mg of 3-[4-(3-acetyl-2,6-dimethyl-pyridine-4-yloxymethyl)-2-ethoxymethyl-phenyl]-5-methyl-thiophene-2-sulphonic acid(4,5-dimethyl-isoxazol-3-yl)-amide

Molecular Formula: C₂₉H₃₃N₃O₆S₂

Molecular Weight: 583.72

¹HNMR (DMSOd₆): 1.07 (t, J=7.2 Hz, 3H) 1.56 (s, 3H) 2.19 (s, 3H) 2.28 (s, 3H) 2.42 (s, 3H) 2.48 (s, 6H) 3.28-3.32 (m, 2H) 4.11 (s, 2H) 5.27 (s, 2H) 6.76 (s, 1H) 7.02-7.07 (m, 2H) 7.27-7.30 (m, 1H) 7.50 (s, 1H) 10.70 (s, 1H)

Mass Spectrum: (m⁻¹) 582

### Example 13

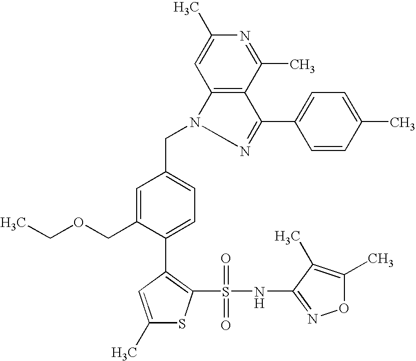

### 3-[4-(4,6-Dimethyl-3-para-tolyl-pyrazolo[4,3-c]pyridin-1-ylmethyl)-2-ethoxymethyl-phenyl]-5-methyl-thiophene-2-sulphonic acid (4,5-dimethyl-isoxazol-3-yl)-amide

### STEP 01: Synthesis of 1-p-tolyl-butane-1,3-dione

To 50 ml of N,N-dimethyl formamide cooled to 0° C., sodium hydride (60% in mineral oil) (7.49 gm, 0.31 mol) was added, followed by addition of a solution of 4-methyl acetophenone (38 gm, 0.284 mol) in dry ethyl acetate (56 ml, 0.56 mol). After the completion of the addition, the temperature of the reaction mixture was slowly raised to room temperature and stirred at room temperature for 12 hrs. The reaction mixture was then acidified with 1N hydrochloric acid and extracted with ethyl acetate (100 ml×2). The combined organic extracts were washed with water and brine solution. Finally the organic layer was dried over sodium sulphate and evaporated under vacuum to give 41 gm of a yellow colored solid of 1-p-tolyl-butane-1,3-dione.

### STEP 02: Synthesis of 3-amino-1-p-tolyl-but-2-en-1-one

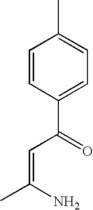

A mixture of 1-p-tolyl-butane-1,3-dione (41 gm, 0.23 mol) and ammonium acetate (71.8 gm, 0.93 mol) in dry methanol (200 ml) was stirred at room temperature for 24 hrs. The reaction mixture was then concentrated completely under vacuum and chilled water was added to the residue and basified to pH 8 using saturated sodium bicarbonate solution, followed by extraction with ethyl acetate (100 ml×2). The combined extracts were washed with water and brine solution. Finally the organic layer was dried over sodium sulphate and evaporated under vacuum to give 25 gm of 3-amino-1-p-tolyl-but-2-en-1-one.

### STEP 03: Synthesis of ethyl 4-bromo-3-(bromo methyl)benzoate

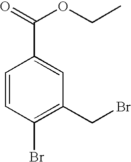

To a solution of 4-bromo-3-methyl benzoic acid ethyl ester (28 gm, 0.11 mol) in 100 ml of carbon tetrachloride was added 22.65 gm (0.12 mol) of N-bromosuccinimide and benzoyl peroxide (1.4 gm, 0.005 mol, 75% in water) and the mixture was refluxed for 10 hrs. The reaction mixture was cooled to room temperature and filtered. The filtrate was then concentrated and the residue thus obtained was purified by triturating with hexane (100 ml) to give the solid product, which was filtered and suction dried to give 17.5 gm of ethyl 4-bromo-3-(bromo methyl)benzoate

### STEP 04: Synthesis of 4-bromo-3-ethoxymethyl-benzoic acid ethyl ester

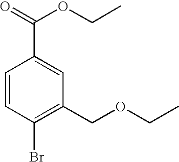

To a cooled solution of ethyl 4-bromo-3-(bromo methyl)benzoate (17.5 gm, 0.054 mol) in ethanol (30 ml) was added sodium ethoxide (7 gm, 0.074 mol) and 12 ml of N,N-dimethyl formamide. The reaction mixture was stirred for 4 hrs at room temperature and then concentrated under vacuum. The residue thus obtained was dissolved in ethyl acetate (100 ml). The ethyl acetate layer was washed with water and brine solution and finally dried over sodium sulphate and evaporated under vacuum to give 12.5 gm of 4-bromo-3-ethoxymethyl-benzoic acid ethyl ester.

### STEP 05: Synthesis of 6-dimethyl-3-(4-methyl-benzoyl)-1H-pyridin-4-one

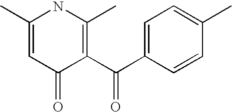

A mixture of 2,2,6-trimethyl-[1,3] dioxin-4-one (24.36 gm, 0.17 mol) and 3-amino-1-p-tolyl-but-2-en-1-one (15 gm, 0.086 mol) was heated at reflux at 120° C. for 6 hrs. The reaction mixture was directly purified by column chromatography on a silica gel column using 10% methanol:ethyl acetate as an eluent to provide 3.2 gm of 6-dimethyl-3-(4-methyl-benzoyl)-1H-pyridin-4-one.

### STEP 06: Synthesis of (4-chloro-2,6-dimethyl-pyridin-3-yl)-para-tolyl-methanone

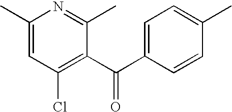

To cold (0° C.) 20 ml of phosphorous oxychloride was added (3.2 gm, 0.013 mol) of 6-dimethyl-3-(4-methyl-benzoyl)-1H-pyridin-4-one. The reaction mixture was stirred and heated at 100° C. for 8 hours. Then the reaction mixture was evaporated under vacuum and the residue thus obtained was basified to pH 8 with saturated sodium carbonate solution, followed by extraction with methylene dichloride (50 ml×2). The combined organic extracts were washed with water and brine solution. Finally the organic layer was dried over anhydrous sodium sulphate and concentrated under vacuum to give 3.2 gm of (4-chloro-2,6-dimethyl-pyridin-3-yl)-p-tolyl-methanone.

### STEP 07: Synthesis of 4,6-Dimethyl-3-p-tolyl-1H-pyrazolo[4,3-c] pyridine

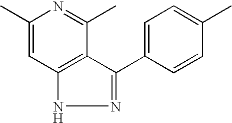

To (4-chloro-2,6-dimethyl-pyridin-3-yl)-para-tolyl-methanone (3.2 gm, 0.01 mol) in ethanol (10 ml, hydrazine hydrate (5.8 ml, 0.185 mol) was added followed by the addition of two drops of acetic acid to the. After the addition was over, the temperature of the reaction mixture was slowly raised to reflux and maintained there for 6 hrs. The reaction mixture was cooled to room temperature and then evaporated under vacuum. The crude mass was dumped onto ice, and the solid thus obtained was filtered off and suction dried to provide 1.6 gm of 4,6-dimethyl-3-p-tolyl-1H-pyrazolo[4,3-c]pyridine.

### STEP 08: Synthesis of (5-methyl-isoxazol-3-yl) carbamic acid tert-butyl ester

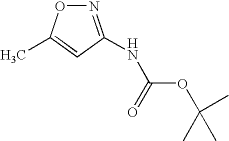

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 09: Synthesis of (4,5-dimethyl-isoxazol-3-yl) carbamic acid tert-butyl ester

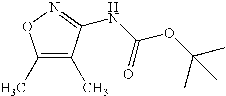

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 10: Synthesis of 4,5-dimethyl-isoxazol-3-yl-amine

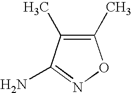

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 11: Synthesis of 5-methyl thiophene-2-sulphonyl chloride

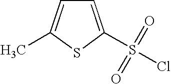

A solution of 2-methyl thiophene (50 gm, 0.51 mol) in chloroform was added to a solution of chloro sulphonic acid (105 ml, 1.53 mol) in chloroform at −5° C. to 0° C. The reaction mixture was then maintained at 0° C. for 3 hrs. The crude reaction mass was slowly dumped into ice cold water, followed by extraction with chloroform (100 ml×2). The combined extract was washed with water and brine. Finally organic layer was dried over anhydrous sodium sulphate, evaporated under vacuum to give 16 gm of 5-methyl thiophene-2-sulphonyl chloride as brown colored liquid.

### STEP 12: Synthesis of 5-methyl-thiophene-2-sulphonic acid (4,5-dimethyl-isoxazol-3yl) amide

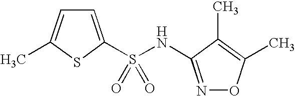

The solution of 5-Methyl thiophene-2-sulphonyl chloride (16.0 gm, 0.081 mol) in (25 ml) methylene chloride was added to the solution of 3-amino-4,5-dimethylisoxazole (6.2 gm, 0.055 mol) and dimethylaminopyridine (500 mg) in pyridine (40 ml) at 0° C. After the completion of the addition the temperature of the reaction mixture was slowly raised to room temperature and stirred it for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 18 gm of 5-methyl-thiophene-2-sulphonic acid (4,5-dimethyl-isoxazol-3yl) amide as brown colored solid.

### STEP 13: Synthesis of 5-methyl-thiophene-2-sulphonic acid (4,5 dimethyl-isoxazol-3-yl)-(2-methoxy-ethoxymethyl)-amide

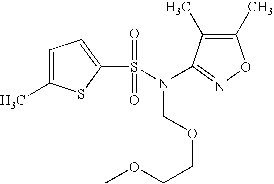

Under the flow of dry nitrogen and stirring, sodium hydride (3.4 gm, 0.07 mol, 60% dispersion in mineral oil) was added in to N,N-dimethyl formamide (40 ml) at 0° C., followed by addition of 5-methyl-thiophene-2-sulphonic acid (4,5-dimethyl-isoxazol-3yl) amide (16.5 gm, 0.060 mol) to it. After completion of the addition, temperature of the reaction mixture was slowly raised and stirred it at ambient temperature for 30 minutes then recooled the reaction mixture to 0° C., followed by the drop wise addition of methoxy ethoxy methyl chloride (8.03 gm, 0.064 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 18.2 gm of 5-methyl-thiophene-2-sulphonic acid (4,5-dimethyl-isoxazol-3yl) amide as yellowish oil.

### STEP 14: Synthesis of 3-borono-N-(4,5-dimethyl-3-isoxazolyl)-N-[(2-methoxy-ethoxy)methyl]-5-methyl-thiophene sulphonamide

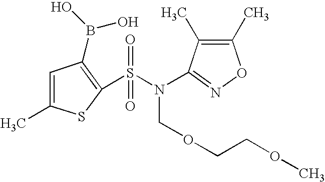

Under the dry nitrogen atmosphere the solution of (14 gm, 0.038 mol) 5-methyl-thiophene-2-sulphonic acid (4,5 dimethyl-isoxazol-3-yl)-(2-methoxy-ethoxymethyl)-amide in tetrahydrofuran (80 ml) was cooled to −78° C. To this n-Butyl lithium (60 ml, 0.097 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (15 ml, 0.062 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×3). The combined extract was washed with water and brine solution. The organic layer was dried over sodium sulphate and concentrated under vacuum to give 15 gm of 3-borono-N-(4,5-dimethyl-3-isoxazolyl)-N-[(2-methoxy-ethoxy)methyl]-5-methyl-thiophene sulphonamide as thick oily mass.

### STEP 15: Synthesis of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester

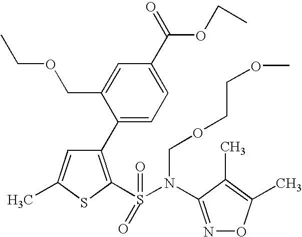

To a stirred solution of 4-bromo-3-ethoxymethyl-benzoic acid ethyl ester (4 gm, 0.0139 mol) in dimethoxy ethane (50 ml) under flow of dry nitrogen bis(triphenylphosphine)palladium(II)chloride (1 gm, 0.00142 mol) was added followed by addition of a 2M aqueous sodium carbonate solution (4.3 gm in 20 ml water). The reaction mixture was stirred at room temperature for 10 min and then heated at 60° C. To this solution of 3-borono-N-(4,5-dimethyl-3-isoxazolyl)-N-[(2-methoxy-ethoxy)methyl]-5-methyl-thiophene sulphonamide (5.5 gm, 0.0136 mol in 25 ml dimethoxy ethane) was added drop wise within 45 min and the reaction was refluxed for 60 min. After 60 min the same procedure was repeated with further addition of 3-borono-N-(4,5-dimethyl-3-isoxazolyl)-N-[(2-methoxy-ethoxy)methyl]-5-methyl-thiophene sulphonamide (5.5 gm, 0.0136 mol in 25 ml dimethoxy ethane) within 45 min. The reaction mixture was then refluxed for 4 hrs and stirred at room temperature for 12 hrs. The mixture was cooled to room temperature and ethyl acetate (100 ml) was added to it followed by addition of water. Then the organic layers were separated, and the aqueous layer further extracted with ethyl acetate (50 ml×2). The combined organic extract was washed with water and brine. Finally the organic layer was dried over sodium sulphate and concentrated under vacuum. The crude compound was purified on a silica gel column using 4:1 hexane/ethyl acetate as an eluent to provide 7 gm of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester as a pale yellow oily mass.

### STEP 16: Synthesis of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

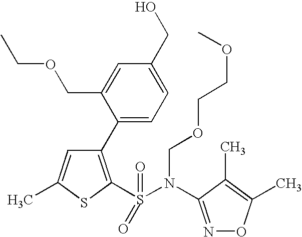

Lithium aluminium hydride (1.4 gm, 0.037 mol) was added to a stirred solution of tetrahydrofuran (80 ml) at 0° C. under flow of nitrogen, followed by addition of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester (8 gm, 0.014 mol) in 35 ml of tetrahydrofuran. The reaction mixture was stirred at 0° C. for 1 hr and then the temperature was raised to room temperature and the mixture stirred for 4 hrs. The excess lithium aluminium hydride was destroyed by addition of sodium hydroxide solution (1 gm dissolved in 100 ml water) at 0° C. followed by extraction with ethyl acetate (25 ml×2). The organic layer was dried over sodium sulphate and concentrated under vacuum to give 4.7 gm of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

### STEP 17: Synthesis of methane sulphonic acid 4-{2-[(4,5-dimethyl-isoxazol-3yl)-(2-methoxy-ethoxy methyl)-sulphamoyl]-5-methylthiophene-3-yl}-3-ethoxy methyl-benzyl ester

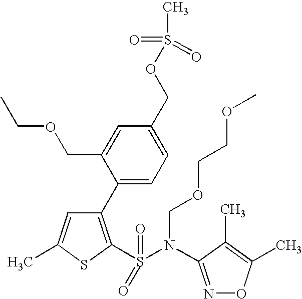

N-Ethyl diisopropyl amine (2.13 ml, 0.012 mol) was added to a solution of 3-(4-hydroxymethyl-phenyl)-5-methyl-thiophene-2-sulphonic acid (4,5-dimethyl-isoxazol-3-yl)-(2-methoxy-ethoxymethyl)-amide (3.2 gm, 0.0060 mol) in 30 ml of dichloromethane. The reaction mixture was cooled to 0° C., where after slowly methane sulphonyl chloride (0.6 ml, 0.0073 mol) was added into the reaction mixture. The reaction mixture was maintained at room temperature for 3 hrs and was then dumped into ice-cold water followed by extraction with methylene chloride (50 ml×2). The combined extracts were washed with dilute hydrochloric acid followed by water and brine solution and the organic layer was dried over sodium sulphate and concentrated to give 3.3 gm of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester as a brown oil.

### STEP 18: Synthesis of 3-[4-(4,6-dimethyl-3-p-tolyl-pyrazolo[4,3-c]pyridin-1-ylmethyl)-2-ethoxymethyl-phenyl]-5-methyl-thiophene-2-sulphonic acid (4,5-dimethyl-isoxazol-3-yl)-(2-methoxy-ethoxymethyl)-amide

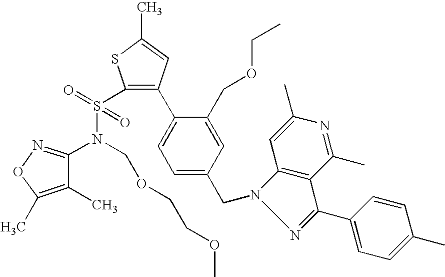

To the stirred solution of 4,6-dimethyl-3-p-tolyl-1H-pyrazolo[4,3-c]pyridine (0.78 gm, 3.3 mmol) in N,N-dimethyl formamide (15 ml) at −15° C. under the flow of dry nitrogen sodium hydride (60% in mineral oil) (0.24 gm, 5 mmol) was added portion wise. After the addition was completed, the reaction mixture was warmed to ambient temperature and maintained there for 30 min. The reaction mixture was re-cooled to 0° C. and a solution of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester (2 gm, 3.3 mmol) in 10 ml of N,N-dimethyl formamide was added drop wise and the mixture was then stirred at room temperature for 24 hrs. The mixture was then cooled to 0° C. and diluted with ethyl acetate (40 ml), followed by (10 ml) of cold water. The organic layer was separated, the aqueous layer was extracted with ethyl acetate (50 ml×2) and the combined organic layer was washed with water and brine solution. Finally the organic layer was dried over sodium sulphate and evaporated under vacuum to afford 2.2 gm crude 3-[4-(4,6-dimethyl-3-p-tolyl-pyrazolo[4,3-c]pyridin-1-ylmethyl)-2-ethoxymethyl-phenyl]-5-methyl-thiophene-2-sulphonic acid (4,5-dimethyl-isoxazol-3-yl)-(2-methoxy-ethoxymethyl-amide as a viscous oily mass.

### STEP 19: Synthesis of 3-[4-(4,6-dimethyl-3-p-tolyl-pyrazolo[4,3-c]pyridin-1-ylmethyl)-2-ethoxymethyl-phenyl]-5-methyl-thiophene-2-sulphonic acid (4,5-dimethyl-isoxazol-3-yl)-amide

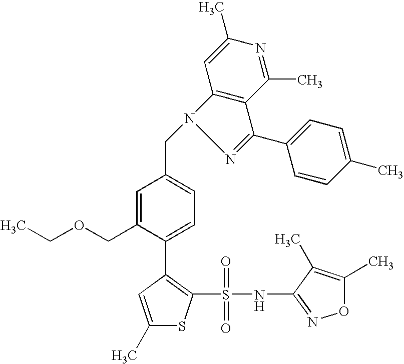

To (2.2 gm, 2.95 mmol) of crude 3-[4-(4,6-dimethyl-3-p-tolyl-pyrazolo[4,3-c]pyridin-1-ylmethyl)-2-ethoxymethyl-phenyl]-5-methyl-thiophene-2-sulphonic acid (4,5-dimethyl-isoxazol-3-yl)-(2-methoxy-ethoxymethyl-amide, 95% ethanol (10 ml) and 6N aqueous hydrochloric acid (8 ml) was added at room temperature. The reaction mixture was stirred and heated for 3 hrs and was then the reaction mixture was concentrated under vacuum. The residue thus obtained was diluted with water and the pH of the solution was adjusted to 8 using a saturated solution of sodium bicarbonate, and the mixture was extracted with ethyl acetate (25 ml×3). The combined organic extract was washed with water and brine solution. Finally the organic layer was dried over sodium sulphate and concentrated under vacuum. The residue was purified by column chromatography on a silica gel column using hexane:ethyl acetate as an eluent to provide 300 mg of 3-[4-(4,6-dimethyl-3-p-tolyl-pyrazolo[4,3-c]pyridin-1-ylmethyl)-2-ethoxymethyl-phenyl]-5-methyl-thiophene-2-sulphonic acid (4,5-dimethyl-isoxazol-3-yl)-amide.

Molecular Formula: C₃₅H₃₇N₅O₄S₂

Molecular Weight: 655.84

¹HNMR (DMSOd₆): 1.01 (t, J=7.2 Hz, 3H) 1.47 (s, 3H) 2.11 (s, 3H) 2.40 (s, 3H) 2.47 (s, 3H) 2.48 (s, 3H) 2.53 (s, 3H) 3.24-3.27 (m, 2H) 4.05 (s, 2H) 5.68 (s, 2H) 6.70 (s, 1H) 6.93-6.95 (m, 1H) 7.08-7.10 (m, 1H) 7.32-7.34 (m, 3H) 7.51-7.54 (m, 3H) 10.75 (s, 1H)

Mass Spectrum: (m⁺¹) 656.2

### Example 14

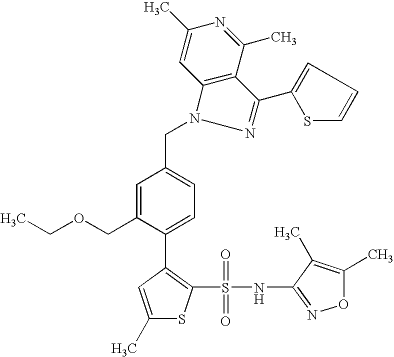

### 3-[4-(4,6-Dimethyl-3-thiophene-2-yl-pyrazolo[4,3-c]pyridin-1-ylmethyl)-2-ethoxymethyl-phenyl]-5-methyl-thiophene-2-sulphonic acid (4,5-dimethyl-isoxazol-3-yl)-amide

### STEP 01: Synthesis of 1-thiophene-2-yl-butane-1,3-dione

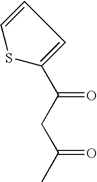

Sodium hydride (60% in mineral oil) (4.18 gm, 0.17 mol) was added to 40 ml of N,N-dimethyl formamide at 0° C. and under the flow of dry nitrogen. To this mixture a solution of 2-acetyl thiophene (20 gm, 0.16 mol) in dry ethyl acetate (31 ml, 0.32 mol) was added. After the completion of the addition the reaction mixture was stirred at room temperature for 12 hrs. The reaction mixture was then acidified with 1N hydrochloric acid and extracted with ethyl acetate (100 ml×2). The combined organic extracts were washed with water and brine solution. The organic layer was dried over sodium sulphate and evaporated to give 27 gm of 1-thiophene-2-yl-butane-1,3-dione.

### STEP 02: Synthesis of 3-amino-1-thiophene-2yl-but-2-en-1-one

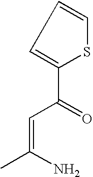

A mixture of 1-thiophene-2-yl-butane-1,3-dione (26.5 gm, 0.16 mol) and ammonium acetate (48.6 gm, 0.63 mol) in dry methanol (260 ml) was stirred at room temperature for 24 hrs. The reaction mixture was concentrated completely under vacuum and chilled water was added to the residue. The reaction mixture was basified to pH 8 using saturated sodium bicarbonate solution, followed by extraction with ethyl acetate (100 ml×2). The combined organic extracts were washed with water and brine solution. Finally the organic layer was dried over sodium sulphate and evaporated to give 16.8 gm of 3-amino-1-thiophene-2yl-but-2-en-1-one.

### STEP 03: Synthesis of ethyl 4-bromo-3-(bromo methyl)benzoate

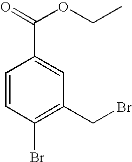

To a solution of 4-bromo-3-methyl benzoic acid ethyl ester (28 gm, 0.11 mol) in 100 ml of carbon tetrachloride was added 22.65 gm (0.12 mol) of N-bromosuccinimide and benzoyl peroxide (1.4 gm, 0.005 mol, 75% in water) and the mixture was refluxed for 10 hrs. The reaction mixture was cooled to room temperature and filtered and the filtrate was concentrated. The residue thus obtained was purified by triturating with hexane (100 ml) which gave the solid product, which was filtered and suction dried to give 17.5 gm of ethyl 4-bromo-3-(bromo methyl)benzoate

### STEP 04: Synthesis of 4-bromo-3-ethoxymethyl-benzoic acid ethyl ester

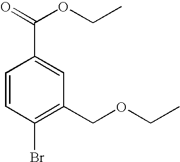

To a (0° C.) cooled solution of ethyl 4-bromo-3-(bromo methyl)benzoate (17.5 gm, 0.054 mol) in ethanol (30 ml) was added sodium ethoxide (7 gm, 0.074 mol) and 12 ml of N,N-dimethyl formamide. The reaction mixture was stirred for 4 hrs at room temperature and then concentrated under vacuum. The residue thus obtained was dissolved in ethyl acetate (100 ml). The ethyl acetate layer was washed with water and brine solution. Finally the organic layer was dried over sodium sulphate and evaporated under vacuum to give 12.5 gm of 4-bromo-3-ethoxymethyl-benzoic acid ethyl ester.

### STEP 05: Synthesis of 2,6-dimethyl-3-(thiophene-2-carbonyl)-1H-pyridin-4-one

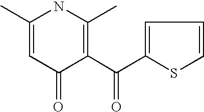

A mixture of 2,2,6-trimethyl-[1,3] dioxin-4-one (28.6 gm, 0.20 mol) and 3-amino-1-thiophene-2yl-but-2-en-1-one (16.8 gm, 0.10 mol) was heated to reflux at 120° C. for 6 hrs. The residue was directly purified by column chromatography on a silica gel column using 10% methanol: ethyl acetate as an eluent to provide 4.6 gm of 2,6-dimethyl-3-(thiophene-2-carbonyl)-1H-pyridin-4-one.

### STEP 06: Synthesis of (4-chloro-2,6-dimethyl-pyridin-3-yl)-thiophene-2-yl-methanone

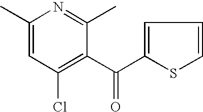

2,6-dimethyl-3-(thiophene-2-carbonyl)-1H-pyridin-4-one (4.6 gm, 0.01 mol) was added to 30 ml of phosphorous oxychloride at 0° C. The mixture was stirred and heated at 100° C. for 8 hours. Then the reaction mixture was evaporated under vacuum and the residue thus obtained was basified to pH 8 with saturated sodium carbonate solution, followed by extraction with methylene dichloride (50 ml×2). The combined organic extracts were washed with water and brine solution. Finally the organic layer was dried over anhydrous sodium sulphate and concentrated under vacuum to give 4.35 gm of (4-chloro-2,6-dimethyl-pyridin-3-yl)-thiophene-2-yl-methanone.

### STEP 07: Synthesis of 4,6-dimethyl-3-thiophene-2yl-1H-pyrazolo[4,3-c] pyridine

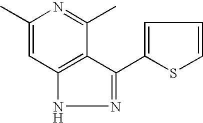

(4-Chloro-2,6-dimethyl-pyridin-3-yl)-thiophene-2-yl-methanone (4.35 gm, 0.02 mol) was taken in ethanol (20 ml). To the mixture hydrazine hydrate (6 ml, 0.185 mol) was added followed by two drops of acetic acid. The temperature was raised slowly and the mixture heated to reflux, and maintained there for 6 hrs. The reaction mixture was evaporated under vacuum. The crude mass was dumped into ice, the solid thus obtained was filtered off and suction dried to provide 4.48 gm of 4,6-dimethyl-3-thiophene-2yl-1H-pyrazolo[4,3-c]pyridine.

### STEP 08: Synthesis of (5-methyl-isoxazol-3-yl) carbamic acid tert-butyl ester

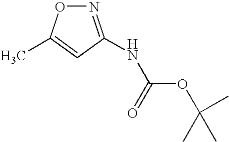

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 09: Synthesis of (4,5-dimethyl-isoxazol-3-yl) carbamic acid tert-butyl ester

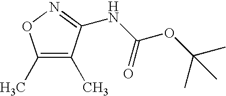

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 10: Synthesis of 4,5-dimethyl-isoxazol-3-yl-amine

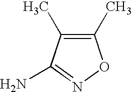

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 11: Synthesis of 5-methyl thiophene-2-sulphonyl chloride

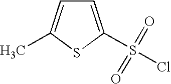

The solution of 2-methyl thiophene (50 gm, 0.51 mol) in chloroform (100 ml) was added to a solution of chloro sulphonic acid (105 ml, 1.53 mol) in chloroform at −5° C. to 0° C. The reaction mixture was maintained at 0° C. for 3 hrs. The crude reaction mass was slowly dumped into the ice cold water, followed by extraction with chloroform (100 ml×2). The combined extract was washed with water and brine. Finally the organic layer was dried over anhydrous sodium sulphate, evaporated under vacuum to give 16 gm of 5-methyl thiophene-2-sulphonyl chloride as a brown colored liquid.

### STEP 12: Synthesis of 5-methyl-thiophene-2-sulphonic acid (4,5-dimethyl-isoxazol-3yl) amide

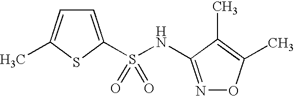

The solution of 5-Methyl thiophene-2-sulphonyl chloride (16.0 gm, 0.081 mol) in (25 ml) methylene chloride was added to the solution of 3-amino-4,5-dimethylisoxazole (6.2 gm, 0.055 mol) and dimethylaminopyridine (500 mg) in pyridine (40 ml) at 0° C. After the completion of the addition the temperature of the reaction mixture was slowly raised to room temperature and stirred it for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 18 gm of 5-methyl-thiophene-2-sulphonic acid (4,5-dimethyl-isoxazol-3yl) amide as brown colored solid.

### STEP 13: Synthesis of 5-methyl-thiophene-2-sulphonic acid (4,5 dimethyl-isoxazol-3-yl)-(2-methoxy-ethoxymethyl)-amide

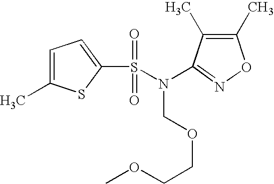

Under the flow of dry nitrogen and stirring, sodium hydride (3.4 gm, 0.07 mol, 60% dispersion in mineral oil) was added in to N,N-dimethyl formamide (40 ml) at 0° C., followed by addition of 5-methyl-thiophene-2-sulphonic acid (4,5-dimethyl-isoxazol-3yl) amide (16.5 gm, 0.060 mol) to it. After completion of the addition, temperature of the reaction mixture was slowly raised and stirred it at ambient temperature for 30 minutes then recooled the reaction mixture to 0° C., followed by the drop wise addition of methoxy ethoxy methyl chloride (8.03 gm, 0.064 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 18.2 gm of 5-methyl-thiophene-2-sulphonic acid (4,5-dimethyl-isoxazol-3yl) amide as yellowish oil.

### STEP 14: Synthesis of 3-borono-N-(4,5-dimethyl-3-isoxazolyl)-N-[(2-methoxy-ethoxy)methyl]-5-methyl-thiophene sulphonamide

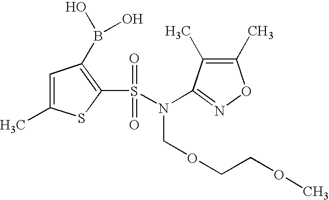

Under the dry nitrogen atmosphere the solution of (14 gm, 0.038 mol) 5-methyl-thiophene-2-sulphonic acid (4,5 dimethyl-isoxazol-3-yl)-(2-methoxy-ethoxymethyl)-amide in tetrahydrofuran (70 ml) was cooled to −78° C. To this n-Butyl lithium (60 ml, 0.097 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (15 ml, 0.062 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×3). The combined extract was washed with water and brine solution. The organic layer was dried over sodium sulphate and concentrated under vacuum to give 15 gm of 3-borono-N-(4,5-dimethyl-3-isoxazolyl)-N-[(2-methoxy-ethoxy)methyl]-5-methyl-thiophene sulphonamide as thick oily mass.

### STEP 15: Synthesis of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester

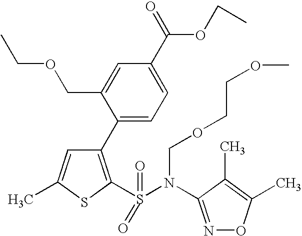

To a stirred solution of 4-bromo-3-ethoxymethyl-benzoic acid ethyl ester (4 gm, 0.0139 mol) in dimethoxy ethane (50 ml) under flow of dry nitrogen bis(triphenylphosphine)palladium(II)chloride (1 gm, 0.00142 mol) was added followed by addition of a 2M aqueous sodium carbonate solution (4.3 gm in 20 ml water). The reaction mixture was stirred at room temperature for 10 min and then heated at 60° C. To this a solution of 3-borono-N-(4,5-dimethyl-3-isoxazolyl)-N-[(2-methoxy-ethoxy)methyl]-5-methyl-thiophene sulphonamide (5.5 gm, 0.0136 mol in 25 ml dimethoxy ethane) was added drop wise within 45 min and the reaction was then refluxed for 60 min. After 60 min the same procedure was repeated with further addition of 3-borono-N-(4,5-dimethyl-3-isoxazolyl)-N-[(2-methoxy-ethoxy)methyl]-5-methyl-thiophene sulphonamide (5.5 gm, 0.0136 mol in 25 ml dimethoxy ethane) within 45 min. The reaction mixture was refluxed for 4 hrs and stirred at room temperature for 12 hrs. The mixture was then cooled to room temperature and ethyl acetate (100 ml) was added to it followed by addition of water. The organic layers were separated, and the aqueous layer further extracted with ethyl acetate (50 ml×2). The combined organic extract was washed with water and brine. Finally the organic layer was dried over sodium sulphate and concentrated under vacuum. The crude compound was purified by column chromatography on a silica gel column using hexane:ethyl acetate as an eluent to provide 7 gm of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester as pale yellow oily mass.

### STEP 16: Synthesis of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

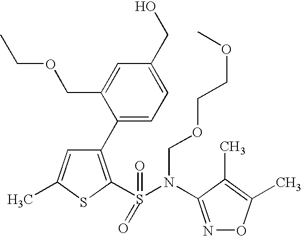

Lithium aluminium hydride (1.4 gm, 0.037 mol) was added to a stirred solution of tetrahydrofuran (70 ml) at 0° C. under flow of nitrogen, followed by addition of 4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester 8 gm (0.014 mol) in 35 ml of tetrahydrofuran. The reaction mixture was stirred at 0° C. for 1 hr and then the temperature was raised to room temperature and the mixture stirred for 4 hrs. The excess lithium aluminium hydride was destroyed by addition of sodium hydroxide solution (1 gm dissolved in 100 ml water) at 0° C. followed by extraction with ethyl acetate (25 ml×2). The organic layer was dried over sodium sulphate and concentrated under vacuum to give 4.7 gm of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

### STEP 17: Synthesis of methane sulphonic acid 4-{2-[(4,5-dimethyl-isoxazol-3yl)-(2-methoxy-ethoxy methyl)-sulphamoyl]-5-methylthiophene-3-yl}-3-ethoxy methyl-benzyl ester

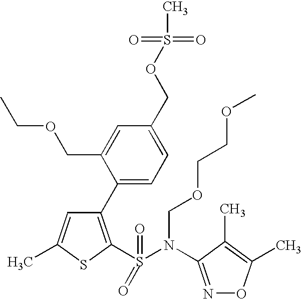

N-Ethyl diisopropyl amine (2.13 ml, 0.012 mol) was added to a solution of 3-(4-hydroxymethyl-phenyl)-5-methyl-thiophene-2-sulphonic acid (4,5-dimethyl-isoxazol-3-yl)-(2-methoxy-ethoxymethyl)-amide (3.2 gm, 0.0060 mol) in 30 ml of dichloromethane. The reaction mixture was cooled to 0° C., where after methane sulphonyl chloride (0.6 ml, 0.0073 mol) was slowly added. The reaction mixture was maintained at room temperature for 3 hrs and was then dumped into ice-cold water followed by extraction with methylene chloride (50 ml×2). The combined extracts were washed with dilute hydrochloric acid followed by water and brine solution and the organic layer was dried over sodium sulphate and concentrated to give 3.3 gm of methane sulphonic acid 4-{2-[(4,5-dimethyl-isoxazol-3yl)-(2-methoxy-ethoxy methyl)-sulphamoyl]-5-methylthiophene-3-yl}-3-ethoxy methyl-benzyl ester as a brown oil.

### STEP 18: Synthesis of 3-[4-(4,6-dimethyl-3-thiophene-2-yl-pyrazolo[4,3-c]pyridin-1-ylmethyl)-2-ethoxymethyl-phenyl]-5-methyl-thiophene-2-sulphonic acid (4,5-dimethyl-isoxazol-3-yl)-(2-methoxy-ethoxymethyl)-amide

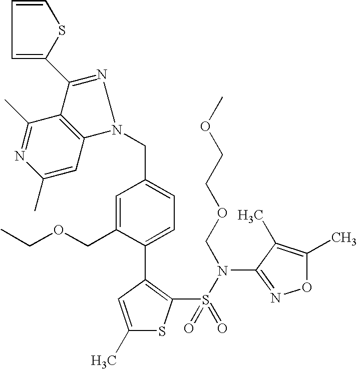

To a stirred solution of 4,6-dimethyl-3-thiophene-2yl-1H-pyrazolo[4,3-c]pyridine (0.76 gm, 3.3 mmol) in N,N-dimethyl formamide (15 ml) at −15° C. under the flow of dry nitrogen, sodium hydride (60% in mineral oil) (0.24 gm, 5 mmol) was added portion wise. After the completion of the addition, the reaction mixture was warmed to ambient temperature and stirred for 30 min. Then the reaction mixture was re-cooled to 0° C. and a solution of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester (2 gm, 3.3 mmol) in 10 ml of N,N-dimethyl formamide was added drop wise and the mixture was then stirred at room temperature for 24 hrs. The mixture was then diluted with ethyl acetate (40 ml), followed by addition of 10 ml of cold water. The organic layer was separated, washed with water and brine solution and finally dried over sodium sulphate and evaporated under vacuum to afford crude 2.1 gm of 3-[4-(4,6-Dimethyl-3-thiophene-2-yl-pyrazolo[4,3-c]pyridin-1-ylmethyl)-2-ethoxymethyl-phenyl]-5-methyl-thiophene-2-sulphonic acid (4,5-dimethyl-isoxazol-3-yl)-(2-methoxy-ethoxymethyl)-amide as a viscous oily mass.

### STEP 19: Synthesis of 3-[4-(4,6-dimethyl-3-thiophene-2-yl-pyrazolo[4,3-c]pyridin-1-ylmethyl)-2-ethoxymethyl-phenyl]-5-methyl-thiophene-2-sulphonic acid (4,5-dimethyl-isoxazol-3-yl)-amide

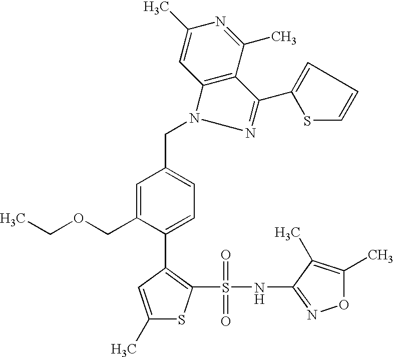

To (2.1 gm, 2.8 mmol) of crude 3-[4-(4,6-dimethyl-3-thiophene-2-yl-pyrazolo[4,3-c]pyridin-1-ylmethyl)-2-ethoxymethyl-phenyl]-5-methyl-thiophene-2-sulphonic acid (4,5-dimethyl-isoxazol-3-yl)-(2-methoxy-ethoxymethyl)-amide was added ethanol (10 ml) and 6N aqueous hydrochloric acid (8 ml) at room temperature. The reaction mixture was stirred and heated for 3 hrs. Then the reaction mixture was concentrated under vacuum and the residue thus obtained was diluted with water and the pH of the solution was adjusted to 8 using a saturated solution of sodium bicarbonate, and the mixture was extracted with ethyl acetate (30 ml×3). The combined organic extract was washed with water and brine solution. Finally the organic layer was dried over sodium sulphate and concentrated under vacuum. The crude compound was purified on a silica gel column using hexane:ethyl acetate as an eluent to provide 320 mg of 3-[4-(4,6-dimethyl-3-thiophene-2-yl-pyrazolo[4,3-c]pyridin-1-ylmethyl)-2-ethoxymethyl-phenyl]-5-methyl-thiophene-2-sulphonic acid (4,5-dimethyl-isoxazol-3-yl)-amide.

Molecular Formula: C₃₂H₃₃N₅O₄S₃

Molecular Weight: 647.84

¹HNMR (DMSOd₆): 1.01 (t, J=7.2 Hz, 3H) 1.47 (s, 3H) 2.12 (s, 3H) 2.47 (s, 3H) 2.53 (s, 3H) 2.66 (s, 3H) 3.22-3.27 (m, 2H) 4.05 (s, 2H) 5.69 (s, 2H) 6.70-6.71 (m, 1H) 6.93-6.95 (m, 1H) 7.06-7.09 (m, 1H) 7.23-7.25 (m, 1H) 7.34 (s, 1H) 7.52-7.53 (m, 2H) 7.71-7.73 (m, 1H) 10.76 (s, 1H)

Mass Spectrum: (m⁺¹) 648.1

### Example 15

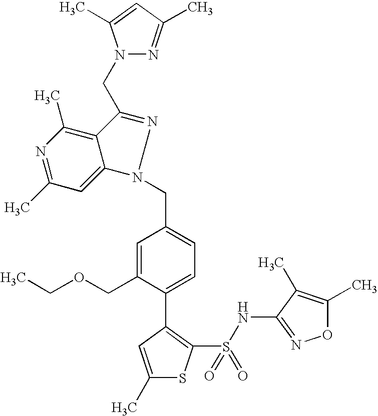

### 3-{4-[3-(3,5-Dimethyl-pyrazol-1-ylmethyl)-4,6-dimethyl-pyrazolo[4,3-c]pyridin-1-ylmethyl]2-ethoxy methyl-phenyl}-5-methyl-thiophene-2-sulphonic acid (4,5-dimethyl-isoxazol-3-yl)-amide

### STEP 01: Synthesis of 1-(3,5-dimethyl-pyrazol-1-yl)pentane-2,4dione

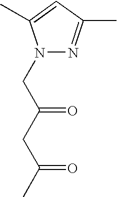

Sodium hydride (60% in mineral oil) (8.6 gm, 0.179 mol) at 0° C. was added to N,N-dimethyl formamide (100 ml) under flow of dry nitrogen. To this mixture a solution of (3,5-dimethyl-pyrazol-1-yl)-acetic acid ethyl ester (30 gm, 0.164 mol) in dry acetone (11.4 ml, 0.196 mol) was added. The reaction mixture was stirred at room temperature for 12 hrs, and was then acidified with 1N hydrochloric acid and extracted with ethyl acetate (100 ml×2). The combined extracts were washed with water and brine solution. Finally the organic layer was dried over sodium sulphate and evaporated to give 26 gm of 1-(3,5-dimethyl-pyrazol-1-yl) pentane-2,4dione.

### STEP 02: Synthesis of 4-amino-1-(3,5-dimethyl-pyrazol-1-yl)-pent-3-en-2-one

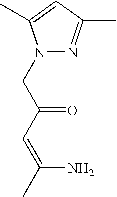

A mixture of 1-(3,5-dimethyl-pyrazol-1-yl) pentane-2,4 dione (26 gm, 0.134 mol) and ammonium acetate (52 gm, 0.675 mol) in dry methanol (200 ml) was stirred at room temperature for 24 hrs. The reaction mixture was concentrated completely under vacuum and chilled water was added to the residue. The reaction mixture was basified to pH 8 using saturated sodium bicarbonate solution, followed by extraction with ethyl acetate (100 ml×2). The combined organic extracts were washed with water and brine solution. Finally organic layer was dried over sodium sulphate and evaporated to give 20 gm of 4-amino-1-(3,5-dimethyl-pyrazol-1-yl)-pent-3-en-2-one.

### STEP 03: Synthesis of ethyl 4-bromo-3-(bromo methyl)benzoate

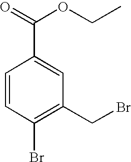

To a solution of 4-bromo-3 methyl benzoic acid ethyl ester (28 gm, 0.1 mmol) in 100 ml of carbon tetrachloride was added 22.65 gm (0.12 mol) of N-bromosuccinimide and benzoyl peroxide (1.4 gm, 0.005 mol, 75% in water) and the mixture was refluxed for 10 hrs. The mixture was then cooled to room temperature and filtered and the filtrate was concentrated. The residue thus obtained was purified by triturating with hexane (100 ml) which gave the solid product, which was filtered and suction dried to give 17.5 gm of ethyl 4-bromo-3-(bromo methyl)benzoate

### STEP 04: Synthesis of 4-bromo-3-ethoxymethyl-benzoic acid ethyl ester

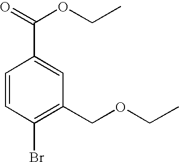

To a (0° C.) cooled solution of ethyl 4-bromo-3-(bromo methyl)benzoate (17.5 gm, 0.054 mol) in ethanol (30 ml), sodium ethoxide (7 gm, 0.074 mol) and (12 ml) of N,N-dimethyl formamide was added. The reaction mixture was stirred for 4 hrs at room temperature and then concentrated under vacuum. The residue thus obtained was dissolved in ethyl acetate (100 ml). The ethyl acetate layer was washed with water and brine solution and finally dried over sodium sulphate and evaporated under vacuum to give 12.5 gm of 4-bromo-3-ethoxymethyl-benzoic acid ethyl ester.

### STEP 05: Synthesis of 3-[2-(3,5-dimethyl-pyrazol-1-yl)acetyl]-2,6-dimethyl-1H-pyridin-4-one

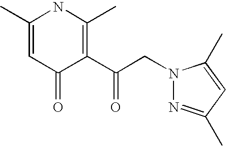

A mixture of 2,2,6-trimethyl-[1,3] dioxin-4-one (30 gm, 0.21 mol) and 4-amino-1-(3,5-dimethyl-pyrazol-1-yl)-pent-3-en-2-one (20 gm, 0.10 mol) was heated to reflux at 120° C. for 6 hrs. The reaction mixture was cooled to room temperature and directly purified on a silica gel column using 10% methanol:ethyl acetate as an eluent to provide 6 gm of 3-[2-(3,5-dimethyl-pyrazol-1-yl)acetyl]-2,6-dimethyl-1H-pyridin-4-one.

### STEP 06: Synthesis of 1-(4-chloro-2,6-dimethyl-pyridin-3-yl)-2-(3,5-dimethyl-pyrazol-1-yl)-ethanone

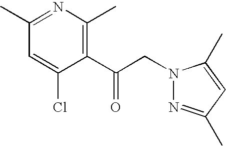

3-[2-(3,5-dimethyl-pyrazol-1-yl)acetyl]-2,6-dimethyl-1H-pyridin-4-one (6 gm, 0.023 mol) was added to 30 ml of phosphorous oxychloride at 0° C. The reaction mixture was stirred at 30° C. for 8 hours. The reaction mixture was evaporated under vacuum and the residue basified to pH 8 with saturated sodium carbonate solution, followed by extraction with methylene dichloride (50 ml×2). The combined organic extracts were washed with water and brine solution. Finally the organic layer was dried over anhydrous sodium sulphate and concentrated to give 1.8 gm of 1-(4-chloro-2,6-dimethyl-pyridin-3-yl)-2-(3,5-dimethyl-pyrazol-1-yl)-ethanone.

### STEP 07: Synthesis of 3-(3,5-dimethyl-pyrazol-1-ylmethyl)-4,6-dimethyl-1H-pyrazolo[4,3-c]pyridine

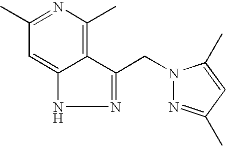

1-(4-chloro-2,6-dimethyl-pyridin-3-yl)-2-(3,5-dimethyl-pyrazol-1-yl)-ethanone (1.8 gm, 0.0065 mol) was dissolved in ethanol (10 ml). To the mixture was added hydrazine hydrate (3.12 ml, 0.0624 mol) followed by the addition of two drops of acetic. The temperature was slowly raised and heated to reflux, and then maintained at reflux for 6 hrs. The reaction mixture was cooled to room temperature and then evaporated under vacuum. The crude mass was dumped in to ice, the solid obtained was filtered off and suction dried to provide 0.5 gm of 3-(3,5-dimethyl-pyrazol-1-ylmethyl)-4,6-dimethyl-1H-pyrazolo[4,3-c]pyridine.

### STEP 08: Synthesis of (5-methyl-isoxazol-3-yl) carbamic acid tert-butyl ester

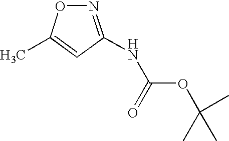

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 09: Synthesis of (4,5-dimethyl-isoxazol-3-yl) carbamic acid tert-butyl ester

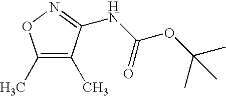

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 10: Synthesis of 4,5-dimethyl-isoxazol-3-yl-amine

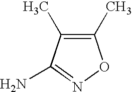

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 11: Synthesis of 5-methyl thiophene-2-sulphonyl chloride

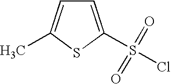

A solution of 2-methyl thiophene (50 gm, 0.51 mol) in chloroform was added to a solution of chloro sulphonic acid (105 ml, 1.53 mol) in chloroform at −5° C. to 0° C. The reaction mixture was then maintained at 0° C. for 3 hrs. The crude reaction mass was slowly dumped into the ice cold water, followed by extraction with chloroform (100 ml×2). The combined extract was washed with water and brine. Finally the organic layer was dried over anhydrous sodium sulphate, and evaporated under vacuum to give 16 gm of 5-methyl thiophene-2-sulphonyl chloride as a brown colored liquid.

### STEP 12: Synthesis of 5-methyl-thiophene-2-sulphonic acid (4,5-dimethyl-isoxazol-3yl) amide

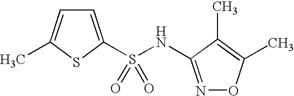

The solution of 5-Methyl thiophene-2-sulphonyl chloride (16.0 gm, 0.081 mol) in (25 ml) methylene chloride was added to the solution of 3-amino-4,5-dimethylisoxazole (6.2 gm, 0.055 mol) and dimethylaminopyridine (500 mg) in pyridine (40 ml) at 0° C. After the completion of the addition the temperature of the reaction mixture was slowly raised to room temperature and stirred it for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 18 gm of 5-methyl-thiophene-2-sulphonic acid (4,5-dimethyl-isoxazol-3yl) amide as brown colored solid.

### STEP 13: Synthesis of 5-methyl-thiophene-2-sulphonic acid (4,5 dimethyl-isoxazol-3-yl)-(2-methoxy-ethoxymethyl)-amide

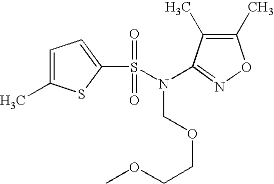

Under the flow of dry nitrogen and stirring, sodium hydride (3.4 gm, 0.07 mol, 60% dispersion in mineral oil) was added in to N,N-dimethyl formamide (40 ml) at 0° C., followed by addition of 5-methyl-thiophene-2-sulphonic acid (4,5-dimethyl-isoxazol-3yl) amide (16.5 gm, 0.060 mol) to it. After completion of the addition, temperature of the reaction mixture was slowly raised and stirred it at ambient temperature for 30 minutes then recooled the reaction mixture to 0° C., followed by the drop wise addition of methoxy ethoxy methyl chloride (8.03 gm, 0.064 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 18.2 gm of 5-methyl-thiophene-2-sulphonic acid (4,5-dimethyl-isoxazol-3yl) amide as yellowish oil.

### STEP 14: Synthesis of 3-borono-N-(4,5-dimethyl-3-isoxazolyl)-N-[(2-methoxy-ethoxy)methyl]-5-methyl-thiophene sulphonamide

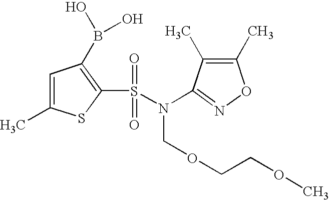

Under the dry nitrogen atmosphere the solution of (14 gm, 0.038 mol) 5-methyl-thiophene-2-sulphonic acid (4,5 dimethyl-isoxazol-3-yl)-(2-methoxy-ethoxymethyl)-amide in tetrahydrofuran (80 ml) was cooled to −78° C. To this n-Butyl lithium (60 ml, 0.097 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (15 ml, 0.062 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×3). The combined extract was washed with water and brine solution. The organic layer was dried over sodium sulphate and concentrated under vacuum to give 15 gm of 3-borono-N-(4,5-dimethyl-3-isoxazolyl)-N-[(2-methoxy-ethoxy)methyl]-5-methyl-thiophene sulphonamide as thick oily mass.

### STEP 15: Synthesis of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester

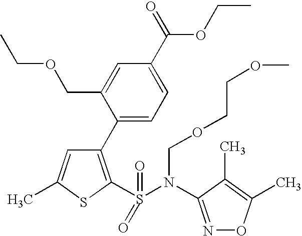

To a stirred solution of 4-bromo-3-ethoxymethyl-benzoic acid ethyl ester (4 gm, 0.0139 mol) in dimethoxy ethane (50 ml) under flow of dry nitrogen bis(triphenylphosphine)palladium(II)chloride (1 gm, 0.00142 mol) was added followed by addition of a 2M aqueous sodium carbonate solution (4.3 gm in 20 ml water). The reaction mixture was stirred at room temperature for 10 min and then heated at 60° C. To this mixture a solution of 3-borono-N-(4,5-dimethyl-3-isoxazolyl)-N-[(2-methoxy-ethoxy)methyl]-5-methyl-thiophene sulphonamide (5.5 gm, 0.0136 mol in 25 ml dimethoxy ethane) was added within 45 min and the reaction was refluxed for 60 min. After 60 min the same procedure was repeated with further addition of 3-borono-N-(4,5-dimethyl-3-isoxazolyl)-N-[(2-methoxy-ethoxy)methyl]-5-methyl-thiophene sulphonamide (5.5 gm, 0.0136 mol in 25 ml dimethoxy ethane) within 45 min. The reaction mixture was refluxed for 4 hrs and stirred at room temperature for 12 hrs and was then cooled to room temperature and ethyl acetate (100 ml) was added followed by addition of water. Then the organic layers were separated, the aqueous layer further extracted with ethyl acetate (50 ml×2). The combined organic extracts was washed with water and brine and finally organic layer was dried over sodium sulphate and concentrated under vacuum. The crude compound was purified by column chromatography on a silica gel column using hexane:ethyl acetate as an eluent to provide 7 gm of (4-{2-[(4,5-dimethyl-isoxazol-3-yl)-(2-methoxy-ethoxymethyl)-sulphamoyl]-5-methyl-thiophene-3-yl}-3-ethoxymethyl-benzoic acid ethyl ester as pale yellow oily mass.

### STEP 16: Synthesis of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

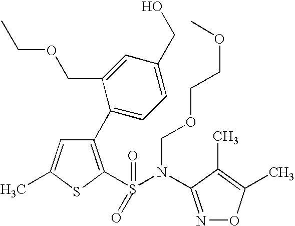

Lithium aluminium hydride (1.4 gm, 0.037 mol) was added with stirring to tetrahydrofuran (20 ml) at 0° C. under flow of nitrogen, followed by addition of (4-{2-[(4,5-dimethyl-isoxazol-3-yl)-(2-methoxy-ethoxymethyl)-sulphamoyl]-5-methyl-thiophene-3-yl}-3-ethoxymethyl-phenyl)-acetic acid ethyl ester (8 gm, 0.014 mol) in 35 ml of tetrahydrofuran. The reaction mixture was stirred at 0° C. for 1 hr and then the temperature was raised to room temperature and the mixture stirred for 4 hrs. The excess lithium aluminium hydride was destroyed by addition of sodium hydroxide solution (1 gm dissolved in 100 ml water) at 0° C. followed by extraction with ethyl acetate (25 ml×2). The organic layer was dried over sodium sulphate and concentrated under vacuum to give 4.7 gm of 3-(2-ethoxymethyl-4-hydroxy methyl-phenyl)-5-methyl-thiophene-2-sulphonic acid-(4,5-dimethyl-isoxazol-3-yl)-2-methoxy-ethoxymethyl) amide

### STEP 17: Synthesis of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester

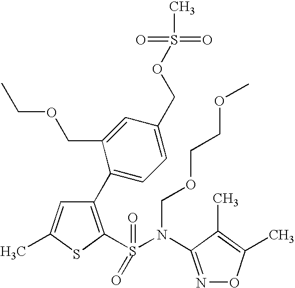

N-Ethyl diisopropyl amine (2.13 ml, 0.012 mol) was added to a solution of 3-(4-hydroxymethyl-phenyl)-5-methyl-thiophene-2-sulphonic acid (4,5-dimethyl-isoxazol-3-yl)-(2-methoxy-ethoxymethyl)-amide (3.2 gm, 0.0060 mol) in 30 ml of dichloro methane. The reaction mixture was cooled to 0° C., where after methane sulphonyl chloride (0.6 ml, 0.0073 mol) was slowly added. The reaction mixture was maintained at room temperature for 3 hrs and was then dumped into ice-cold water followed by extraction with methylene chloride (50 ml×2). The combined extracts were washed with dilute hydrochloric acid followed by water and brine solution and the organic layer was dried over sodium sulphate and concentrated to give 3.3 gm of methane sulphonic acid 4-{2-[(4,5-dimethyl-isoxazol-3yl)-(2-methoxy-ethoxy methyl)-sulphamoyl]-5-methylthiophene-3-yl}-3-ethoxy methyl-benzyl ester as a brown oil.

### STEP 18: Synthesis of 3-{4-[3-(3,5-dimethyl-pyrazol-1-ylmethyl)-4,6-dimethyl-pyrazolo[4,3-c]pyridin-1-ylmethyl]2-ethoxy methyl-phenyl}-5-methyl-thiophene-2-sulphonic acid (4,5-dimethyl-isoxazol-3-yl)-(2-methoxy-ethoxy methyl)-amide

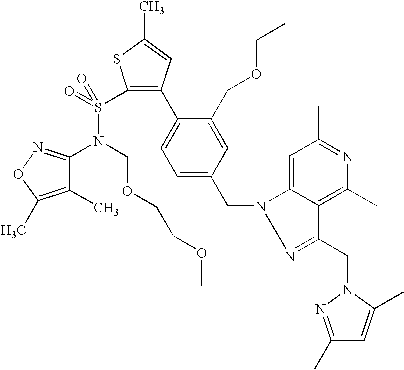

To a stirred solution of 3-(3,5-dimethyl-pyrazol-1-ylmethyl)-4,6-dimethyl-1H-pyrazolo[4,3-c]pyridine (0.45 gm, 0.0017 mol) in N,N-dimethyl formamide (10 ml) at −15° C. under the flow of dry nitrogen was added portion wise sodium hydride (60% in mineral oil) (0.127 gm, 0.0026 mol). After the completion of the addition, the reaction mixture was warmed to ambient temperature and maintained for 30 min. The reaction mixture was re-cooled to 0° C. and a solution of methane sulphonic acid 4-{2-[(4,5-dimethyl-isoxazol-3-yl)-(2-methoxy-ethoxy methyl)-sulphamoyl]-5-methylthiophene-3-yl}-3-ethoxy methyl-benzyl ester (1 gm, 0.0016 mol) in 10 ml of N,N-dimethyl formamide was added drop wise to the reaction mixture and stirred at room temperature for 24 hrs. The mixture was then diluted with ethyl acetate (40 ml), followed by 10 ml of cold water. The organic layer was separated, washed with water and brine solution. Finally the organic layer was dried over sodium sulphate and evaporated under vacuum to afford crude 1.5 gm of 3-{4-[3-(3,5-dimethyl-pyrazol-1-ylmethyl)-4,6-dimethyl-pyrazolo[4,3-c]pyridin-1-ylmethyl]2-ethoxy methyl-phenyl}-5-methyl-thiophene-2-sulphonic acid (4,5-dimethyl-isoxazol-3-yl)-(2-methoxy-ethoxy methyl)-amide a viscous oily mass.

### STEP 19: Synthesis of 3-{4-[3-(3,5-dimethyl-pyrazol-1-ylmethyl)-4,6-dimethyl-pyrazolo[4,3-c]pyridin-1-ylmethyl]2-ethoxy methyl-phenyl}-5-methyl-thiophene-2-sulphonic acid (4,5-dimethyl-isoxazol-3-yl)-amide

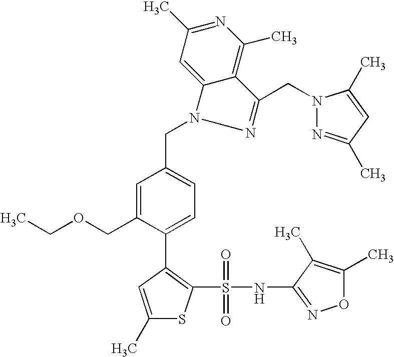

To (1.5 gm, 1.96 mmol) of crude 3-{4-[3-(3,5-dimethyl-pyrazol-1-ylmethyl)-4,6-dimethyl-pyrazolo[4,3-c]pyridin-1-ylmethyl]2-ethoxy methyl-phenyl}-5-methyl-thiophene-2-sulphonic acid (4,5-dimethyl-isoxazol-3-yl)-(2-methoxy-ethoxy methyl)-amide was added 95% ethanol (10 ml) and 6N aqueous hydrochloric acid (6 ml) at room temperature. The reaction mixture was refluxed for 3 hrs. Then the reaction mixture was concentrated under vacuum. The residue thus obtained was diluted with water and the pH of the solution was adjusted to 8 using a saturated solution of sodium bicarbonate, and the mixture was extracted with ethyl acetate (30 ml×3). The combined organic extract was washed with water and brine solution. Finally the organic layer was dried over sodium sulphate and concentrated under vacuum. The crude compound was purified on a silica gel column using hexane:ethyl acetate as an eluent to provide 300 mg of 3-{4-[3-(3,5-Dimethyl-pyrazol-1-ylmethyl)-4,6-dimethyl-pyrazolo[4,3-c]pyridin-1-ylmethyl]2-ethoxy methyl-phenyl}-5-methyl-thiophene-2-sulphonic acid (4,5-dimethyl-isoxazol-3-yl)-amide.

Molecular Formula: C₃₄H₃₉N₇O₄S₂

Molecular Weight: 673.86

¹HNMR (DMSOd₆): 1.02 (t, J=7.2 Hz, 3H) 1.51 (s, 3H) 2.02 (s, 3H) 2.13 (s, 3H) 2.29 (s, 3H) 2.47 (s, 3H) 2.48 (s, 3H) 2.76 (s, 3H) 3.22-3.27 (m, 2H) 4.04 (s, 2H) 5.59 (s, 2H) 5.60 (s, 2H) 5.82 (s, 1H) 6.69 (s, 1H) 6.91-6.93 (m, 1H) 7.00-7.02 (m, 1H) 7.29 (s, 1H) 7.43 (s, 1H) 10.70 (s, 1H)

Mass Spectrum: (m⁻¹) 674.2

### Example 16

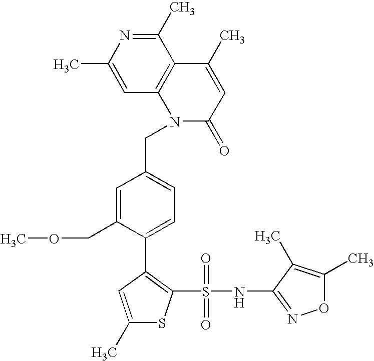

### 3-[2-Methoxymethyl-4-(4,5,7-trimethyl-2-oxo-2H-[1,6]naphthyridin-1-ylmethyl)-phenyl]-5-methyl-thiophene-2-sulphonic acid (4,5-dimethoxy-isoxazol-3-yl)-amide

### STEP 01: Synthesis of N-(3-acetyl-2,6-dimethyl-pyridin-4-yl)-4-methyl-benzenesulphonamide

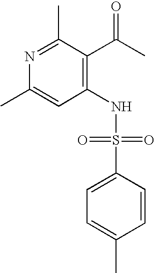

Tosyl isocyanate (52 gm, 0.26 mol) was added to a stirred suspension of 3-acetyl-2,6-dimethyl-1H-pyridin-4-one (20 gm, 0.12 mol) in acetonitrile (200 ml). After the initial exotherm had subsided the mixture was refluxed for 2 hours. The reaction mixture was cooled to room temperature and the suspended solid was collected by filtration to give 30 gm of N-(3-acetyl-2,6-dimethyl-pyridin-4-yl)-4-methyl-benzenesulphonamide

### STEP 02: Synthesis of 1-(4-amino-2,6-Dimethyl-pyridin-3-yl-ethanone

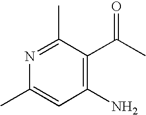

N-(3-Acetyl-2,6-dimethyl-pyridin-4-yl)-4-methyl-benzenesulphonamide (30 g, 0.10 mol) was added to concentrated sulphuric acid (30 ml) at 0° C. and then the reaction mixture was stirred at 50° C. for 1 hour. The reaction mixture was cooled to room temperature and poured into crushed ice. The mixture was the adjusted to pH 8 by the addition of solid sodium carbonate and extracted with dichloro methane (100 ml×3), The combined organic phases were washed with water and brine solution. Finally the organic layer was dried over sodium sulphate and concentrated to give 9 gm of 1-(4-amino-2,6-dimethyl-pyridin-3-yl-ethanone as a low melting solid.

### STEP 03: Synthesis of 4,5,7-Trimethyl-1H-[1,6] naphthyridin-2-one

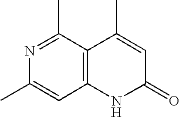

1-(4-amino-2,6-Dimethyl-pyridin-3-yl-ethanone (9 g, 0.054 mol) and (carbethoxymethylene) triphenylphosphorane (21 g, 0.06 mol) was added to xylene (100 ml) and the mixture was stirred and heated at reflux for 6 hrs. The reaction mixture was evaporated under vacuum and to the residue thus obtained was added sodium ethoxide (2.1 gm) and 20 ml of ethanol. The mixture was stirred and heated at reflux for 6 hrs and was then evaporated under vacuum, and the residue was diluted with water followed by addition of concentrated hydrochloric acid under stirring. This mixture was then extracted with ether. The ether extract was discarded. The aqueous phase was basified with solid potassium carbonate and extracted with ethyl acetate, the organic layer were washed with water and brine and dried over sodium sulphate and concentrated to yield 0.662 gm of 4,5,7-trimethyl-1H-[1,6]naphthyridin-2-one.

### STEP 04: Synthesis of ethyl 4-bromo-3-(bromo methyl)benzoate

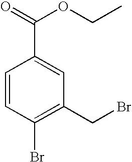

To the solution of 4-bromo-3 methyl benzoic acid ethyl ester (28 gm, 0.1 mmol) in 100 ml of carbon tetrachloride was added (22.65 gm, 0.12 mol) of N-bromosuccinimide and benzoyl peroxide (1.4 gm, 0.005 mol, 75% in water) and the mixture was refluxed for 10 hrs. The reaction mixture was cooled to room temperature and filtered. The filtrate was concentrated and the residue thus obtained was purified by triturating with hexane (100 ml) which gave the solid product, which was filtered and suction dried to give 17.5 gm of ethyl 4-bromo-3-(bromo methyl)benzoate

### STEP 05: Synthesis of 4-bromo-3-methoxymethyl-benzoic acid ethyl ester

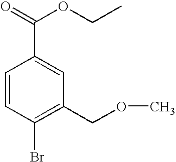

To a (0° C.) cooled solution of 40 ml of methanol and 3 gm (0.05 mol) of sodium methoxide at 0° C. was added a solution of (13 gm, 0.04 mol) of 4-bromo-3-bromomethyl-benzoic acid ethyl ester in 12 ml of N,N-dimethyl formamide. The reaction mixture was stirred at room temperature (28-30° C.) for 2 hrs and then evaporated under vacuum. The residue obtained was acidified to pH 1 with dilute hydrochloric acid and extracted with ethyl acetate (100 ml×2). The combined extracts were washed with water and brine solution. Finally the organic layer was dried over sodium sulphate and evaporated under vacuum to give 9 gm of 4-bromo-3-methoxymethyl-benzoic acid ethyl ester.

### STEP 06: Synthesis of (5-methyl-isoxazol-3-yl) carbamic acid tert-butyl ester

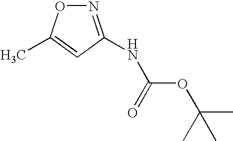

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 07: Synthesis of (4,5-dimethyl-isoxazol-3-yl) carbamic acid tert-butyl ester

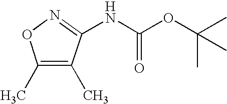

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 08: Synthesis of 4,5-dimethyl-isoxazol-3-yl-amine

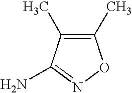

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 09: Synthesis of 5-methyl thiophene-2-sulphonyl chloride

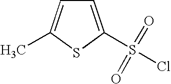

The solution of 2-methyl thiophene (50 gm, 0.51 mol) in chloroform was added to a solution of chloro sulphonic acid (105 ml, 1.53 mol) in chloroform at −5° C. to 0° C. Then the reaction mixture was maintained at 0° C. for 3 hrs. The crude reaction mass was then slowly dumped into the ice cold water, followed by extraction with chloroform (100 ml×2). The combined extract was washed with water and brine. Finally the organic layer was dried over anhydrous sodium sulphate, evaporated under vacuum to give 16 gm of 5-methyl thiophene-2-sulphonyl chloride as brown colored liquid.

### STEP 10: Synthesis of 5-methyl-thiophene-2-sulphonic acid (4,5-dimethyl-isoxazol-3yl) amide

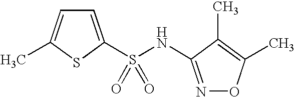

The solution of 5-Methyl thiophene-2-sulphonyl chloride (16.0 gm, 0.081 mol) in (25 ml) methylene chloride was added to the solution of 3-amino-4,5-dimethylisoxazole (6.2 gm, 0.055 mol) and dimethylaminopyridine (500 mg) in pyridine (40 ml) at 0° C. After the completion of the addition the temperature of the reaction mixture was slowly raised to room temperature and stirred it for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 18 gm of 5-methyl-thiophene-2-sulphonic acid (4,5-dimethyl-isoxazol-3yl) amide as brown colored solid.

### STEP 11: Synthesis of 5-methyl-thiophene-2-sulphonic acid (4,5 dimethyl-isoxazol-3-yl)-(2-methoxy-ethoxymethyl)-amide

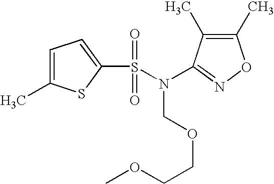

Under the flow of dry nitrogen and stirring, sodium hydride (3.4 gm, 0.07 mol, 60% dispersion in mineral oil) was added in to N,N-dimethyl formamide (40 ml) at 0° C., followed by addition of 5-methyl-thiophene-2-sulphonic acid (4,5-dimethyl-isoxazol-3yl) amide (16.5 gm, 0.060 mol) to it. After completion of the addition, temperature of the reaction mixture was slowly raised and stirred it at ambient temperature for 30 minutes then recooled the reaction mixture to 0° C., followed by the drop wise addition of methoxy ethoxy methyl chloride (8.03 gm, 0.064 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 18.2 gm of 5-methyl-thiophene-2-sulphonic acid (4,5-dimethyl-isoxazol-3yl) amide as yellowish oil.

### STEP 12: Synthesis of 3-borono-N-(4,5-dimethyl-3-isoxazolyl)-N-[(2-methoxy-ethoxy)methyl]-5-methyl-thiophene sulphonamide

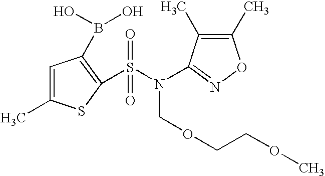

Under the dry nitrogen atmosphere the solution of (14 gm, 0.038 mol) 5-methyl-thiophene-2-sulphonic acid (4,5 dimethyl-isoxazol-3-yl)-(2-methoxy-ethoxymethyl)-amide in tetrahydrofuran (80 ml) was cooled to −78° C. To this n-Butyl lithium (60 ml, 0.097 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (15 ml, 0.062 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×3). The combined extract was washed with water and brine solution. The organic layer was dried over sodium sulphate and concentrated under vacuum to give 15 gm of 3-borono-N-(4,5-dimethyl-3-isoxazolyl)-N-[(2-methoxy-ethoxy)methyl]-5-methyl-thiophene sulphonamide as thick oily mass.

### STEP 13: Synthesis of (4-{2-[(4,5-Dimethyl-isoxazol-3-yl)-(2-methoxy-ethoxymethyl)-sulphamoyl]-5-methyl-thiophene-3-yl}-3-methoxymethyl-benzoic acid ethyl ester

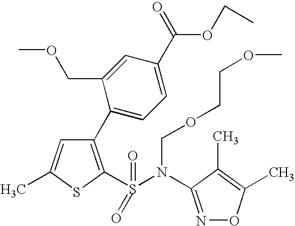

To a stirred solution of 4-bromo-3-methoxymethyl-benzoic acid ethyl ester (4 gm, 0.014 mol) in dimethoxy ethane (50 ml) under nitrogen, bis(triphenylphosphine)palladium(II)chloride (1 gm, 0.0014 mol) was added followed by addition of a 2M aqueous sodium carbonate solution (4.3 gm in 20 ml water). The reaction mixture was stirred at room temperature for 10 min and then heated at 60° C. To this reaction mixture the solution of 3-borono-N-(4,5-dimethyl-3-isoxazolyl)-N-[(2-methoxy-ethoxy)methyl]-5-methyl-thiophene sulphonamide (3.1 gm, 0.0076 mol in 25 ml dimethoxy ethane) was added drop wise within 45 min and the reaction mixture was then refluxed for 60 min. After 1 hr the same procedure was repeated with further addition of 3-borono-N-(4,5-Dimethyl-3-isoxazolyl)-N-[(2-methoxy-ethoxy)methyl]-5-methyl-thiophene sulphonamide (3.1 gm, 0.0076 mol in 25 ml dimethoxy ethane) within 45 min. After the completion of the addition the reaction mixture was refluxed for 4 hrs and stirred at room temperature for 12 hrs. The reaction mixture was cooled to room temperature and then diluted with ethyl acetate (100 ml) and water. The organic layer was separated and the aqueous layer further extracted with ethyl acetate. The combined extracts was washed with water and brine solution. Finally the organic layer was dried over sodium sulphate and concentrated under vacuum. The crude compound was purified on a silica gel column using 4:1 hexane/ethyl acetate as an eluent to provide 5.5 gm of (4-{2-[(4,5-dimethyl-isoxazol-3-yl)-(2-methoxy-ethoxymethyl)-sulphamoyl]-5-methyl-thiophene-3-yl}-3-methoxymethyl-benzoic acid ethyl ester as a pale yellow oily mass.

### STEP 14: Synthesis of 3-(2-ethoxymethyl-4-hydroxy methyl-phenyl)-5-methyl-thiophene-2-sulphonic acid-(4,5-dimethyl-isoxazol-3-yl)-2-methoxy-methyl) amide

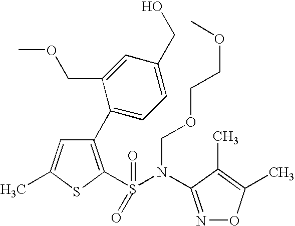

Lithium aluminium hydride (0.7 gm, 0.018 mol) was added to a stirred solution of tetrahydrofuran (15 ml) at 0° C. under flow of dry nitrogen, followed by addition of a solution of the (4-{2-[(4,5-dimethyl-isoxazol-3-yl)-(2-methoxy-ethoxymethyl)-sulphamoyl]-5-methyl-thiophene-3-yl}-3-methoxymethyl-benzoic acid ethyl ester (5.5 gm, 0.009 mol) in 30 ml of tetrahydrofuran. The reaction mixture was stirred at 0° C. for 1 hr and then the temperature was raised to room temperature and stirred for 4 hrs. The reaction mixture was then cooled to 0° C., and a sodium hydroxide solution 50 ml (1 gm dissolved in 100 ml water) was added drop wise while maintaining the temperature at 0° C. This was followed by extraction with ethyl acetate (25 ml×2). The organic layer was separated and washed with water and brine solution. Finally the organic layer was dried over sodium sulphate and concentrated under vacuum to give 2.7 gm 3-(2-ethoxymethyl-4-hydroxy methyl-phenyl)-5-methyl-thiophene-2-sulphonic acid-(4,5-dimethyl-isoxazol-3-yl)-2-methoxy-methyl) amide.

### STEP 15: Synthesis of methane sulphonic acid 4-{2-[(4,5-dimethyl-isoxazol-3yl)-(2-methoxy-ethoxy methyl)-sulphamoyl]-5-methylthiophene-3-yl}-3-methoxy methyl-benzyl ester

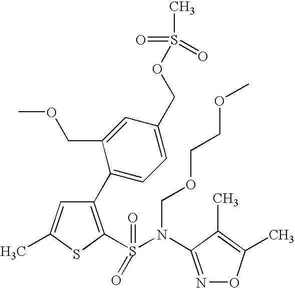

N-Ethyl diisopropyl amine (2 ml, 0.011 mol) was added to a solution of 3-(2-ethoxymethyl-4-hydroxy methyl-phenyl)-5-methyl-thiophene-2-sulphonic acid-(4,5-dimethyl-isoxazol-3-yl)-2-methoxy-methyl) amide (2.6 gm, 0.0051 mol) in 30 ml of dichloro methane. The reaction mixture was cooled to 0° C., and then methane sulphonyl chloride (0.5 ml, 0.0061 mol) was slowly added into the reaction mixture. After the completion of the addition the reaction mixture was maintained at room temperature for 3 hrs, and was then dumped into ice-cold water followed by extraction with methylene chloride (50 ml×2). The combined organic extract was washed with dilute hydrochloric acid followed by washings with water and brine solution. Finally the organic layer was dried over sodium sulphate and concentrated to give 2.7 gm of methane sulphonic acid 4-{2-[(4,5-dimethyl-isoxazol-3-yl)-(2-methoxy-ethoxymethyl)-sulphamoyl]-5-methylthiophene-3-yl}-3-methoxy methyl-benzyl ester.

### STEP 16: Synthesis of 3-[2-methoxymethyl-4-(4,5,7-trimethyl-2-oxo-2H-[1,6]naphthyridin-1-ylmethyl)-phenyl]-5-methyl-thiophene-2-sulphonic acid (4,5-dimethoxy-isoxazol-3-yl)-(2-methoxy-ethoxymethyl)-amide

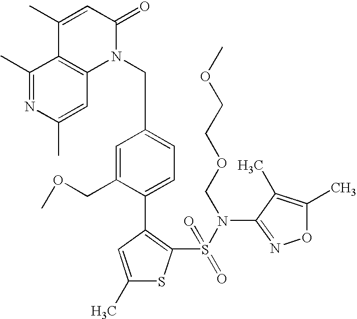

To a stirred solution of 4,5,7-trimethyl-1H-[1,6]naphthyridin-2-one (0.065 gm, 0.0034 mol) in N,N-dimethyl formamide (10 ml) at −15° C. under the flow of dry nitrogen was added portion wise sodium hydride (60% in mineral oil) (0.25 gm, 0.0052 mol). After the completion of the addition, the reaction mixture was warmed to ambient temperature and stirred for 30 min. The reaction mixture was re-cooled to 0° C. and a solution of methane sulphonic acid 4-{2-[(4,5-dimethyl-isoxazol-3-yl)-(2-methoxy-ethoxy methyl)-sulphamoyl]-5-methylthiophene-3-yl}-3-methoxymethyl-benzyl ester (2.1 gm, 0.0035 mmol) in 10 ml of dimethyl formamide was added drop wise. The reaction mixture was stirred at room temperature for 24 hrs and was then diluted with ethyl acetate (40 ml), followed by 10 ml of cold water. The organic layer was separated, washed with water and brine solution. Finally the organic layer was dried over sodium sulphate and evaporated under vacuum to afford crude compound, which was purified by column chromatography on a silica gel column using hexane:ethyl acetate as an eluent to provide 1 gm of 3-[2-methoxymethyl-4-(4,5,7-trimethyl-2-oxo-2H-[1,6]naphthyridin-1-ylmethyl)-phenyl]-5-methyl-thiophene-2-sulphonic acid (4,5-dimethoxy-isoxazol-3-yl)-(2-methoxy-ethoxymethyl)-amide as a viscous oily mass.

### STEP 17: Synthesis of 3-[2-methoxymethyl-4-(4,5,7-trimethyl-2-oxo-2H-[1,6]naphthyridin-1-ylmethyl)-phenyl]-5-methyl-thiophene-2-sulphonic acid (4,5-dimethoxy-isoxazol-3-yl)-amide

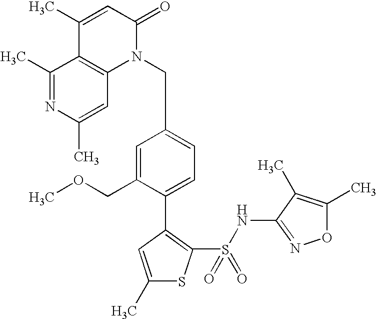

To (1.0 gm, 1.46 mmol) of 3-[2-methoxymethyl-4-(4,5,7-trimethyl-2-oxo-2H-[1,6]naphthyridin-1-ylmethyl)-phenyl]-5-methyl-thiophene-2-sulphonic acid (4,5-dimethoxy-isoxazol-3-yl)-(2-methoxy-ethoxymethyl)-amide was added 95% ethanol (10 ml) and 6N aqueous hydrochloric acid (8 ml) at room temperature. The reaction mixture was refluxed for 3 hrs and was then concentrated under vacuum and the residue thus obtained was diluted with water. The pH of the solution was adjusted to 8 using a saturated solution of sodium bicarbonate, and the mixture was extracted with ethyl acetate (25 ml×3). The combined organic extracts were washed with water and brine then dried over sodium sulphate and concentrated under vacuum. The crude compound was purified by column chromatography on a silica gel column using hexane:ethyl acetate as an eluent to provide 100 mg of 3-[2-methoxymethyl-4-(4,5,7-trimethyl-2-oxo-2H-[1,6]naphthyridin-1-ylmethyl)-phenyl]-5-methyl-thiophene-2-sulphonic acid (4,5-dimethoxy-isoxazol-3-yl)-amide.

Molecular Formula: C₃₀H₃₂N₄O₅S₂

Molecular Weight: 592.74

¹HNMR (DMSOd₆): 1.46 (s, 3H) 2.11 (s, 3H) 2.40 (s, 3H) 2.46 (s, 3H) 2.69 (s, 3H) 2.89 (s, 3H) 3.11 (s, 3H) 4.01 (s, 2H) 5.49 (s, 2H) 6.61 (s, 1H) 6.68 (s, 1H) 6.95 (s, 2H) 7.16 (s, 1H) 7.29 (s, 1H) 10.66 (s, 1H)

Mass Spectrum: (m⁺¹) 593.1

### Example 17

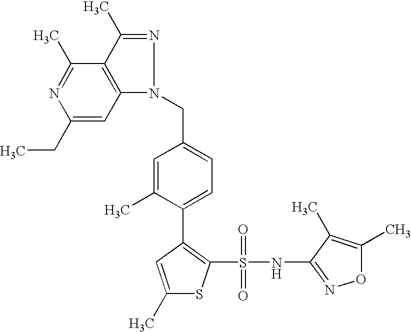

### 3-[4-(6-Ethyl-3,4-dimethyl-pyrazolo[4,3-c]pyridin-1-ylmethyl)-2-methyl-phenyl]-5-methyl-thiophene-2-sulphonic acid (4,5-dimethyl-isoxazol-3-yl)-amide

### STEP 01: Synthesis of 5-(1-hydroxy propylidine)2,2-dimethyl-1,3-dioxane-4,6-dione

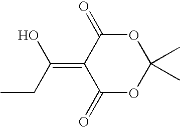

Propionyl chloride (35 ml, 0.381 mol) was added to a solution of Meldrum's acid (50 gm, 0.345 mol) in pyridine (60 ml, 0.690 mol) and methylene chloride (200 ml) at 0° C. within 30 min. and the temperature of the reaction mixture was allowed to rise to ambient temperature and stirred for 1 hr. The reaction mixture was then acidified using 1N hydrochloric acid and extracted with methylene chloride (200 ml×2). The combined extracts were washed with water and brine. The organic layer was dried over sodium sulphate and concentrated under vacuum to give 50 gm of 5-(1-hydroxy propylidine) 2,2-dimethyl-1,3-dioxane-4,6-dione as a crystalline solid.

### STEP 02: Synthesis of 3-Acetyl-6-ethyl-2-methyl-1H-pyridin-4-one

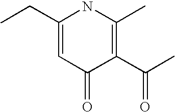

A mixture of 5-(1-hydroxy propylidine) 2,2-dimethyl-1,3-dioxane-4,6-dione (37.8 gm, 0.189 mol) and 4-amino-pent-3-en-2-one (12.5 gm, 0.126 mol) was heated to reflux at 120° C. for 2 hrs. The reaction mixture was cooled and the crude compound was purified by column chromatography on a silica gel column using 10% methanol: ethyl acetate as an eluent to provide 6 gm of 3-Acetyl-6-ethyl-2-methyl-1H-pyridin-4-one as a yellow colored solid.

### STEP 03: Synthesis of 1-(4-chloro-6-ethyl-2-methyl-pyridin-3-yl)ethanone

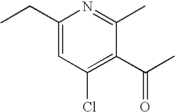

3-Acetyl-6-ethyl-2-methyl-1H-pyridin-4-one (5 gm, 0.027 mol) was added to 10 ml of phosphorous oxychloride at 0° C. The reaction mixture was heated with stirring to 50° C. and maintained at this temperature for 8 hours. The reaction mixture was then evaporated under vacuum and the residue thus obtained was basified to pH 8 with saturated sodium bicarbonate solution, followed by extraction with methylene dichloride (50 ml×2). The combined organic extracts were washed with water and brine and dried over anhydrous sodium sulphate and concentrated to give 3.2 gm of 1-(4-chloro-6-ethyl-2-methyl-pyridin-3-yl)ethanone as a yellow oily liquid.

### STEP 04: Synthesis of 6-ethyl-3,4 dimethyl-1H-pyrazolo[4,3-c]pyridine

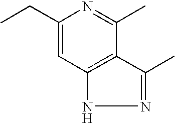

1-(4-Chloro-6-ethyl-2-methyl-pyridin-3-yl)ethanone (1.7 gm, 0.0086 mol) was dissolved in ethanol (20 ml) and hydrazine hydrate (2.15 ml, 0.038 mol) was added. The reaction mixture was slowly heated to reflux, and maintained at reflux for 4 hours. The reaction mixture was then evaporated under vacuum and the residue of the crude compound was dumped onto ice. The solid thus obtained was filtered off and suction dried to provide 1 gm of 6-ethyl-3,4 dimethyl-1H-pyrazolo[4,3-c]pyridine.

### STEP 05: Synthesis of (5-methyl-isoxazol-3-yl) carbamic acid tert-butyl ester

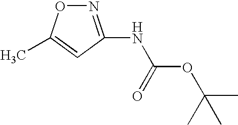

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 06: Synthesis of (4,5-dimethyl-isoxazol-3-yl) carbamic acid tert-butyl ester

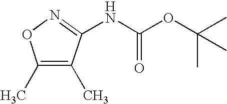

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 07: Synthesis of 4,5-dimethyl-isoxazol-3-yl-amine

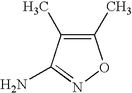

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 08: Synthesis of 5-methyl thiophene-2-sulphonyl chloride

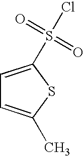

A solution of 2-methyl thiophene (50 gm, 0.51 mol) in chloroform was added to a solution of chloro sulphonic acid (105 ml, 1.53 mol) in chloroform at −5° C. to 0° C. The reaction mixture was maintained at 0° C. for 3 hrs and the crude reaction mass was then slowly dumped into ice cold water, followed by extraction with chloroform (100 ml×2). The combined extract was washed with water and brine. Finally the organic layer was dried over anhydrous sodium sulphate, evaporated under vacuum to give 16 gm of 5-methyl thiophene-2-sulphonyl chloride as a brown colored liquid.

### STEP 09: Synthesis of 5-methyl-thiophene-2-sulphonic acid (4,5-dimethyl-isoxazol-3yl) amide

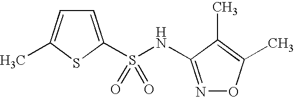

The solution of 5-Methyl thiophene-2-sulphonyl chloride (16.0 gm, 0.081 mol) in (25 ml) methylene chloride was added to the solution of 3-amino-4,5-dimethylisoxazole (6.2 gm, 0.055 mol) and dimethylaminopyridine (500 mg) in pyridine (40 ml) at 0° C. After the completion of the addition the temperature of the reaction mixture was slowly raised to room temperature and stirred it for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 18 gm of 5-methyl-thiophene-2-sulphonic acid (4,5-dimethyl-isoxazol-3yl) amide as brown colored solid.

### STEP 10: Synthesis of 5-methyl-thiophene-2-sulphonic acid (4,5 dimethyl-isoxazol-3-yl)-(2-methoxy-ethoxymethyl)-amide

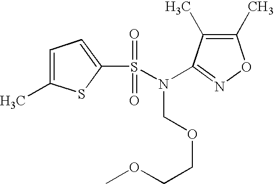

Under the flow of dry nitrogen and stirring, sodium hydride (3.4 gm, 0.07 mol, 60% dispersion in mineral oil) was added in to N,N-dimethyl formamide (40 ml) at 0° C., followed by addition of 5-methyl-thiophene-2-sulphonic acid (4,5-dimethyl-isoxazol-3yl) amide (16.5 gm, 0.060 mol) to it. After completion of the addition, temperature of the reaction mixture was slowly raised and stirred it at ambient temperature for 30 minutes then recooled the reaction mixture to 0° C., followed by the drop wise addition of methoxy ethoxy methyl chloride (8.03 gm, 0.064 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 18.2 gm of 5-methyl-thiophene-2-sulphonic acid (4,5-dimethyl-isoxazol-3yl) amide as yellowish oil.

### STEP 11: Synthesis of 3-borono-N-(4,5-dimethyl-3-isoxazolyl)-N-[(2-methoxy-ethoxy)methyl]-5-methyl-thiophene sulphonamide

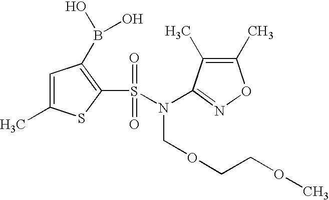

Under the dry nitrogen atmosphere the solution of (14 gm, 0.038 mol) 5-methyl-thiophene-2-sulphonic acid (4,5 dimethyl-isoxazol-3-yl)-(2-methoxy-ethoxymethyl)-amide in tetrahydrofuran (80 ml) was cooled to −78° C. To this n-Butyl lithium (60 ml, 0.097 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (15 ml, 0.062 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×3). The combined extract was washed with water and brine solution. The organic layer was dried over sodium sulphate and concentrated under vacuum to give 15 gm of 3-borono-N-(4,5-dimethyl-3-isoxazolyl)-N-[(2-methoxy-ethoxy)methyl]-5-methyl-thiophene sulphonamide as thick oily mass.

### STEP 12: Synthesis of (4-{2-[(4,5-dimethyl-isoxazol-3-yl)-(2-methoxy-ethoxymethyl)-sulphamoyl]-5-methyl-thiophene-3-yl}-3-methyl-benzoic acid methyl ester

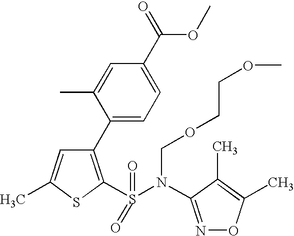

To a stirred solution of 4-bromo-3-methyl-benzoic acid methyl ester (4.72 gm, 0.0247 mol) in dimethoxy ethane (50 ml) under nitrogen, bis(triphenylphosphine)palladium(II)chloride (1.45 gm, 0.002 mol) was added followed by addition of a 2M aqueous sodium carbonate solution (6.5 gm in 30 ml water), The reaction mixture was stirred at room temperature for 10 min and then heated at 60° C. To this mixture a solution of 3-borono-N-(4,5-Dimethyl-3-isoxazolyl)-N-[(2-methoxy-ethoxy)methyl]-5-methyl-thiophene sulphonamide (5 gm, 0.012 mol in 25 ml dimethoxy ethane) was added drop wise within 45 min and the reaction was then refluxed for 60 min. After 1 hr the same procedure was repeated with further addition of 3-borono-N-(4,5-dimethyl-3-isoxazolyl)-N-[(2-methoxy-ethoxy)methyl]-5-methyl-thiophene sulphonamide (5 gm, 0.012 mol in 25 ml dimethoxy ethane) within 45 min. The reaction mixture was refluxed for 4 hrs and stirred at room temperature for 12 hrs. The mixture was cooled and diluted with ethyl acetate (100 ml) and water, the organic layer was separated, and the aqueous layer further extracted with ethyl acetate (50 ml×2). The combined extract was washed with water and brine. Finally the organic layer was dried over sodium sulphate and concentrated under vacuum. The crude compound was purified on a silica gel column using 4:1 hexane/ethyl acetate as an eluent to provide 9.7 gm of (4-{2-[(4,5-dimethyl-isoxazol-3-yl)-(2-methoxy-ethoxymethyl)-sulphamoyl]-5-methyl-thiophene-3-yl}-3-methyl-benzoic acid methyl ester as pale yellow oily mass.

### STEP 13: Synthesis of 3-(4-hydroxy methyl-2-methyl-phenyl)-5-methyl-thiophene-2-sulphonic acid-(4,5-dimethyl-isoxazol-3-yl)-(2-methoxy-ethoxymethyl) amide

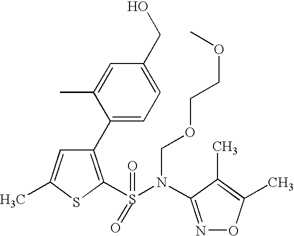

Lithium aluminium hydride (1 gm, 0.026 mol) was added to a stirred solution of tetrahydrofuran (15 ml) at 0° C. under flow of nitrogen, followed by addition of (4-{2-[(4,5-dimethyl-isoxazol-3-yl)-(2-methoxy-ethoxymethyl)-sulphamoyl]-5-methyl-thiophene-3-yl}-3-methyl-benzoic acid methyl ester (9.7 gm, 0.019 mol) in 75 ml of tetrahydrofuran. The reaction mixture was stirred at 0° C. for 1 hr and then the temperature was raised to room temperature and stirred for 4 hrs. The reaction mixture was cooled to 0° C., and a sodium hydroxide solution (50 ml) (1 gm dissolved in 100 ml water) was added drop wise while maintaining the temperature at 0° C.—The mixture was extracted with ethyl acetate (25 ml×2) and the organic layer was washed with water and brine solution. Finally organic layer was dried over sodium sulphate and concentrated under vacuum. The crude compound was purified by column chromatography on a silica gel column using 1:3 hexane/ethyl acetate as an eluent to provide 5.7 gm of 3-(4-hydroxy methyl-2-methyl-phenyl)-5-methyl-thiophene-2-sulphonic acid-(4,5-dimethyl-isoxazol-3-yl)-(2-methoxy-ethoxymethyl) amide

### STEP 14: Synthesis of methane sulphonic acid 4-{2-[(4,5-dimethyl-isoxazol-3yl)-(2-methoxy-ethoxy methyl)-sulphamoyl]-5-methylthiophene-3-yl}-3-methyl-benzyl ester

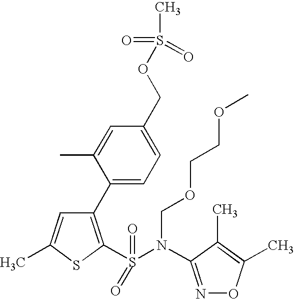

N-Ethyl diisopropyl amine (3 ml, 0.017 mol) was added to a solution of 3-(4-hydroxy methyl-2-methyl-phenyl)-5-methyl-thiophene-2-sulphonic acid-(4,5-Dimethyl-isoxazol-3-yl)-(2-methoxy-ethoxymethyl) amide (5.7 gm, 0.012 mol) in 40 ml of dichloro methane. The reaction mixture was cooled to 0° C., and then methane sulphonyl chloride (1.16 ml, 0.014 mol) was added slowly. After the completion of the addition the reaction mixture was maintained at room temperature for 3 hrs. The reaction mixture was then dumped into ice-cold water followed by extraction with methylene chloride (50 ml×2). The combined extract was washed with dilute hydrochloric acid followed by washings with water and brine solution. Finally the organic layer was dried over sodium sulphate and concentrated under vacuum to give 6 gm of methane sulphonic acid 4-{2-[(4,5-dimethyl-isoxazol-3-yl)-(2-methoxy-ethoxy methyl)-sulphamoyl]-5-methylthiophene-3-yl}-3-methyl-benzyl ester.

### STEP 15: Synthesis of 3-[4-(6-ethyl-3,4-dimethyl-pyrazolo[4,3-c]pyridin-1ylmethyl)-2-methyl-phenyl]-5-methyl-thiophene-2-sulphonic acid (4,5-dimethyl-isoxazol-3-yl)-(2-methoxy-ethoxymethyl)-amide

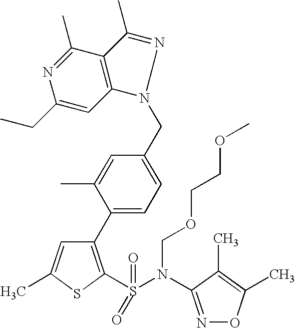

To a stirred solution of 6-ethyl-3,4 dimethyl-1H-pyrazolo[4,3-c]pyridine (0.59 gm, 3.5 mmol) in N,N-dimethyl formamide (5 ml) at −15° C. under the flow of dry nitrogen, sodium hydride (60% in mineral oil) (0.26 gm, 5.4 mmol) was added portion wise. After the completion of the addition, the reaction mixture was warmed to ambient temperature and stirred for 30 min. The reaction mixture was then cooled to 0° C. and a solution of methane sulphonic acid 4-{2-[(4,5-dimethyl-isoxazol-3-yl)-(2-methoxy-ethoxy methyl)-sulphamoyl]-5-methylthiophene-3-yl}-3-methyl-benzyl ester (2 gm, 3.5 mmol) in 5 ml of N,N-dimethyl formamide was added drop wise The mixture was then stirred at room temperature for 24 hrs and was then diluted with ethyl acetate (40 ml), followed by 10 ml of cold water. The organic layer was separated and washed with water and brine solution. Finally the organic layer was then dried over sodium sulphate and evaporated under vacuum to afford 2 gm of 3-[4-(6-ethyl-3,4-dimethyl-pyrazolo[4,3-c]pyridin-1-ylmethyl)-2-methyl-phenyl]-5-methyl-thiophene-2-sulphonic acid (4,5-dimethyl-isoxazol-3-yl)-(2-methoxy-ethoxymethyl)-amide as a viscous oily mass.

### STEP 16: Synthesis of 3-[4-(6-ethyl-3,4-dimethyl-pyrazolo[4,3-c]pyridin-1-ylmethyl)-2-methyl-phenyl]-5-methyl-thiophene-2-sulphonic acid (4,5-dimethyl-isoxazol-3-yl)-amide

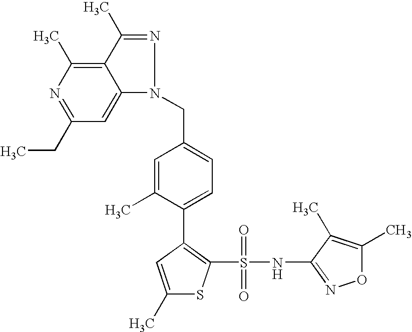

To 3-[4-(6-ethyl-3,4-dimethyl-pyrazolo[4,3-c]pyridin-1-ylmethyl)-2-methyl-phenyl]-5-methyl-thiophene-2-sulphonic acid (4,5-dimethyl-isoxazol-3-yl)-(2-methoxy-ethoxymethyl)-amide (2 gm, 3.13 mmol) was added 95% ethanol (10 ml) and 6N aqueous hydrochloric acid (8 ml) at room temperature. The reaction mixture was refluxed for 3 hrs. The reaction mixture was then concentrated under vacuum, and the residue thus obtained was diluted with water and the pH of the solution was adjusted to 8 using a saturated solution of sodium bicarbonate. After that the pH of the solution was finally adjusted to 5 with acetic acid, and then the mixture was extracted with ethyl acetate (25 ml×2). The combined organic extract was washed with water and brine solution. Finally the organic layer was dried over sodium sulphate and concentrated under vacuum. The crude compound was purified by column chromatography on a silica gel column using hexane:ethyl acetate as an eluent to provide 300 mg of 3-[4-(6-ethyl-3,4-dimethyl-pyrazolo[4,3-c]pyridin-1-ylmethyl)-2-methyl-phenyl]-5-methyl-thiophene-2-sulphonic acid (4,5-dimethyl-isoxazol-3-yl)-amide

Molecular Formula: C₂₈H₃₁N₅O₃S₂

Molecular Weight: 549.72

¹HNMR (DMSOd₆): 1.26 (t, J=7.2 Hz, 3H) 1.49 (s, 3H) 1.93 (s, 3H) 2.14 (s, 3H) 2.47 (s, 3H) 2.63 (s, 3H) 2.75-2.81 (m, 5H) 5.49 (s, 2H) 6.68 (s, 1H) 6.89 (s, 2H) 7.10 (s, 1H) 7.37 (s, 1H) 10.80 (s, 1H)

Mass Spectrum: (m⁺¹) 550.2

### Example 18

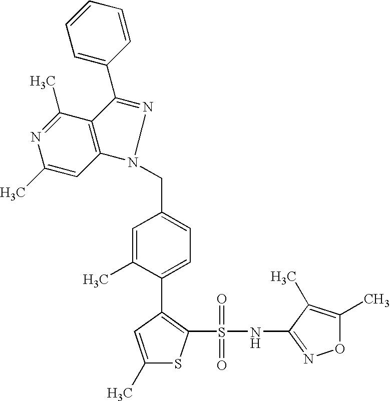

### 3-(4,6-Dimethyl-3-phenyl-pyrazolo[4,3-c]pyridin-1-ylmethyl)-2-methyl-phenyl]-5-methyl-thiophene-2-sulphonic acid (4,5-dimethyl-isoxazol-3-yl)-amide

### STEP 01: Synthesis of 3-benzoyl-2,6-dimethyl-1H-pyridin-4-one

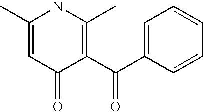

A mixture of 2,2,6-trimethyl-[1,3] dioxin-4-one (15 ml, 0.10 mol) and 3-amino-1-phenyl-but-2-en-1-one (5.8 gm, 0.036 mol) was heated to reflux at 120° C. for 6 hrs. The reaction mixture was purified by column chromatography over silica gel, eluting the desired product with 10% methanol and ethyl acetate to give 2 gm of 3-benzoyl-2,6-dimethyl-1H-pyridin-4-one

### STEP 02: Synthesis of (4-chloro-2,6-dimethyl-pyridin-3-yl)-phenyl-methanone

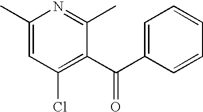

3-Benzoyl-2,6-dimethyl-1H-pyridin-4-one (2 gm, 0.008 mol) was added to 15 ml phosphorous oxychloride at 0° C. The mixture was heated with stirring the to 100° C. and maintained like that for 8 hours. Work-up was done by evaporating the phosphorus oxy chloride under vacuum. The resulting residue was basified to pH 8 with saturated sodium carbonate solution, followed by extraction with methylene dichloride (50 ml×2). The combined extracts were washed with water and brine and dried over anhydrous sodium sulphate and concentrated to give 2 gm of (4-chloro-2,6-dimethyl-pyridin-3-yl)-phenyl-methanone

### STEP 03: Synthesis of 4,6-dimethyl-3-phenyl-1H-pyrazolo[4,3-c]pyridine

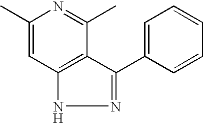

(4-chloro-2,6-dimethyl-pyridin-3-yl)-phenyl methanone (2 gm, 0.008 mol) was taken in ethanol (15 ml) where after hydrazine hydrate (3 ml, 0.06 mol) and two drops of acetic acid was added. The temperature of the reaction mixture was slowly raised to reflux, where it was maintained for 6 hours. The reaction mixture was completely evaporated under vacuum. The crude mass was dumped onto ice, and the solid obtained was filtered off and suction dried to provide 1.61 gm of 4,6-dimethyl-3-phenyl-1H-pyrazolo[4,3-c]pyridine.

### STEP 04: Synthesis of (5-methyl-isoxazol-3-yl) carbamic acid tert-butyl ester

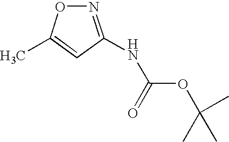

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 05: Synthesis of (4,5-dimethyl-isoxazol-3-yl) carbamic acid tert-butyl ester

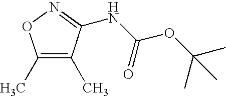

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 06: Synthesis of 4,5-dimethyl-isoxazol-3-yl-amine

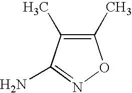

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 07: Synthesis of 5-methyl thiophene-2-sulphonyl chloride

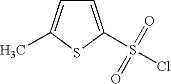

A solution of 2-methyl thiophene (50 gm, 0.51 mol) in chloroform was added to a solution of chloro sulphonic acid (105 ml, 1.53 mol) in chloroform at −5° C. to 0° C. The reaction mixture was maintained at 0° C. for 3 hrs. The crude reaction mass was slowly dumped into ice cold water, followed by extraction with chloroform (100 ml×2). The combined extract was washed with water and brine. Finally the organic layer was dried over anhydrous sodium sulphate, and evaporated under vacuum to give 16 gm of 5-methyl thiophene-2-sulphonyl chloride as a brown colored liquid.

### STEP 08: Synthesis of 5-methyl-thiophene-2-sulphonic acid (4,5-dimethyl-isoxazol-3yl) amide

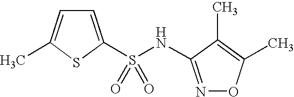

The solution of 5-Methyl thiophene-2-sulphonyl chloride (16.0 gm, 0.081 mol) in (25 ml) methylene chloride was added to the solution of 3-amino-4,5-dimethylisoxazole (6.2 gm, 0.055 mol) and dimethylaminopyridine (500 mg) in pyridine (40 ml) at 0° C. After the completion of the addition the temperature of the reaction mixture was slowly raised to room temperature and stirred it for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 18 gm of 5-methyl-thiophene-2-sulphonic acid (4,5-dimethyl-isoxazol-3yl) amide as brown colored solid.

### STEP 09: Synthesis of 5-methyl-thiophene-2-sulphonic acid (4,5 dimethyl-isoxazol-3-yl)-(2-methoxy-ethoxymethyl)-amide

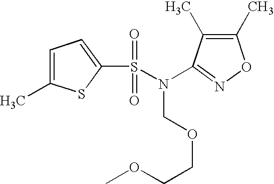

Under the flow of dry nitrogen and stirring, sodium hydride (3.4 gm, 0.07 mol, 60% dispersion in mineral oil) was added in to N,N-dimethyl formamide (40 ml) at 0° C., followed by addition of 5-methyl-thiophene-2-sulphonic acid (4,5-dimethyl-isoxazol-3yl) amide (16.5 gm, 0.060 mol) to it. After completion of the addition, temperature of the reaction mixture was slowly raised and stirred it at ambient temperature for 30 minutes then recooled the reaction mixture to 0° C., followed by the drop wise addition of methoxy ethoxy methyl chloride (8.03 gm, 0.064 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 18.2 gm of 5-methyl-thiophene-2-sulphonic acid (4,5-dimethyl-isoxazol-3yl) amide as yellowish oil.

### STEP 10: Synthesis of 3-borono-N-(4,5-dimethyl-3-isoxazolyl)-N-[(2-methoxy-ethoxy)methyl]-5-methyl-thiophene sulphonamide

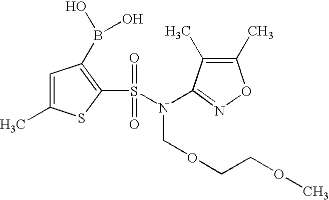

Under the dry nitrogen atmosphere the solution of (14 gm, 0:038 mol) 5-methyl-thiophene-2-sulphonic acid (4,5 dimethyl-isoxazol-3-yl)-(2-methoxy-ethoxymethyl)-amide in tetrahydrofuran (80 ml) was cooled to −78° C. To this n-Butyl lithium (60 ml, 0.097 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (15 ml, 0.062 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×3). The combined extract was washed with water and brine solution. The organic layer was dried over sodium sulphate and concentrated under vacuum to give 15 gm of 3-borono-N-(4,5-dimethyl-3-isoxazolyl)-N-[(2-methoxy-ethoxy)methyl]-5-methyl-thiophene sulphonamide as thick oily mass.

### STEP 11: Synthesis of (4-{2-[(4,5-dimethyl-isoxazol-3-yl)-(2-methoxy-ethoxymethyl)-sulphamoyl]-5-methyl-thiophene-3-yl}-3-methyl-benzoic acid methyl ester

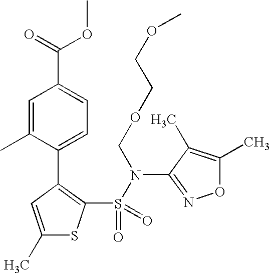

To a stirred solution of 4-bromo-3-methyl-benzoic acid methyl ester (4.72 gm, 0.0247 mol) in dimethoxy ethane (50 ml) under nitrogen, bis(triphenylphosphine)palladium(II)chloride (1.45 gm, 0.002 mol) was added followed by the addition of a 2M aqueous sodium carbonate solution (6.5 gm in 30 ml water). The reaction mixture was stirred at room temperature for 10 min and then heated at 60° C. To this a solution of 3-borono-N-(4,5-dimethyl-3-isoxazolyl)-N-[(2-methoxy-ethoxy)methyl]-5-methyl-thiophene sulphonamide (5 gm, 0.012 mol in 25 ml dimethoxy ethane) was added drop wise within 45 min, and then the reaction was refluxed for 60 min. After 1 hr the same procedure was repeated with further addition of 3-borono-N-(4,5-dimethyl-3-isoxazolyl)-N-[(2-methoxy-ethoxy)methyl]-5-methyl-thiophene sulphonamide (5 gm, 0.012 mol in 25 ml dimethoxy ethane) within 45 min. The reaction mixture was refluxed for 4 hrs and then stirred at room temperature for 12 hrs. The mixture was cooled and diluted with ethyl acetate (100 ml) and water, the organic layers were separated, and the aqueous layer further extracted with ethyl acetate (50 ml×2). The combined extract was washed with water and brine. Finally the organic layer was dried over sodium sulphate and concentrated under vacuum. The crude compound was purified on a silica gel column using 4:1 hexane/ethyl acetate as an eluent to provide 9.7 gm of (4-{2-[(4,5-dimethyl-isoxazol-3-yl)-(2-methoxy-ethoxymethyl)-sulphamoyl]-5-methyl-thiophene-3-yl}-3-methyl-benzoic acid methyl ester as pale yellow oily mass.

### STEP 12: Synthesis of 3-(4-hydroxy methyl-2-methyl-phenyl)-5-methyl-thiophene-2-sulphonic acid-(4,5-Dimethyl-isoxazol-3-yl)-(2-methoxy-ethoxymethyl) amide

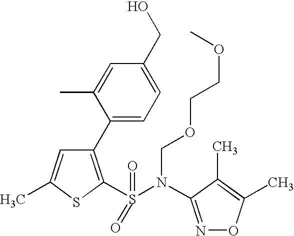

Lithium aluminium hydride (1 gm, 0.026 mol) was added to a stirred solution of tetrahydrofuran (15 ml) at 0° C. under flow of nitrogen, followed by the addition of (4-{2-[(4,5-dimethyl-isoxazol-3-yl)-(2-methoxy-ethoxymethyl)-sulphamoyl]-5-methyl-thiophene-3-yl}-3-methyl-benzoic acid methyl ester (9.7 gm, 0.019 mol) in 75 ml of tetrahydrofuran. The reaction mixture was stirred at 0° C. for 1 hr and then the temperature was raised to room temperature and the mixture stirred for 4 hrs. The reaction mixture was cooled to 0° C., and drop a sodium hydroxide solution (50 ml) (1 gm dissolved in 100 ml water) was added drop wise while maintaining the temperature at 0° C., followed by extraction with ethyl acetate (25 ml×2). The organic layer was washed with water and brine solution. Finally the organic layer was dried over sodium sulphate and concentrated under vacuum. The crude compound was purified on a silica gel column using 1:3 hexane/ethyl acetate as an eluent to provide 5.7 gm of 3-(4-hydroxy methyl-2-methyl-phenyl)-5-methyl-thiophene-2-sulphonic acid-(4,5-dimethyl-isoxazol-3-yl)-(2-methoxy-ethoxymethyl) amide

### STEP 13: Synthesis of methane sulphonic acid 4-{2-[(4,5-dimethyl-isoxazol-3)-(2-methoxy-ethoxy methyl)-sulphamoyl]-5-methylthiophene-3-yl}-3-methyl-benzyl ester

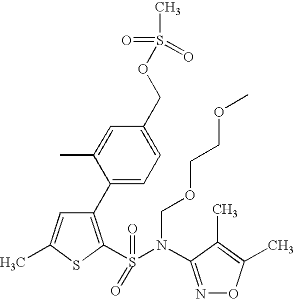

N-Ethyl diisopropyl amine (3 ml, 0.017 mol) was added to a solution of 3-(4-hydroxy methyl-2-methyl-phenyl)-5-methyl-thiophene-2-sulphonic acid-(4,5-dimethyl-isoxazol-3-yl)-(2-methoxy-ethoxymethyl) amide (5.7 gm, 0.012 mol) in 40 ml of dichloro methane. The reaction mixture was cooled to 0° C., and then methane sulphonyl chloride (1.16 ml, 0.014 mol) was slowly added into the reaction mixture. After the completion of the addition the reaction mixture was maintained at room temperature for 3 hrs. The reaction mixture was dumped into ice-cold water followed by extraction with methylene chloride (50 ml×2). The combined extract was washed with dilute hydrochloric acid followed by washings with water and brine solution. Finally the organic layer was dried over sodium sulphate and concentrated under vacuum to give 6 gm of methane sulphonic acid 4-{2-[(4,5-dimethyl-isoxazol-3-yl)-(2-methoxy-ethoxy methyl)-sulphamoyl]-5-methylthiophene-3-yl}-3-methyl-benzyl ester.

### STEP 14: Synthesis of 3-[4-(4,6-dimethyl-3-phenyl-pyrazolo[4,3-c]pyridin-1-ylmethyl)-2-methyl-phenyl]-5-methyl-thiophene-2-sulphonic acid (4,5-dimethyl-isoxazol-3-yl)-(2-methoxy-ethoxymethyl)-amide

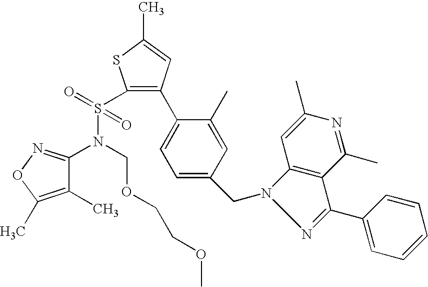

To a stirred solution of 4,6-dimethyl-3-phenyl-1H-pyrazolo[4,3-c]pyridine (749 mg, 3.5 mmol) in dimethyl formamide (15 ml) at −15° C. under nitrogen, sodium hydride (60% in mineral oil) (0.24 gm, 5 mmol) was added. After the addition, the reaction mixture was warmed to ambient temperature and maintained for 30 min. The reaction mixture was re-cooled to 0° C. and a solution of methane sulphonic acid 4-{2-[(4,5-dimethyl-isoxazol-3-yl)-(2-methoxy-ethoxy methyl)-sulphamoyl]-5-methylthiophene-3-yl}-3-methyl-benzyl ester (2 gm, 3.5 mmol) in 10 ml of N,N-dimethyl formamide was added drop wise to the reaction mixture and stirred at room temperature for 24 hrs. The mixture was then diluted with ethyl acetate (40 ml), followed by 10 ml of cold water. The organic layer was separated and washed with water and brine. Finally the organic layer was dried over sodium sulphate and evaporated under vacuum to afford crude 2.2 gm of 3-[4-(4,6-Dimethyl-3-phenyl-pyrazolo[4,3-c]pyridin-1-ylmethyl)-2-methyl-phenyl]-5-methyl-thiophene-2-sulphonic acid (4,5-dimethyl-isoxazol-3-yl)-(2-methoxy-ethoxymethyl)-amide as a viscous oily mass.

### STEP 15: Synthesis of 3-(4,6-dimethyl-3-phenyl-pyrazolo[4,3-c]pyridin-1-ylmethyl)-2-methyl-phenyl]-5-methyl-thiophene-2-sulphonic acid (4,5-dimethyl-isoxazol-3-yl)-amide

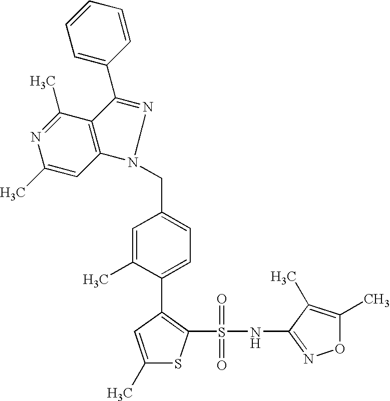

To (2.2 gm, 2.95 mmol) of crude 3-(4,6-Dimethyl-3-phenyl-pyrazolo[4,3-c]pyridin-1-ylmethyl)-2-methyl-phenyl]-5-methyl-thiophene-2-sulphonic acid (4,5-dimethyl-isoxazol-3-yl)-(2-methoxy-ethoxymethyl)-amide was added ethanol (10 ml) and 6N aqueous hydrochloric acid (8 ml) at room temperature. The reaction mixture was stirred and heated for 3 hrs was then concentrated under vacuum. The residue thus obtained was diluted with water and then the pH of the solution was adjusted to 8 using a saturated solution of sodium bicarbonate. The mixture was then extracted with ethyl acetate (25 ml×3) and the combined organic extract was washed with water and brine solution. Finally the organic layer was dried over sodium sulphate and concentrated under vacuum. The crude compound was purified on a silica gel column using 1:1 hexane/ethyl acetate as an eluent to provide 300 mg of 3-(4,6-dimethyl-3-phenyl-pyrazolo[4,3-c]pyridin-1-ylmethyl)-2-methyl-phenyl]-5-methyl-thiophene-2-sulphonic acid (4,5-dimethyl-isoxazol-3-yl)-amide

Molecular Formula: C₃₂H₃₁N₅O₃S₂

Molecular Weight: 597.76

¹HNMR (DMSOd₆): 1.46 (s, 3H) 1.93 (s, 3H) 2.11 (s, 3H) 2.47 (s, 3H) 2.48 (s, 3H) 2.55 (s, 3H) 5.63 (s, 2H) 6.69 (s, 1H) 6.89-6.91 (m, 1H) 6.98-7.00 (m, 1H) 7.18 (s, 1H) 7.50-7.68 (m, 6H) 10.75 (s, 1H)

Mass Spectrum: (m⁺¹) 598.2

### Example 19

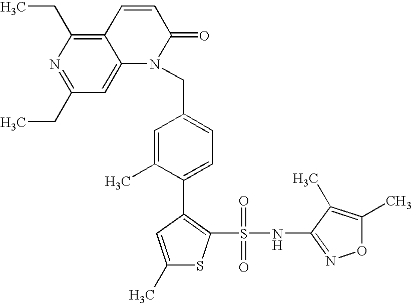

### 3-[4-(5,7-Diethyl-2-oxo-2H-[1,6]naphthyridin-1-ylmethyl)-2-methyl-phenyl]-5-methyl-thiophene-2-sulphonic acid(4,5-dimethyl-isoxazol-3-yl)-(2-methoxy-ethoxymethyl)amide

### STEP 01: Synthesis of (5-methyl-isoxazol-3-yl) carbamic acid tert-butyl ester

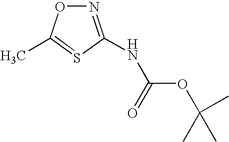

5-methyl-isoxazol-3-ylamine (100 gm, 1.019 mol) was dissolved in pyridine (200 ml, 2.545 mol) and then cooled this mixture to 0° C. To this reaction mixture di-tert-butyl dicarbonate (245 gm, 1.12 mol) was added in 1 hr. After the completion of the addition, the reaction mixture was stirred at room temperature for 12 hrs. Then the reaction mixture was concentrated at 60-70° C. under vacuum. The residue thus obtained was dissolved in (500 ml) ethyl acetate. The ethyl acetate layer was washed with dilute hydrochloric acid followed by water and brine washings. Finally organic layer was dried over sodium sulphate and evaporated under vacuum to give crude product. The crude product was dissolved in hot toluene (200 ml) and upon standing for 2 hrs at room temperature the solid crystallized out, which was filtered off, washed with cold toluene (50 ml) and suction dried to give (130 gm) of (5-methyl-isoxazol-3-yl)carbamic acid tert-butyl ester.

### STEP 02: Synthesis of (4,5-dimethyl-isoxazol-3-yl) carbamic acid tert-butyl ester

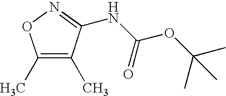

Under the dry nitrogen atmosphere (5-methyl-isoxazol-3-yl) carbamic acid tert-butyl ester (20 gm, 0.10 mol) was dissolved in hexane (150 ml) and N,N,N′,N′-tetra methyl ethylene diamine (35 ml, 0.221 mol) was added to it. This reaction mixture was then cooled to −78° C. To the reaction mixture n-butyl lithium (106 ml, 0.250 mol, 15% solution in hexane) was charged in 30 minutes maintaining the temperature of the reaction mixture at −78° C. The reaction mixture was stirred for 1 hr and methyl iodide (12 ml, 0.15 mol) was added to it. After the completion of the addition, the reaction mixture was stirred at −10° C. for 4 hrs. Then the reaction mixture was quenched with the saturated solution of ammonium chloride (60 ml). The solid thus obtained was filtered off, washed with cold hexane (50 ml) and suction dried to give 22 gm of (4,5-dimethyl-isoxazol-3-yl) carbamic acid tert-butyl ester.

### STEP 03: Synthesis of 4,5-dimethyl-isoxazol-3-yl-amine

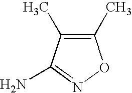

(4,5-dimethyl-isoxazol-3-yl) carbamic acid tert-butyl ester (22 gm, 0.1036 mol) was portion wise added to the trifluoro acetic acid (22 ml 0.3108 mol) at 0° C. After the completion of the addition, the reaction mixture was warmed to 60° C. and stirred it for 2 hrs. The reaction mixture was cooled to room temperature and basified with a saturated solution of sodium bicarbonate. The product was extracted with methylene dichloride (100 ml×2). The organic layer was washed with water and brine solution. Finally organic layer was dried over sodium sulphate and evaporated under vacuum to give 9 gm of 4,5-dimethyl-isoxazol-3-ylamine as yellow solid.

### STEP 04: Synthesis of 5-methyl thiophene-2-sulphonyl chloride

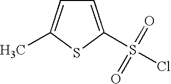

A solution of 2-methyl thiophene (50 gm, 0.51 mol) in chloroform was added to a solution of chloro sulphonic acid (105 ml, 1.53 mol) in chloroform at −5° C. to 0° C. Then the reaction mixture was maintained at 0° C. for 3 hrs. The crude reaction mass was slowly dumped into the ice cold water, followed by extraction with chloroform (100 ml×2). The combined extract was washed with water and brine. Finally the organic layer was dried over anhydrous sodium sulphate, and evaporated under vacuum to give 16 gm of 5-Methyl thiophene-2-sulphonyl chloride as a brown colored liquid.

### STEP 05: Synthesis of 5-methyl-thiophene-2-sulphonic acid (4,5-dimethyl-isoxazol-3yl) amide

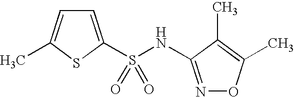

The solution of 5-Methyl thiophene-2-sulphonyl chloride (16.0 gm, 0.081 mol) in (25 ml) methylene chloride was added to the solution of 3-amino-4,5-dimethylisoxazole (6.2 gm, 0.055 mol) and dimethylaminopyridine (500 mg) in pyridine (40 ml) at 0° C. After the completion of the addition the temperature of the reaction mixture was slowly raised to room temperature and stirred it for 6 hours. The reaction mixture was then concentrated under vacuum, the residue thus obtained was acidified using 1N hydrochloric acid followed by extraction with methylene chloride (100 ml×2). The combined extracts were washed with water and brine solution. Organic layer was then dried over sodium sulphate and concentrated to give 18 gm of 5-methyl-thiophene-2-sulphonic acid (4,5-dimethyl-isoxazol-3yl) amide as brown colored solid.

### STEP 06: Synthesis of 5-methyl-thiophene-2-sulphonic acid (4,5 dimethyl-isoxazol-3-yl)-(2-methoxy-ethoxymethyl)-amide

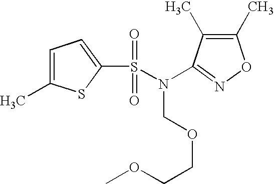

Under the flow of dry nitrogen and stirring, sodium hydride (3.4 gm, 0.07 mol, 60% dispersion in mineral oil) was added in to N,N-dimethyl formamide (40 ml) at 0° C., followed by addition of 5-methyl-thiophene-2-sulphonic acid (4,5-dimethyl-isoxazol-3yl) amide (16.5 gm, 0.060 mol) to it. After completion of the addition, temperature of the reaction mixture was slowly raised and stirred it at ambient temperature for 30 minutes then recooled the reaction mixture to 0° C., followed by the drop wise addition of methoxyethoxymethyl chloride (8.03 gm, 0.064 mol) to the reaction mixture. After completion of the addition, the temperature of the reaction mixture was slowly raised to ambient temperature and stirred for 3 hrs. Then the reaction mixture was cooled to 0° C. and to it (90 ml) ethyl acetate was added and stirred the reaction mixture for 20 min, followed by addition of (25 ml) ice water to the reaction mixture. The organic layer was separated; the aqueous layer was again extracted with ethyl acetate (50 ml×2). The combined extracts were washed with water and brine solution and dried over sodium sulphate. The organic layer was concentrated under vacuum. The crude product was purified by column chromatography on a silica gel column using ethyl acetate:hexane as an eluent to provide 18.2 gm of 5-methyl-thiophene-2-sulphonic acid (4,5-dimethyl-isoxazol-3yl) amide as yellowish oil.

### STEP 07: Synthesis of 3-borono-N-(4,5-dimethyl-3-isoxazolyl)-N-[(2-methoxy-ethoxy)methyl]-5-methyl-thiophene sulphonamide

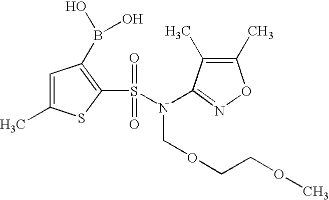

Under the dry nitrogen atmosphere the solution of (14 gm, 0.038 mol) 5-methyl-thiophene-2-sulphonic acid (4,5 dimethyl-isoxazol-3-yl)-(2-methoxy-ethoxymethyl)-amide in tetrahydrofuran (80 ml) was cooled to −78° C. To this n-Butyl lithium (60 ml, 0.097 mol, 15% solution in n-hexane) was added slowly. After the completion of the addition the reaction mixture was stirred at −78° C. for 1 hr and then the temperature of the reaction mixture was slowly raised to 0° C. and then reaction mixture stirred for 30 min. Again the reaction mixture was cooled to −78° C., and then tri isopropyl borate (15 ml, 0.062 mol) was added in to it. After the completion of the addition the temperature was slowly raised to 0° C. and the reaction mixture stirred for 1 hr. Then after reaction mixture was cooled to −10° C. and saturated ammonium chloride solution was added slowly to the reaction mixture, followed by extraction with ethyl acetate (50 ml×3). The combined extract was washed with water and brine solution. The organic layer was dried over sodium sulphate and concentrated under vacuum to give 15 gm of 3-borono-N-(4,5-dimethyl-3-isoxazolyl)-N-[(2-methoxy-ethoxy)methyl]-5-methyl-thiophene sulphonamide as thick oily mass.

### STEP 08: Synthesis of (4-{2-[(4,5-dimethyl-isoxazol-3-yl)-(2-methoxy-ethoxymethyl)-sulphamoyl]-5-methyl-thiophene-3-yl}-3-methyl-benzoic acid methyl ester

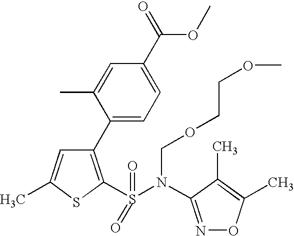

To a stirred solution of 4-bromo-3-methyl-benzoic acid methyl ester (4.72 gm, 0.0247 mol) in dimethoxy ethane (50 ml) under nitrogen bis(triphenylphosphine)palladium(II)chloride (1.45 gm, 0.002 mol) was added, followed by addition of a 2M aqueous sodium carbonate solution (6.5 gm in 30 ml water). The reaction mixture was stirred at room temperature for 10 min and then heated at 60° C. To this mixture a solution of 3-borono-N-(4,5-Dimethyl-3-isoxazolyl)-N-[(2-methoxy-ethoxy)methyl]-5-methyl-thiophene sulphonamide (5 gm, 0.012 mol in 25 ml dimethoxy ethane) was added drop wise within 45 min and the reaction was then refluxed for 60 min. After 1 hr the same procedure was repeated with further addition of 3-borono-N-(4,5-dimethyl-3-isoxazolyl)-N-[(2-methoxy-ethoxy)methyl]-5-methyl-thiophene sulphonamide (5 gm, 0.012 mol in 25 ml dimethoxy ethane) within 45 min. The reaction mixture was refluxed for 4 hrs and stirred at room temperature for 12 hrs and was then cooled and diluted with ethyl acetate (100 ml) and water. The organic layers were separated, the aqueous layer further extracted with ethyl acetate (50 ml×2) and the combined extracts was washed with water and brine. Finally the organic layer was dried over sodium sulphate and concentrated under vacuum. The crude compound was purified on a silica gel column using 4:1 hexane/ethyl acetate as an eluent to provide 9.7 gm of (4-{2-[(4,5-dimethyl-isoxazol-3-yl)-(2-methoxy-ethoxymethyl)-sulphamoyl]-5-methyl-thiophene-3-yl}-3-methyl-benzoic acid methyl ester as a pale yellow oily mass.

### STEP 09: Synthesis of 3-(4-hydroxy methyl-2-methyl-phenyl)-5-methyl-thiophene-2-sulphonic acid-(4,5-Dimethyl-isoxazol-3-yl)-(2-methoxy-ethoxymethyl) amide

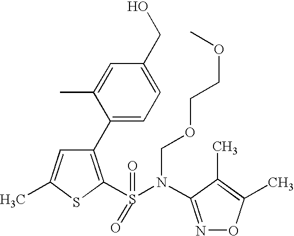

Lithium aluminium hydride (1 gm, 0.026 mol) was added to a stirred solution of tetrahydrofuran (15 ml) at 0° C. under flow of nitrogen, followed by the addition of (4-{2-[(4,5-dimethyl-isoxazol-3-yl)-(2-methoxy-ethoxymethyl)-sulphamoyl]-5-methyl-thiophene-3-yl}-3-methyl-benzoic acid methyl ester (9.7 gm, 0.019 mol) in 75 ml of tetrahydrofuran. The reaction mixture was stirred at 0° C. for 1 hr and then the temperature was raised to room temperature and stirred for 4 hrs. The reaction mixture was cooled to 0° C., and a sodium hydroxide solution (50 ml) (1 gm dissolved in 100 ml water) was added drop wise while maintaining the temperature at 0° C. This was followed by extraction with ethyl acetate (25 ml×2) and the organic layer was washed with water and brine solution. Finally organic layer was dried over sodium sulphate and concentrated under vacuum. The crude compound was purified on a silica gel column using 1:3 hexane/ethyl acetate as an eluent to provide 5.7 gm of 3-(4-hydroxy methyl-2-methyl-phenyl)-5-methyl-thiophene-2-sulphonic acid-(4,5-dimethyl-isoxazol-3-yl)-(2-methoxy-ethoxymethyl) amide

### STEP 10: Synthesis of methane sulphonic acid 4-{2-[(4,5-dimethyl-isoxazol-3-yl)-(2-methoxy-ethoxy methyl)-sulphamoyl]-5-methylthiophene-3-yl}-3-methyl-benzyl ester

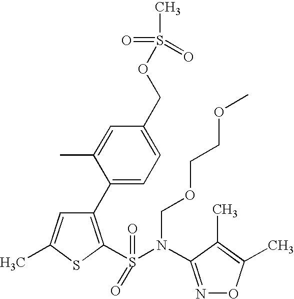

N-Ethyl diisopropyl amine (3 ml, 0.017 mol) was added to a solution of 3-(4-hydroxy methyl-2-methyl-phenyl)-5-methyl-thiophene-2-sulphonic acid-(4,5-dimethyl-isoxazol-3-yl)-(2-methoxy-ethoxymethyl) amide (5.7 gm, 0.012 mol) in 40 ml of dichloro methane. The reaction mixture was cooled to 0° C., and then methane sulphonyl chloride (1.16 ml, 0.014 mol) was slowly added into the reaction mixture. After the completion of the addition the reaction mixture was maintained at room temperature for 3 hrs. The reaction mixture was then dumped into ice-cold water followed by extraction with methylene chloride (50 ml×2). The combined extract was washed with dilute hydrochloric acid followed by washings with water and brine solution. Finally the organic layer was dried over sodium sulphate and concentrated under vacuum to give 6 gm of methane sulphonic acid 4-{2-[(4,5-dimethyl-isoxazol-3-yl)-(2-methoxy-ethoxy methyl)-sulphamoyl]-5-methylthiophene-3-yl}-3-methyl-benzyl ester.

### STEP 11: Synthesis of 3-[4-(5,7-diethyl-2-oxo-2H-[1,6]naphthyridin-1-ylmethyl)-2-methyl-phenyl]-5-methyl-thiophene-2-sulphonic acid(4,5-dimethyl-isoxazol-3yl)-(2-methoxy-ethoxymethyl)amide

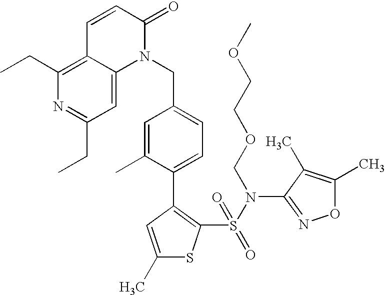

To a stirred solution of 5,7-diethyl-1H-[1,6] naphthyridin-2-one (0.74 gm, 3.5 mol) in dimethyl formamide (10 ml) at −15° C. under flow of dry nitrogen, sodium hydride (60% in mineral oil) (260 mg, 5.4 mmol) was added portion wise. After the addition, the reaction mixture was warmed to ambient temperature and maintained for 30 min. The reaction mixture was then re-cooled to 0° C. and a solution of methane sulphonic acid 4-{2-[(4,5-dimethyl-isoxazol-3yl)-(2-methoxy-ethoxy methyl)-sulphamoyl]-5-methyl-thiophen-3-yl}-3-methyl-benzyl ester. (2 gm, 3.5 mmol) in (10 ml) N,N-dimethyl formamide was added drop wise and the mixture was then stirred at room temperature for 24 hrs. The mixture was then diluted with ethyl acetate (40 ml), followed by 10 ml of cold water. The organic layer was separated and washed with water and brine solution. Finally the organic layer was dried over sodium sulphate and evaporated under vacuum. The crude compound was purified on a silica gel column using hexane:ethyl acetate as an eluent to provide 1.6 gm of 3-[4-(5,7-diethyl-2-oxo-2H-[1,6]naphthyridin-1-ylmethyl)-2-methyl-phenyl]-5-methyl-thiophene-2-sulphonic acid(4,5-dimethyl-isoxazol-3yl)-(2-methoxy-ethoxymethyl)amide as a viscous oily mass.

### STEP 12: Synthesis of 3-[4-(5,7-diethyl-2-oxo-2H-[1,6]naphthyridin-1-ylmethyl)-2-methyl-phenyl]-5-methyl-thiophene-2-sulphonic acid(4,5-dimethyl-isoxazol-3yl)-amide

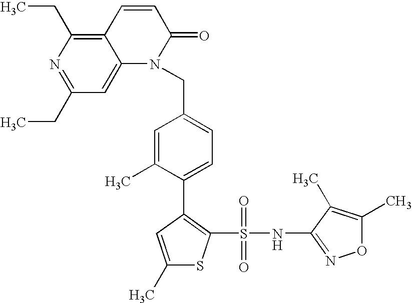

To 3-[4-(5,7-diethyl-2-oxo-2H-[1,6]naphthyridin-1-ylmethyl)-2-methyl-phenyl]-5-methyl-thiophene-2-sulphonic acid(4,5-dimethyl-isoxazol-3yl)-(2-methoxy-ethoxymethyl)amide (1.6 gm, 2.40 mmol) was added ethanol (10 ml) and 6N aqueous hydrochloric acid (8 ml) at room temperature. The reaction mixture was refluxed for 3 hrs and was then concentrated under vacuum and the residue thus obtained was diluted with water. The pH of the solution was adjusted to 8 using a saturated solution of sodium bicarbonate. Then the mixture was extracted with ethyl acetate (25 ml×2) and the combined organic extracts were washed with water and brine solution. Finally the organic layer was dried over sodium sulphate and concentrated under vacuum. The crude compound was purified by column chromatography on a silica gel column using 1:1 hexane/ethyl acetate as an eluent to provide 200 mg of 3-[4-(5,7-diethyl-2-oxo-2H-[1,6]naphthyridin-1-ylmethyl)-2-methyl-phenyl]-5-methyl-thiophene-2-sulphonic acid(4,5-dimethyl-isoxazol-3yl)-(2-methoxy-ethoxymethyl)amide

Molecular Formula: C₃₀H₃₂N₄O₄S₂

Molecular Weight: 576.74

¹HNMR (DMSOd₆): 1.20 (t, J=7.6 Hz, 3H) 1.25 (t, J=7.6 Hz, 3H) 1.46 (s, 3H) 1.92 (s, 3H) 2.10 (s, 3H) 2.47 (s, 3H) 2.70-2.76 (m, 2H) 3.04-3.10 (m, 2H) 5.46 (s, 2H) 6.70-6.74 (m, 2H) 6.85-6.87 (m, 1H) 6.91-6.92 (m, 1H) 7.14-7.16 (m, 2H) 8.22-8.25 (m, 1H) 10.62 (s, 1H)

Mass Spectrum: (m⁺¹) 577.2

### Example 20

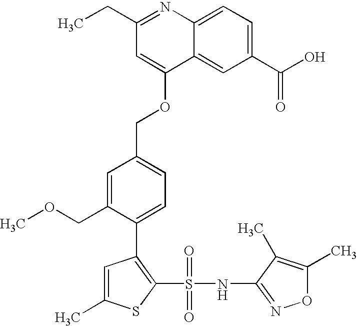

### 4-{4-[2-(4,5-dimethyl-isoxazol-3-yl sulfamoyl)-5-methyl-thiophen-3-yl]-3-methoxymethyl-benzyloxy}-2-ethyl-quinoline-6-carboxylic acid

### STEP 01: Synthesis of 4-[1-methoxycarbonylmethyl-propylideneamino]-benzoic acid ethyl ester

p-amino benzoic acid ethyl ester (25 gm, 0.15 mol) was added to a stirred solution of methyl propionyl acetate (24 gm, 0.18 mol) in toluene (200 ml) followed by addition of acetic acid (0.1 ml). After that the reaction mixture was refluxed for 24 hrs with continuous removal of water with help of dean stark apparatus. Then the reaction mixture was cooled to room temperature and evaporated under vacuum. The crude product was purified by column chromatography over silica gel column, using ethyl acetate:hexane as eluent to give 3.0 gm of 4-[1-methoxycarbonylmethyl-propylideneamino]-benzoic acid ethyl ester as oil.

### STEP 02: Synthesis of 2-ethyl-4-oxo-1,4-dihydro-quinoline-6-carboxylic acid ethyl ester

4-[1-methoxycarbonylmethyl-propylideneamino]-benzoic acid ethyl ester (3.0 g, 0.012 mol) was added to diphenyl ether (15 ml) and reaction mixture was stirred and heated at 250° C. for 3 hours. The reaction mixture was cooled to room temperature and to it was added (250 ml) hexane and cooled to 15° C. The crystallized product was filtered under vacuum, washed with (100 ml) hexane and suction dried to give 450 mg of 2-ethyl-4-oxo-1,4-dihydro-quinoline-6-carboxylic acid ethyl ester as crystalline solid.

### STEP 03: Synthesis of 4-bromo-3-bromomethyl-benzoic acid ethyl ester

Synthesis of 4-bromo-3-bromomethyl-benzoic acid ethyl ester was carried out as per STEP 02 of Example 01

### STEP 04: Synthesis of 4-Bromo-3-methoxymethyl-benzoic acid ethyl ester

Synthesis of 4-Bromo-3-methoxymethyl-benzoic acid ethyl ester was carried out as per STEP 05 of Example 16

### STEP 05: Synthesis of (5-methyl-isoxazol-3-yl) carbamic acid tert-butyl ester

Synthesis of (5-methyl-isoxazol-3-yl) carbamic acid tert-butyl ester was carried out as per STEP 04 of Example 01

### STEP 06: Synthesis of (4,5-dimethyl-isoxazol-3-yl) carbamic acid tert-butyl ester

Synthesis of (4,5-dimethyl-isoxazol-3-yl) carbamic acid tert-butyl ester was carried out as per STEP 05 of Example 01

### STEP 07: Synthesis of 4,5-dimethyl-isoxazol-3-ylamine

Synthesis of 4,5-dimethyl-isoxazol-3-ylamine was carried out as per STEP 06 of Example 01

### Step 08: Synthesis of 5-methyl thiophene-2-sulphonyl chloride

Synthesis of 5-methyl thiophene-2-sulphonyl chloride was carried out as per STEP 07 of Example 01

### Step 09: Synthesis of 5-methyl-thiophene-2-sulfonic acid (4,5-dimethyl-isoxazol-3yl) amide

Synthesis of 5-methyl-thiophene-2-sulfonic acid (4,5-dimethyl-isoxazol-3yl) amide was carried out as per STEP 08 of Example 01

### Step 10: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 11: Synthesis of 3-borono-N-(4,5-dimethyl-3-isoxazolyl)-N-[(2-methoxy-ethoxy)methyl]-5-methyl-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### STEP 12: Synthesis of (4-{2-[(4,5-dimethyl-isoxazol-3-yl)-(2-methoxy-ethoxymethyl)-sulfamoyl]-5-methyl-thiophene-3-yl}-3-methoxymethyl-benzoic acid ethyl ester

To a stirred solution of 4-bromo-3-methoxymethyl-benzoic acid ethyl ester (2.25 gm, 8.2 mmol) in dimethoxy ethane (25 ml) under nitrogen was added bis(triphenylphosphine)palladium(II)chloride (522 mg, 0.7 mmol) followed by addition of 2M aqueous sodium carbonate (2.62 gm in 13 ml water) Reaction mixture was stirred at room temperature for 10 min and then heated at 60° C. To this drop wise added the solution of 3-Borono-N-(4,5-dimethyl-3-isoxazolyl)-N-[(2-methoxy-ethoxy)methyl]-5-methyl-thiophene sulphonamide (1.5 gm, 3.7 mmol in 25 ml dimethoxy ethane) within 45 min and reaction was refluxed for 60 min. After 1 hr the same was repeated with further addition of 3-Borono-N-(4,5-dimethyl-3-isoxazolyl)-N-[(2-methoxy-ethoxy)methyl]-5-methyl-thiophene sulphonamide (1.5 gm, 3.7 mmol in 25 ml dimethoxy ethane) within 45 min, reaction mixture was refluxed for 4 hrs and stirred at room temperature for 12 hrs. Reaction mixture was diluted with ethyl acetate 100 ml and water, layers were separated, aqueous layer further extracted with ethyl acetate. Combine extracts was washed with water and brine. Dried over sodium sulphate and concentrated under vacuum. Crude compound was purified by column chromatography on a silica gel to yield 2.8 gm of (4-{2-[(4,5-dimethyl-isoxazol-3-yl)-(2-methoxy-ethoxymethyl)-sulfamoyl]-5-methyl-thiophene-3-yl}-3-methoxymethyl-benzoic acid ethyl ester as pale yellow oily mass.

### STEP 13: Synthesis of 3-(2-ethoxymethyl-4-hydroxy methyl-phenyl)-5-methyl-thiophene-2-sulfonic acid-(4,5-dimethyl-isoxazol-3-yl)-2-methoxy-methyl) amide

Synthesis of 3-(2-ethoxymethyl-4-hydroxy methyl-phenyl)-5-methyl-thiophene-2-sulfonic acid-(4,5-dimethyl-isoxazol-3-yl)-2-methoxy-methyl) amide was carried out as per STEP 12 of Example 01

### STEP 14: Synthesis of methane sulfonic acid 4-{2-[(4,5-dimethyl-isoxazol-3yl)-(2-methoxy-ethoxy methyl)-sulfamoyl]-5-methylthiophene-3-yl}-3-methoxy methyl-benzyl ester

Synthesis of methane sulfonic acid 4-{2-[(4,5-dimethyl-isoxazol-3-yl)-(2-methoxy-ethoxy methyl)-sulfamoyl]-5-methylthiophene-3-yl}-3-methoxy methyl-benzyl ester was carried out as per STEP 13 of Example 01

### Step 15: Synthesis of 4-(4-{2-[(4,5-dimethyl-isoxazol-3-yl)-(2-methoxy-ethoxymethyl)-sulfamoyl]-5-methyl-thiophen-3-yl}-3-methoxymethyl-benzyloxy)-2-ethyl-quinoline-6-carboxylic acid ethyl ester

To the stirred solution of 2-Ethyl-4-oxo-1,4-dihydro-quinoline-6-carboxylic acid ethyl ester (450 mg, 1.8 mmol) in dimethyl formamide (10 ml) at 0° C. under nitrogen was added portion wise sodium hydride (60% in mineral oil) (130 mg, 2.7 mmol). After the addition, the reaction mixture was warmed to ambient temperature and maintained for 30 min. Reaction mixture was cooled to 0° C. and a solution of methane sulfonic acid 4-{2-[(4,5-dimethyl-isoxazol-3-yl)-(2-methoxy-ethoxy methyl)-sulfamoyl]-5-methylthiophene-3-yl}-3-methoxymethyl-benzyl ester (1 gm, 1.8 mmol) in (10 ml) N,N-dimethyl formamide was added drop wise to the reaction mixture and stirred at room temperature for 24 hrs. The mixture was then diluted with ethyl acetate (30 ml), followed by 10 ml of cold water, organic layer washed with water and brine then dried over sodium sulphate and evaporated under vacuum to afford crude 1 gm of 4-(4-{2-[(4,5-dimethyl-isoxazol-3-yl)-(2-methoxy-ethoxymethyl)-sulfamoyl]-5-methyl-thiophen-3-yl}-3-methoxymethyl-benzyloxy)-2-ethyl-quinoline-6-carboxylic acid ethyl ester as a viscous oily mass.

### Step 16: Synthesis of 4-{-4-[2-(4,5-dimethyl-isoxazol-3-ylsulfamoyl)-5-methyl-thiophen-3-yl]-3-methoxymethyl-benzyloxy}-2-ethyl-quinoline-6-carboxylic acid ethyl ester

To, 1.0 gm of 4-(4-{2-[(4,5-dimethyl-isoxazol-3-yl)-(2-methoxy-ethoxymethyl)-sulfamoyl]-5-methyl-thiophen-3-yl}-3-methoxymethyl-benzyloxy)-2-ethyl-quinoline-6-carboxylic acid ethyl ester was added 95% ethanol (10 ml) and 6N aqueous hydrochloric acid (8 ml) at room temperature. Reaction mixture was refluxed for 3 hrs. The reaction mixture was concentrated under vacuum and pH of the solution was adjusted to 8 using a saturated solution of sodium bicarbonate, and the mixture was extracted with ethyl acetate (25 ml×3). The combined organic extract were washed with water and brine then dried over sodium sulphate and concentrated under vacuum. The residue was purified by column chromatography over Silica Gel column using hexane:ethyl acetate to afford 200 mg of 4-{4-[2-(4,5-dimethyl-isoxazol-3-ylsulfamoyl)-5-methyl-thiophen-3-yl]-3-methoxymethyl-benzyloxy}-2-ethyl-quinoline-6-carboxylic acid ethyl ester.

### Step 17: Synthesis of 4-{4-[2-(4,5-dimethyl-isoxazol-3-ylsulfamoyl)-5-methyl-thiophen-3-yl]-3-methoxymethyl-benzyloxy}-2-ethyl-quinoline-6-carboxylic acid

To, 4-{4-[2-(4,5-dimethyl-isoxazol-3-ylsulfamoyl)-5-methyl-thiophen-3-yl]-3-methoxymethyl-benzyloxy}-2-ethyl-quinoline-6-carboxylic acid ethyl ester (200 mg, 0.3 m mol) was added solution of sodium hydroxide (50 mg in 2 ml of water) followed by addition of (5 ml) methanol. This reaction mixture was stirred at room temperature for 6 hrs. Then after the reaction mixture was evaporated under vacuum to the residue ml of water was added and this was extracted with ml of diethyl ether. Aqueous layer was separated, cooled to 5° C. and acidified to pH 2 with dilute hydrochloric acid and extracted with ethyl acetate (50 ml×2). The organic layer was washed with water and brine then dried over sodium sulphate and concentrated under vacuum to give brown solid which was triturated with hexane filtered under vacuum washed with hexane and suction dried to provide 45 mg of 4-{4-[2-(4,5-dimethyl-isoxazol-3-ylsulfamoyl)-5-methyl-thiophen-3-yl]-3-methoxymethyl-benzyloxy}-2-ethyl-quinoline-6-carboxylic acid as brown solid.

Molecular Formula: C₃₁H₃₁N₃O₇S₂

Molecular Weight: 621.73

¹HNMR (DMSOd₆): 1.25 (t, J=7.6 Hz, 3H), 1.36 (s, 3H), 2.15 (s, 3H), 2.47 (s, 3H), 2.91-2.97 (q, J=7.6 Hz, 2H), 3.18 (s, 3H), 4.12 (s, 2H), 5.44 (s, 2H), 6.94 (s, 1H), 7.21 (s, 2H), 7.41 (s, 1H) 7.53 (s, 1H), 7.94-7.96 (d, J=8.8 Hz, 1H) 8.15-8.18 (dd, J=8.8 Hz, 1 h), 8.76 (s, 1H) 10.71 (br, 1H), 13.05 (br, 1H).

Mass Spectrum: (m⁺¹) 622.1

### Example 21

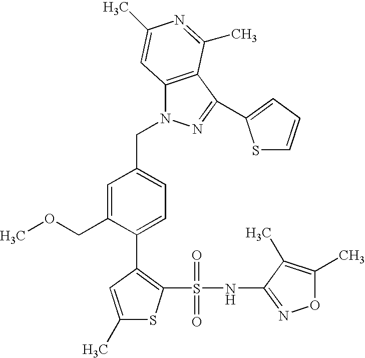

### 3-[4-(4,6-dimethyl-3-thiophen-2-yl-pyrazolo[4,3-c]pyridin-1-ylmethyl)-2-methoxymethyl-phenyl]-5-methyl-thiophene-2-sulfonic acid (4,5-dimethyl-isoxazol-3-yl)-amide

### STEP 01: Synthesis of 1-thiophene-2-yl-butane-1,3-dione

In, 40 ml N,N-dimethyl formamide was added sodium hydride (60% in mineral oil) (4.18 gm, 0.17 mol) at 0° C. Then the solution prepared by dissolving 2-Acetyl thiophene (20 gm, 0.16 mol) in dry ethyl acetate (31 ml, 0.32 mol) was added to the reaction mixture. This reaction was stirred at room temperature for 12 hrs. Reaction mixture was acidified with 1N hydrochloric acid and extracted with ethyl acetate (100 ml×2). Combined extracts were washed with water and brine. Dried over sodium sulphate and evaporated to give 27 gm of 1-thiophene-2-yl-butane-1,3-dione.

### STEP 02: Synthesis of 3-Amino-1-thiophene-2yl-but-2-en-1-one

A mixture of 1-Thiophene-2-yl-butane-1,3-dione (26.5 gm, 0.16 mol) and ammonium acetate (48.6 gm, 0.63 mol) in dry methanol (260 ml) was stirred at room temperature for 24 hrs. The reaction mixture was concentrated completely under vacuum and chilled water was added to the residue. The reaction mixture was basified to pH 8 using saturated sodium bicarbonate solution, followed by extraction with ethyl acetate (100 ml×2). Combine extracts were washed with water and brine. Dried over sodium sulphate and evaporated to give 16.8 gm of 3-Amino-1-thiophene-2yl-but-2-en-1-one.

### STEP 03: Synthesis of 2,6-dimethyl-3-(thiophene-2-carbonyl)-1H-pyridin-4-one

A mixture of 2,2,6-trimethyl-[1,3] dioxin-4-one (28.6 gm, 0.20 mol) and 3-amino-1-thiophene-2yl-but-2-en-1-one (16.8 gm, 0.10 mol) was heated to reflux at 120° C. for 6 hrs. Reaction mixture was purified by column chromatography over silica gel, eluting the desired product with 10% methanol and ethyl acetate to give 4.6 gm of 2,6-Dimethyl-3-(thiophene-2-carbonyl)-1H-pyridin-4-one.

### STEP 04: Synthesis of (4-chloro-2,6-dimethyl-pyridin-3-yl)-thiophene-2-yl-methanone

2,6-dimethyl-3-(thiophene-2-carbonyl)-1H-pyridin-4-one (4.6 gm, 0.01 mol) was added to 30 ml phosphorous oxychloride at 0° C. Stirred and heated the reaction mixture at 100° C. and maintained for 8 hours. Work-up was done by evaporating the phosphorus oxy chloride under vacuum and basified the residue to pH 8 with saturated Sodium carbonate solution, followed by extraction with methylene dichloride (50 ml×2). Combined extracts were washed with water and brine. Dried over anhydrous Sodium sulphate and concentrated to give 4.35 gm of (4-Chloro-2,6-dimethyl-pyridin-3-yl)-thiophene-2-yl-methanone

### STEP 05: Synthesis of 4,6-dimethyl-3-thiophene-2yl-1H-pyrazolo[4,3-c]pyridine

(4-chloro-2,6-dimethyl-pyridin-3-yl)-Thiophene-2-yl-methanone (4.35 gm, 0.02 mol) was taken in ethanol (20 ml) and hydrazine hydrate (6 ml, 0.185 mol) and two drops of acetic acid was added to the reaction mixture. Slowly raised the temperature and heated to reflux, maintained reflux for 6 hours. Reaction mixture was completely evaporated under vacuum. The crude mass was dumped in to ice, solid obtained was filtered off and suck dried to provide 4.48 gm of 4,6-dimethyl-3-thiophene-2yl-1H-pyrazolo[4,3-c]pyridine.

### STEP 06: Synthesis of 4-bromo-3-bromomethyl-benzoic acid ethyl ester

Synthesis of 4-bromo-3-bromomethyl-benzoic acid ethyl ester was carried out as per STEP 02 of Example 01

### STEP 07: Synthesis of 4-bromo-3-methoxymethyl-benzoic acid ethyl ester

Synthesis of 4-Bromo-3-methoxymethyl-benzoic acid ethyl ester was carried out as per STEP 05 of Example 16

### STEP 08: Synthesis of (5-methyl-isoxazol-3-yl) carbamic acid tert-butyl ester

Synthesis of (5-methyl-isoxazol-3-yl) carbamic acid tert-butyl ester was carried out as per STEP 04 of Example 01

### STEP 09: Synthesis of (4,5-dimethyl-isoxazol-3-yl) carbamic acid tert-butyl ester

Synthesis of (4,5-dimethyl-isoxazol-3-yl) carbamic acid tert-butyl ester was carried out as per STEP 05 of Example 01

### STEP 10: Synthesis of 4,5-dimethyl-isoxazol-3-ylamine

Synthesis of 4,5-dimethyl-isoxazol-3-ylamine was carried out as per STEP 06 of Example 01

### Step 11: Synthesis of 5-methyl thiophene-2-sulphonyl chloride

Synthesis of 5-methyl thiophene-2-sulphonyl chloride was carried out as per STEP 07 of Example 01

### Step 12: Synthesis of 5-methyl-thiophene-2-sulfonic acid (4,5-dimethyl-isoxazol-3yl) amide

Synthesis of 5-methyl-thiophene-2-sulfonic acid (4,5-dimethyl-isoxazol-3yl) amide was carried out as per STEP 08 of Example 01

### Step 13: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 14: Synthesis of 3-borono-N-(4,5-dimethyl-3-isoxazolyl)-N-[(2-methoxy-ethoxy)methyl]-5-meth 1-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### STEP 15: Synthesis of (4-{2-[(4,5-dimethyl-isoxazol-3-yl)-(2-methoxy-ethoxymethyl)-sulfamoyl]-5-methyl-thiophene-3-yl}-3-methoxymethyl-benzoic acid ethyl ester

Synthesis of (4-{2-[(4,5-dimethyl-isoxazol-3-yl)-(2-methoxy-ethoxymethyl)-sulfamoyl]-5-methyl-thiophene-3-yl}-3-methoxymethyl-benzoic acid ethyl ester was carried out as per STEP 13 of Example 16

### STEP 16: Synthesis of 3-(2-ethoxymethyl-4-hydroxy methyl-phenyl)-5-methyl-thiophene-2-sulfonic acid-(4,5-dimethyl-isoxazol-3-yl)-2-methoxy-methyl) amide

Synthesis of 3-(2-ethoxymethyl-4-hydroxy methyl-phenyl)-5-methyl-thiophene-2-sulfonic acid-(4,5-dimethyl-isoxazol-3-yl)-2-methoxy-methyl) amide was carried out as per STEP 14 of Example 16

### STEP 17: Synthesis of methane sulfonic acid 4-{2-[(4,5-dimethyl-isoxazol-3yl)-(2-methoxy-ethoxy methyl)-sulfamoyl]-5-methylthiophene-3-yl}-3-methoxy methyl-benzyl ester

Methane sulfonic acid 4-{2-[(4,5-dimethyl-isoxazol-3-yl)-(2-methoxy-ethoxy methyl)-sulfamoyl]-5-methylthiophene-3-yl}-3-methoxy methyl-benzyl ester was carried out as per STEP 15 of Example 16

### Step 18: Synthesis of 3-[4-(4,6-dimethyl-3-thiophen-2-yl-pyrazolo[4,3-c]pyridin-1-ylmethyl)-2-methoxymethyl-phenyl]-5-methyl-thiophene-2-sulfonic acid (4,5-dimethyl-isoxazol-3-yl)-(2-methoxy-ethoxymethyl)-amide

To the stirred solution of 4,6-dimethyl-3-thiophene-2yl-1H-pyrazolo[4,3-c]pyridine (433 mg, 1.9 mmol) in N,N-dimethyl formamide (10 ml) at 0° C. under nitrogen was added portion wise sodium hydride (60% in mineral oil) (140 mg, 3 mmol). After the addition, the reaction mixture was warmed to ambient temperature and maintained for 30 min. Reaction mixture was cooled to 0° C. and a solution of methane sulfonic acid 4-{2-[(4,5-dimethyl-isoxazol-3-yl)-(2-methoxy-ethoxy methyl)-sulfamoyl]-5-methylthiophene-3-yl}-3-methoxymethyl-benzyl ester (1 gm, 1.8 mmol) in (10)ml dimethyl formamide was added drop wise to the reaction mixture and stirred at room temperature for 24 hrs. The mixture was then diluted with ethyl acetate (40 ml), followed by 10 ml of cold water, organic layer washed with water and brine then dried over sodium sulphate and evaporated under vacuum to afford 2 gm, crude compound which was purified by column chromatography on a silica gel to yield 1 gm of 3-[4-(4,6-Dimethyl-3-thiophen-2-yl-pyrazolo[4,3-c]pyridin-1-ylmethyl)-2-methoxymethyl-phenyl]-5-methyl-thiophene-2-sulfonic acid (4,5-dimethyl-isoxazol-3-yl)-(2-methoxy-ethoxymethyl)-amide as a viscous oily mass.

### Step 19: Synthesis of 3-[4-(4,6-dimethyl-3-thiophen-2-yl-pyrazolo[4,3-c]pyridin-1-ylmethyl)-2-methoxymethyl-phenyl]-5-methyl-thiophene-2-sulfonic acid (4,5-dimethyl-isoxazol-3-yl)-amide

To, 1.0 gm of 3-[4-(4,6-dimethyl-3-thiophen-2-yl-pyrazolo[4,3-c]pyridin-1-ylmethyl)-2-methoxymethyl-phenyl]-5-methyl-thiophene-2-sulfonic acid (4,5-dimethyl-isoxazol-3-yl)-(2-methoxy-ethoxymethyl)-amide was added 95% ethanol (10 ml) and 6 N aqueous hydrochloric acid (8 ml) at room temperature. Reaction mixture was refluxed for 3 hrs. The reaction mixture was concentrated under vacuum and pH of the solution was adjusted to 8 using a saturated solution of sodium bicarbonate, and the mixture was extracted with ethyl acetate (25 ml×3). The combined organic extract were washed with water and brine then dried over sodium sulphate and concentrated under vacuum. The residue was purified by column chromatography over a silica gel using hexane:ethyl acetate to afford 100 mg of 3-[4-(4,6-dimethyl-3-thiophen-2-yl-pyrazolo[4,3-c]pyridin-1-ylmethyl)-2-methoxymethyl-phenyl]-5-methyl-thiophene-2-sulfonic acid (4,5-dimethyl-isoxazol-3-yl)-amide.

Molecular Formula: C₃₁H₃₁N₅O₄S₃

Molecular Weight: 633.80

¹HNMR (DMSOd₆): 1.46 (s, 3H), 2.12 (s, 3H), 2.47 (s, 3H), 2.49 (s, 3H), 2.66 (s, 3H), 3.14 (s, 3H), 4.04 (m, 2H), 5.68 (s, 2H), 6.69-7.73 (m, 8H), 10.66 (br, 1H).

Mass Spectrum: (m⁺¹) 634.3

### Example 22

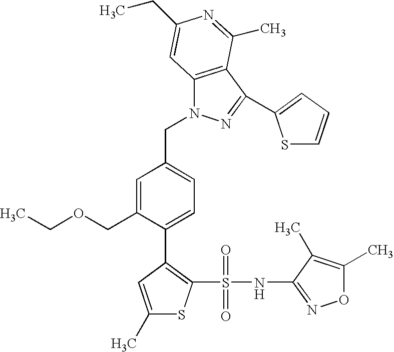

### 3-[2-Ethoxymethyl-4-(6-ethyl-4-methyl-3-thiophen-2-yl-pyrazolo[4,3-c]pyridin-1-ylmethyl)-phenyl]-5-methyl-thiophene-2-sulfonic acid (4,5-dimethyl-isoxazol-3-yl)-amide

### STEP 01: Synthesis of 1-Thiophene-2-yl-butane-1,3-dione

In 40 ml N,N-Dimethyl formamide was added Sodium hydride (60% in mineral oil) (4.18 gm, 0.17 mol) at 0° C. Then the solution prepared by dissolving 2-Acetyl thiophene (20 gm, 0.16 mol) in dry ethyl acetate (31 ml, 0.32 mol) was added to the reaction mixture This reaction was stirred at room temperature for 12 hrs. Reaction mixture was acidified with 1N hydrochloric acid and extracted with ethyl acetate (100 ml×2). Combined extracts were washed with water and brine. Dried over sodium sulphate and evaporated to give 27 gm of 1-Thiophene-2-yl-butane-1,3-dione.

### STEP 02: Synthesis of 3-Amino-1-thiophene-2yl-but-2-en-1-one

A mixture of 1-Thiophene-2-yl-butane-1,3-dione (26.5 gm, 0.16 mol) and ammonium acetate (48.6 gm, 0.63 mol) in dry methanol (260 ml) was stirred at room temperature for 24 hrs. The reaction mixture was concentrated completely under vacuum and chilled water was added to the residue. The reaction mixture was basified to pH8 using saturated sodium bicarbonate solution, followed by extraction with ethyl acetate (100 ml×2). Combine extracts were washed with water and brine. Dried over sodium sulphate and evaporated to give 16.8 gm of 3-Amino-1-thiophene-2yl-but-2-en-1-one.

### STEP 03: Synthesis of 4-bromo-3-bromomethyl-benzoic acid ethyl ester

Synthesis of 4-bromo-3-bromomethyl-benzoic acid ethyl ester was carried out as per STEP 02 of Example 01

### STEP 04: Synthesis of 4-Bromo-3-ethoxymethyl-benzoic acid ethyl ester

Synthesis of 4-Bromo-3-ethoxymethyl-benzoic acid ethyl ester was carried out as per STEP 03 of Example 01

### STEP 05: Synthesis of 6-Ethyl-2-methyl-3-(thiophene-2-carbonyl)-1H-pyridin-4-one

The mixture of 2,2-dimethyl-5-propionyl-[1,3] dioxane-4,6-dione (15.8 gm, 0.08 mol) and 3-amino-1-thiophene-2yl-but-2-en-1-one (11 gm, 0.066 mol) was heated to reflux at 120° C. for 6 hrs. Reaction mixture was cooled to room temperature and then triturated with ether, product was filtered under vacuum washed with ether and suction dried to give 6 gm of 6-Ethyl-2-methyl-3-(thiophene-2-carbonyl)-1H-pyridin-4-one

### STEP 06: Synthesis of (4-chloro-6-ethyl-2-methyl-pyridin-3-yl)-thiophen-2-yl-methanone

6-Ethyl-2-methyl-3-(thiophene-2-carbonyl)-1H-pyridin-4-one (6 gm, 0.024 mol) was added to 30 ml phosphorous oxychloride at 0° C. Stirred and heated the reaction mixture at 100° C. and maintained for 8 hours. Work-up was done by evaporating the phosphorus oxy chloride under vacuum and basified the residue to pH 8 with saturated sodium carbonate solution, followed by extraction with methylene dichloride (50 ml×2). Combined extracts were washed with water and brine. Dried over anhydrous Sodium sulphate and concentrated to give 3 gm of (4-Chloro-6-ethyl-2-methyl-pyridin-3-yl)-thiophen-2-yl-methanone as brown oil.

### STEP 07: Synthesis of 6-Ethyl-4-methyl-3-thiophen-2-yl-1H-pyrazolo[4,3-c]pyridine

(4-Chloro-6-ethyl-2-methyl-pyridin-3-yl)-Thiophen-2-yl-methanone (3 gm, 0.011 mol) was taken in ethanol (20 ml) and hydrazine hydrate (9 ml, 0.185 mol) and two drops of acetic acid was added to the reaction mixture. Slowly raised the temperature and heated to reflux, maintained reflux for 6 hours. Reaction mixture was completely evaporated under vacuum. The crude mass was dumped in to ice, solid obtained was filtered off and suck dried to provide 1.7 gm of 6-Ethyl-4-methyl-3-thiophen-2-yl-1H-pyrazolo[4,3-c]pyridine.

### STEP 08: Synthesis of (5-methyl-isoxazol-3-yl) carbamic acid tert-butyl ester

Synthesis of (5-methyl-isoxazol-3-yl) carbamic acid tert-butyl ester was carried out as per STEP 04 of Example 01

### STEP 09: Synthesis of (4,5-dimethyl-isoxazol-3-yl) carbamic acid tert-butyl ester

Synthesis of (4,5-dimethyl-isoxazol-3-yl) carbamic acid tert-butyl ester was carried out as per STEP 05 of Example 01

### STEP 10: Synthesis of 4,5-dimethyl-isoxazol-3-ylamine

Synthesis of 4,5-dimethyl-isoxazol-3-ylamine was carried out as per STEP 06 of Example 01

### Step 11: Synthesis of 5-methyl thiophene-2-sulphonyl chloride

Synthesis of 5-methyl thiophene-2-sulphonyl chloride was carried out as per STEP 07 of Example 01

### Step 12: Synthesis of 5-methyl-thiophene-2-sulfonic acid (4,5-dimethyl-isoxazol-3yl) amide

Synthesis of 5-methyl-thiophene-2-sulfonic acid (4,5-dimethyl-isoxazol-3yl) amide was carried out as per STEP 08 of Example 01

### Step 13: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 14: Synthesis of 3-borono-N-(4,5-dimethyl-3-isoxazolyl)-N-[(2-methoxy-ethoxy)methyl]-5-methyl-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### STEP 15: Synthesis of (4-{2-[(4,5-dimethyl-isoxazol-3-yl)-(2-methoxy-ethoxymethyl)-sulfamoyl]-5-methyl-thiophene-3-yl}-3-ethoxymethyl-benzoic acid ethyl ester

Synthesis of (4-{2-[(4,5-dimethyl-isoxazol-3-yl)-(2-methoxy-ethoxymethyl)-sulfamoyl]-5-methyl-thiophene-3-yl}-3-ethoxymethyl-benzoic acid ethyl ester was carried out as per STEP 15 of Example 15

### STEP 16: Synthesis of 3-(2-ethoxymethyl-4-hydroxy methyl-phenyl)-5-methyl-thiophene-2-sulfonic acid-(4,5-dimethyl-isoxazol-3-yl)-2-methoxy-ethoxymethyl) amide

Synthesis of 3-(2-ethoxymethyl-4-hydroxy methyl-phenyl)-5-methyl-thiophene-2-sulfonic acid-(4,5-dimethyl-isoxazol-3-yl)-2-methoxy-ethoxymethyl) amide was carried out as per STEP 16 of Example 15

### STEP 17: Synthesis of methane sulfonic acid 4-{2-[(4,5-dimeth 1-isoxazol-3-yl)-(2-methoxy-ethoxy methyl)-sulfamoyl]-5-methylthiophene-3-yl}-3-ethoxy methyl-benzyl ester

Synthesis of methane sulfonic acid 4-{2-[(4,5-dimethyl-isoxazol-3-yl)-(2-methoxy-ethoxy methyl)-sulfamoyl]-5-methylthiophene-3-yl}-3-ethoxy methyl-benzyl ester was carried out as per STEP 17 of Example 15

### Step 18: Synthesis of 3-[2-Ethoxymethyl-4-(6-ethyl-4-methyl-3-thiophen-2-yl-pyrazolo[4,3-c] pyridin-1-ylmethyl)-phenyl]-5-methyl-thiophene-2-sulfonic acid (4,5-dimethyl-isoxazol-3-yl)-(2-methoxy-ethoxymethyl)-amide

To the stirred solution of 6-Ethyl-4-methyl-3-thiophen-2-yl-1H-pyrazolo[4,3-c]pyridine (448 mg, 1.8 mmol) in N,N-dimethyl formamide (10 ml) at 0° C. under nitrogen was added portion wise sodium hydride (60% in mineral oil) (132 mg, 2.5 mmol). After the addition, the reaction mixture was warmed to ambient temperature and maintained for 30 min. Reaction mixture was cooled to 0° C. and a solution of methane sulfonic acid 4-{2-[(4,5-dimethyl-isoxazol-3-yl)-(2-methoxy-ethoxy methyl)-sulfamoyl]-5-methylthiophene-3-yl}-3-ethoxy methyl-benzyl ester (1 gm, 1.8 mmol) in (8 ml) dimethyl formamide was added drop wise to the reaction mixture and stirred at room temperature for 24 hrs. The mixture was then diluted with ethyl acetate (40 ml), followed by 10 ml of cold water, Organic layer washed with water and brine then dried over sodium sulphate and evaporated under vacuum to afford crude 2 gm of 3-[2-Ethoxymethyl-4-(6-ethyl-4-methyl-3-thiophen-2-yl-pyrazolo[4,3-c]pyridin-1-ylmethyl)-phenyl]-5-methyl-thiophene-2-sulfonic acid (4,5-dimethyl-isoxazol-3-yl)-(2-methoxy-ethoxymethyl)-amide as a viscous oily mass.

### Step 19: Synthesis of 3-[2-Ethoxymethyl-4-(6-ethyl-4-methyl-3-thiophen-2-yl-pyrazolo[4,3-c] pyridin-1-ylmethyl)-phenyl]-5-methyl-thiophene-2-sulfonic acid (4,5-dimethyl-isoxazol-3-yl)-amide

To, crude 2 gm of 3-[2-Ethoxymethyl-4-(6-ethyl-4-methyl-3-thiophen-2-yl-pyrazolo[4,3-c]pyridin-1-ylmethyl)-phenyl]-5-methyl-thiophene-2-sulfonic acid (4,5-dimethyl-isoxazol-3-yl)-(2-methoxy-ethoxymethyl)-amide was added ethanol (10 ml) and 6N aqueous hydrochloric acid (8 ml) at room temperature. Reaction mixture was stirred and heated for 3 hrs. The reaction mixture was concentrated under vacuum and pH of the solution was adjusted to 8 using a saturated solution of sodium bicarbonate, and the mixture was extracted with ethyl acetate (30 ml×3). The combined organic extract were washed with water and brine then dried over sodium sulphate and concentrated under vacuum. The residue was purified by column chromatography over Silica Gel column using hexane:ethyl acetate to afford 200 mg of 3-[2-Ethoxymethyl-4-(6-ethyl-4-methyl-3-thiophen-2-yl-pyrazolo[4,3-c]pyridin-1-ylmethyl)-phenyl]-5-methyl-thiophene-2-sulfonic acid (4,5-dimethyl-isoxazol-3-yl)-amide.

Molecular Formula: C₃₃H₃₅N₅O₄S₃

Molecular Weight: 661.87

¹HNMR (DMSOd₆): 1.10 (t, J=6.8 Hz, 3H), 1.242-1.31 (m, 6H), 1.47 (s, 3H), 2.12 (s, 3H), 2.47 (s, 3H), 2.67 (s, 3H), 2.80-2.85 (q, J=7.6 Hz, 2H), 3.23-3.28 (q, J=6.8 Hz, 2H), 4.06 (s, 2H), 5.71 (s, 2H), 6.69 (s, 1H), 6.94-6.96 (d, J=7.6 Hz, 1H) 7.08-7.10 (d, J=7.6 Hz 1H) 7.23-7.25 (m, 1H) 7.35 (s, 1H) 7.45 (s, 2H), 7.71-7.73 (dJ=5.2 Hz, 1H), 10.63 (br, 1H).

Mass Spectrum: (m⁺¹) 662.3

### Example 23

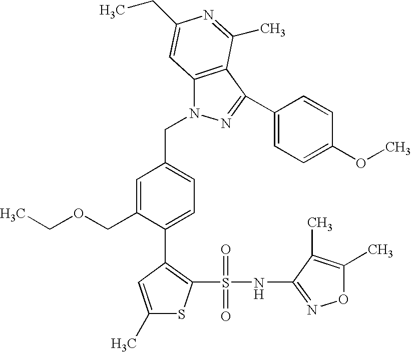

### 3-{2-Ethoxymethyl-4-[6-ethyl-3-(4-methoxy-phenyl)-4-methyl-pyrazolo[4,3-c]pyridin-1-ylmethyl]-phenyl}-5-methyl-thiophene-2-sulfonic acid (4,5-dimethyl-isoxazol-3-yl)-amide

### STEP 01: Synthesis of 1-(4-methoxy-phenyl)-butane-1,3-dione

In 30 ml N,N-Dimethyl formamide was added Sodium hydride (60% in mineral oil) (8.8 gm, 0.219.mol) at 0° C. Followed by addition of solution of dry ethyl acetate (15.5 gm, 0.175 mol) and 1-(4-methoxy-phenyl)-ethanone (22 gm, 0145.mol). This reaction was stirred at room temperature for 6 hrs. Reaction mixture was acidified with 1N hydrochloric acid and extracted with ethyl acetate (100 ml×2). Combined extracts were washed with water and brine. Dried over sodium sulphate and evaporated to give 22 gm of 1-(4-methoxy-phenyl)-butane-1,3-dione.

### STEP 02: Synthesis of 3-Amino-1-(4-methoxy-phenyl)-but-2-en-1-one

A mixture of 1-(4-methoxy-phenyl)-butane-1,3-dione (22 gm, 0.114 mol) and ammonium acetate (26.3 gm, 0.342 mol) in dry methanol (150 ml) was stirred at room temperature for 24 hrs. The reaction mixture was concentrated completely under vacuum and chilled water was added to the residue. The reaction mixture was basified to pH8 using saturated sodium bicarbonate solution, followed by extraction with ethyl acetate (100 ml×2). Combine extracts were washed with water and brine. Dried over sodium sulphate and evaporated to give 20 gm of 3-Amino-1-(4-methoxy-phenyl)-but-2-en-1-one

### STEP 03: Synthesis of 4-bromo-3-bromomethyl-benzoic acid ethyl ester

Synthesis of 4-bromo-3-bromomethyl-benzoic acid ethyl ester was carried out as per STEP 02 of Example 01

### STEP 04: Synthesis of 4-Bromo-3-ethoxymethyl-benzoic acid ethyl ester

Synthesis of 4-Bromo-3-ethoxymethyl-benzoic acid ethyl ester was carried out as per STEP 03 of Example 01

### STEP 05: Synthesis of 6-Ethyl-3-(4-methoxy-benzoyl)-2-methyl-1H pyridin-4-one

A mixture of 2,2,6-trimethyl-5-propionyl-[1,3]dioxin-4-one (15 gm, 0.075.mol) and 3-Amino-1-(4-methoxy-phenyl)-but-2-en-1-one (12 gm, 0.062.mol) was heated to refluxed at 120° C. for 6 hrs. Reaction mixture was purified by triturating crude reaction mixture with ether, solid thus separated was filtered under vacuum, washed with ether and suction dried to give 2.4 gm of 6-Ethyl-3-(4-methoxy-benzoyl)-2-methyl-1H-pyridin-4-one

### STEP 06: Synthesis of (4-chloro-6-ethyl-2-methyl-pyridin-3-yl)-(4-methoxy-phenyl)-methanone

6-Ethyl-3-(4-methoxy-benzoyl)-2-methyl-1H-pyridin-4-one (2.4 gm) was added to (15 ml) phosphorous oxychloride at 0° C. Stirred and heated the reaction mixture at 100° C. and maintained for 8 hours. Work-up was done by evaporating the phosphorus oxy chloride under vacuum and basified the residue to pH 8 with saturated Sodium carbonate solution, followed by extraction with methylene dichloride (50 ml×2). Combined extracts were washed with water and brine. Dried over anhydrous Sodium sulphate and concentrated to give 2.44 gm of (4-Chloro-6-ethyl-2-methyl-pyridin-3-yl)-(4-methoxy-phenyl)-methanone

### STEP 07: Synthesis of 6-Ethyl-3-(4-methoxy-phenyl)-4-methyl-1H-pyrazolo[4,3-c] pyridine

(4-chloro-6-ethyl-2-methyl-pyridin-3-yl)-(4-methoxy-phenyl)-methanone (2.44 gm, 0.0084 mol) was taken in ethanol (20 ml) and hydrazine hydrate (3 ml) and two drops of acetic acid was added to the reaction mixture. Slowly raised the temperature and heated to reflux, maintained reflux for 6 hours. Reaction mixture was completely evaporated under vacuum. The crude mass was dumped in to ice, solid obtained was filtered off and suck dried to provide 1.7 gm of 6-Ethyl-3-(4-methoxy-phenyl)-4-methyl-1H-pyrazolo[4,3-c]pyridine.

### STEP 08: Synthesis of (5-methyl-isoxazol-3-yl) carbamic acid tert-butyl ester

Synthesis of (5-methyl-isoxazol-3-yl) carbamic acid tert-butyl ester was carried out as per STEP 04 of Example 01

### STEP 08: Synthesis of (4,5-dimethyl-isoxazol-3-yl) carbamic acid tert-butyl ester

Synthesis of (4,5-dimethyl-isoxazol-3-yl) carbamic acid tert-butyl ester was carried out as per STEP 05 of Example 01

### STEP 09: Synthesis of 4,5-dimethyl-isoxazol-3-ylamine

Synthesis of 4,5-dimethyl-isoxazol-3-ylamine was carried out as per STEP 06 of Example 01

### Step 10: Synthesis of 5-methyl thiophene-2-sulphonyl chloride

Synthesis of 5-methyl thiophene-2-sulphonyl chloride was carried out as per STEP 07 of Example 01

### Step 11: Synthesis of 5-methyl-thiophene-2-sulfonic acid (4,5-dimethyl-isoxazol-3yl) amide

Synthesis of 5-methyl-thiophene-2-sulfonic acid (4,5-dimethyl-isoxazol-3yl) amide was carried out as per STEP 08 of Example 01

### Step 12: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 13: Synthesis of 3-borono-N-(4,5-dimethyl-3-isoxazolyl)-N-[(2-methoxy-ethoxy)methyl]-5-methyl-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### STEP 14: Synthesis of (4-{2-[(4,5-dimethyl-isoxazol-3-yl)-(2-methoxy-ethoxymethyl)-sulfamoyl]-5-methyl-thiophene-3-yl}-3-ethoxymethyl-benzoic acid ethyl ester

Synthesis of (4-{2-[(4,5-dimethyl-isoxazol-3-yl)-(2-methoxy-ethoxymethyl)-sulfamoyl]-5-methyl-thiophene-3-yl}-3-ethoxymethyl-benzoic acid ethyl ester was carried out as per STEP 15 of Example 15

### STEP 15: Synthesis of 3-(2-ethoxymethyl-4-hydroxy methyl-phenyl)-5-methyl-thiophene-2-sulfonic acid-(4,5-dimethyl-isoxazol-3-yl)-2-methoxy-ethoxymethyl) amide

Synthesis of 3-(2-ethoxymethyl-4-hydroxy methyl-phenyl)-5-methyl-thiophene-2-sulfonic acid-(4,5-dimethyl-isoxazol-3-yl)-2-methoxy-ethoxymethyl) amide was carried out as per STEP 16 of Example 15

### STEP 16: Synthesis of methane sulfonic acid 4-{2-[(4,5-dimethyl-isoxazol-3yl)-(2-methoxy-ethoxy methyl)-sulfamoyl]-5-methylthiophene-3-yl}-3-ethoxy methyl-benzyl ester

Synthesis of methane sulfonic acid 4-{2-[(4,5-dimethyl-isoxazol-3-yl)-(2-methoxy-ethoxy methyl)-sulfamoyl]-5-methylthiophene-3-yl}-3-ethoxy methyl-benzyl ester was carried out as per STEP 17 of Example 15

### Step 17: Synthesis of 3-{2-Ethoxymethyl-4-[6-ethyl-3-(4-methoxy-phenyl)-4-methyl-pyrazolo [4,3-c] pyridin-1-ylmethyl]-phenyl}-5-methyl-thiophene-2-sulfonic acid (4,5-dimethyl-isoxazol-3-yl)-(2-methoxy-ethoxymethyl)-amide

To the stirred solution of 6-Ethyl-3-(4-methoxy-phenyl)-4-methyl-1H-pyrazolo[4,3-c]pyridine (492 mg, 1.8 mmol) in dimethyl formamide (10 ml) at 0° C. under nitrogen was added portion wise sodium hydride (60% in mineral oil) (132 mg, 2.5 mmol). After the addition, the reaction mixture was warmed to ambient temperature and maintained for 30 min. Reaction mixture was cooled to 0° C. and a solution of Methane sulfonic acid 4-{2-[(4,5-dimethyl-isoxazol-3-yl)-(2-methoxy-ethoxy methyl)-sulfamoyl]-5-methylthiophene-3-yl}-3-ethoxy methyl-benzyl ester (1 gm, 1.8 mmol) in (10)ml dimethyl formamide was added drop wise to the reaction mixture and stirred at room temperature for 24 hrs. The mixture was then diluted with ethyl acetate (40 ml), followed by 10 ml of cold water, Organic layer washed with water and brine then dried over sodium sulphate and evaporated under vacuum to afford crude 2 gm of 3-{2-Ethoxymethyl-4-[6-ethyl-3-(4-methoxy-phenyl)-4-methyl-pyrazolo[4,3-c]pyridin-1-ylmethyl]-phenyl}-5-methyl-thiophene-2-sulfonic acid (4,5-dimethyl-isoxazol-3-yl)-(2-methoxy-ethoxymethyl)-amide as a viscous oily mass.

### Step 18: Synthesis of 3-{2-Ethoxymethyl-4-[6-ethyl-3-(4-methoxy-phenyl)-4-methyl-pyrazolo [4,3-c] pyridin-1-ylmethyl]-phenyl}-5-methyl-thiophene-2-sulfonic acid (4,5-dimethyl-isoxazol-3-yl)-amide

To, crude 2 gm of 3-{2-Ethoxymethyl-4-[6-ethyl-3-(4-methoxy-phenyl)-4-methyl-pyrazolo[4,3-c]pyridin-1-ylmethyl]-phenyl}-5-methyl-thiophene-2-sulfonic acid (4,5-dimethyl-isoxazol-3-yl)-(2-methoxy-ethoxymethyl)-amide was added ethanol (13 ml) and 6N aqueous hydrochloric acid (10 ml) at room temperature. Reaction mixture was stirred and heated for 3 hrs. The reaction mixture was concentrated under vacuum and pH of the solution was adjusted to 8 using a saturated solution of sodium bicarbonate, and the mixture was extracted with ethyl acetate (30 ml×3). The combined organic extract were washed with water and brine then dried over sodium sulphate and concentrated under vacuum. The residue was purified over column chromatography over silica gel column using hexane:ethyl acetate to afford 70 mg of 3-{2-Ethoxymethyl-4-[6-ethyl-3-(4-methoxy-phenyl)-4-methyl-pyrazolo[4,3-c]pyridin-1-ylmethyl]-phenyl}-5-methyl-thiophene-2-sulfonic acid (4,5-dimethyl-isoxazol-3-yl)-amide.

Molecular Formula: C₃₆H₃₉N₅O₅S₂

Molecular Weight: 685.87

¹HNMR (DMSOd₆): 1.02 (t, J=7.2 Hz, 3H), 1.27 (t, J=7.2 Hz, 3H), 1.48 (s, 3H), 2.12 (s, 3H), 2.45 (s, 3H), 2.48 (s, 3H) 2.79-2.85 (q, J=7.6 Hz, 2H), 3.23-3.29 (q, J=6.8 Hz, 2H), 3.84 (s, 3H), 4.07 (s, 2H), 5.70 (s, 2H), 6.70-7.59 (m, 10H), 10.75 (br, 1H).

Mass Spectrum: (m⁺¹) 686.3

### Example 24

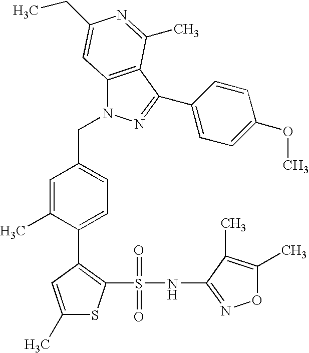

### 3-{4-[6-Ethyl-3-(4-methoxy-phenyl)-4-methyl-pyrazolo[4,3-c]pyridin-1-ylmethyl]-2-methyl-phenyl}-5-methyl-thiophene-2-sulfonic acid (4,5-dimethyl-isoxazol-3-yl)-amide

### STEP 01: Synthesis of 1-(4-methoxy-phenyl)-butane-1,3-dione

Synthesis of 1-(4-methoxy-phenyl)-butane-1,3-dione was carried out as per STEP 01 of Example 23

### STEP 02: Synthesis of 3-Amino-1-(4-methoxy-phenyl)-but-2-en-1-one

Synthesis of 3-Amino-1-(4-methoxy-phenyl)-but-2-en-1-one was carried out as per STEP 02 of Example 23

### STEP 03: Synthesis of 6-Ethyl-3-(4-methoxy-benzoyl)-2-methyl-1Hpyridin-4-one

Synthesis of 6-Ethyl-3-(4-methoxy-benzoyl)-2-methyl-1H-pyridin-4-one was carried out as per STEP 05 of Example 23

### STEP 04: Synthesis of (4-Chloro-6-ethyl-2-methyl-pyridin-3-yl)-(4-methoxy-phenyl)-methanone

Synthesis of (4-Chloro-6-ethyl-2-methyl-pyridin-3-yl)-(4-methoxy-phenyl)-methanone was carried out as per STEP 06 of Example 23

### STEP 05: Synthesis of 6-Ethyl-3-(4-methoxy-phenyl)-4-methyl-1H-pyrazolo[4,3-c] pyridine

Synthesis of 6-Ethyl-3-(4-methoxy-phenyl)-4-methyl-1H-pyrazolo[4,3-c]pyridine was carried out as per STEP 07 of Example 23

### STEP 06: Synthesis of (5-methyl-isoxazol-3-yl) carbamic acid tert-butyl ester

Synthesis of (5-methyl-isoxazol-3-yl) carbamic acid tert-butyl ester was carried out as per STEP 04 of Example 01

### STEP 07: Synthesis of (4,5-dimethyl-isoxazol-3-yl) carbamic acid tert-butyl ester

Synthesis of (4,5-dimethyl-isoxazol-3-yl) carbamic acid tert-butyl ester was carried out as per STEP 05 of Example 01

### STEP 08: Synthesis of 4,5-dimethyl-isoxazol-3-ylamine

Synthesis of 4,5-dimethyl-isoxazol-3-ylamine was carried out as per STEP 06 of Example 01

### Step 09: Synthesis of 5-methyl thiophene-2-sulphonyl chloride

Synthesis of 5-methyl thiophene-2-sulphonyl chloride was carried out as per STEP 07 of Example 01

### Step 10: Synthesis of 5-methyl-thiophene-2-sulfonic acid (4,5-dimethyl-isoxazol-3yl) amide

Synthesis of 5-methyl-thiophene-2-sulfonic acid (4,5-dimethyl-isoxazol-3yl) amide was carried out as per STEP 08 of Example 01

### Step 11: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 12: Synthesis of 3-borono-N-(4,5-dimethyl-3-isoxazolyl)-N-[(2-methoxy-ethoxy)methyl]-5-methyl-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### STEP 13: Synthesis of (4-{2-[(4,5-dimethyl-isoxazol-3-yl)-(2-methoxy-ethoxymethyl)-sulfamoyl]-5-methyl-thiophene-3-yl}-3-methyl-benzoic acid methyl ester

Synthesis of (4-{2-[(4,5-dimethyl-isoxazol-3-yl)-(2-methoxy-ethoxymethyl)-sulfamoyl]-5-methyl-thiophene-3-yl}-3-methyl-benzoic acid methyl ester was carried out as per STEP 08 of Example 19

### STEP 14: Synthesis of 3-(4-hydroxy methyl-2-methyl-phenyl)-5-methyl-thiophene-2-sulfonic acid-(4,5-dimethyl-isoxazol-3-yl)-(2-methoxy-ethoxymethyl) amide

Synthesis of 3-(4-hydroxy methyl-2-methyl-phenyl)-5-methyl-thiophene-2-sulfonic acid-(4,5-dimethyl-isoxazol-3-yl)-(2-methoxy-ethoxymethyl) amide was carried out as per STEP 09 of Example 19

### STEP 15: Synthesis of methane sulfonic acid 4-{2-[(4,5-dimethyl-isoxazol-3-yl)-(2-methoxy-ethoxy methyl)-sulfamoyl]-5-methylthiophene-3-yl}-3-methyl-benzyl ester

Synthesis of methane sulfonic acid 4-{2-[(4,5-dimethyl-isoxazol-3-yl)-(2-methoxy-ethoxy methyl)-sulfamoyl]-5-methylthiophene-3-yl}-3-methyl-benzyl ester was carried out as per STEP 10 of Example 19

### Step 16: Synthesis of 3-{4-[6-Ethyl-3-(4-methoxy-phenyl)-4-methyl-pyrazolo[4,3-c]pyridin-1-ylmethyl]-2-methyl-phenyl}-5-methyl-thiophene-2-sulfonic acid (4,5-dimethyl-isoxazol-3-yl)-(2-methoxy-ethoxymethyl)-amide

To the stirred solution of 6-Ethyl-3-(4-methoxy-phenyl)-4-methyl-1H-pyrazolo[4,3-c]pyridine (642 mg, 2.4 mmol) in dimethyl formamide (10 ml) at 0° C. under nitrogen was added portion wise sodium hydride (60% in mineral oil) (173 mg, 3.6 mmol). After the addition, the reaction mixture was warmed to ambient temperature and maintained for 30 min. Reaction mixture was cooled to 0° C. and a solution of methane sulfonic acid 4-{2-[(4,5-dimethyl-isoxazol-3-yl)-(2-methoxy-ethoxy methyl)-sulfamoyl]-5-methylthiophene-3-yl}-3-methyl-benzyl ester (1.2 gm, 2.4 mmol) in 10 ml dimethyl formamide was added drop wise to the reaction mixture and stirred at room temperature for 24 hrs. The mixture was then diluted with ethyl acetate (40 ml), followed by 10 ml of cold water, Organic layer washed with water and brine then dried over sodium sulphate and evaporated under vacuum to afford 2 gm of 3-{4-[6-Ethyl-3-(4-methoxy-phenyl)-4-methyl-pyrazolo[4,3-c]pyridin-1-ylmethyl]-2-methyl-phenyl}-5-methyl-thiophene-2-sulfonic acid (4,5-dimethyl-isoxazol-3-yl)-(2-methoxy-ethoxymethyl)-amide as a viscous oily mass.

### Step 17: Synthesis of 3-{4-[6-Ethyl-3-(4-methoxy-phenyl)-4-methyl-pyrazolo[4,3-c]pyridin-1-ylmethyl]-2-methyl-phenyl}-5-methyl-thiophene-2-sulfonic acid (4,5-dimethyl-isoxazol-3-yl)-amide

To, 3-{4-[6-Ethyl-3-(4-methoxy-phenyl)-4-methyl-pyrazolo[4,3-c]pyridin-1-ylmethyl]-2-methyl-phenyl}-5-methyl-thiophene-2-sulfonic acid (4,5-dimethyl-isoxazol-3-yl)-(2-methoxy-ethoxymethyl)-amide (2 gm) was added 95% ethanol (10 ml) and 6N aqueous hydrochloric acid (8 ml) at room temperature. Reaction mixture was refluxed for 3 hrs. The reaction mixture was concentrated under vacuum and pH of the solution was adjusted to 8 using a saturated solution of sodium bicarbonate. The reaction solution was then acidified to pH5 with acetic acid, and the mixture was extracted with ethyl acetate (25 ml×2). The combined organic extract were washed with water and brine then dried over sodium sulphate and concentrated under vacuum. The residue was purified by column chromatography over Silica Gel column using ethyl acetate:hexane as eluent to afford 170 mg of 3-{4-[6-Ethyl-3-(4-methoxy-phenyl)-4-methyl-pyrazolo[4,3-c]pyridin-1-ylmethyl]-2-methyl-phenyl}-5-methyl-thiophene-2-sulfonic acid (4,5-dimethyl-isoxazol-3-yl)-amide.

Molecular Formula: C₃₄H₃₅N₅O₄S₂

Molecular Weight: 641.8

¹HNMR (DMSOd₆): 1.29 (m, 3H), 1.46 (s, 3H), 1.94 (s, 3H), 2.11 (s, 3H), 2.47 (s, 3H), 2.79-2.85 (m, 2H), 3.85 (s, 4H), 5.62 (s, 2H), 6.68-7.59 (m, 11H), 10.68 (br, 1H).

Mass Spectrum: (m⁺¹) 642.3

### Example 25

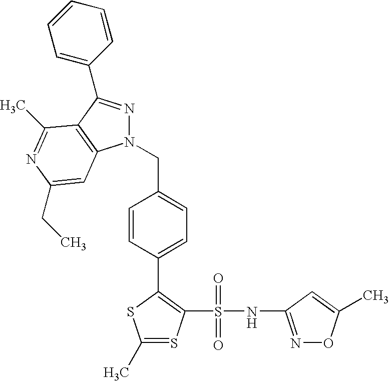

### 2-[4-(6-Ethyl-4-methyl-3-phenyl-pyrazolo[4,3-c]pyridin-1-ylmethyl)-phenyl]-5-methyl-thiophene-3-sulfonic acid (5-methyl-isoxazol-3-yl)-amide

### STEP 01: Synthesis of 1-Phenyl-butane-1,3 dione

Synthesis of 1-Phenyl-butane-1,3 dione was carried out as per STEP 01 of Example 9

### STEP 02: Synthesis of 3-amino-1-phenyl-but-2-en-1-one

Synthesis of 3-amino-1-phenyl-but-2-en-1-one was carried out as per STEP 02 of Example 9

### STEP 03: Synthesis of 5-(1-hydroxy propylidine) 2,2-dimethyl-1,3-dioxane-4,6-dione

Synthesis of 5-(1-hydroxy propylidine) 2,2-dimethyl-1,3-dioxane-4,6-dione was carried out as per STEP 03 of Example 9

### STEP 04: Synthesis of 3-Benzoyl-6-ethyl-2-methyl-1H-Pyridin-4-one

Synthesis of 3-Benzoyl-6-ethyl-2-methyl-1H-Pyridin-4-one was carried out as per STEP 04 of Example 9

### STEP 05: Synthesis of (4-chloro-6-ethyl-2-methyl-pyridin-3-yl) phenyl-methanone

Synthesis of (4-chloro-6-ethyl-2-methyl-pyridin-3-yl)phenyl-methanone was carried out as per STEP 05 of Example 9

### STEP 06: Synthesis of 6-Ethyl-4-methyl-3-phenyl-1H-pyrazolo [4,3-c] pyridine

Synthesis of 6-Ethyl-4-methyl-3-phenyl-1H-pyrazolo[4,3-c]pyridine was carried out as per STEP 06 of Example 9

### Step 07: Synthesis of 2-Bromo-5-methyl-thiophene-3-sulfonyl chloride

2-Bromo-5-methyl-thiophene (5 gm, 0.0282 mol) in was added to a mixture of chloro sulfonic acid (4.7 ml, 0.0705 mol) and phosphorous pentachloride (gm, mol) at 15° C. Maintained the reaction mixture at 15° C. for 10 min. Crude reaction mass was slowly dumped into the ice cold water, followed by extraction with diisopropyl ether (100 ml×2). Combined organic extract was washed with water and brine. Dried other organic layer over anhydrous sodium sulphate and evaporated under vacuum to give 6.3 gm of 2-bromo-5-methyl-thiophene-3-sulfonyl chloride as brown colored liquid.

### Step 08: Synthesis of 2-bromo-5-methyl-thiophene-3-sulfonic acid (5-methyl-isoxazol-3-yl)-amide

2-Bromo-5-methyl-thiophene-3-sulfonyl chloride (6.3 gm, 0.0229 mol) in methylene chloride (50 ml) was added to a solution of 3-amino-5-methylisoxazole (3.37 gm, 0.0344 mol) in pyridine (50 ml) and dimethylaminopyridine (280 mg) at 0° C. The temperature was slowly raised to room temperature and stirred for 6 hours. Concentrated the reaction mixture completely under vacuum, acidified the reaction mixture using 1N hydrochloric acid followed by extraction with methylene chloride (100 ml×2). Combine extracts was given washing with water and brine. Dried over sodium sulphate and concentrated to give 6 gm of 2-Bromo-5-methyl-thiophene-3-sulfonic acid (5-methyl-isoxazol-3-yl)-amide as brown colored solid.

### Step 09: Synthesis of 2-Bromo-5-methyl-thiophene-3-sulfonic acid ethoxymethyl-(5-methyl-isoxazol-3-yl)-amide

Sodium hydride (0.64 gm, 0.0267 mol) was added to a stirred solution of dimethyl formamide (15 ml) at 0° C. followed by addition of 2-Bromo-5-methyl-thiophene-3-sulfonic acid (5-methyl-isoxazol-3-yl)-amide (6.0 gm, 0.0178 mol). Slowly raised the temperature and maintained at ambient temperature for 30 minutes then cooled to 0° C. followed by addition of ethoxy methyl chloride (2.02 gm, 0.0214 mol) at 0° C. After the completion of addition, slowly raised to ambient temperature and stirred for 3 hrs. Charged 90 ml ethyl acetate followed by 25 ml ice water to the reaction mixture. Organic layer was separated; aqueous was again extract with ethyl acetate (50 ml×2). Combine extracts was washed with water and brine solution. Dried under sodium sulphate and concentrated under vacuum. Residue was purified by Column Chromatography on a Silica Gel column to give 4.45 gm of 2-Bromo-5-methyl-thiophene-3-sulfonic acid ethoxymethyl-(5-methyl-isoxazol-3-yl)-amide as yellowish oil.

### STEP 10: Synthesis of 2-(4-formyl-phenyl)-5-methyl-thiophene-3-sulfonic acid ethoxymethyl-(5-methyl-isoxazol-3-yl)-amide

To a stirred solution of 2-Bromo-5-methyl-thiophene-3-sulfonic acid ethoxymethyl-(5-methyl-isoxazol-3-yl)-amide (3 gm, 0.00076 mol) and 4-formyl phenyl boronic acid (1.026 gm, 0.00684 mol) in toluene (60 ml) and ethanol (50 ml) under nitrogen was added 2M aqueous sodium carbonate (2.417 gm in 11.4 ml water). Stirred the reaction mixture under nitrogen atmosphere for 15 minutes then added tetrakis triphenyl phosphine palladium (0) (0.791 gm, 0.00068 mol) into the reaction mixture. The reaction mixture was heated to 85° C. for 6 hrs. The reaction mixture was concentrated under vacuum. Ethyl acetate (25 ml) was added to the residue followed by chilled water and extraction with ethyl acetate (100 ml×2). Combine extracts was washed with water and brine. Dried over sodium sulphate and concentrated completely under vacuum. Crude compound was purified by column chromatography on a silica gel to yield 2.3 gm of 2-(4-formyl-phenyl)-5-methyl-thiophene-3-sulfonic acid ethoxymethyl-(5-methyl-isoxazol-3-yl)-amide as oily mass.

### STEP 11: Synthesis of 2-(4-hydroxymethyl-phenyl)-5-methyl-thiophene-3-sulfonic acid ethoxymethyl-(5-methyl-isoxazol-3-yl)-amide

To a stirred solution of 2-(4-formyl-phenyl)-5-methyl-thiophene-3-sulfonic acid ethoxymethyl-(5-methyl-isoxazol-3-yl)-amide (2.3 gm, 0.0055 mol) in 35 ml Tetrahydrofuran at 0° C. under flow of nitrogen, Sodium borohydride (0.249 gm, 0.0066 mol) was added, followed by addition of 2 drops of water. Stirred the reaction mixture at rt for 1 hr. Reaction mixture was evaporated under vacuum to the residue 1N hydrochloric acid solution was added followed by extraction with ethylacetate (25 ml×2). Organic layer was dried over sodium sulphate and concentrated completely under vacuum. The crude product was purified by column chromatography over silica gel column using ethyl acetate:hexane as eluent to give 740 mg of 2-(4-hydroxymethyl-phenyl)-5-methyl-thiophene-3-sulfonic acid ethoxymethyl-(5-methyl-isoxazol-3-yl)-amide

### STEP 12: Synthesis of methanesulfonic acid 4-{3-[ethoxymethyl-(5-methyl-isoxazol-3-yl)-sulfamoyl]-5-methyl-thiophen-2-yl}-benzyl ester

N-Ethyl diisopropyl amine (0.6 ml, 0.00344 mol) was added to a solution of 2-(4-Hydroxymethyl-phenyl)-5-methyl-thiophene-3-sulfonic acid ethoxymethyl-(5-methyl-isoxazol-3-yl)-amide (740 mg, 0.00175 mol) in 15 ml of dichloro methane. Cooled the reaction mixture to 0° C., added slowly methane sulfonyl chloride (0.2 ml, 0.00251 mol) into the reaction mixture. The reaction mixture was maintained at room temperature for 3 hrs. Reaction mixture was dumped into ice-cold water followed by extraction with methylene chloride (50 ml×2). Combine extracts was given washing with dilute hydrochloric acid followed by water and brine solution. Dried the organic layer over sodium sulphate and concentrated to give 1.24 gm of methanesulfonic acid 4-{3-[ethoxymethyl-(5-methyl-isoxazol-3-yl)-sulfamoyl]-5-methyl-thiophen-2-yl}-benzyl ester

### Step 13: Synthesis of 2-[4-(6-Ethyl-4-methyl-3-phenyl-pyrazolo[4,3-c]pyridin-1-ylmethyl)-phenyl]-5-methyl-thiophene-3-sulfonic acid ethoxymethyl-(5-methyl-isoxazol-3-yl)-amide

To the stirred solution of 6-Ethyl-4-methyl-3-phenyl-1Hpyrazolo [4,3-c] pyridine (452 mg, 0.00191 mol) in dimethyl formamide (6 ml) at −0° C. under nitrogen was added portion wise sodium hydride (60% in mineral oil) (69 mg, 0.00287 mol). After the addition, the reaction mixture was warmed to ambient temperature and maintained for 30 min. Reaction mixture was cooled to 0° C. and a solution of methanesulfonic acid 4-{3-[ethoxymethyl-(5-methyl-isoxazol-3-yl)-sulfamoyl]-5-methyl-thiophen-2-yl}-benzyl ester (1.24 gm, 0.00248 mol) in (6 ml) dimethyl formamide was added drop wise to the reaction mixture and stirred at room temperature for 5 hrs. The mixture was then diluted with ethyl acetate (40 ml), followed by 10 ml of cold water, Organic layer washed with water and brine then dried over sodium sulphate and evaporated under vacuum to afford 1.35 gm of 2-[4-(6-Ethyl-4-methyl-3-phenyl-pyrazolo[4,3-c]pyridin-1-ylmethyl)-phenyl]-5-methyl-thiophene-3-sulfonic acid ethoxymethyl-(5-methyl-isoxazol-3-yl)-amide as a viscous oily mass.

### Step 14: Synthesis of 2-[4-(6-Ethyl-4-methyl-3-phenyl-pyrazolo [4,3-c]pyridin-1-ylmethyl)-phenyl]-5-methyl-thiophene-3-sulfonic acid (5-methyl-isoxazol-3-yl)-amide

To, 2-[4-(6-Ethyl-4-methyl-3-phenyl-pyrazolo[4,3-c]pyridin-1-ylmethyl)-phenyl]-5-methyl-thiophene-3-sulfonic acid ethoxymethyl-(5-methyl-isoxazol-3-yl)-amide (1.35 gm) was added 95% ethanol (10 ml) and 6N aqueous hydrochloric acid (6 ml) at room temperature. Reaction mixture was refluxed for 3 hrs. The reaction mixture was concentrated under vacuum and pH of the solution was adjusted to 8 using a saturated solution of sodium bicarbonate followed by extraction with ethyl acetate (25 ml×2). The combined organic extract were washed with water and brine then dried over sodium sulphate and concentrated under vacuum. The residue was purified by column chromatography over Silica Gel column using hexane/ethyl acetate as eluent to afford 182 mg of 2-[4-(6-Ethyl-4-methyl-3-phenyl-pyrazolo[4,3-c]pyridin-1-ylmethyl)-phenyl]-5-methyl-thiophene-3-sulfonic acid (5-methyl-isoxazol-3-yl)-amide.

Molecular Formula: C₃₁H₂₉N₅O₃S₂

Molecular Weight: 583.72

¹HNMR (DMSOd₆): 1.28 (t, J=7.6 Hz, 3H), 2.29 (s, 3H), 2.48 (s, 3H), 2.61 (s, 3H), 2.74-2.84 (q, J=7.2 Hz, 2H), 5.69 (s, 2H), 7.31-7.65 (m, 12H), 11.55 (br, 1H).

Mass Spectrum: (m⁺¹) 584.3

### Example 26

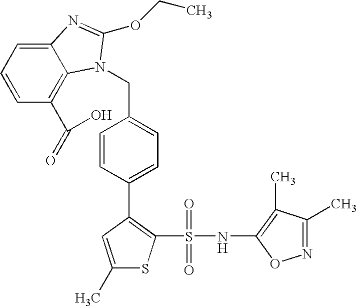

### 3-{4-[2-(3,4-dimethyl-isoxazol-5-ylsulfamoyl)-5-methyl-thiophen-3-yl]-benzyl}-2-ethoxy-3H-benzoimidazole-4-carboxylic acid

### Step 01: Synthesis of 5-methyl thiophene-2-sulphonyl chloride

Synthesis of 5-methyl thiophene-2-sulphonyl chloride was carried out as per STEP 07 of Example 01

### Step 02: Synthesis of 5-Methyl-thiophene-2-sulfonic acid (3,4-dimethyl-isoxazol-5-yl)-amide

5-Methyl thiophene-2-sulphonyl chloride (10.5 gm, 0.053 mol) in methylene chloride (50 ml) was added to a solution of 3,4-dimethyl-isoxazol-5-ylamine (5 gm, 0.044 mol) in pyridine (20 ml) and dimethylaminopyridine (500 mg) at 0° C. The temperature was slowly raised to room temperature and stirred for 6 hours. Concentrated the reaction mixture completely under vacuum, acidified the reaction mixture using 1N hydrochloric acid followed by extraction with methylene chloride (100 ml×2). Combine extracts was given washing with water and brine. Dried over sodium sulphate and concentrated to give 10 gm mixture of 5-Methyl-thiophene-2-sulfonic acid (3,4-dimethyl-isoxazol-5-yl)-amide and as brown colored solid.

### Step 03: Synthesis of 5-methyl-thiophene-2-sulfonic acid (3,4-dimethyl-isoxazol-5-yl)-(2-trimethylsilanyl-ethoxymethyl)-amide

Sodium hydride (0.726 gm, 0.0182 mol) was added to a stirred solution of dimethyl formamide (30 ml) at 0° C. followed by addition of 5-methyl-thiophene-2-sulfonic acid (3,4-dimethyl-isoxazol-5-yl)-amide (3.3 gm, 0.012 mol). Slowly raised the temperature and maintained at ambient temperature for 30 minutes then cooled to 0° C. followed by addition of (2-Chloromethoxy-ethyl)-trimethyl-silane (2.6 gm, 0.014 mol) at 0° C. After the completion of addition, reaction temperature was slowly raised to ambient temperature and stirred for 3 hrs. Charged 90 ml ethyl acetate followed by 25 ml ice water to the reaction mixture. Organic layer was separated; aqueous was again extract with ethyl acetate (50 ml×2). Combine extracts was washed with water and brine solution. Dried under sodium sulphate and concentrated under vacuum. Residue was purified by column chromatography on a Silica Gel column using hexane:ethyl acetate as an eluent to give 4.6 gm of 5-methyl-thiophene-2-sulfonic acid (3,4-dimethyl-isoxazol-5-yl)-(2-trimethylsilanyl-ethoxymethyl)-amide as yellowish oil.

### Step 04: Synthesis of 3-Borono-N-(3,4-dimethyl-3-isoxazolyl)-N-[(2-trimethylsilanyl-ethoxymethyl)]-5-methyl-thiophene sulphonamide

5-Methyl-thiophene-2-sulfonic acid (3,4-dimethyl-isoxazol-5-yl)-(2-trimethylsilanyl-ethoxymethyl)-amide (4.6 gm, 0.016 mol) was dissolved in 35 ml tetrahydrofuran and the reaction mixture was cooled to −78° C. under nitrogen atmosphere. n-butyl lithium (18 ml, 0.028 mol, 15% solution in n-hexane) was added slowly into the reaction mixture by using a syringe. After the completion of addition the reaction mixture was stirred at −78° C. for 1 hr and slowly raised the temperature to 0° C. and stirred for 30 min. Again the reaction mixture cooled to −78° C. then trimethyl borate (2 ml, 0.017 mol) was added. After the completion of addition slowly raised the temperature to 0° C. and stirred for 1 hr. Reaction mixture was cooled to −10° C. and added saturated ammonium chloride solution followed by extraction with ethyl acetate (50 ml×2). Combine extract was washed with water and brine. Dried under sodium sulphate and concentrated under vacuum to give 6.1 gm of 3-Borono-N-(3,4-dimethyl-3-isoxazolyl)-N-[(2-trimethylsilanyl-ethoxymethyl)]-5-methyl-thiophene sulphonamide as thick oily mass.

### STEP 05: Synthesis of 3-nitro phthalic anhydride

With stirring place (50 gm, 0.236 mol) of 3-nitro phthalic acid in (50 ml, 0.53 mol) of acetic anhydride. This reaction mixture was heated at 100-120° C. for 20 min. to get the clear solution, reaction mixture was cooled to room temperature to give the crystalline solid, thus the crystalline product was slurried with hexane and filtered under vacuum, washed with hexane to give 50 gm of 3-nitro phthalic anhydride.

### STEP 06: Synthesis of 3-Nitro-phthalamic acid

To a chilled solution of aqueous ammonia (30-35%, 200 ml) 3-nitro phthalic anhydride (45 gm, 0.23 mol) was charged portion wise within 15 min and stirred this reaction mixture for 30 min. Reaction mixture was acidified to pH-2 with external cooling by dilute hydrochloric acid. After 20 min the solid separated which was filtered under vacuum and washed with hexane. Product was dried under vacuum to give 50 gm of 3-Nitro-phthalamic acid.

### STEP 07: Synthesis of 2-amino-3-nitro-benzoic acid

To a stirred cold solution of potassium hydroxide (113 gm, 2.01 mol) in water (550 ml), Bromine (11.5 ml, 0.22 mol) was added slowly at 5-10° C. Reaction mixture was stirred at this temperature for 15 minutes. 3-Nitro-phthalamic acid (45 gm, 0.21 mol) was charged in one portion. Reaction mixture was heated and stirred at 60-70° C. for 4 hours. Reaction mixture was acidified with external cooling with dilute hydrochloric acid to pH 2. The solid product was filtered under vacuum, dried in hot air oven at 60-70° C. to give 40 gm of 2-amino-3-nitro-benzoic acid.

### STEP 08: Synthesis of 2-amino-3-nitro-benzoic acid methyl ester

To a stirred cold solution of 2-amino-3-nitro-benzoic acid (20 gm, 0.11 mol) in methanol (300 ml), sulfuric acid (48 ml) was added at 0-5° C. Reaction mixture was refluxed for 12 hours, cooled down to room temperature and methanol was evaporated under vacuum. Reaction mixture was basified with saturated sodium bicarbonate solution (pH˜7-8). Extracted with ethyl acetate (500 ml×2), dried over sodium sulphate and evaporated under vacuum to get 11.0 gm of 2-amino-3-nitro-benzoic acid methyl ester as a yellow crystalline solid.

### STEP 09: Synthesis of 3-Nitro-2-(2,2,2-trifluoro-acetylamino)-benzoic acid methyl ester

To a stirred cold solution of 2-amino-3-nitro-benzoic acid methyl ester (9.0 gm, mol) in pyridine (90 ml), trifluoroacetic anhydride (9.0 ml, 0.06 mol) was added drop wise over a period of 30 minutes at 0-5° C. Reaction mixture was stirred at room temperature for 1 hour. Reaction mixture was quenched on ice-cold water (100 ml) with stirring. Adjust pH 7-8 by saturated sodium bicarbonate solution. Obtained solid was filtered under vacuum and washed with hexane. Solid was dissolved in ethyl acetate, dried over sodium sulphate and evaporated under vacuum to get 7.5 gm of 3-Nitro-2-(2,2,2-trifluoro-acetylamino)-benzoic acid methyl ester as a solid.

### STEP 10: Synthesis of 2-[(4-Bromo-benzyl)-(2,2,2-trifluoro-acetyl)amino]-3-nitro benzoic acid methyl ester

To a stirred solution of 3-Nitro-2-(2,2,2-trifluoro-acetylamino)-benzoic acid methyl ester (7.0 gm, 0.024 mol) in acetone (50 ml), anhydrous potassium carbonate (6.6 gm, 0.048 mol) was charged. 4-Bromobenzyl bromide (8.9 gm, 0.035 mol) in acetone (20 ml) was added drop wise at room temperature over a period of 10 minutes. Reaction mixture was stirred at room temperature for 24 hours. The reaction mixture was evaporated under vacuum. Crude was taken into ethyl acetate (200 ml) and washed with water (100 ml×2). Organic layer was dried over sodium sulphate and evaporated under vacuum to get oil. Oil was cooled down to 0-5° C. to get solid. Solid was stirred in hexane (70 ml), filtered and suck dried under vacuum for 30 minutes to give 6.4 gm of 2-[(4-Bromo-benzyl)-(2,2,2-trifluoro-acetyl)amino]-3-nitro benzoic acid methyl ester.

### STEP 11: Synthesis of 2-(4-Bromo-benzylamino)-3-nitro-benzoic acid methyl ester

To a stirred cold solution of 2-[(4-Bromo-benzyl)-(2,2,2-trifluoro-acetyl)amino]-3-nitro benzoic acid methyl ester (6.4 gm, 0.01 mol) in Tetrahydrofuran (70 ml), sodium hydroxide solution (5 gm in 20 ml water) was added. Reaction mixture was stirred at room temperature for 7 hours. Tetrahydrofuran was evaporated and reaction mixture was acidified with external cooling by dilute aqueous hydrochloric acid pH-2. Extracted with ethyl acetate (200 ml×2), dried over sodium sulphate and evaporated under vacuum to give 3.6 gm of 2-(4-Bromo-benzylamino)-3-nitro-benzoic acid methyl ester as a brown solid.

### STEP 12: Synthesis of 3-Amino-2-(4-bromo-benzylamino)-benzoic acid methyl ester

To a stirred solution of 2-(4-Bromo-benzylamino)-3-nitro-benzoic acid methyl ester (2.6 gm, 0.006 mol) in ethyl acetate (65 ml), Tin (II) chloride dihydrate (6.5 gm, 0.028 mol) was charged and reaction mixture was heated at 70° C. for 1 hour. Reaction mixture was cooled, quenched on saturated aqueous sodium carbonate solution. Organic layer was separated and aqueous layer was extracted with ethyl acetate (100 ml×2). Combined organic layer was dried over sodium sulphate and evaporated to get 2.0 gm of 3-Amino-2-(4-bromo-benzylamino)-benzoic acid methyl ester as a brown oil.

### STEP 13: Synthesis of 3-(4-Bromo-benzyl)-2-ethoxy-3H-benzoimidazole-4-carboxylic acid methyl ester

To a stirred solution of 3-Amino-2-(4-bromo-benzylamino)-benzoic acid methyl ester (0.9 gm, 0.003 mol) in acetic acid (0.3 ml, 0.005 mol), Tetra ethyl orthocarbonate (0.7 gm, 0.003 mol) was charged and reaction mixture was heated at 70-80° C. for 1 hour. Cooled down to room temperature and Saturated sodium bicarbonate solution was charged in reaction mixture. The reaction mixture was extracted with ethyl acetate (50 ml×2). Organic layer was dried over sodium sulphate and evaporated under vacuum to get 1 gm of 3-(4-bromo-benzyl)-2-ethoxy-3H-benzoimidazole-4-carboxylic acid methyl ester as a greenish solid.

### STEP 14: Synthesis of 3-(4-{2-[(3,4-dimethyl-isoxazol-5-yl)-(2-trimethylsilanyl-ethoxymethyl)-sulfamoyl]-5-methyl-thiophen-3-yl}-benzyl)-2-ethoxy-3H-benzoimidazole-4-carboxylic acid methyl ester

Under the flow of dry nitrogen placed (2.4 gm, 0.0062 mol) of 3-(4-bromo-benzyl)-2-ethoxy-3H-benzoimidazole-4-carboxylic acid methyl ester followed by addition of sodium carbonate solution prepared by dissolving (1.1 gm) in 8 ml of water. Then under stirring charged (320 mg, 0.00055 mol) of bis(triphenylphosphine) palladium (II) Chloride, followed by addition of 10 ml dimethoxy ethane. To this reaction mixture drop wise added the solution of 3-Borono-N-(3,4-dimethyl-3-isoxazolyl)-N-[(2-trimethylsilanyl-ethoxymethyl)]-5-methyl-thiophene sulphonamide (3.15 gm, 0.0068 mol) in 15 ml dimethoxy ethane in 30 min. Reaction mixture was stirred and refluxed for 6 hrs. Reaction mixture was cooled to room temperature and 50 ml of ethyl acetate was added to it followed by evaporation under vacuum. The residue obtained was dissolved in 100 ml of ethyl acetate and washed with water and brine, dried over sodium sulphate and evaporated under vacuum to give 2 gm of crude product as a brown oil which was purified by column chromatography over Silica Gel using Hexane:Ethyl acetate as an eluent to give 2.5 gm of 3-(4-{2-[(3,4-dimethyl-isoxazol-5-yl)-(2-trimethylsilanyl-ethoxymethyl)-sulfamoyl]-5-methyl-thiophen-3-yl}-benzyl)-2-ethoxy-3H-benzoimidazole-4-carboxylic acid methyl ester

### STEP 15: Synthesis of 3-{4-[2-(3,4-dimethyl-isoxazol-5-ylsulfamoyl)-5-methyl-thiophen-3-yl]-benzyl}-2-ethoxy-3H-benzoimidazole-4-carboxylic acid methyl ester

(0.5 gm, 0.00069 mmol) of 3-(4-{2-[(3,4-Dimethyl-isoxazol-5-yl)-(2-trimethylsilanyl-ethoxymethyl)-sulfamoyl]-5-methyl-thiophen-3-yl}-benzyl)-2-ethoxy-3H-benzoimidazole-4-carboxylic acid methyl ester dissolved in 15 ml tetrahydrofuran followed by addition of tetrabutylammonium fluoride solution (2 ml, 1 molar solution in tetrahydrofuran) in it. The reaction mixture was heated at 90° C. for 3 hrs. After that the reaction mixture was cooled to room temperature and to it dilute hydrochloric acid was added and extracted with ethyl acetate (50 ml×2), ethyl acetate layer was washed with water and brine, dried over sodium sulphate and evaporated under vacuum to give 0.5 gm brawn colored oily mass of 3-{4-[2-(3,4-dimethyl-isoxazol-5-ylsulfamoyl)-5-methyl-thiophen-3-yl]-benzyl}-2-ethoxy-3H-benzoimidazole-4-carboxylic acid methyl ester.

### STEP 16: Synthesis of 3-{4-[2-(3,4-dimethyl-isoxazol-5-ylsulfamoyl)-5-methyl-thiophen-3-yl]-benzyl}-2-ethoxy-3H-benzoimidazole-4-carboxylic acid

To 3-{4-[2-(3,4-dimethyl-isoxazol-5-ylsulfamoyl)-5-methyl-thiophen-3-yl]-benzyl}-2-ethoxy-3H-benzoimidazole-4-carboxylic acid methyl ester (0.5 gm, 0.0010 mol)) was added solution of lithium hydroxide (0.15 gm in 2.5 ml of water) followed by addition of (2.5 ml) methanol. This reaction mixture was stirred at room temp. for 6 hrs. Then after the reaction mixture was evaporated under vacuum, to the residue thus obtained ml of water was added and this was extracted with ml of diethyl ether. Aqueous layer was separated, cooled to 5° C. and acidified to pH 2 with dilute hydrochloric acid and extracted with ethyl acetate (50 ml×2). The organic layer was washed with water and brine then dried over sodium sulphate and concentrated under vacuum to give brown solid which was triturated with hexane filtered under vacuum washed with hexane and suction dried to provide 108 mg of 3-{4-[2-(3,4-dimethyl-isoxazol-5-ylsulfamoyl)-5-methyl-thiophen-3-yl]-benzyl}-2-ethoxy-3H-benzoimidazole-4-carboxylic acid.

Molecular Formula: C₂₇H₂₆N₄O₆S₂

Molecular Weight: 566.65

¹HNMR (DMSOd₆): 1.42 (t, J=7.2 Hz 3H), 1.46 (s, 3H), 1.94 (s, 3H), 2.47 (s, 3H), 4.58-4.64 (q, J=7.2 Hz, 2H), 5.68 (s, 2H), 6.68 (s, 1H), 6.94-6.96 (d, J=8 Hz, 2H), 7.19 (t, J=8 Hz, 1H), 7.28 (br, 2H), 7.55-7.70 (m, 2H), 11.07 (br, 1H), 13.18 (br, 1H).

Mass Spectrum: (m⁺¹) 567.1

### Example 27

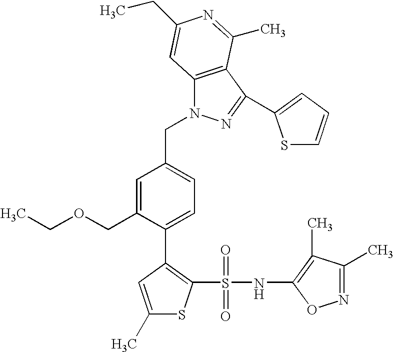

### 3-[2-Ethoxymethyl-4-(6-ethyl-4-methyl-3-thiophen-2-yl-pyrazolo[4,3-c]pyridin-1-ylmethyl)-phenyl]-5-methyl-thiophene-2-sulfonic acid (3,4-dimethyl-isoxazol-5-yl)-amide

### STEP 01: Synthesis of 1-Thiophene-2-yl-butane-1,3-dione

Synthesis of 1-Thiophene-2-yl-butane-1,3-dione was carried out as per STEP 01 of Example 22

### STEP 02: Synthesis of 3-Amino-1-thiophene-2yl-but-2-en-1-one

Synthesis of 3-Amino-1-thiophene-2yl-but-2-en-1-one was carried out as per STEP 01 of Example 22

### STEP 03: Synthesis of 6-Ethyl-2-methyl-3-(thiophene-2-carbonyl)-1H-pyridin-4-one

Synthesis of 6-Ethyl-2-methyl-3-(thiophene-2-carbonyl)-1H-pyridin-4-one was carried out as per STEP 05 of Example 22

### STEP 04: Synthesis of (4-Chloro-6-ethyl-2-methyl-pyridin-3-yl)-thiophen-2-yl-methanone

Synthesis of (4-Chloro-6-ethyl-2-methyl-pyridin-3-yl)-thiophen-2-yl-methanone was carried out as per STEP 06 of Example 22

### STEP 05: Synthesis of 6-Ethyl-4-methyl-3-thiophen-2-yl-1H-pyrazolo[4,3-c]pyridine

Synthesis of 6-Ethyl-4-methyl-3-thiophen-2-yl-1H-pyrazolo[4,3-c]pyridine was carried out as per STEP 07 of Example 22

### STEP 06: Synthesis of 4-Bromo-3-bromomethyl-benzoic acid methyl ester

28 gm (0.11 mol) 4-Bromo-3-methyl-benzoic acid ethyl ester was dissolved in 120 ml of carbon tetrachloride, to it was added 22.65 gm (0.12 mol) N-Bromosuccinimide followed by addition of 1.4 gm (0.005 mol) Benzoyl peroxide (75% in water). This reaction mixture was stirred and reflux for 2 to 3 hrs. Reaction mixture was cooled to 0° C. to get the crystalline solid, which was filtered under vacuum and was washed with 30 ml of carbon tetrachloride. Thus filtrate obtained was evaporated under vacuum. Residue was diluted with 300 ml hexane and cooled to 0° C. to get the crystalline solid, which was filtered under vacuum washed with hexane to give 17.5 gm 4-Bromo-3-bromomethyl-benzoic acid ethyl ester.

### STEP 07: Synthesis of 4-Bromo-3-ethoxymethyl-benzoic acid methyl ester

To cooled solution of 30 ml ethanol and 7 gm (0.10 mol) sodium ethoxide at 0° C. was added the solution of 17.5 gm (0.054 mol) in 12 ml N,N-Dimethyl formamide. The reaction mixture was stirred at room temperature (28-30° C.) for 2 hrs and then evaporated under vacuum. The residue obtained was acidified to pH 1 with dilute hydrochloric acid and extracted with ethyl acetate (100 ml×2). Combine extracts were washed with water and brine. Dried over sodium sulphate and evaporated under vacuum to give 12.5 gm of 4-Bromo-3-ethoxymethyl-benzoic acid ethyl ester.

### Step 08: Synthesis of 5-methyl thiophene-2-sulphonyl chloride

Synthesis of 5-methyl thiophene-2-sulphonyl chloride was carried out as per STEP 07 of Example 01

### Step 09: Synthesis of 5-methyl-thiophene-2-sulfonic acid (3,4-dimethyl-isoxazol-5-yl)-amide

Synthesis of 5-methyl-thiophene-2-sulfonic acid (3,4-dimethyl-isoxazol-5-yl)-amide was carried out as per STEP 02 of Example 74

### Step 10: Synthesis of 5-methyl-thiophene-2-sulfonic acid (3,4-dimethyl-isoxazol-5-yl)-(2-trimethylsilanyl-ethoxymethyl)-amide

Synthesis of 5-methyl-thiophene-2-sulfonic acid (3,4-dimethyl-isoxazol-5-yl)-(2-trimethylsilanyl-ethoxymethyl)-amide was carried out as per STEP 03 of Example 74

### Step 11: Synthesis of 3-Borono-N-(3,4-dimethyl-3-isoxazolyl)-N-[(2-trimethylsilanyl-ethoxymethyl)]-5-methyl-thiophene sulphonamide

Synthesis of 3-Borono-N-(3,4-dimethyl-3-isoxazolyl)-N-[(2-trimethylsilanyl-ethoxymethyl)]-5-methyl-thiophene sulphonamide was carried out as per STEP 04 of Example 74

### STEP 12: Synthesis of 4-{2-[(3,4-dimethyl-isoxazol-5-yl)-(2-methoxy-ethoxymethyl)-sulfamoyl]-5-methyl-thiophen-3-yl}-3-ethoxymethyl-benzoic acid methyl ester

To a stirred solution of 4-bromo-3-ethoxymethyl-benzoic acid methyl ester (3 gm, 0.011 mol) in dimethoxy ethane (50 ml) under nitrogen was added bis(triphenylphosphine)palladium(II)chloride (695 mg, 0.00099 mol) followed by addition of 2M aqueous sodium carbonate (3.15 gm in 15 ml water) Reaction mixture was stirred at room temperature for 10 min and then heated at 60° C. To this drop wise added the solution of 3-Borono-N-(3,4-dimethyl-3-isoxazolyl)-N-[(2-trimethylsilanyl-ethoxymethyl)]-5-methyl-thiophene sulphonamide (2 gm, 0.005 mol in 25 ml dimethoxy ethane) within 45 min and reaction was refluxed for 60 min. After 1 hr further addition of 3-Borono-N-(3,4-dimethyl-3-isoxazolyl)-N-[(2-trimethylsilanyl-ethoxymethyl)]-5-methyl-thiophene sulphonamide (2 gm, 0.005 mol in 25 ml dimethoxy ethane) within 45 min was repeated and the reaction mixture was refluxed for 4 hrs and stirred at room temperature for 12 hrs. Reaction mixture was diluted with ethyl acetate 100 ml and water, layers were separated, aqueous layer further extracted with ethyl acetate. Combine extracts was washed with water and brine. Dried over sodium sulphate and concentrated under vacuum. Crude compound was purified by column chromatography on a silica gel to yield 2.6 gm of 4-{2-[(3,4-Dimethyl-isoxazol-5-yl)-(2-methoxy-ethoxymethyl)-sulfamoyl]-5-methyl-thiophen-3-yl}-3-ethoxymethyl-benzoic acid methyl ester as pale yellow oily mass.

### STEP 13: Synthesis of 3-(2-Ethoxymethyl-4-hydroxymethyl-phenyl)-5-methyl-thiophene-2-sulfonic acid (3,4-dimethyl-isoxazol-5-yl)-(2-methoxy-ethoxymethyl)-amide

Lithium aluminum hydride (350 mg, 0.9.2 mmol) was added to a stirred solution of tetrahydrofuran at 0° C. under flow of nitrogen, followed by addition of 4-{2-[(3,4-Dimethyl-isoxazol-5-yl)-(2-methoxy-ethoxymethyl)-sulfamoyl]-5-methyl-thiophen-3-yl}-3-ethoxymethyl-benzoic acid methyl ester (2.6 gm, 4.7 mmol) in ml tetrahydrofuran. Stirred the reaction mixture at 0° C. for 1 hr and raised the temperature of the reaction mixture to room temperature and stirred for 4 hrs. Reaction mixture was cooled to 0° C., and drop wise added sodium hydroxide solution 30 ml (1 gm dissolved in 100 ml water) maintaining the temperature at 0° C., followed by extraction with ethylacetate (25 ml×2). Organic layer was dried over sodium sulphate and concentrated completely under vacuum to give 2.2 gm of 3-(2-Ethoxymethyl-4-hydroxymethyl-phenyl)-5-methyl-thiophene-2-sulfonic acid (3,4-dimethyl-isoxazol-5-yl)-(2-methoxy-ethoxymethyl)-amide

### STEP 14: Synthesis of methanesulfonic acid 4-{2-[(3,4-dimethyl-isoxazol-5-yl)-(2-methoxy-ethoxymethyl)-sulfamoyl]-5-methyl-thiophen-3-yl}-3-ethoxymethyl-benzyl ester

N-Ethyl diisopropyl amine (2.13 ml, 0.012 mol) was added to a solution of 3-(2-ethoxymethyl-4-hydroxymethyl-phenyl)-5-methyl-thiophene-2-sulfonic acid (3,4-dimethyl-isoxazol-5-yl)-(2-methoxy-ethoxymethyl)-amide (2.2 gm, 4.1 mol) in 30 ml of dichloro methane. Cooled the reaction mixture to 0° C., added slowly methane sulfonyl chloride (0.45 ml, 5 mmol) into the reaction mixture. Maintained the reaction mixture at room temperature for 3 hrs. The reaction mixture was dumped into ice-cold water followed by extraction with methylene chloride (50 ml×2). Combine extracts was given washing with dilute hydrochloric acid followed by water and brine solution. Dried the organic layer over sodium sulphate and concentrated to give 2.2 gm of methanesulfonic acid 4-{2-[(3,4-dimethyl-isoxazol-5-yl)-(2-methoxy-ethoxymethyl)-sulfamoyl]-5-methyl-thiophen-3-yl}-3-ethoxymethyl-benzyl ester

### Step 15: Synthesis of 3-[2-Ethoxymethyl-4-(6-ethyl-4-methyl-3-thiophen-2-yl-pyrazolo[4,3-c]pyridin-1-ylmethyl)-phenyl]-5-methyl-thiophene-2-sulfonic acid (3,4-dimethyl-isoxazol-5-yl)-(2-methoxy-ethoxymethyl)-amide

To the stirred solution of 6-Ethyl-4-methyl-3-thiophen-2-yl-1H-pyrazolo[4,3-c]pyridine (0.447 gm, 1.8 mmol) in dimethyl formamide (10 ml) at 0° C. under nitrogen was added portion wise sodium hydride (60% in mineral oil) (132 mg, 3.3 mmol). After the addition, the reaction mixture was warmed to ambient temperature and maintained for 30 min. Reaction mixture was cooled to 0° C. and a solution of Methanesulfonic acid 4-{2-[(3,4-dimethyl-isoxazol-5-yl)-(2-methoxy-ethoxymethyl)-sulfamoyl]-5-methyl-thiophen-3-yl}-3-ethoxymethyl-benzyl ester (1 gm, 1.8 mmol) in (10)ml dimethyl formamide was added drop wise to the reaction mixture and stirred at room temperature for 24 hrs. The mixture was then diluted with ethyl acetate (40 ml), followed by 10 ml of cold water, Organic layer washed with water and brine then dried over sodium sulphate and evaporated under vacuum to afford crude 0.6 gm of 3-[2-Ethoxymethyl-4-(6-ethyl-4-methyl-3-thiophen-2-yl-pyrazolo[4,3-c]pyridin-1-ylmethyl)-phenyl]-5-methyl-thiophene-2-sulfonic acid (3,4-dimethyl-isoxazol-5-yl)-(2-methoxy-ethoxymethyl)-amide as a viscous oily mass.

### Step 16: Synthesis of 3-[2-Ethoxymethyl-4-(6-ethyl-4-methyl-3-thiophen-2-yl-pyrazolo [4,3-c] pyridin-1-ylmethyl)-phenyl]-5-methyl-thiophene-2-sulfonic acid (3,4-dimethyl-isoxazol-5-yl)-amide

To, crude 600 mg of 3-[2-Ethoxymethyl-4-(6-ethyl-4-methyl-3-thiophen-2-yl-pyrazolo[4,3-c]pyridin-1-ylmethyl)-phenyl]-5-methyl-thiophene-2-sulfonic acid (3,4-dimethyl-isoxazol-5-yl)-(2-methoxy-ethoxymethyl)-amide was added ethanol (10 ml) and 6N aqueous hydrochloric acid (8 ml) at room temperature. Reaction mixture was stirred and heated to 120° C. for 6 hrs. The reaction mixture was concentrated under vacuum and pH of the solution was adjusted to 8 using a saturated solution of sodium bicarbonate, and the mixture was extracted with ethyl acetate (30 ml×3). The combined organic extract were washed with water and brine then dried over sodium sulphate and concentrated under vacuum. The residue was purified by column chromatography over Silica Gel column using hexane:ethyl acetate to afford 80 mg of 3-[2-Ethoxymethyl-4-(6-ethyl-4-methyl-3-thiophen-2-yl-pyrazolo [4,3-c] pyridin-1-ylmethyl)-phenyl]-5-methyl-thiophene-2-sulfonic acid (3,4-dimethyl-isoxazol-5-yl)-amide

Molecular Formula: C₂₇H₂₆N₄O₆S₂

Molecular Weight: 661.87

¹HNMR (DMSOd₆): 1.00-1.05 (m, 3H), 1.30-1.32 (m, 3H), 1.42 (s, 3H), 2.00 (s, 3H), 2.46 (s, 3H), 2.82-2.88 (q, J=7.6 Hz, 2H), 3.25-3.28 (q, J=7.6 Hz, 2H) 4.03 (s, 2H), 5.72 (s, 2H), 6.66-7.74 (m, 8H).

Mass Spectrum: (m⁺¹) 662.2

### Example 28

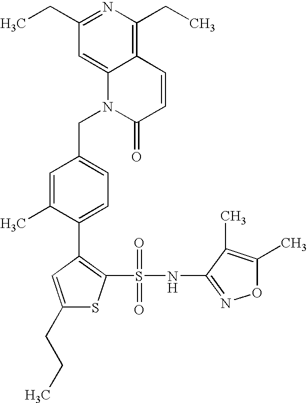

### 3-[4-(5,7-diethyl-2-oxo-2H-[1,6] naphthyridin-1-ylmethyl)-2-methyl-phenyl]-5-Propyl-thiophene-2-sulfonic acid (4,5-dimethyl-isoxazol-3-yl)-amide

### Step 01: Synthesis of sodium salt of 3-Hydroxy-2-methyl-but-2-enenitrile. (sodium salt of 3-cyano-2-butanone)

Under the flow of dry nitrogen to a stirred solution of Propionitrile (200 gm, 3.6 mol) in Toluene (1000 ml) n-butyl acetate (538 gm, 6.46 mol) was added in to it, followed by addition of Sodium Methoxide (197 gm, 3.64 mol) in to the reaction mixture. After completion of the addition reaction mixture was heated and stirred at 90° C. for 24 hrs. Then after reaction mixture was cooled to room temperature (25° C.). The separated solid was filtered and washed with Hexane and dried under vacuum at 60°-65° C. to yield 198 gm sodium salt of 3-Hydroxy-2-methyl-but-2-enenitrile.(sodium salt of 3-cyano-2-butanone)

### Step 02: Synthesis of 2,2-(2-Methyl-[1,3] dioxolan-2-yl)-Propionitrile

To a stirred solution of Sodium salt of cyanobutanone (70 gm, 0.588 mo) in Toluene (780 ml), Sulphuric acid (52 gm 0.5349 mol) was added slowly at 0-5° C. and further Ethylene glycol (72.93 gm, 1.172 mol) was added. The reaction mixture was refluxed for 8.0 hrs with Dean-Stark apparatus. Reaction mixture was cooled to 0° C. followed by addition of 2 M NaOH solution in to it and toluene layer was separated and washed with saturated sodium chloride Solution and dried over sodium sulphate and evaporated under vacuum to yield 59 gm of 2-(2-methyl-[1,3] dioxolan-2-yl)-propionitrile as yellowish liquid.

### Step 03: Synthesis of N-hydroxy-2-(2-methyl-[1,3] dioxolan-2-yl)-propionamidine OR 2-(β-ethylene dioxy aceto) propionamideoxime

To a stirred solution of 4 M sodium hydroxide (310 ml) in methanol (267 ml), hydroxylamine hydrochloride (75 gm, 3.0 mol) was charged slowly at 10° C. after completion of the addition, 2-(β-ethylene dioxy aceto) propionamideoxime (65 gm, 0.460 mol) was charged in to the reaction mixture at room temperature. The reaction mixture was then refluxed for 5 hrs. The reaction mixture was cooled to room temperature and then methanol was distilled under vacuum, the residue thus obtained was extracted with ethyl acetate (100 ml×3). The ethyl acetate layer was washed with brine and dried over sodium sulphate and evaporation of ethyl acetate layer under reduced pressure afforded 34 gm 2-(β-ethylene dioxy aceto) propionamideoxime as oil.

### Step 04: Synthesis of 3-Amino-4,5 dimethyl isoxazole

To a stirred solution of 2-(β-ethylene dioxy aceto) propionamideoxime (105 gm, 0.60 mol) in n-propanol (1050 ml), Sulphuric Acid (96 gm, 0.979 mol) was added slowly at 10-20° C. Reaction mixture was refluxed for 4 hrs. The n-propanol was distilled under vacuum and Ethylacetate (600 ml) was added to the residue and neutralized with aqueous sodium bicarbonate solution. Then after organic layer was separated washed with water and brine, dried over sodium sulphate and evaporated under reduced pressure to yield 40 gm of crude 3-Amino-4,5 dimethyl isoxazole which was recrystalised from toluene/hexane to give 32 gm of pure 3-Amino-4,5 dimethyl isoxazole.

### Step 05: Synthesis of 5-Propyl thiophene-2-sulphonyl chloride

2-Propyl thiophene (5.0 gm, 0.0396 mol) was added to the suspension of Chloro sulfonic acid (6.6 ml, 0.099 mol) and phosphorous pentachloride (8.25 gm, 0.0396 mol) at 10° C. to 15° C. Maintained the reaction mixture at 15° C. for 10 min. Crude reaction mass was slowly dumped into the ice cold water, followed by extraction with diisopropyl ether (50 ml×2). Combined extract was washed with water and brine, dried over anhydrous sodium sulphate, evaporated under vacuum to give 7.0 gm of 5-Propyl thiophene-2-sulphonyl chloride as brown colored liquid.

### Step 06: Synthesis of 5-Propylthiophene-2-sulfonic acid (4,5-dimethyl-isoxazol-3-yl) amide

5-Propyl thiophene-2-sulphonyl chloride (7.0 gm, 0.0312 mol) in methylene chloride (25 ml) was added to a solution of 3-amino-4,5-dimethylisoxazole (3.18 gm, 0.0284 mol) in pyridine (5.8 ml) and dimethylaminopyridine (0.347 gm) at 0° C. The temperature of the reaction mixture was slowly raised to room temperature and stirred for 6 hours. Concentrated the reaction mixture completely under vacuum, acidified the reaction mixture using 1N hydrochloric acid followed by extraction with methylene chloride (100 ml×2). Combine extracts was given washing with water and brine. Dried over sodium sulphate and concentrated to give 8.1 gm of 5-Propylthiophene-2-sulfonic acid (4,5-dimethyl-isoxazol-3yl) amide as brown colored solid.

### Step 07: Synthesis of 5-Propyl-thiophene-2-sulphonic acid (4,5-dimethyl-isoxazol-3-yl)-(2-methoxy-ethoxymethyl)-amide

Potassium carbonate (6.14 gm, 0.044 mol) was added to a stirred solution of dimethyl formamide (30 ml) at 0° C. followed by addition of 5-Propyl-thiophene-2-sulfonic acid (4,5-dimethyl-isoxazol-3yl)amide (5.38 gm, 0.0178 mol). Slowly raised the temperature and maintained at ambient temperature for 30 minutes then cooled to 0° C. followed by addition of methoxy ethoxy methyl chloride (2.67 gm, 0.0214 mol) at 0° C. After the completion of addition, slowly raised to ambient temperature and stirred for 3 hrs. Charged 90 ml ethyl acetate followed by 25 ml ice water to the reaction mixture. Organic layer was separated; aqueous was again extract with ethyl acetate (50 ml×2). Combine extracts was washed with water and brine solution. Dried under sodium sulphate and concentrated under vacuum. Residue was purified by Column Chromatography over Silica Gel column to give 4.2 gm of 5-Propylthiophene-2-sulphonic acid (4,5 dimethyl-isoxazol-3-yl)-(2-methoxy-ethoxymethyl)-amide as yellowish oil.

### Step 08: Synthesis of 3-Borono-N-(4,5-dimethyl-3-isoxazolyl)-N-[(2-methoxy-ethoxy)methyl]-5-Propyl-thiophene sulphonamide

5-Propyl-thiophene-2-sulphonic acid (4,5 dimethyl-isoxazol-3-yl)-(2-methoxy-ethoxymethyl)-amide (4.2 gm, 0.011 mol) was dissolved in tetrahydrofuran (20 ml) and the reaction mixture was cooled to −78° C. under nitrogen atmosphere. n-butyl lithium (17 ml, 0.0275 mol, 1.6 M solution in n-hexane) was added slowly into the reaction mixture by using a syringe. After the completion of addition the reaction mixture was stirred at −78° C. for 1 hr and slowly raised the temperature to 0° C. and stirred for 30 min. Again the reaction mixture cooled to −78° C. then triisopropyl borate (3 ml, 0.0132 mol) was added. After the completion of addition slowly raised the temperature to 0° C. and stirred for 1 hr. Reaction mixture was cooled to −10° C. and added saturated ammonium chloride solution followed by extraction with ethyl acetate (50 ml×2). Combine extract was washed with water and brine. Dried under sodium sulphate and concentrated under vacuum to give 4.5 gm of 3-Borono-N-(4,5-Dimethyl-3-isoxazolyl)-N-[(2-methoxy-ethoxy)methyl]-5-Propyl-thiophene sulphonamide as thick oily mass.

### Step 09: Synthesis of 3-[4-(5,7-diethyl-2-oxo-2H-[1,6] naphthyridin-1-ylmethyl)-2-methyl-phenyl]-5-methyl-thiophene-2-sulfonic acid (4,5-dimethyl-isoxazol-3-yl)-(2-methoxy-ethoxy)amide

To a stirred solution of 1-(4-Bromo-3-methyl-benzyl)-5,7-diethyl-1H-[1,6]naphthyridin-2-one (1.0 gm, 0.0026 mol) in dimethoxy ethane (20 ml) under nitrogen was added Bis(triphenylphosphine)palladium(II)chloride (0.183 gm, 0.00026 mol) followed by addition of 2M aqueous sodium carbonate (0.827 gm in 3.9 ml water) Reaction mixture was stirred at room temperature for 10 min and then heated at 60° C. To this drop wise added the solution of 3-Borono-N-(4,5-Dimethyl-3-isoxazolyl)-N-[(2-methoxy-ethoxy)methyl]-5-Propyl-thiophene sulphonamide (0.5616 gm, 0.0013 mol in 10 ml dimethoxy ethane) within 45 min and reaction was refluxed for 60 min. After 1 hr the same was repeated with further addition of 3-Borono-N-(4,5-Dimethyl-3-isoxazolyl)-N-[(2-methoxy-ethoxy)methyl]-5-Propyl-thiophene sulphonamide (0.5616 gm, 0.0013 mol in 10 ml dimethoxy ethane) within 45 min, reaction mixture was refluxed for 4 hrs and stirred at room temperature for 12 hrs. Reaction mixture was diluted with ethyl acetate (50 ml) and water, layers were separated, aqueous layer further extracted with ethyl acetate. Combine extracts was washed with water and brine. Dried over sodium sulphate and concentrated under vacuum to yield 1.2 gm of 3-[4-(5,7-Diethyl-2-oxo-2H-[1,6]naphthyridin-1-ylmethyl)-2-methyl-phenyl]-5-methyl-thiophene-2-sulfonic acid (4,5-dimethyl-isoxazol-3-yl)-(2-methoxy-ethoxymethyl)-amide as pale yellow oily mass.

### Step 10: Synthesis of 3-[4-(5,7-diethyl-2-oxo-2H-[1,6] naphthyridin-1-ylmethyl)-2-methyl-phenyl]-5-Propyl-thiophene-2-sulfonic acid (4,5-dimethyl-isoxazol-3-yl)-amide

To, 3-[4-(5,7-diethyl-2-oxo-2H-[1,6]naphthyridin-1-ylmethyl)-2-methyl-phenyl]-5-methyl-thiophene-2-sulfonic acid (4,5-dimethyl-isoxazol-3-yl)-(2-methoxy-ethoxymethyl)-amide (1.2 gm) was added ethanol (10 ml) and 6N aqueous hydrochloric acid (4 ml) at room temperature. Reaction mixture was refluxed for 3 hrs. The reaction mixture was concentrated under vacuum and pH of the solution was adjusted to 8 using a saturated solution of sodium bicarbonate and the mixture was extracted with ethyl acetate (25 ml×2). The combined organic extract were washed with water and brine then dried over sodium sulphate and concentrated under vacuum. The residue was purified over Silica Gel chromatography using hexane:ethyl acetate to afford 190 mg of 3-[4-(5,7-diethyl-2-oxo-2H-[1,6] naphthyridin-1-ylmethyl)-2-methyl-phenyl]-5-Propyl-thiophene-2-sulfonic acid (4,5-dimethyl-isoxazol-3-yl)-amide

### STEP 11: Synthesis of methyl 3-amino pentenoate

A mixture of methyl propionyl acetate (50 gm, 0.3842 mol) and ammonium acetate (148 gm, 1.921 mol) in dry methanol (500 ml) was stirred at room temperature for 3 days. The reaction mixture was concentrated. Residue was basified to pH 8 and extracted with ethyl acetate. Ethyl acetate layer washed with water, brine and dried over sodium sulphate and concentrated to give 50 gm of Methyl 3-amino pentenoate as pale yellow liquid.

### STEP 12: Synthesis of 2,6-diethyl-4-oxo-1,4-dihydro-pyridine-3-carboxylic acid methyl ester

A mixture of methyl 3-amino pentenoate (50 gm, 0.387 mol) and methyl propionyl acetate (50 gm, 0.384 mol) in 200 ml of o-xylene and 50 gm of 40 A⁰ molecular sieve were heated to reflux in a dean stark apparatus for 5 days. Molecular sieves was filtered off and the filtrate was concentrated and purified over Silica Gel column to elute 20 gm of 2,6-Diethyl-4-oxo-1,4-dihydro-pyridine-3-carboxylic acid methyl ester with 50% dichloromethane: methanol, as off white solid

### STEP 13: Synthesis of 2,6-diethyl-4-(toluene-4-sulfonylamino) nicotinic acid methyl ester

Tosyl isocyanate (39 gm, 0.197 mol) was added to a stirred suspension of methyl 2,6-diethyl-4-oxo-1,4-dihydropyridine-3-carboxylate (25 gm, 0.119 mol) in acetonitrile (250 ml). After the initial exotherm had subsided the mixture was refluxed for 2 hours. Mixture was cooled to room temperature and the suspended solid was collected by filtration to give 20 gm of 2,6-diethyl-4-(toluene-4-sulfonylamino) nicotinic acid methyl ester.

### STEP 14: Synthesis of 4-amino-2,6-diethyl-nicotinic acid methyl ester

Methyl 2,6-diethyl-4-(4-tosylamino) pyridine-3-carboxylate (40 g, 0.110 mol) was added to concentrated sulfuric acid (57 ml, 1.10 mol) at 0° C. and then the reaction mixture was stirred at 50° C. for 1 hour. Reaction mixture was cooled to room temperature and poured into crushed ice. The mixture was the adjusted to pH 8 by solid sodium carbonate and extracted with DCM combined organic phase were washed with water and brine. Dried over sodium sulfate and concentrated to yield 19 gm of methyl 4-amino-2,6-diethyl pyridine-3-carboxylate as white solid.

### STEP 15: Synthesis of (4-Amino-2,6-diethyl-pyridin-3-yl)-methanol

A solution of methyl 4-amino-2,6-diethyl pyridine-3-carboxylate (20 gm, 0.0962 mol) in tetrahydrofuran (150 ml) was added drop wise to a stirred suspension of lithium aluminium hydride (7.3 gm, 0.1923 mol) in tetrahydrofuran (150 ml) over 30 minute reaction mixture was then stirred and heated under reflux for 5 hrs. The reaction mixture was cooled in an ice-bath 2M aqueous sodium hydroxide solution (50 ml) was added cautiously, followed by water (20 ml) the resulting mixture was stirred for 1 hr then after tetrahydrofuran (150 ml) was added. Insoluble material was removed by filtration and washed with ethyl acetate. Combined organic layers were dried over sodium sulphate concentrated under vacuum to yield 12.1 gm of (4-amino-2,6-diethyl-pyridin-3-yl)-methanol.

### STEP 16: Synthesis of 4-Amino-2,6-diethyl-pyridine-3-carbaldehyde

A mixture of (4-Amino-2,6-diethyl-pyridin-3-yl)-methanol (12.0 gm, 0.0667 mol) and manganese dioxide (17.39 gm, 0.2 mol) in toluene (150 ml) was stirred and heated at reflux for 10 hrs. The hot reaction mixture was filtered and the solid washed with ethyl acetate (150 ml). The combined filtrate was concentrated by evaporation under vacuum to give 11.7 gm of 4-Amino-2,6-diethyl-pyridine-3-carbaldehyde as yellow solid.

### STEP 17: Synthesis of 5,7-diethyl-1H-[1,6]naphthyridin-2-one

A mixture of 4-Amino-2,6-diethyl-pyridine-3-carbaldehyde (11.7 gm, 0.657 mol) and (carethoxymethylene) triphenylphosphorane (27.44 gm, 0.07884 mol) in toluene (150 ml) was stirred and heated at reflux for 5 hrs. The reaction mixture was cooled to room temperature and the solvent was removed by evaporation. A solution of sodium methoxide (12.42 gm, 0.23 mol) in methanol (150 ml) was added to the residue and the resulting solution was heated at reflux for 4 hrs. Methanol was removed by evaporation and water (100 ml) was added. The mixture was acidified to pH 2 by addition of concentrated hydrochloric acid. The mixture was then extracted with ethyl acetate (100 ml×2) The organic extract was discarded. The aqueous phase was then basified by addition of sodium carbonate. This was then extracted with dichloromethane (200 ml×3). The organic layer was washed with water, brine and dried over sodium sulphate and concentrated to give 5.5 gm of 5,7-diethyl-1H-[1,6] naphthyridin-2-one as white solid.

### STEP 18: Synthesis of 1-(4-Bromo-3-methyl-benzyl)-5,7-diethyl-1H-[1,6] naphthyridin-2-one

To the stirred solution of potassium carbonate (6.16 gm, 0.04455 mol) in dimethyl formamide (10 ml) at 0° C. under nitrogen was added 5,7-diethyl-1H-[1,6] naphthyridin-2-one (3.0 gm, 0.01485 mol). After the addition, the reaction mixture was warmed to ambient temperature and maintained for 30 min. Then reaction mixture was cooled to 0° C. and a solution of methanesulfonic acid 4-bromo-3-methyl-benzyl ester (5.39 gm, 0.01931 mol) in 10 ml dimethyl formamide was added drop wise to the reaction mixture and stirred at room temperature for 8 hrs. The mixture was then diluted with ethyl acetate (40 ml), followed by 10 ml of cold water, Organic layer washed with water and brine then dried over sodium sulphate and evaporated under vacuum to afford 4.0 gm of 1-(4-Bromo-3-methyl-benzyl)-5,7-diethyl-1H-[1,6]naphthyridin-2-one as a brown colored solid.

Molecular Formula: C₃₂H₃₆N₄O₄S₂

Molecular Weight: 604

¹HNMR (DMSOd₆): 0.91 (t, J=7.2 Hz, 3H), 1.18-1.27 (m, 6H), 1.45 (s, 3H), 1.61-1.66 (q, J=7.6 Hz, 2H), 1.92 (s, 3H), 2.11 (s, 3H), 2.70-2.80 (m, 4H) 3.04-3.10 (q, J=7.2 Hz, 2H), 5.46 (s, 2H), 6.71-7.16 (m, 6H), 8.22-8.25 (d, J=9.36 Hz, 1H), 10.59 (s, 1H).

Mass Spectrum: (m⁻¹) 603.2

### Example 29

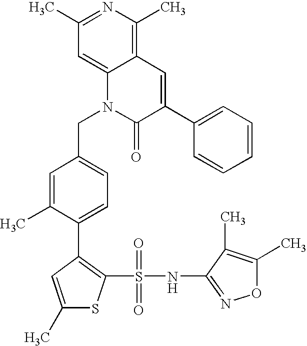

### 3-[4-(5,7-dimethyl-2-oxo-3-phenyl-2H-[1,6] naphthyridin-1-ylmethyl)-2-methyl-phenyl]-5-methyl-thiophene-2-sulfonic acid (4,5-dimethyl-isoxazol-3-yl)-amide

### Step 01: Synthesis of sodium salt of 3-Hydroxy-2-methyl-but-2-enenitrile.(sodium salt of 3-cyano-2-butanone)

Synthesis of sodium salt of 3-Hydroxy-2-methyl-but-2-enenitrile (sodium salt of 3-cyano-2-butanone) was carried out as per STEP 01 of Example 28

### Step 02: Synthesis of 2,2-(2-Methyl-[1,3] dioxolan-2-yl)-Propionitrile

Synthesis of 2,2-(2-Methyl-[1,3] dioxolan-2-yl)-Propionitrile was carried out as per STEP 02 of Example 28

### Step 03: Synthesis of N-Hydroxy-2-(2-methyl-[1,3]dioxolan-2-yl)-propionamidine OR 2-(β-ethylene dioxy aceto) propionamideoxime

Synthesis of N-Hydroxy-2-(2-methyl-[1,3] dioxolan-2-yl)-propionamidine OR 2-(β-ethylene dioxy aceto) propionamideoxime was carried out as per STEP 03 of Example 28

### Step 04: Synthesis of 3-Amino-4,5 dimethyl isoxazole

Synthesis of 3-Amino-4,5 dimethyl isoxazole was carried out as per STEP 04 of Example 28

### Step 05: Synthesis of 5-methyl thiophene-2-sulphonyl chloride

Synthesis of 5-methyl thiophene-2-sulphonyl chloride was carried out as per STEP 07 of Example 01

### Step 06: Synthesis of 5-methyl-thiophene-2-sulfonic acid (4,5-dimethyl-isoxazol-3yl) amide

Synthesis of 5-methyl-thiophene-2-sulfonic acid (4,5-dimethyl-isoxazol-3yl) amide was carried out as per STEP 08 of Example 01

### Step 07: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 08: Synthesis of 3-borono-N-(4,5-dimethyl-3-isoxazolyl)-N-[(2-methoxy-ethoxy)methyl]-5-methyl-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### STEP 09: Synthesis of (4-{2-[(4,5-Dimethyl-isoxazol-3-yl)-(2-methoxy-ethoxymethyl)-sulfamoyl]-5-methyl-thiophene-3-yl}-3-methyl-benzoic acid methyl ester

Synthesis of (4-{2-[(4,5-dimethyl-isoxazol-3-yl)-(2-methoxy-ethoxymethyl)-sulfamoyl]-5-methyl-thiophene-3-yl}-3-methyl-benzoic acid methyl ester was carried out as per STEP 08 of Example 19

### STEP 10: Synthesis of 3-(4-hydroxy methyl-2-methyl-phenyl)-5-methyl-thiophene-2-sulfonic acid-(4,5-dimethyl-isoxazol-3-yl)-(2-methoxy-ethoxymethyl) amide

Synthesis of 3-(4-hydroxy methyl-2-methyl-phenyl)-5-methyl-thiophene-2-sulfonic acid-(4,5-Dimethyl-isoxazol-3-yl)-(2-methoxy-ethoxymethyl) amide was carried out as per STEP 09 of Example 19

### STEP 11: Synthesis of methane sulfonic acid 4-{2-[(4,5-dimethyl-isoxazol-3-yl)-(2-methoxy-ethoxy methyl)-sulfamoyl]-5-methylthiophene-3-yl}-3-methyl-benzyl ester

Synthesis of methane sulfonic acid 4-{2-[(4,5-dimethyl-isoxazol-3-yl)-(2-methoxy-ethoxy methyl)-sulfamoyl]-5-methylthiophene-3-yl}-3-methyl-benzyl ester was carried out as per STEP 10 of Example 19

### Step 12: Synthesis of 3-[4-(5,7-dimethyl-2-oxo-3-phenyl-2H-[1,6]naphthyridin-1-ylmethyl)-2-methyl-phenyl]-5-methyl-thiophene-2-sulfonic acid (4,5-dimethyl-isoxazol-3-yl)-(2-methoxy-ethoxymethyl)-amide

To the stirred solution of 5,7-dimethyl-3-phenyl-1H-[1,6] naphthyridin-2-one (0.5 gm, 0.002 mol) in dimethyl formamide (10 ml) at 0° C. under nitrogen was added portion wise sodium hydride (60% in mineral oil) (150 mg, 0.0031 mol). After the addition, the reaction mixture was warmed to ambient temperature and maintained for 30 min. Reaction mixture was cooled to 0° C. and a solution of methane sulfonic acid 4-{2-[(4,5-dimethyl-isoxazol-3yl)-(2-methoxy-ethoxy methyl)-sulfamoyl]-5-methyl-thiophen-3-yl}-3-methyl-benzyl ester. (1 gm, 0.002 mol) in (10 ml) dimethyl formamide was added drop wise to the reaction mixture and stirred at room temperature for 24 hrs. The mixture was then diluted with ethyl acetate (40 ml), followed by 10 ml of cold water, Organic layer washed with water and brine then dried over sodium sulphate and evaporated under vacuum to afford 1.3 gm of 3-[4-(5,7-Dimethyl-2-oxo-3-phenyl-2H-[1,6]naphthyridin-1-ylmethyl)-2-methyl-phenyl]-5-methyl-thiophene-2-sulfonic acid (4,5-dimethyl-isoxazol-3-yl)-(2-methoxy-ethoxymethyl)-amide as a viscous oily mass.

### Step 13: Synthesis of 3-[4-(5,7-dimethyl-2-oxo-2H-[1,6]naphthyridin-1-ylmethyl)-2-methyl-phenyl]-5-methyl-thiophene-2-sulfonic acid(4,5-dimethyl-isoxazol-3-yl)-amide

To, 3-[4-(5,7-Dimethyl-2-oxo-3-phenyl-2H-[1,6]naphthyridin-1-ylmethyl)-2-methyl-phenyl]-5-methyl-thiophene-2-sulfonic acid (4,5-dimethyl-isoxazol-3-yl)-(2-methoxy-ethoxymethyl)-amide (1.3 gm) was added ethanol (10 ml) and 6N aqueous hydrochloric acid (6 ml) at room temperature. Reaction mixture was refluxed for 3 hrs. The reaction mixture was concentrated under vacuum and pH of the solution was adjusted to 8 using a saturated solution of sodium bicarbonate and the mixture was extracted with ethyl acetate (25 ml×2). The combined organic extract were washed with water and brine then dried over sodium sulphate and concentrated under vacuum. The residue was purified by column Chromatography over Silica Gel column using hexane:ethyl acetate to afford 200 mg of 3-[4-(5,7-Dimethyl-2-oxo-3-phenyl-2H-[1,6]naphthyridin-1-ylmethyl)-2-methyl-phenyl]-5-methyl-thiophene-2-sulfonic acid (4,5-dimethyl-isoxazol-3-yl)-amide as a white solid.

### STEP 14: Synthesis of 2,6-Dimethyl-4-oxo-1,4-dihydro-pyridine-3-carboxylic acid ethyl ester

A mixture of methyl 3-Amino-but-2-enoic acid ethyl ester (24.0 gm, 0.184 mol) and 3-Oxo-butyric acid methyl ester (22.0 gm, 0.17 mol) in 200 ml of o-xylene and 50 gm of 3 A⁰ molecular sieve were heated to reflux with a dean stark apparatus for 2 days. A molecular sieve was filtered off and the filtrate was concentrated to give semi-solid which was slurried in 25 ml ethyl acetate and filtered to give 9.0 gm of 2,6-Dimethyl-4-oxo-1,4-dihydro-pyridine-3-carboxylic acid ethyl ester as off white solid

### STEP 15: Synthesis of 2,6-Dimethyl-4-(toluene-4-sulfonylamino)-nicotinic acid ethyl ester

Tosyl isocyanate (36.0 gm, 0.18 mol) was added to a stirred suspension of 2,6-Dimethyl-4-oxo-1,4-dihydro-pyridine-3-carboxylic acid ethyl ester (20.0 gm, 0.10 mol) in acetonitrile (200 ml) after the initial exotherm had subsided the mixture was refluxed for 2 hours. Mixture was cooled to room temperature and the suspended solid was collected by filtration to give 37.0 gm of 2,6-diethyl-4-(toluene-4-sulfonylamino)-nicotinic acid ethyl ester.

### STEP 16: Synthesis of 4-amino-2,6-dimethyl-nicotinic acid ethyl ester

2,6-Dimethyl-4-(toluene-4-sulfonylamino)-nicotinic acid ethyl ester (37.0 gm, 0.10 mol) was added to concentrated sulfuric acid (57 ml, 1.15 mol) at 0° C. and then the reaction mixture was stirred at 50° C. for 1 hour. Reaction mixture was cooled to room temperature and poured into crushed ice. The mixture was the adjusted to pH 8 by solid sodium carbonate and extracted with dichloromethane (100 ml×2) combined organic phase were washed with water and brine. Dried over sodium sulfate and concentrated to yield 20.6 gm of 4-amino-2,6-dimethyl-nicotinic acid ethyl ester.

### STEP 17: Synthesis of (4-Amino-2,6-dimethyl-pyridin-3-yl)-methanol

To a stirred cold solution of 4-amino-2,6-dimethyl-nicotinic acid ethyl ester (18.0 gm, 0.0928 mol) in tetrahydrofuran (130 ml), lithium aluminium hydride (7.044 gm, 0.1856 mol) was charged portion wise at 0° C. The reaction mixture was stirred at room temperature for 15 min then refluxed for 6 hours. Cooled the reaction mixture to 0° C. and to it, drop wise added aqueous sodium hydroxide solution (10 gm in 100 ml water). Organic layer was decanted and aqueous layer was extracted with tetrahydrofuran (500 ml×2). Combined organic layer was evaporated to give 12.0 gm of (4-Amino-2,6-dimethyl-pyridin-3-yl)-methanol as a crystalline solid.

### STEP 18: Synthesis of 4-Amino-2,6-dimethyl-pyridine-3-carbaldehyde

To a solution of (4-Amino-2,6-dimethyl-pyridin-3-yl)-methanol (5.0 gm, 0.0329 mol) in toluene (80 ml), manganese dioxide (8.58 gm, 0.0987 mol) was charged. Reaction mixture was refluxed for 6 hours. Cooled the reaction mixture to room temperature and filtered through hyflow bed and residue was washed with toluene. Organic filtrate was evaporated to give 5.29 gm of 4-Amino-2,6-dimethyl-pyridine-3-carbaldehyde as a white solid.

### STEP 19: Synthesis of 5,7-dimethyl-3-phenyl-[1,6]naphthyridin-2-ylamine

A mixture of 4-Amino-2,6-dimethyl-pyridine-3-carbaldehyde (3 gm, 0.02 mol), sodium methoxide (2.14 gm, 0.04 mol) and phenylacetonitrile (2.34 gm, 0.02 mol) was heated at 60° C. for 2 hrs. Volatile material was removed by evaporation and the residue was partitioned between ethyl acetate and water. The organic phase was separated, dried over sodium sulphate and concentrated under vacuum to give 4.3 gm of 5,7-Dimethyl-3-phenyl-[1,6]naphthyridin-2-ylamine.

### STEP 20: Synthesis of 5,7-dimethyl-3-phenyl-1H-[1,6]naphthyridin-2-one

A solution of sodium nitrite (6.0 gm) in water (20 ml) was added drop wise over 45 minutes to a solution of 5,7-Dimethyl-3-phenyl-[1,6]naphthyridin-2-ylamine (4.3 gm, 0.02 mol), 30 ml water and 11 N hydrochloric acid (10 ml). The reaction mixture was stirred for a further 1 hr and then the suspended white solid was collected by filtration and dried under vacuum to give 4.35 gm of 5,7-Dimethyl-3-phenyl-1H-[1,6]naphthyridin-2-one

Molecular Formula: C₃₄H₃₂N₄O₄S₂

Molecular Weight: 624

¹HNMR (DMSOd₆): 1.49 (s, 3H), 1.93 (s, 3H), 2.12 (s, 3H), 2.47 (s, 3H), 2.48 (s, 3H), 2.78 (s, 3H) 5.54 (s, 2H), 6.71 (s, 1H), 6.88-6.94 (m, 2H) 7.15 (s, 1H) 7.21 (s, 1H), 7.40-7.49 (m, 3H), 7.79-7.81 (m, 2H), 8.24 (s, 1H), 10.62 (br, 1H).

Mass Spectrum: (m⁺¹) 623.2

### Example 30

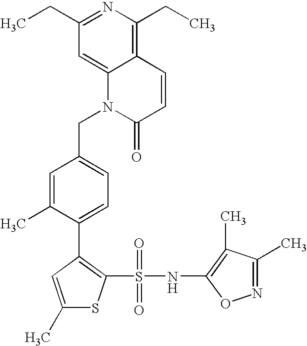

### 3-[4-(5,7-diethyl-2-oxo-2H-[1,6] naphthyridin-1-ylmethyl)-2-methyl-phenyl]-5-methyl-thiophene-2-sulfonic acid (3,4-dimethyl-isoxazol-5-yl)-amide

### Step 01: Synthesis of 5-methyl thiophene-2-sulphonyl chloride

Synthesis of 5-methyl thiophene-2-sulphonyl chloride was carried out as per STEP 07 of Example 01

### Step 02: Synthesis of 5-Methyl-thiophene-2-sulfonic acid (3,4-dimethyl-isoxazol-5-yl)-amide

Synthesis of 5-methyl-thiophene-2-sulfonic acid (3,4-dimethyl-isoxazol-5-yl)-amide was carried out as per STEP 02 of Example 26

### Step 03: Synthesis of 5-methyl-thiophene-2-sulfonic acid (3,4-dimethyl-isoxazol-5-yl)-(2-trimethylsilanyl-ethoxymethyl)-amide

Synthesis of 5-Methyl-thiophene-2-sulfonic acid (3,4-dimethyl-isoxazol-5-yl)-(2-trimethylsilanyl-ethoxymethyl)-amide was carried out as per STEP 03 of Example 26

### Step 04: Synthesis of 3-Borono-N-(3,4-dimethyl-3-isoxazolyl)-N-[(2-trimethylsilanyl-ethoxymethyl)]-5-methyl-thiophene sulphonamide

Synthesis of 3-Borono-N-(3,4-dimethyl-3-isoxazolyl)-N-[(2-trimethylsilanyl-ethoxymethyl)]-5-methyl-thiophene sulphonamide was carried out as per STEP 04 of Example 26

### STEP 05: Synthesis of methyl 3-amino pentenoate

Synthesis of Methyl 3-amino pentenoate was carried out as per STEP 11 of Example 28

### STEP 06: Synthesis of 2,6-diethyl-4-oxo-1,4-dihydro-pyridine-3-carboxylic acid methyl ester

Synthesis of 2,6-diethyl-4-oxo-1,4-dihydro-pyridine-3-carboxylic acid methyl ester was carried out as per STEP 12 of Example 28

### STEP 07: Synthesis of 2,6-diethyl-4-(toluene-4-sulfonylamino) nicotinic acid methyl ester

Synthesis of 2,6-Diethyl-4-(toluene-4-sulfonylamino) nicotinic acid methyl ester was carried out as per STEP 13 of Example 28

### STEP 08: Synthesis of 4-amino-2,6-diethyl-nicotinic acid methyl ester

Synthesis of 4-amino-2,6-diethyl-nicotinic acid methyl ester was carried out as per STEP 14 of Example 28

### STEP 09: Synthesis of (4-Amino-2,6-diethyl-pyridin-3-yl)-methanol

Synthesis of (4-Amino-2,6-diethyl-pyridin-3-yl)-methanol was carried out as per STEP 14 of Example 28

### STEP 10: Synthesis of 4-Amino-2,6-diethyl-pyridine-3-carbaldehyde

Synthesis of 4-Amino-2,6-diethyl-pyridine-3-carbaldehyde was carried out as per STEP 15 of Example 28

### STEP 11: Synthesis of 5,7-Diethyl-1H-[1,6] naphthyridin-2-one

Synthesis of 5,7-Diethyl-1H-[1,6] naphthyridin-2-one was carried out as per STEP 16 of Example 28

### STEP 12: Synthesis of 1-(4-Bromo-3-methyl-benzyl)-5,7-diethyl-1H-[1,6] naphthyridin-2-one

At 0° C. under nitrogen was added 5,7-diethyl-1H-[1,6] naphthyridin-2-one (3.0 gm, 0.01485 mol). After the addition, the reaction mixture was warmed to ambient temperature and maintained for 30 min. Then reaction mixture was cooled to 0° C. and a solution of Methanesulfonic acid 4-bromo-3-methyl-benzyl ester (5.39 gm, 0.01931 mol) in 10 ml dimethyl formamide was added drop wise to the reaction mixture and stirred at room temperature for 8 hrs. The mixture was then diluted with ethyl acetate (40 ml), followed by 10 ml of cold water, Organic layer washed with water and brine then dried over sodium sulphate and evaporated under vacuum to afford 4.0 gm of 1-(4-Bromo-3-methyl-benzyl)-5,7-diethyl-1H-[1,6]naphthyridin-2-one as a brown colored solid.

### STEP 13: Synthesis of 3-[4-(5,7-diethyl-2-oxo-2H-[1,6]naphthyridin-1-ylmethyl)-2-methyl-phenyl]-5-methyl-thiophene-2-sulfonic acid (3,4-dimethyl-isoxazol-5-yl)-(2-trimethylsilanyl-ethoxymethyl)-amide

To a stirred solution of 1-(4-Bromo-3-methyl-benzyl)-5,7-diethyl-1H-[1,6]naphthyridin-2-one (1.0 gm, 0.0026 mol) in dimethoxy ethane (20 ml) under nitrogen was added Bis(triphenylphosphine)palladium(II)chloride (0.183 gm, 0.00026 mol) followed by addition of 2M aqueous sodium carbonate (0.827 gm in 3.9 ml water) Reaction mixture was stirred at room temperature for 10 min and then heated at 60° C. To this drop wise added the solution of 3-Borono-N-(3,4-dimethyl-3-isoxazolyl)-N-[(2-trimethylsilanyl-ethoxymethyl)]-5-methyl-thiophene sulphonamide (0.58 gm, 0.0013 mol in 10 ml dimethoxy ethane) within 45 min and reaction was refluxed for 60 min. After 1 hr the same was repeated with further addition of 3-Borono-N-(3,4-dimethyl-3-isoxazolyl)-N-[(2-trimethylsilanyl-ethoxymethyl)]-5-methyl-thiophene sulphonamide (0.58 gm, 0.0013 mol in 10 ml dimethoxy ethane) within 45 min, reaction mixture was refluxed for 4 hrs and stirred at room temperature for 12 hrs. Reaction mixture was diluted with ethyl acetate (50 ml) and water, layers were separated, aqueous layer further extracted with ethyl acetate. Combine extracts was washed with water and brine. Dried over sodium sulphate and concentrated under vacuum to yield 1.9 gm of 3-[4-(5,7-Diethyl-2-oxo-2H-[1,6]naphthyridin-1-ylmethyl)-2-methyl-phenyl]-5-methyl-thiophene-2-sulfonic acid (3,4-dimethyl-isoxazol-5-yl)-(2-trimethylsilanyl-ethoxymethyl)-amide as pale yellow oily mass.

### STEP 14: Synthesis of 3-[4-(5,7-diethyl-2-oxo-2H-[1,6] naphthyridin-1-ylmethyl)-2-methyl-phenyl]-5-methyl-thiophene-2-sulfonic acid (3,4-dimethyl-isoxazol-5-yl)-amide

(1.9 gm, 0.00269 mol) of 3-[4-(5,7-diethyl-2-oxo-2H-[1,6]naphthyridin-1-ylmethyl)-2-methyl-phenyl]-5-methyl-thiophene-2-sulfonic acid (3,4-dimethyl-isoxazol-5-yl)-(2-trimethylsilanyl-ethoxymethyl)-amide dissolved in 10 ml tetrahydrofuran followed by addition of tetrabutylammonium fluoride solution (8.1 ml, 1 molar solution in tetrahydrofuran) in it. The reaction mixture was heated at 55° C. for 3 hrs. After that the reaction mixture was cooled to room temperature and to it dilute hydrochloric acid was added and extracted with ethyl acetate (50 ml×2), ethyl acetate layer was washed with water and brine, dried over sodium sulphate and evaporated under vacuum to give 1.0 gm brown colored oily mass. The Crude compound was purified by column chromatography on a silica gel column to yield 122 mg of 3-[4-(5,7-diethyl-2-oxo-2H-[1,6] naphthyridin-1-ylmethyl)-2-methyl-phenyl]-5-methyl-thiophene-2-sulfonic acid (3,4-dimethyl-isoxazol-5-yl)-amide.

Molecular Formula: C₃₀H₃₂N₄O₄S₂

Molecular Weight: 576

¹HNMR (DMSOd₆): 1.20 (t, J=7.2 Hz, 3H), 1.25 (t, J=7.2 Hz, 3H), 1.42 (s, 3H), 1.93 (s, 3H), 1.98 (s, 3H), 2.45 (s, 3H), 2.70-2.74 (q, J=7.6 Hz, 2H), 3.04-3.10 (q, J=7.6 Hz, 2H), 5.46 (s, 2H), 6.70-7.35 (m, 6H), 8.22-8.25 (dJ=8.8 Hz, 1H), 11.07 (br, 1H).

Mass Spectrum: (m⁻¹) 575.2

### Example 31

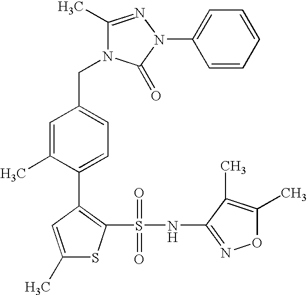

### 5-Methyl-3-[2-methyl-4-(3-methyl-5-oxo-1-phenyl-1,5-dihydro-[1,2,4] triazol-4-ylmethyl)-phenyl]-thiophene-2-sulfonic acid (4,5-dimethyl-isoxazol-3-yl)-amide

### Step 01: Synthesis of sodium salt of 3-Hydroxy-2-methyl-but-2-enenitrile.(sodium salt of 3-cyano-2-butanone)

Synthesis of sodium salt of 3-Hydroxy-2-methyl-but-2-enenitrile (sodium salt of 3-cyano-2-butanone) was carried out as per STEP 01 of Example 28

### Step 02: Synthesis of 2,2-(2-Methyl-[1,3] dioxolan-2-yl)-Propionitrile

Synthesis of 2,2-(2-Methyl-[1,3] dioxolan-2-yl)-Propionitrile was carried out as per STEP 02 of Example 28

### Step 03: Synthesis of N-Hydroxy-2-(2-methyl-[1,3]dioxolan-2-yl)-propionamidine OR 2-(β-ethylene dioxy aceto) propionamideoxime

Synthesis of N-Hydroxy-2-(2-methyl-[1,3] dioxolan-2-yl)-propionamidine OR 2-(β-ethylene dioxy aceto) propionamideoxime was carried out as per STEP 03 of Example 28

### Step 04: Synthesis of 3-Amino-4,5 dimethyl isoxazole

Synthesis of 3-Amino-4,5 dimethyl isoxazole was carried out as per STEP 04 of Example 28

### Step 05: Synthesis of 5-methyl thiophene-2-sulphonyl chloride

Synthesis of 5-methyl thiophene-2-sulphonyl chloride was carried out as per STEP 07 of Example 01

### Step 06: Synthesis of 5-methyl-thiophene-2-sulfonic acid (4,5-dimethyl-isoxazol-3yl) amide

Synthesis of 5-methyl-thiophene-2-sulfonic acid (4,5-dimethyl-isoxazol-3yl) amide was carried out as per STEP 08 of Example 01

### Step 07: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 08: Synthesis of 3-borono-N-(4,5-dimethyl-3-isoxazolyl)-N-[(2-methoxy-ethoxy) methyl]-5-methyl-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### Step 09: Synthesis of 5-methyl-3-[2-methyl-4-(3-methyl-5-oxo-1-phenyl-1,5-dihydro-[1,2,4]triazol-4-ylmethyl)-phenyl]-thiophene-2-sulfonic acid (4,5-dimethyl-isoxazol-3-yl)-(2-methoxy-ethoxymethyl)-amide

To a stirred solution of 4-(4-Bromo-3-methyl-benzyl)-5-methyl-2-phenyl-2,4-dihydro-[1,2,4]triazol-3-one (2 gm, 0.00558 mol) in dimethoxy ethane (10 ml) under nitrogen was added Bis(triphenylphosphine)palladium(II)chloride (0.39 gm, 0.000558 mol) followed by addition of 2M aqueous sodium carbonate (1.77 gm in 8.3 ml water) Reaction mixture was stirred at room temperature for 10 min and then heated at 60° C. To this drop wise added the solution of 3-Borono-N-(4,5-Dimethyl-3-isoxazolyl)-N-[(2-methoxy-ethoxy)methyl]-5-methyl-thiophene sulphonamide (1.125 gm, 0.00558 mol. in 5 ml dimethoxy ethane) within 45 min and reaction was refluxed for 60 min. After 1 hr the same was repeated with further addition of 3-Borono-N-(4,5-Dimethyl-3-isoxazolyl)-N-[(2-methoxy-ethoxy)methyl]-5-methyl-thiophene sulphonamide (1.125 gm, 0.00558 mol. in 5 ml dimethoxy ethane) within 45 min, reaction mixture was refluxed for 4 hrs and stirred at room temperature for 12 hrs. Reaction mixture was diluted with ethyl acetate (50 ml) and water, layers were separated, aqueous layer further extracted with ethyl acetate. Combine extracts was washed with water and brine. Dried over sodium sulphate and concentrated under vacuum. Crude compound was purified by column chromatography on a silica gel to yield 3.5 gm of 5-Methyl-3-[2-methyl-4-(3-methyl-5-oxo-1-phenyl-1,5-dihydro-[1,2,4]triazol-4-ylmethyl)-phenyl]-thiophene-2-sulfonic acid (4,5-dimethyl-isoxazol-3-yl)-(2-methoxy-ethoxymethyl)-amide

### Step 10: Synthesis of 5-Methyl-3-[2-methyl-4-(3-methyl-5-oxo-1-phenyl-1,5-dihydro-[1,2,4]triazol-4-ylmethyl)-phenyl]-thiophene-2-sulfonic acid (4,5-dimethyl-isoxazol-3-yl)-amide

To, 5-Methyl-3-[2-methyl-4-(3-methyl-5-oxo-1-phenyl-1,5-dihydro-[1,2,4]triazol-4-ylmethyl)-phenyl]-thiophene-2-sulfonic acid (4,5-dimethyl-isoxazol-3-yl)-(2-methoxy-ethoxymethyl)-amide (2 gm) was added ethanol (10 ml) and 6N aqueous hydrochloric acid (8 ml) at room temperature. Reaction mixture was refluxed for 3 hrs. The reaction mixture was concentrated under vacuum and pH of the solution was adjusted to 8 using a saturated solution of sodium bicarbonate and the mixture was extracted with ethyl acetate (25 ml×2). The combined organic extract were washed with water and brine then dried over sodium sulphate and concentrated under vacuum. The residue was purified by column Chromatography over Silica Gel column using hexane:ethyl acetate to afford 400 mg of 5-Methyl-3-[2-methyl-4-(3-methyl-5-oxo-1-phenyl-1,5-dihydro-[1, 2, 4] triazol-4-ylmethyl)-phenyl]-thiophene-2-sulfonic acid (4,5-dimethyl-isoxazol-3-yl)-amide as a white solid.

### Step 11: Synthesis of Acetimidic Acid Ethyl Ester Hydrochloride

To a 0° C. cooled solution of acetonitrile (50 gm, 1.21 mol) and ethanol (55.66 gm, 1.21 mol) dry HCl gas was passed (44.5 gm) for 2 hr. after completion of this reaction mixture was stirred at 0° C. for 6 hrs and then kept at room temperature for 6 hrs. The reaction mixture was evaporated under vacuum to give 30 gm of white crystalline solid of Acetimidic acid ethyl ester Hydrochloride. This was further used as such.

### Step 12: Synthesis of [1-Ethoxy-ethylidene]-carbamic acid ethyl ester

To a 0° C. cooled solution of Acetimidic acid ethyl ester Hydrochloride in dichloro methane (30 gm, 0.248 mol) Under the flow of nitrogen, Diisopropyl ethyl amine (80 gm, 0.642 mol) was added and the reaction mixture was stirred at 0° C. for 30 min. then after ethyl Chloro formate (26.3 gm, 0.2492 mol) was added drop wise in to the reaction mixture within 45 minutes. Then after the reaction mixture was stirred at room temperature for 3 hrs. The reaction mixture was filtered under vacuum and filtrate was concentrated under vacuum to give 50 gm of [1-Ethoxy-ethylidene]-carbamic acid ethyl ester as oil.

### Step 13: Synthesis of 5-Methyl-2-phenyl-2,4-dihydro-[1,2,4]triazol-3-one

To (20 gm, 0.125 mol) of [1-Ethoxy-ethylidene]-carbamic acid ethyl ester was added (100 ml) toluene to this reaction mixture (12.4 gm, 0.114 mol) of phenyl hydrazine was added and refluxed the reaction mixture for 2 hr at 450° C. then the reaction mixture was cooled to room temperature and then added (12.7 gm, 0.125 mol) of triethylamine then after reaction mixture was refluxed for 6 hrs. The reaction mixture was evaporated under vacuum and crude product was recrystalised from diethyl ether to give crystalline solid which was filtered under vacuum and suction dried to give 9.5 gm of 5-Methyl-2-phenyl-2,4-dihydro-[1,2,4]triazol-3-one

### Step 14: Synthesis of 4-(4-Bromo-3-methyl-benzyl)-5-methyl-2-phenyl-2,4-dihydro-[1,2,4]triazol-3-one

To the stirred solution of 5-Methyl-2-phenyl-2,4-dihydro-[1,2,4]triazol-3-one (1.85 gm, 0.01 mol) in dimethyl formamide (10 ml) at 0° C. under nitrogen was added portion wise sodium hydride (60% in mineral oil) (617 mg, 0.0017 mol). After the addition, the reaction mixture was warmed to ambient temperature and maintained for 30 min. Then after reaction mixture was cooled to 0° C. and a solution of Methanesulfonic acid 4-bromo-3-methyl-benzyl ester (2.86 gm, 0.010 mol) in (10 ml) dimethyl formamide was added drop wise to the reaction mixture and stirred at room temperature for 24 hrs. The mixture was then diluted with ethyl acetate (40 ml), followed by 10 ml of cold water, Organic layer washed with water and brine then dried over sodium sulphate and evaporated under vacuum to afford 3.4 gm of 4-(4-Bromo-3-methyl-benzyl)-5-methyl-2-phenyl-2,4-dihydro-[1,2,4]triazol-3-one as a brown colored solid.

Molecular Formula: C₂₇H₂₇N₅O₄S₂

Molecular Weight: 549

¹HNMR (DMSOd₆): 1.55 (s, 3H), 1.96 (s, 3H), 2.18 (s, 3H), 2.27 (s, 3H), 4.90 (s, 2H), 6.74 (s, 1H), 6.95-6.97 (d, J=7.6 Hz, 1H), 7.04-7.06 (d, J=7.6 Hz, 1H), 7.17 (s, 1H), 7.23 (t, J=7.6 Hz, 1H), 7.47 (t, J=7.6 Hz, 2H), 7.92-7.94 (d, J=7.6 Hz, 2H), 10.58 (br, 1H).

Mass Spectrum: (m⁻¹) 548.1

### Example 32

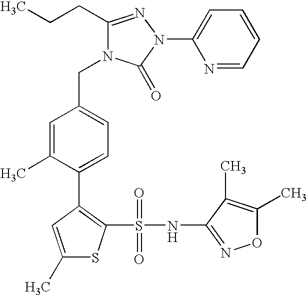

### 5-Methyl-3-[2-methyl-4-(5-oxo-3-propyl-1-pyridin-2-yl-1,5-dihydro-[1,2,4] triazol-4-ylmethyl)-phenyl]-thiophene-2-sulfonic acid (4,5-dimethyl-isoxazol-3-yl)-amide

### Step 01: Synthesis of sodium salt of 3-Hydroxy-2-methyl-but-2-enenitrile.(sodium salt of 3-cyano-2-butanone)

Synthesis of sodium salt of 3-Hydroxy-2-methyl-but-2-enenitrile (sodium salt of 3-cyano-2-butanone) was carried out as per STEP 01 of Example 28

### Step 02: Synthesis of 2,2-(2-Methyl-[1,3] dioxolan-2-yl)-Propionitrile

Synthesis of 2,2-(2-Methyl-[1,3] dioxolan-2-yl)-Propionitrile was carried out as per STEP 02 of Example 28

### Step 03: Synthesis of N-Hydroxy-2-(2-methyl-[1,3]dioxolan-2-yl)-propionamidine OR 2-(β-ethylene dioxy aceto) propionamideoxime

Synthesis of N-Hydroxy-2-(2-methyl-[1,3] dioxolan-2-yl)-propionamidine OR 2-(β-ethylene dioxy aceto) propionamideoxime was carried out as per STEP 03 of Example 28

### Step 04: Synthesis of 3-Amino-4,5 dimethyl isoxazole

Synthesis of 3-Amino-4,5 dimethyl isoxazole was carried out as per STEP 04 of Example 28

### Step 05: Synthesis of 5-methyl thiophene-2-sulphonyl chloride

Synthesis of 5-methyl thiophene-2-sulphonyl chloride was carried out as per STEP 07 of Example 01

### Step 06: Synthesis of 5-methyl-thiophene-2-sulfonic acid (4,5-dimethyl-isoxazol-3yl) amide

Synthesis of 5-methyl-thiophene-2-sulfonic acid (4,5-dimethyl-isoxazol-3yl) amide was carried out as per STEP 08 of Example 01

### Step 07: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 08: Synthesis of 3-borono-N-(4,5-dimethyl-3-isoxazolyl)-N-[(2-methoxy-ethoxy)methyl]-5-methyl-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### Step 09: Synthesis of 5-methyl-3-[2-methyl-4-(3-methyl-5-oxo-1-phenyl-1,5-dihydro-[1,2,4]triazol-4-ylmethyl)-phenyl]-thiophene-2-sulfonic acid (4,5-dimethyl-isoxazol-3-yl)-(2-methoxy-ethoxymethyl)-amide

To a stirred solution of 4-(4-Bromo-3-methyl-benzyl)-5-propyl-2-pyridin-2-yl-2,4-dihydro-[1,2,4]triazol-3-one (0.5 gm, 0.001 mol) in dimethoxy ethane (5 ml) under nitrogen was added Bis(triphenylphosphine)palladium(II)chloride (0.09 gm, 0.00012 mol) followed by addition of 2M aqueous sodium carbonate (0.4 gm in 2.16 ml water) Reaction mixture was stirred at room temperature for 10 min and then heated at 60° C. To this drop wise added the solution of 3-Borono-N-(4,5-Dimethyl-3-isoxazolyl)-N-[(2-methoxy-ethoxy)methyl]-5-methyl-thiophene sulphonamide (0.26 gm, 0.0001 mol in ml dimethoxy ethane) within 45 min and reaction was refluxed for 60 min. After 1 hr the same was repeated with further addition of 3-Borono-N-(4,5-Dimethyl-3-isoxazolyl)-N-[(2-methoxy-ethoxy)methyl]-5-methyl-thiophene sulphonamide (0.26 gm, 0.0001 mol in ml dimethoxy ethane) within 45 min, reaction mixture was refluxed for 4 hrs and stirred at room temperature for 12 hrs. Reaction mixture was diluted with ethyl acetate (20 ml) and water, layers were separated, aqueous layer further extracted with ethyl acetate. Combine extracts was washed with water and brine. Dried over sodium sulphate and concentrated under vacuum. Crude compound was purified by column chromatography on a silica gel column to yield 0.78 gm of 5-Methyl-3-[2-methyl-4-(3-methyl-5-oxo-1-phenyl-1,5-dihydro-[1,2,4]triazol-4-ylmethyl)-phenyl]-thiophene-2-sulfonic acid (4,5-dimethyl-isoxazol-3-yl)-(2-methoxy-ethoxymethyl)-amide as brown oil.

### Step 10: Synthesis of 5-Methyl-3-[2-methyl-4-(5-oxo-3-propyl-1-pyridin-2-yl-1,5-dihydro-[1,2,4]triazol-4-ylmethyl)-phenyl]-thiophene-2-sulfonic acid (4,5-dimethyl-isoxazol-3-yl)-amide

To, 5-Methyl-3-[2-methyl-4-(3-methyl-5-oxo-1-phenyl-1,5-dihydro-[1,2,4]triazol-4-ylmethyl)-phenyl]-thiophene-2-sulfonic acid (4,5-dimethyl-isoxazol-3-yl)-(2-methoxy-ethoxymethyl)-amide (0.78 gm) was added ethanol (10 ml) and 6N aqueous hydrochloric acid (8 ml) at room temperature. Reaction mixture was refluxed for 3 hrs. The reaction mixture was concentrated under vacuum and pH of the solution was adjusted to 8 using a saturated solution of sodium bicarbonate and the mixture was extracted with ethyl acetate (25 ml×2). The combined organic extract were washed with water and brine then dried over sodium sulphate and concentrated under vacuum. The residue was purified by column Chromatography over Silica Gel column using hexane:ethyl acetate to afford 40 mg of 5-Methyl-3-[2-methyl-4-(5-oxo-3-propyl-1-pyridin-2-yl-1,5-dihydro-[1,2,4]triazol-4-ylmethyl)-phenyl]-thiophene-2-sulfonic acid (4,5-dimethyl-isoxazol-3-yl)-amide as a white solid.

### Step 1: Synthesis of Butyrimidic Acid Ethyl Ester Hydrochloride

To a 0° C. cooled solution of Butyronitrile (50 gm, 0.726 mol) and ethanol (33.33 gm, 0.724 mol) dry HCl gas was passed (till weight of the reaction mixture increased by 26 gm) (26.44 gm, 0.724 mol), after completion of this reaction mixture was stirred at 0° C. for 6 hrs and then kept at room temperature for 6 hrs. The reaction mixture was evaporated under vacuum to give 35 gm of white crystalline solid of Butyrimidic acid ethyl ester Hydrochloride.

This was further used as such.

### Step 12: Synthesis of [1-Ethoxy-butylidene]-carbamic acid ethyl ester

To a 0° C. cooled solution of Butyrimidic acid ethyl ester Hydrochloride (25 gm, 0.18 mol) in dichloro methane (125 ml) Under the flow of nitrogen, Diisopropyl ethyl amine (55 gm, 0.58 mol) was added and the reaction mixture was stirred at 0° C. for 30 min then after ethyl Chloro formate (17 gm, 0.156 mol) was added drop wise in to the reaction mixture within 45 minutes. Then after the reaction mixture was stirred at room temperature for 3 hrs. The reaction mixture was filtered under vacuum and filtrate was concentrated under vacuum to give 24 gm of [1-Ethoxy-butylidene]-carbamic acid ethyl ester as oil.

### Step 13: Synthesis of 5-Propyl-2-pyridin-2-yl-2,4-dihydro-[1,2,4]triazol-3-one

To (10 gm, 0.5 mol) of [1-Ethoxy-but-ylidene]-carbamic acid ethyl ester was added (20 ml) toluene to this reaction mixture (5.3 gm, 0.048 mol) of Pyridin-2-yl-hydrazine was added and heated the reaction mixture at 45° C. for 30 min, then the reaction mixture was cooled to room temperature and then added (5.4 gm, 0.053 mol) of triethylamine then after reaction mixture was refluxed for 6 hrs. The reaction mixture was evaporated under vacuum and crude product was recrystalised from diethyl ether to give crystalline solid which was filtered under vacuum and suction dried to give 2.2 gm of 5-Propyl-2-pyridin-2-yl-2,4-dihydro-[1,2,4]triazol-3-one.

### Step 14: Synthesis of 4-(4-Bromo-3-methyl-benzyl)-5-methyl-2-phenyl-2,4-dihydro-[1,2,4]triazol-3-one

To the stirred solution of 5-Propyl-2-pyridin-2-yl-2,4-dihydro-[1,2,4]triazol-3-one (1 gm, 0.004 mol) in dimethyl formamide (10 ml) at 0° C. under nitrogen was added portion wise sodium hydride (60% in mineral oil) (294 mg, 0.0073 mol). After the addition, the reaction mixture was warmed to ambient temperature and maintained for 30 min. Then after reaction mixture was cooled to 0° C. and a solution of Methanesulfonic acid 4-bromo-3-methyl-benzyl ester (1.64 gm, 0.0058 mol) in (10 ml) dimethyl formamide was added drop wise to the reaction mixture and stirred at room temperature for 24 hrs. The mixture was then diluted with ethyl acetate (20 ml), followed by (10 ml) of cold water, Organic layer washed with water and brine then dried over sodium sulphate and evaporated under vacuum to afford 2 gm of 4-(4-Bromo-3-methyl-benzyl)-5-methyl-2-phenyl-2,4-dihydro-[1,2,4]triazol-3-one as a brown colored solid.

Molecular Formula: C₂₈H₃₀N₆O₄S₂

Molecular Weight: 578

¹HNMR (DMSOd₆): 0.94 (t, J=7.6 Hz, 3H), 1.29 (s, 3H), 1.64-1.69 (q, 2H), 1.96 (s, 3H), 2.18 (s, 3H), 2.55 (t, J=7.6 Hz, 3H), 4.91 (s, 2H), 6.74 (s, 1H), 6.95-7.16 (m, 4H), 7.92-7.99 (m, 2H), 8.49-8.50 (d, 2H), 10.62 (s, 1H).

Mass Spectrum: (m⁻¹) 577.2

### Example 33

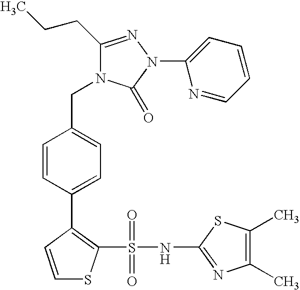

### 3-[4-(5-Oxo-3-propyl-1-pyridin-2-yl-1,5-dihydro-[1, 2, 4] triazol-4-ylmethyl)-phenyl]-thiophene-2-sulfonic acid (4,5-dimethyl-thiazol-2-yl)-amide

### STEP 01: Synthesis of 3-Bromo-thiophene-2-sulfonyl chloride

Synthesis of 3-Bromo-thiophene-2-sulfonyl chloride was carried out as per STEP 10 of Example 02

### STEP 02: Synthesis of 3-Bromo-thiophene-2-sulfonic acid (4,5-dimethyl-thiazol-2-yl)-amide

3-Bromo-thiophene-2-sulfonyl chloride (20 gm, 0.076 mol) was added to a solution of 4,5-dimethyl-thiazol-2-ylamine (15 gm, 0.071 mol) in 40 ml sodium hydroxide solution (6 gm, 0.15 mol), at room temperature, stirred for 6 hours. Concentrated the reaction mixture completely under vacuum, acidified the reaction mixture using 1N Hydrochloric acid, followed by extraction with dichloro methane. (50 ml×3) Combined extract was washed with water and brine. Dried over sodium sulphate and concentrated to give gm of mixture of products, Which was dissolved in methanolic sodium hydroxide solution and this reaction mixture was stirred and heated at 50° C. for 6 hrs. The reaction mixture was cooled to room temperature and evaporated under vacuum. The residue thus obtained was acidified with dilute hydrochloric acid to pH 2 followed by extraction with methylene chloride (10 ml×3). Combined extract was washed with water, brine and dried over anhydrous sodium sulphate, evaporated under vacuum to give 9 gm brown color solid of 3-Bromo-thiophene-2-sulfonic acid (4,5-dimethyl-thiazol-2-yl)-amide.

### STEP 03: Synthesis of 3-Bromo-thiophene-2-sulfonic acid (4,5-dimethyl-thiazol-2-yl)-(2-methoxy-ethoxymethyl)-amide

To the solution of 3-Bromo-thiophene-2-sulfonic acid (4,5-dimethyl-thiazol-2-yl)-amide (8 gm, 0.022 mol) in 40 ml acetone and (10 ml) dimethyl formamide at 0° C., potassium carbonate (5 gm, 0.036 mol) was added. The reaction mixture was stirred at room temperature for 30 min. The reaction mixture was cooled to 0° C. using ice-salt bath. 2-methoxyethoxy methyl chloride (5 gm, 0.040 mol) was added drop wise over 30 min at 0° C. The reaction was stirred with an ice-salt bath for 30 min. and then at room temperature for 4 hrs. The mixture was diluted with ethyl acetate (100 ml) followed by 30 ml of ice cold water and organic layer was separated, washed with water and brine finally dried over sodium sulphate and concentrated under vacuum. The residue was purified by column chromatography on silica gel column using hexane:ethyl acetate to afford 3 gm of 3-Bromo-thiophene-2-sulfonic acid (4,5-dimethyl-thiazol-2-yl)-(2-methoxy-ethoxymethyl)-amide as yellow oil.

### STEP 05: Synthesis of 3-(4-formyl-phenyl)-thiophene-2-sulphonic acid (4,5-dimethyl-isoxazol-3-yl)-(2-methoxy-ethoxymethyl)amide

To a stirred solution (3 gm, 6.8 mmol) of 3-Bromo-thiophene-2-sulfonic acid (4,5-dimethyl-thiazol-2-yl)-(2-methoxy-ethoxymethyl)-amide and (1.2 gm, 8 mmol) 4-formyl boronic acid in dimethoxy ethane (30 ml) under nitrogen atmosphere was added 2M aqueous sodium carbonate (2.16 gm in 10 ml water). Stirred the reaction mixture for 15 minutes then added Bis(triphenylphosphine)palladium(II)chloride (400 mg, 0.68 mmol) in to the reaction mixture. The reaction mixture was heated to 85° C. for 6 hrs. The reaction mixture was brought to room temperature and added (50 ml) ethyl acetate. Concentrated the reaction mixture under vacuum Ethyl acetate (100 ml) was added to the residue followed by chilled water and further extraction with ethyl acetate (100 ml×2). Combine extracts was washed with water and brine. Dried over sodium sulphate and concentrated completely under vacuum. Crude compound was purified by column chromatography over Silica Gel to yield 2.5 gm of 3-(4-formyl-phenyl)-thiophene-2-sulphonic acid (4,5-dimethyl-isoxazol-3-yl)-(2-methoxy-ethoxymethyl)amide as oily mass.

### STEP 06: Synthesis of 3-(4-hydroxymethyl-phenyl)-thiophene-2-sulfonic acid (4,5-dimethyl-thiazol-2-yl)-(2-methoxy-ethoxymethyl)-amide

Sodium borohydride (400 mg, 10 mmol) was added under nitrogen flow to a stirred solution of tetrahydrofuran at 0° C., followed by addition of 3-(4-formyl-phenyl)-thiophene-2-sulphonic acid (4,5-dimethyl-isoxazol-3-yl)-(2-methoxy-ethoxymethyl)amide (2.5 gm, 5.3 mmol) in (15 ml) tetrahydrofuran. Stirred the reaction mixture at 0° C. for 1 hr and raised the temperature of the reaction mixture to room temperature and stirred for 4 hrs. Work up was done by addition of sodium hydroxide solution (1 gm dissolved in 100 ml water) at 0° C. followed by extraction with ethylacetate (50 ml×2). Dried the organic layer over sodium sulphate and concentrated completely under vacuum to give 2.3 gm of 3-(4-Hydroxymethyl-phenyl)-thiophene-2-sulfonic acid (4,5-dimethyl-thiazol-2-yl)-(2-methoxy-ethoxymethyl)-amide as an oily liquid.

### STEP 07: Synthesis of methanesulfonic acid 4-{2-[(4,5-dimethyl-thiazol-2-yl)-(2-methoxy-ethoxymethyl)-sulfamoyl]-thiophen-3-yl}1-benzyl ester

N-Ethyl diisopropyl amine (2 ml, 10.8 m mol) was added to a solution of 3-(4-Hydroxymethyl-phenyl)-thiophene-2-sulfonic acid (4,5-dimethyl-thiazol-2-yl)-(2-methoxy-ethoxymethyl)-amide (2.3 gm, 4.9 mmol) in (15 ml) of dichloro methane. Cooled the reaction mixture to 0° C. then added slowly a solution of methane sulfonyl chloride (0.5 ml, 6.1 mmol) in 10 ml dichloromethane, into the reaction mixture. The reaction mixture was maintained at room temperature for 3 hrs. Workup was done by addition of ice-cold water into the reaction mixture followed by extraction with methylene dichloride (25 ml×2). Combine extracts was given washing with dilute hydrochloric acid followed by water and brine solution. Dried the organic layer over sodium sulphate and concentrated under vacuum to give 1.8 gm of Methanesulfonic acid 4-{2-[(4,5-dimethyl-thiazol-2-yl)-(2-methoxy-ethoxymethyl)-sulfamoyl]-thiophen-3-yl}-benzyl ester as viscous liquid.

### STEP 08: Synthesis of 3-[4-(5-Oxo-3-propyl-1-pyridin-2-yl-1,5-dihydro-[1,2,4]triazol-4-ylmethyl)-phenyl]-thiophene-2-sulfonic acid (4,5-dimethyl-thiazol-2-yl)-(2-methoxy-ethoxymethyl)-amide

To the stirred solution of 5-Propyl-2-pyridin-2-yl-2,4-dihydro-[1,2,4]triazol-3-one (700 mg, 3.4 mmol) in dimethyl formamide (5 ml) at 0° C. under the flow of dry nitrogen gas was added portion wise sodium hydride (60% in mineral oil) (252 mg, 5.2 mmol). After the addition, temperature was raised to room temperature and maintained for 30 min. Re-cooled to 0° C. and a solution of methanesulfonic acid 4-{2-[(4,5-dimethyl-thiazol-2-yl)-(2-methoxy-ethoxymethyl)-sulfamoyl]-thiophen-3-yl}-benzyl ester (1.8 gm, 3.5 mmol) in 5 ml dimethyl formamide was drop wise added to the reaction mixture and stirred at room temperature for 24 hrs. The mixture was then diluted with ethyl acetate (40 ml) followed by 10 ml of water at 0° C. and extracted with ethyl acetate (50 ml×2) Combine extracts was given washing with water and brine. Dried the organic layer over sodium sulphate and concentrated under vacuum to give 2 gm of 3-[4-(5-Oxo-3-propyl-1-pyridin-2-yl-1,5-dihydro-[1,2,4]triazol-4-ylmethyl)-phenyl]-thiophene-2-sulfonic acid (4,5-dimethyl-thiazol-2-yl)-(2-methoxy-ethoxymethyl)-amide as a gum.

### STEP 09: Synthesis of 3-[4-(5-Oxo-3-propyl-1-pyridin-2-yl-1,5-dihydro-[1,2,4]triazol-4-ylmethyl)-phenyl]-thiophene-2-sulfonic acid (4,5-dimethyl-thiazol-2-yl)-amide

To 2 gm of 3-[4-(5-Oxo-3-propyl-1-pyridin-2-yl-1,5-dihydro-[1,2,4]triazol-4-ylmethyl)-phenyl]-thiophene-2-sulfonic acid (4,5-dimethyl-thiazol-2-yl)-(2-methoxy-ethoxymethyl)-amide was added 95% ethanol (15 ml) and 10 ml of 6N aqueous hydrochloric acid at room temperature. Reaction mixture was refluxed for 6 hrs. The reaction mixture was concentrated under vacuum and pH of the solution was adjusted to pH 5 using sodium bicarbonate solution and the mixture was extracted with ethyl acetate (50 ml×2). The combined organic extract were washed with water and brine then dried over sodium sulphate and concentrated under vacuum. The residue was purified over Silica Gel Flash chromatography using hexane:ethyl acetate to afford 180 mg of yellowish solid of 3-[4-(5-Oxo-3-propyl-1-pyridin-2-yl-1,5-dihydro-[1,2,4] triazol-4-ylmethyl)-phenyl]-thiophene-2-sulfonic acid (4,5-dimethyl-thiazol-2-yl)-amide.

Molecular Formula: C₂₆H₂₆N₆O₃S₃

Molecular Weight: 566

¹HNMR (DMSOd₆): 0.93 (3, 3H), 1.64 (m, 2H), 2.07 (s, 3H), 2.12 (s, 3H), 2.55 (t, J=3.2 Hz, 2H), 4.96 (s, 2H), 7.20-8.50 (m, 10H), 12.46 (br, 1H).

Mass Spectrum: (m⁻¹) 565.12

### Example 34

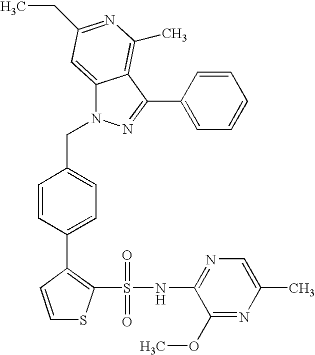

### 3-[4-(6-Ethyl-4-methyl-3-phenyl-pyrazolo[4,3-c]pyridin-1-ylmethyl)-phenyl]-thiophene-2-sulfonic acid isobutoxycarbamoyl-(3-methoxy-5-methyl-pyrazin-2-yl)-amide

### STEP 01: Synthesis of 3-Bromo-thiophene-2-sulfonyl chloride

Synthesis of 3-Bromo-thiophene-2-sulfonyl chloride was carried out as per STEP 10 of Example 02

### Step 02: Synthesis of 3-Bromo-thiophene-2-sulfonic acid (3-methoxy-5-methyl-pyrazin-2-yl)-amide

To a stirred cold solution of 3-methoxy-5-methyl-pyrazine-2-ylamine (2.5 gm, 0.018 mol) in pyridine (30 ml) di-methyl amino pyridine (0.3 gm, 0.002 mol) was charged followed by 3-bromo-thiophene-2-sulfonyl chloride (6.5 gm, 0.025 mol). Reaction mixture was heated and stirred at 60° C. for 24 hrs. Pyridine was evaporated under vacuum. Crude was taken into ethyl acetate and washed with saturated sodium bicarbonate solution. Ethyl acetate layer was dried over sodium sulphate and concentrated under vacuum to give 4.4 gm of 3-Bromo-thiophene-2-sulfonic acid (3-methoxy-5-methyl-pyrazin-2-yl)-amide as a brown solid

### Step 03: Synthesis of 3-Bromo-thiophene-2-sulfonic acid isobutoxycarbamoyl-(3-methoxy-5-methyl-pyrazin-2-yl)-amide

To a stirred cold solution of 3-Bromo-thiophene-2-sulfonic acid (3-methoxy-5-methyl-pyrazin-2-yl)-amide (5.5 gm, 0.015 mol) in dimethyl formamide (30 ml), sodium hydride (60% in mineral oil, 0.870 gm, 0.018 mol) was charged portion wise at 0° C. Reaction mixture was stirred at room temperature for 30 minutes. Isobutyl Chloro formate (2.4 gm, 0.017 mol) was charged at 0° C. Reaction mixture was stirred at room temperature for 3 hrs. To this Ethyl acetate (150 ml) and water (50 ml) was charged. Organic layer was washed with water (50 ml×3). Organic layer was dried over sodium sulphate and concentrate under vacuum to give 5 gm of 3-Bromo-thiophene-2-sulfonic acid isobutoxycarbamoyl-(3-methoxy-5-methyl-pyrazin-2-yl)-amide as brownish oil.

### Step 04: Synthesis of 3-(4-Formyl-phenyl)-thiophene-2-sulfonic acid isobutoxycarbamoyl-(3-methoxy-5-methyl-pyrazin-2-yl)-amide

To 4-formyl boronic acid (1.61 gm, 0.011 mol) in 20 ml ethanol was charged 3-Bromo-thiophene-2-sulfonic acid isobutoxycarbamoyl-(3-methoxy-5-methyl-pyrazin-2-yl)-amide (5 gm, 0.011 mmol) in toluene (20 ml) followed by addition of 2M solution of sodium carbonate (3.4 gm in water 16 ml, 0.032 mol) and Stirred for 15 minutes. Tetrakis triphenyl phosphine Palladium (0) (0.53 gm, 0.00045 mol) was added and reaction mixture was heated and stirred at 110° C.-120° C. for 6 hrs. To reaction mixture ethyl acetate (50 ml) was added and reaction mixture was concentrated under vacuum. To this Water (100 ml) was added and extracted with ethyl acetate (100 ml). Organic layer was dried over sodium sulphate and concentrated under vacuum to give 6 gm of 3-(4-Formyl-phenyl)-thiophene-2-sulfonic acid isobutoxycarbamoyl-(3-methoxy-5-methyl-pyrazin-2-yl)-amide.

### Step 05: Synthesis of 3-(4-Hydroxymethyl-phenyl)-thiophene-2-sulfonic acid isobutoxycarbamoyl-(3-methoxy-5-methyl-pyrazin-2-yl)-amide

To a stirred solution of 3-(4-Formyl-phenyl)-thiophene-2-sulfonic acid isobutoxycarbamoyl-(3-methoxy-5-methyl-pyrazin-2-yl)-amide (5 gm, 0.011 mol) in Tetrahydrofuran (30 ml) at 0° C. sodium borohydride (0.6 gm, 0.017 mol) was added and allowed to Stirred at room temperature for 60 minutes. Reaction mixture was cooled to 0° C. and dilute sodium hydroxide (0.5 gm in 50 ml) solution was added in to the reaction mixture. The reaction mixture was then extracted with ethyl acetate. The organic layer was, washed with water and brine, dried over sodium sulphate and concentrated under vacuum to give 3.9 gm of 3-(4-Hydroxymethyl-phenyl)-thiophene-2-sulfonic acid isobutoxycarbamoyl-(3-methoxy-5-methyl-pyrazin-2-yl)-amide as brown oil.

### Step 06: Synthesis of methanesulfonic acid 4-{2-[isobutoxycarbamoyl-(3-methoxy-5-methyl-pyrazin-2-yl)-sulfamoyl]-thiophen-3-yl}-benzyl ester

To a stirred solution of 3-(4-Hydroxymethyl-phenyl)-thiophene-2-sulfonic acid isobutoxycarbamoyl-(3-methoxy-5-methyl-pyrazin-2-yl)-amide (1.5 gm, 3 mmol) in dichloromethane (20 ml) was added triethylamine (1.0 ml, 6 mmol) at 0° C. and stirred for 5 min, followed by Methane sulfonyl chloride (0.3 ml, 3.7 mmol) at 0° C. The reaction mixture was allowed to stir at room temperature for 3 hrs. Quenched the reaction mixture with water (15 ml). Organic layer was separated and dried over sodium sulphate, concentrated under vacuum to give 1.5 gm of Methanesulfonic acid 4-{2-[isobutoxycarbamoyl-(3-methoxy-5-methyl-pyrazin-2-yl)-sulfamoyl]-thiophen-3-yl}-benzyl ester.

### Step 07: Synthesis of 3-[4-(6-Ethyl-4-methyl-3-phenyl-pyrazolo[4,3-c]pyridin-1-ylmethyl)-phenyl]-thiophene-2-sulfonic acid isobutoxycarbamoyl-(3-methoxy-5-methyl-pyrazin-2-yl)-amide

To a stirred solution of 6-Ethyl-4-methyl-3-phenyl-1H-pyrazolo[4,3-c]pyridine (0.640 gm, 2.7 mmol) in dimethyl formamide (5 ml), at 0° C. under nitrogen was charged sodium hydride (50%, 0.2 gm, 4 mmol) and allowed to Stirred for 30 minutes at room temperature. Then the reaction mixture was cooled to 0° C. and Methanesulfonic acid 4-{2-[isobutoxycarbamoyl-(3-methoxy-5-methyl-pyrazin-2-yl)-sulfamoyl]-thiophen-3-yl}-benzyl ester (1.5 gm, 2.7 mmol) in Dimethyl formamide (5 ml) was added. The reaction mixture was stirred at room temperature for 6 hrs. The reaction mixtures was diluted with ethyl acetate (20 ml) and (10 ml) water. Organic layer was separated and washed with water (10 ml×3), brine dried over sodium sulphate and concentrated under vacuum to give 1 gm of crude 3-[4-(6-Ethyl-4-methyl-3-phenyl-pyrazolo[4,3-c]pyridin-1-ylmethyl)-phenyl]-thiophene-2-sulfonic acid isobutoxycarbamoyl-(3-methoxy-5-methyl-pyrazin-2-yl)-amide as viscous liquid.

### Step 08: Synthesis of 3-[4-(6-Ethyl-4-methyl-3-phenyl-pyrazolo[4,3-c]pyridin-1-ylmethyl)-phenyl]-thiophene-2-sulfonic acid-(3-methoxy-5-methyl-pyrazin-2-yl)-amide

To, 3-[4-(6-Ethyl-4-methyl-3-phenyl-pyrazolo[4,3-c]pyridin-1-ylmethyl)-phenyl]-thiophene-2-sulfonic acid isobutoxycarbamoyl-(3-methoxy-5-methyl-pyrazin-2-yl)-amide (1 gm) methanol (10 ml) and sodium hydroxide (500 mg in 5 ml water) was added. Reaction mixture was heated and stirred at 50° C. for 1 hrs. methanol was evaporated and neutralize with dilute hydrochloric acid (pH˜6.5). Extracted with dichloromethane (50 ml), dried over sodium sulphate and concentrate under vacuum. Crude compound was purified over silica gel column chromatography using ethyl acetate and hexane to give 50 mg of 3-[4-(6-Ethyl-4-methyl-3-phenyl-pyrazolo[4,3-c]pyridin-1-ylmethyl)-phenyl]-thiophene-2-sulfonic acid isobutoxycarbamoyl-(3-methoxy-5-methyl-pyrazin-2-yl)-amide.

Molecular Formula: C₃₂H₃₀N₆O₃S₂

Molecular Weight: 610

¹HNMR (DMSOd₆): 1.24-1.31 (t, J=7.6 Hz 3H), 2.24 (s, 3H), 2.47 (s, 3H), 2.80-2.85 (q, J 7.2 Hz, 2H), 3.72 (s, 3H), 5.71 (s, 2H), 7.08-7.09 (d, J=5.2 Hz 1H) 7.26-7.28 (d, J=8 Hz, 2H) 7.44-7.46 (d, J=8.4 Hz, 2H), 7.48-7.55 (m, 5H), 7.65-7.68 (dd, J=8 Hz, 2H) 7.86-7.87 (d, J=5.2, 1H)

Mass Spectrum: (m⁺¹) 611.27

### Example 35

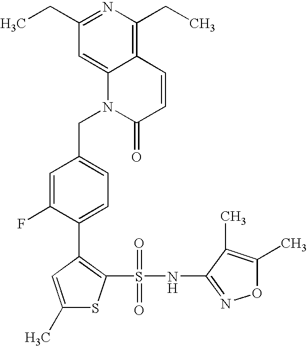

### 3-[4-(5,7-Diethyl-2-oxo-2H-[1,6]naphthyridin-1-ylmethyl)-2-fluoro-phenyl]-5-methyl-thiophene-2-sulfonic acid (4,5-dimethyl-isoxazol-3-yl)-amide

### Step 01: Synthesis of sodium salt of 3-Hydroxy-2-meth 1-but-2-enenitrile.(sodium salt of 3-cyano-2-butanone)

Synthesis of sodium salt of 3-Hydroxy-2-methyl-but-2-enenitrile (sodium salt of 3-cyano-2-butanone) was carried out as per STEP 01 of Example 28

### Step 02: Synthesis of 2,2-(2-Methyl-[1,3]dioxolan-2-yl)-Propionitrile

Synthesis of 2,2-(2-Methyl-[1,3] dioxolan-2-yl)-Propionitrile was carried out as per STEP 02 of Example 28

### Step 03: Synthesis of N-Hydroxy-2-(2-methyl-[1,3]dioxolan-2-yl)-propionamidine OR 2-(β-ethylene dioxy aceto) propionamideoxime

Synthesis of N-Hydroxy-2-(2-methyl-[1,3] dioxolan-2-yl)-propionamidine OR 2-(β-ethylene dioxy aceto) propionamideoxime was carried out as per STEP 03 of Example 28

### Step 04: Synthesis of 3-Amino-4,5 dimethyl isoxazole

Synthesis of 3-Amino-4,5 dimethyl isoxazole was carried out as per STEP 04 of Example 28

### Step 05: Synthesis of 5-methyl thiophene-2-sulphonyl chloride

Synthesis of 5-methyl thiophene-2-sulphonyl chloride was carried out as per STEP 07 of Example 01

### Step 06: Synthesis of 5-methyl-thiophene-2-sulfonic acid (4,5-dimethyl-isoxazol-3yl) amide

Synthesis of 5-methyl-thiophene-2-sulfonic acid (4,5-dimethyl-isoxazol-3yl) amide was carried out as per STEP 08 of Example 01

### Step 07: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 08: Synthesis of 3-borono-N-(4,5-dimethyl-3-isoxazolyl)-N-[(2-methoxy-ethoxy)methyl]-5-methyl-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### STEP 09: Synthesis of 3-[4-(5,7-Diethyl-2-oxo-2H-[1,6]naphthyridin-1-ylmethyl)-2-fluoro-phenyl]-5-methyl-thiophene-2-sulfonic acid (4,5-dimethyl-isoxazol-3-yl)-(2-methoxy-ethoxymethyl)-amide

To a stirred solution of 1-(4-Bromo-3-fluoro-benzyl)-5,7-diethyl-1H-[1,6]naphthyridin-2-one (2 gm, 5.1 mmol) in dimethoxy ethane (20 ml) under nitrogen was added bis(triphenylphosphine)-palladium(II)chloride (370 mg, 0.51 mmol) followed by addition of 2M aqueous sodium carbonate (1.7 gm in 8 ml water) Reaction mixture was stirred at room temperature for 10 min and then heated at 60° C. To this drop wise added the solution of 3-Borono-N-(4,5-Dimethyl-3-isoxazolyl)-N-[(2-methoxy-ethoxy)methyl]-5-methyl-thiophene sulphonamide (1 gm, 2.5 mmol in 15 ml dimethoxy ethane) within 45 min and reaction was refluxed for 60 min. After 1 hr the same was repeated with further addition of 3-Borono-N-(4,5-Dimethyl-3-isoxazolyl)-N-[(2-methoxy-ethoxy)methyl]-5-methyl-thiophene sulphonamide (1 gm, 2.5 mmol in 15 ml dimethoxy ethane) within 45 min, reaction mixture was refluxed for 4 hrs and stirred at room temperature for 12 hrs. Reaction mixture was diluted with ethyl acetate 100 ml and water, layers were separated, aqueous layer further extracted with ethyl acetate. Combine extracts was washed with water and brine. Dried over sodium sulphate and concentrated under vacuum. Crude compound was purified by column chromatography on a silica gel to yield 2 gm of 3-[4-(5,7-diethyl-2-oxo-2H-[1,6]naphthyridin-1-ylmethyl)-2-fluoro-phenyl]-5-methyl-thiophene-2-sulfonic acid (4,5-dimethyl-isoxazol-3-yl)-(2-methoxy-ethoxymethyl)-amide as pale yellow oily mass.

### STEP 010: Synthesis of 3-[4-(5,7-Diethyl-2-oxo-2H-[1,6] naphthyridin-1-ylmethyl)-2-fluoro-phenyl]-5-methyl-thiophene-2-sulfonic acid (4,5-dimethyl-isoxazol-3-yl)-amide

To, 3-[4-(5,7-Diethyl-2-oxo-2H-[1,6]naphthyridin-1-ylmethyl)-2-fluoro-phenyl]-5-methyl-thiophene-2-sulfonic acid (4,5-dimethyl-isoxazol-3-yl)-(2-methoxy-ethoxymethyl)-amide (2 gm) was added ethanol (15 ml) and 6N aqueous hydrochloric acid (10 ml) at room temperature. Reaction mixture was refluxed for 3 hrs. The reaction mixture was concentrated under vacuum and pH of the solution was adjusted to 8 using a saturated solution of sodium bicarbonate and the mixture was extracted with ethyl acetate (25 ml×2). The combined organic extract were washed with water and brine then dried over sodium sulphate and concentrated under vacuum. The residue was purified by column Chromatography over Silica Gel column using hexane:ethyl acetate to afford 200 mg of 3-[4-(5,7-Diethyl-2-oxo-2H-[1,6]naphthyridin-1-ylmethyl)-2-fluoro-phenyl]-5-methyl-thiophene-2-sulfonic acid (4,5-dimethyl-isoxazol-3-yl)-amide as a yellow solid.

### STEP 11: Synthesis of methyl 3-amino pentenoate

Synthesis of Methyl 3-amino pentenoate was carried out as per STEP 11 of Example 28

### STEP 12: Synthesis of 2,6-Diethyl-4-oxo-1,4-dihydro-pyridine-3-carboxylic acid methyl ester

Synthesis of 2,6-diethyl-4-oxo-1,4-dihydro-pyridine-3-carboxylic acid methyl ester was carried out as per STEP 12 of Example 28

### STEP 13: Synthesis of 2,6-diethyl 4-(toluene-4-sulfonylamino) nicotinic acid methyl ester

Synthesis of 2,6-Diethyl-4-(toluene-4-sulfonylamino) nicotinic acid methyl ester was carried out as per STEP 13 of Example 28

### STEP 14: Synthesis of 4-amino-2,6-diethyl-nicotinic acid methyl ester

Synthesis of 4-amino-2,6-diethyl-nicotinic acid methyl ester was carried out as per STEP 14 of Example 28

### STEP 15: Synthesis of (4-Amino-2,6-diethyl-pyridin-3-yl)-methanol

Synthesis of (4-Amino-2,6-diethyl-pyridin-3-yl)-methanol was carried out as per STEP 14 of Example 28

### STEP 16: Synthesis of 4-Amino-2,6-diethyl-pyridine-3-carbaldehyde

Synthesis of 4-Amino-2,6-diethyl-pyridine-3-carbaldehyde was carried out as per STEP 15 of Example 28

### STEP 17: Synthesis of 5,7-diethyl-1H-[1,6]naphthyridin-2-one

Synthesis of 5,7-diethyl-1H-[1,6] naphthyridin-2-one was carried out as per STEP 16 of Example 28

### STEP 18: Synthesis of 1-Bromo-4-bromomethyl-2-fluoro-benzene

To a solution of 1-Bromo-2-fluoro-4-methyl-benzene (5 gm, 0.026 mol) in carbon tetrachloride (40 ml) N-bromosuccinimide (5.6 gm, 0.037 mol) was added followed by addition of 400 mg of Azobisobutyro nitrile (AIBN), then after reaction mixture was refluxed for 6 hrs. The reaction mixture was cooled to 0° C. and filtered under vacuum; filtrate was evaporated under vacuum to give 6 gm of 1-Bromo-4-bromomethyl-2-fluoro-benzene as yellow oil. This was further used as such.

### STEP 19: Synthesis of 1-(4-Bromo-3-fluoro-benzyl)-5,7-diethyl-1H-[1,6]naphthyridin-2-one

To the stirred solution of 5,7-Diethyl-1H-[1,6] naphthyridin-2-one (1 gm, 0.005 mol) in dimethyl formamide (10 ml) at ambient temperature under nitrogen atmosphere was added potassium carbonate (1 mg, 0.0072 mol) followed by addition of 1-Bromo-4-bromomethyl-2-fluoro-benzene (1.4 gm, 0.0052 mol) in 10 ml dimethyl formamide The reaction mixture was stirred at room temperature for 16 hrs. The mixture was then filtered off and residue was washed with ethyl acetate, combined organic phase was washed with water and brine then dried over sodium sulphate and evaporated under vacuum to afford 2 gm of 1-(4-bromo-3-fluoro-benzyl)-5,7-diethyl-1H-[1,6]-naphthyridin-2-one as a yellow colored oil.

Molecular Formula: C₃₂H₃₀N₆O₃S₂

Molecular Weight: 580

¹HNMR (DMSOd₆): 1.20 (t, J=7.6 Hz, 3H), 1.25 (t, J=7.6 Hz, 3H), 1.49 (s, 3H), 2.10 (s, 3H), 2.48 (s, 3H), 2.67-2.75 (q, J=7.2 Hz, 2H), 3.05-3.10 (q, J=7.2 Hz, 2H), 5.51 (s, 2H), 6.72-6.74 (d, J=10 Hz, 1H) 6.81 (s, 1H), 6.95-6.97 (d, J=7.6 Hz, 1H), 7.13-7.16 (m, 3H), 8.24-8.26 (d, J=10 Hz, 1H), 10.72 (br, 1H).

Mass Spectrum: (m⁻¹) 579.13

### Example 36

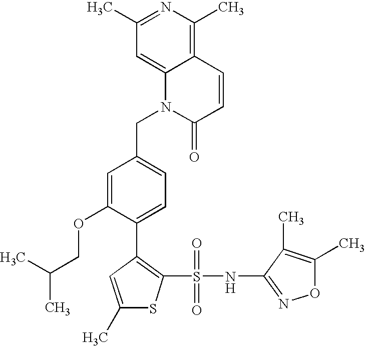

### 3-[4-(5,7-Dimethyl-2-oxo-2H-[1,6]naphthyridin-1-ylmethyl)-2-isobutoxy-phenyl]-5-methyl-thiophene-2-sulfonic acid (4,5-dimethyl-isoxazol-3-yl)-amide

### Step 01: Synthesis of sodium salt of 3-Hydroxy-2-methyl-but-2-enenitrile.(sodium salt of 3-cyano-2-butanone)

Synthesis of sodium salt of 3-Hydroxy-2-methyl-but-2-enenitrile (sodium salt of 3-cyano-2-butanone) was carried out as per STEP 01 of Example 28

### Step 02: Synthesis of 2,2-(2-Methyl-[1,3] dioxolan-2-yl)-Propionitrile

Synthesis of 2,2-(2-Methyl-[1,3] dioxolan-2-yl)-Propionitrile was carried out as per STEP 02 of Example 28

### Step 03: Synthesis of N-Hydroxy-2-(2-methyl-[1,3]dioxolan-2-yl)-propionamidine OR 2-(β-ethylene dioxy aceto) propionamideoxime

Synthesis of N-Hydroxy-2-(2-methyl-[1,3] dioxolan-2-yl)-propionamidine OR 2-(β-ethylene dioxy aceto) propionamideoxime was carried out as per STEP 03 of Example 28

### Step 04: Synthesis of 3-Amino-4,5 dimethyl isoxazole

Synthesis of 3-Amino-4,5 dimethyl isoxazole was carried out as per STEP 04 of Example 28

### Step 05: Synthesis of 5-methyl thiophene-2-sulphonyl chloride

Synthesis of 5-methyl thiophene-2-sulphonyl chloride was carried out as per STEP 07 of Example 01

### Step 06: Synthesis of 5-methyl-thiophene-2-sulfonic acid (4,5-dimethyl-isoxazol-3yl) amide

Synthesis of 5-methyl-thiophene-2-sulfonic acid (4,5-dimethyl-isoxazol-3yl) amide was carried out as per STEP 08 of Example 01

### Step 07: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 08: Synthesis of 3-borono-N-(4,5-dimethyl-3-isoxazolyl)-N-[(2-methoxy-ethoxy)methyl]-5-methyl-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### STEP 09: Synthesis of 4-{2-[(4,5-Dimethyl-isoxazol-3-yl)-(2-methoxy-ethoxymethyl)-sulfamoyl]-5-methyl-thiophen-3-yl}-3-isobutoxy-benzoic acid methyl ester

To a stirred solution of 4-Bromo-3-isobutoxy-benzoic acid methyl ester (1.0 gm, 0.00348 mol) in dimethoxy ethane (15 ml) under nitrogen was added bis(triphenylphosphine)palladium(II)chloride (0.243 gm, 3.4 mmol) followed by addition of 2M aqueous sodium carbonate (0.811 gm in 3.8 ml water) Reaction mixture was stirred at room temperature for 10 min and then heated at 60° C. To this drop wise added the solution of 3-Borono-N-(4,5-Dimethyl-3-isoxazolyl)-N-[(2-methoxy-ethoxy)methyl]-5-methyl-thiophene sulphonamide (700 mg, 0.003465 mol in 15 ml dimethoxy ethane) within 45 min and reaction was refluxed for 60 min. After 1 hr the same was repeated with further addition of 3-Borono-N-(4,5-Dimethyl-3-isoxazolyl)-N-[(2-methoxy-ethoxy)methyl]-5-methyl-thiophene sulphon-amide (700 mg, 0.003465 mol in 15 ml dimethoxy ethane) within 45 min, reaction mixture was refluxed for 4 hrs and stirred at room temperature for 12 hrs. Reaction mixture was diluted with ethyl acetate 100 ml and water, layers were separated, aqueous layer further extracted with ethyl acetate. Combine extracts was washed with water and brine. Dried over sodium sulphate and concentrated under vacuum. Crude compound was purified by column chromatography on a silica gel to yield 1.8 gm of 4-{2-[(4,5-Dimethyl-isoxazol-3-yl)-(2-methoxy-ethoxymethyl)-sulfamoyl]-5-methyl-thiophen-3-yl}-3-isobutoxy-benzoic acid methyl ester as pale yellow oily mass.

### STEP 10: Synthesis of 3-(4-Hydroxymethyl-2-isobutoxy-phenyl)-5-methyl-thiophene-2-sulfonic acid (4,5-dimethyl-isoxazol-3-yl)-(2-methoxy-ethoxymethyl)-amide

Lithium aluminium hydride (0.15 gm, 0.0039 mol) was added to a stirred solution of tetrahydrofuran (15 ml) at 0° C. under flow of nitrogen, followed by addition of 4-{2-[(4,5-Dimethyl-isoxazol-3-yl)-(2-methoxy-ethoxymethyl)-sulfamoyl]-5-methyl-thiophen-3-yl}-3-isobutoxy-benzoic acid methyl ester (1.5 gm, 0.0026 mol) in 20 ml Tetrahydrofuran. The reaction mixture was stirred at 0° C. for 15 mins. And raised the temperature of the reaction mixture to room temperature and stirred for 4 hrs. Reaction mixture was cooled to 0° C., and drop wise added sodium hydroxide solution 50 ml (1 gm dissolved in 100 ml water) maintaining the temperature at 0° C., followed by extraction with ethylacetate (25 ml×2). Organic layer was dried over sodium sulphate and concentrated completely under vacuum to give 1.4 gm of 3-(4-hydroxymethyl-2-isobutoxy-phenyl)-5-methyl-thiophene-2-sulfonic acid (4,5-dimethyl-isoxazol-3-yl)-(2-methoxy-ethoxymethyl)-amide

### STEP 11: Synthesis of methanesulfonic acid 4-{2-[(4,5-dimethyl-isoxazol-3-yl)-(2-methoxy-ethoxymethyl)-sulfamoyl]-5-methyl-thiophen-3-yl}-3-isobutoxy-benzyl ester

N-Ethyl diisopropyl amine (0.67 gm, 0.005204 mol) was added to a solution of 3-(4-Hydroxymethyl-2-isobutoxy-phenyl)-5-methyl-thiophene-2-sulfonic acid (4,5-dimethyl-isoxazol-3-yl)-(2-methoxy-ethoxymethyl)-amide (1.4 gm, 0.0026 mol) in 10 ml of dichloro methane. Cooled the reaction mixture to 0° C., added slowly methane sulfonyl chloride (0.351 gm, 0.003122 mol) into the reaction mixture. The reaction mixture was maintained at room temperature for 3 hrs. Reaction mixture was dumped into ice-cold water followed by extraction with methylene chloride (50 ml×2). Combine extracts was given washing with dilute hydrochloric acid followed by water and brine solution. Dried the organic layer over sodium sulphate and concentrated to give 1.5 gm of methanesulfonic acid 4-{2-[(4,5-dimethyl-isoxazol-3-yl)-(2-methoxy-ethoxymethyl)-sulfamoyl]-5-methyl-thiophen-3-yl}-3-isobutoxy-benzyl ester.

### Step 12: Synthesis of 3-[4-(5,7-Dimethyl-2-oxo-2H-[1,6]naphthyridin-1-ylmethyl)-2-isobutoxy-phenyl]-5-methyl-thiophene-2-sulfonic acid (4,5-dimethyl-isoxazol-3-yl)-(2-methoxy-ethoxymethyl)-amide

To the stirred solution of 5,7-Dimethyl-1H-[1,6]naphthyridin-2-one (0.0423 gm, 0.002435 mol) in dimethyl formamide (10 ml) at 0° C. under nitrogen was added portion wise sodium hydride (60% in mineral oil) (146 mg, 0.00365 mol). After the addition, the reaction mixture was warmed to ambient temperature and maintained for 30 min. Reaction mixture was cooled to 0° C. and a solution of methanesulfonic acid 4-{2-[(4,5-dimethyl-isoxazol-3-yl)-(2-methoxy-ethoxymethyl)-sulfamoyl]-5-methyl-thiophen-3-yl}-3-isobutoxy-benzyl ester (1.5 gm, 0.002435 mol) in (10 ml) dimethyl formamide was added drop wise to the reaction mixture and stirred at room temperature for 24 hrs. The mixture was then diluted with ethyl acetate (40 ml), followed by 10 ml of cold water, Organic layer washed with water and brine then dried over sodium sulphate and evaporated under vacuum to afford gm of crude material which was purified by column chromatography over Silica Gel column using hexane:ethyl acetate to afford 0.7 gm of 3-[4-(5,7-Dimethyl-2-oxo-2H-[1,6]naphthyridin-1-ylmethyl)-2-isobutoxy-phenyl]-5-methyl-thiophene-2-sulfonic acid (4,5-dimethyl-isoxazol-3-yl)-(2-methoxy-ethoxymethyl)-amide as a viscous oily mass.

### Step 13: Synthesis of 3-[4-(5,7-dimethyl-2-oxo-2H-[1,6]naphthyridin-1-ylmethyl)-2-isobutoxy-phenyl]-5-methyl-thiophene-2-sulfonic acid (4,5-dimethyl-isoxazol-3-yl)-amide

To, 3-[4-(5,7-Dimethyl-2-oxo-2H-[1,6]naphthyridin-1-ylmethyl)-2-isobutoxy-phenyl]-5-methyl-thiophene-2-sulfonic acid (4,5-dimethyl-isoxazol-3-yl)-(2-methoxy-ethoxymethyl)-amide (0.7 gm) was added ethanol (10 ml) and 6N aqueous hydrochloric acid (8 ml) at room temperature. Reaction mixture was refluxed for 3 hrs. The reaction mixture was concentrated under vacuum and pH of the solution was adjusted to 8 using a saturated solution of sodium bicarbonate and the mixture was extracted with ethyl acetate (25 ml×2). The combined organic extract were washed with water and brine then dried over sodium sulphate and concentrated under vacuum. The residue was purified by column Chromatography over Silica Gel column using hexane:ethyl acetate to afford 120 mg of 3-[4-(5,7-Dimethyl-2-oxo-2H-[1,6]naphthyridin-1-ylmethyl)-2-isobutoxy-phenyl]-5-methyl-thiophene-2-sulfonic acid (4,5-dimethyl-isoxazol-3-yl)-amide as a white solid.

### STEP 14: Synthesis of 2,6-Dimethyl-4-oxo-1,4-dihydro-pyridine-3-carboxylic acid ethyl ester

Synthesis of 2,6-Dimethyl-4-oxo-1,4-dihydro-pyridine-3-carboxylic acid ethyl ester was carried out as per STEP 14 of Example 29

### STEP 15: Synthesis of 2,6-Dimethyl-4-(toluene-4-sulfonylamino)-nicotinic acid ethyl ester

Synthesis of 2,6-Dimethyl-4-(toluene-4-sulfonylamino)-nicotinic acid ethyl ester was carried out as per STEP 15 of Example 29

### STEP 16: Synthesis of 4-amino-2,6-dimethyl-nicotinic acid ethyl ester

Synthesis of 4-amino-2,6-dimethyl-nicotinic acid ethyl ester was carried out as per STEP 16 of Example 29

### STEP 17: Synthesis of (4-Amino-2,6-dimethyl-pyridin-3-yl)-methanol

Synthesis of (4-Amino-2,6-dimethyl-pyridin-3-yl)-methanol was carried out as per STEP 17 of Example 29

### STEP 18: Synthesis of 4-Amino-2,6-dimethyl-pyridine-3-carbaldehyde

Synthesis of 4-Amino-2,6-dimethyl-pyridine-3-carbaldehyde was carried out as per STEP 18 of Example 29

### STEP 19: Synthesis of 5,7-Dimethyl-1H-[1,6]naphthyridin-2-one

A mixture of 4-Amino-2,6-dimethyl-pyridine-3-carbaldehyde (6.1 gm, 0.041 mol) and (carethoxymethylene) triphenylphosphorane (35.38 gm, 0.101 mol) in xylene (150 ml) was stirred and heated at reflux for 6 hrs. The reaction mixture was cooled to room temp. And the solvent was removed by evaporation. A solution of sodium methoxide (7.75 gm, 0.144 mol) in methanol (150 ml) was added to the residue and the resulting solution was heated at reflux for 4 hrs. Methanol was removed by evaporation and water (200 ml) was added. The mixture was acidified to pH 1-2 by addition of conc. hydrochloric acid. The mixture was then extracted with ethyl acetate (ml) and extract was discarded. The aqueous phase was then basified by addition of sodium carbonate This was then extracted with dichloromethane (200 ml×3). The organic layer was washed with water, brine and dried over sodium sulphate and concentrated to give 1.6 gm of 5,7-Dimethyl-1H-[1,6]naphthyridin-2-one as white solid.

### STEP 20: Synthesis of 4-Bromo-3-hydroxy-benzoic acid

To a (4.7 gm copper bromide was dissolved in to 5 ml of 48% hydrobromic acid and reflux it for 30 mins. Then to another flask 6 ml of 48% hydrobromic acid was cooled to 0° C. to this (5 gm, 0.032679 mol) 4-amino-3-hydroxy benzoic acid was added, under stirring (2.61 gm, 0.03782 mol) of sodium nitrite solution was added (dissolved in 20 ml water). Stir the reaction mixture for 30 min at 0° C. This solution was then added to the activated solution of copper bromide drop wise within 30 min. After completion of the addition reaction mixture was refluxed for 1 hr. Reaction mixture was cooled to room Temperature and then filtered though hyflow bed the filtrate was extracted with ethyl acetate (50 ml×3). The organic layer was washed with water, brine and dried over sodium sulphate and concentrated to give 1 gm of 4-Bromo-3-hydroxy-benzoic acid

### STEP 21: Synthesis of 4-Bromo-3-hydroxy-benzoic acid methyl ester

4-Bromo-3-hydroxy-benzoic acid (1 gm, 0.004 mol) was dissolved in 20 ml methanol and this solution was cooled to 00 C and then to this 0.2 ml concentrated Sulphuric acid was added. After completion of the addition reaction mixture was refluxed for 6 hrs then after methanol was evaporated under vacuum. To the residue water was added and extracted with diethyl ether. The organic layer was washed with sodium bicarbonate solution followed by water and brine finally organic layer was dried over sodium sulphate and concentrated to give 1.3 gm of 4-Bromo-3-hydroxy-benzoic acid methyl ester.

### STEP 22: Synthesis of 4-Bromo-3-isobutoxy-benzoic acid methyl ester

4-Bromo-3-hydroxy-benzoic acid methyl ester (1.3 gm, 0.005 mol) was dissolved in 10 ml dimethyl formamide followed by addition of potassium carbonate (1.7 gm, 0.01 mol). Then after to the reaction mixture (1 gm, 0.007 mol) isobutyl bromide was added and reaction mixture was heated and stirred at 90° C. for 10 hrs. The reaction mixture was cooled to room temperature and reaction mixture was filtered under vacuum to the filtrate water was added and extracted with ethyl acetate (20 ml×3). The organic layer was washed with water, brine and dried over sodium sulphate and concentrated to give 1 gm of 4-Bromo-3-isobutoxy-benzoic acid methyl ester as oil.

Molecular Formula: C₃₁H₃₄N₄O₅S₂

Molecular Weight: 606

¹HNMR (DMSOd₆): 0.812-0.82 (d, J=6.78 Hz, 6H), 1.47 (s, 3H), 1.79-1.82 (m, 1H), 2.10 (s, 3H), 2.44 (s, 3H), 2.46 (s, 3H), 2.70 (s, 3H), 3.59-3.61 (d, J=6.4 Hz, 2H), 5.46 (s, 2H), 6.53-6.55 (d, J=8 Hz, 1H), 6.71-6.74 (m, 2H), 7.00-7.04 (m, 2H), 7.22 (s, 2H), 8.19-8.21 (d, 1H), 10.55 (br, 1H).

Mass Spectrum: (m⁻¹) 605.1

### Example 37

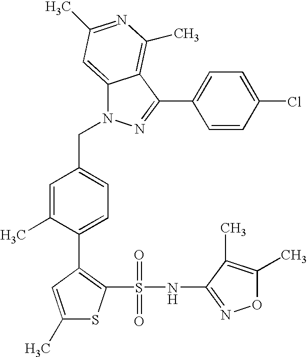

### 3-{4-[3-(4-Chloro-phenyl)-4,6-dimethyl-pyrazolo[4,3-c]pyridin-1-ylmethyl]-2-methyl-phenyl}-5-methyl-thiophene-2-sulfonic acid (4,5-dimethyl-isoxazol-3-yl)-amide

### Step 01: Synthesis of sodium salt of 3-Hydroxy-2-methyl-but-2-enenitrile.(sodium salt of 3-cyano-2-butanone)

Synthesis of sodium salt of 3-Hydroxy-2-methyl-but-2-enenitrile (sodium salt of 3-cyano-2-butanone) was carried out as per STEP 01 of Example 28

### Step 02: Synthesis of 2,2-(2-Methyl-[1,3] dioxolan-2-yl)-Propionitrile

Synthesis of 2,2-(2-Methyl-[1,3] dioxolan-2-yl)-Propionitrile was carried out as per STEP 02 of Example 28

### Step 03: Synthesis of N-Hydroxy-2-(2-methyl-[1,3]dioxolan-2-yl)-propionamidine OR 2-(β-ethylene dioxy aceto) propionamideoxime

Synthesis of N-Hydroxy-2-(2-methyl-[1,3] dioxolan-2-yl)-propionamidine OR 2-(β-ethylene dioxy aceto) propionamideoxime was carried out as per STEP 03 of Example 28

### Step 04: Synthesis of 3-Amino-4,5 dimethyl isoxazole

Synthesis of 3-Amino-4,5 dimethyl isoxazole was carried out as per STEP 04 of Example 28

### Step 05: Synthesis of 5-methyl thiophene-2-sulphonyl chloride

Synthesis of 5-methyl thiophene-2-sulphonyl chloride was carried out as per STEP 07 of Example 01

### Step 06: Synthesis of 5-methyl-thiophene-2-sulfonic acid (4,5-dimethyl-isoxazol-3-yl) amide

Synthesis of 5-methyl-thiophene-2-sulfonic acid (4,5-dimethyl-isoxazol-3yl) amide was carried out as per STEP 08 of Example 01

### Step 07: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 08: Synthesis of 3-borono-N-(4,5-dimethyl-3-isoxazolyl)-N-[(2-methoxy-ethoxy)methyl]-5-methyl-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### STEP 13: Synthesis of (4-{2-[(4,5-Dimethyl-isoxazol-3-yl)-(2-methoxy-ethoxymethyl)-sulfamoyl]-5-methyl-thiophene-3-yl}-3-methyl-benzoic acid methyl ester

Synthesis of (4-{2-[(4,5-dimethyl-isoxazol-3-yl)-(2-methoxy-ethoxymethyl)-sulfamoyl]-5-methyl-thiophene-3-yl}-3-methyl-benzoic acid methyl ester was carried out as per STEP 08 of Example 19

### STEP 14: Synthesis of 3-(4-hydroxy methyl-2-methyl-phenyl)-5-methyl-thiophene-2-sulfonic acid-(4,5-Dimethyl-isoxazol-3-yl)-(2-methoxy-ethoxymethyl) amide

Synthesis of 3-(4-hydroxy methyl-2-methyl-phenyl)-5-methyl-thiophene-2-sulfonic acid-(4,5-dimethyl-isoxazol-3-yl)-(2-methoxy-ethoxymethyl) amide was carried out as per STEP 09 of Example 19

### STEP 15: Synthesis of Methane sulfonic acid 4-{2-[(4,5-dimethyl-isoxazol-3yl)-(2-methoxy-ethoxy methyl)-sulfamoyl]-5-methylthiophene-3-yl}-3-methyl-benzyl ester

Synthesis of Methane sulfonic acid 4-{2-[(4,5-dimethyl-isoxazol-3-yl)-(2-methoxy-ethoxy methyl)-sulfamoyl]-5-methylthiophene-3-yl}-3-methyl-benzyl ester was carried out as per STEP 10 of Example 19

### Step 12: Synthesis of 3-{4-[3-(4-Chloro-phenyl)-4,6-dimethyl-pyrazolo[4,3-c]pyridin-1-ylmethyl]-2-methyl-phenyl}-5-methyl-thiophene-2-sulfonic acid (4,5-dimethyl-isoxazol-3-yl)-(2-methoxy-ethoxymethyl)-amide

To the stirred solution of 3-(4-Chloro-phenyl)-4,6-dimethyl-1H-pyrazolo[4,3-c]pyridine (516 mg, 0.002 mol) in dimethyl formamide (10 ml) at 0° C. under nitrogen was added portion wise sodium hydride (60% in mineral oil) (150 mg, 0.0031 mol). After the addition, the reaction mixture was warmed to ambient temperature and maintained for 30 min. Reaction mixture was cooled to 0° C. and a solution of Methane sulfonic acid 4-{2-[(4,5-dimethyl-isoxazol-3-yl)-(2-methoxy-ethoxy methyl)-sulfamoyl]-5-methyl-thiophen-3-yl}-3-methyl-benzyl ester. (1.0 gm, 0.002 mol) in (10 ml) dimethyl formamide was added drop wise to the reaction mixture and stirred at room temperature for 24 hrs. The mixture was then diluted with ethyl acetate (40 ml), followed by 10 ml of cold water, Organic layer washed with water and brine then dried over sodium sulphate and evaporated under vacuum to afford 1.6 gm of 3-{4-[3-(4-Chloro-phenyl)-4,6-dimethyl-pyrazolo[4,3-c]pyridin-1-ylmethyl]-2-methyl-phenyl}-5-methyl-thiophene-2-sulfonic acid (4,5-dimethyl-isoxazol-3-yl)-(2-methoxy-ethoxymethyl)-amide as a viscous oily mass.

### Step 13: Synthesis of 3-{4-[3-(4-Chloro-phenyl)-4,6-dimethyl-pyrazolo[4,3-c]pyridin-1-ylmethyl]-2-methyl-phenyl}-5-methyl-thiophene-2-sulfonic acid (4,5-dimethyl-isoxazol-3-yl)-amide

To, 3-{4-[3-(4-Chloro-phenyl)-4,6-dimethyl-pyrazolo[4,3-c]pyridin-1-ylmethyl]-2-methyl-phenyl}-5-methyl-thiophene-2-sulfonic acid (4,5-dimethyl-isoxazol-3-yl)-(2-methoxy-ethoxymethyl)-amide (1.6 gm) was added ethanol (10 ml) and 6N aqueous hydrochloric acid (8 ml) at room temperature. Reaction mixture was refluxed for 3 hrs. The reaction mixture was concentrated under vacuum and pH of the solution was adjusted to 8 using a saturated solution of sodium bicarbonate and the mixture was extracted with ethyl acetate (25 ml×2). The combined organic extract were washed with water and brine then dried over sodium sulphate and concentrated under vacuum. The residue was purified by column Chromatography over Silica Gel column using hexane:ethyl acetate to afford 200 mg of 3-{4-[3-(4-Chloro-phenyl)-4,6-dimethyl-pyrazolo[4,3-c]pyridin-1-ylmethyl]-2-methyl-phenyl}-5-methyl-thiophene-2-sulfonic acid (4,5-dimethyl-isoxazol-3-yl)-amide as a white solid.

### STEP 14: Synthesis of 1-(4-Chloro-phenyl)-butane-1,3-dione

In (50 ml) N,N-Dimethyl formamide was added Sodium hydride (60% in mineral oil) (3.88 gm, 0.097 mol) at 0° C., followed by addition of solution of dry ethyl acetate (6.8 gm, 0.07727 mol) and 4-chloro acetophenone (10.0 gm, 0.065 mol). This reaction was stirred at room temperature for 12 hrs. Reaction mixture was acidified with 1N Hydrochloric acid and extracted with ethyl acetate (100 ml×2). Combined extracts were washed with water and brine. Dried over sodium sulphate and evaporated to give 13.0 gm of yellow coloured solid of 1-(4-Chloro-phenyl)-butane-1,3-dione.

### STEP 15: Synthesis of 3-Amino-1-(4-chloro-phenyl)-but-2-en-1-one

A mixture of 1-(4-Chloro-phenyl)-butane-1,3-dione. (27.0 gm, 0.136 mol) and ammonium acetate (31.6 gm, 0.41 mol) in dry methanol (200 ml) was stirred at room temperature for 24 hrs. The reaction mixture was concentrated completely under vacuum and chilled water was added to the residue. The reaction mixture was basified to pH8 using saturated sodium bicarbonate solution, followed by extraction with ethyl acetate (100 ml×2). Combine extracts were washed with water and brine. Dried over sodium sulphate and evaporated to give 32.0 gm of 3-Amino-1-(4-chloro-phenyl)-but-2-en-1-one.

### STEP 16: Synthesis of 3-(4-Chloro-benzoyl)-2,6-dimethyl-1H-pyridin-4-one

A mixture of 2,2,6-Trimethyl-[1,3] dioxin-4-one (11.56 gm, 0.081 mol) and 3-Amino-1-(4-chloro-phenyl)-but-2-en-1-one (10.0 gm, 0.059 mol) was heated to reflux at 120° C. for 6 hrs. Reaction mixture was purified by column chromatography over silica gel column, eluting the desired product with 10% methanol and ethyl acetate to give 3.1 gm of 3-(4-Chloro-benzoyl)-2,6-dimethyl-1H-pyridin-4-one.

### STEP 17: Synthesis of (4-Chloro-2,6-dimethyl-pyridin-3-yl)-(4-chloro-phenyl)-methanone

3-(4-Chloro-benzoyl)-2,6-dimethyl-1H-pyridin-4-one (3.1 gm, 0.012 mol) was added to 20 ml phosphorous oxychloride at 0° C. Stirred and heated the reaction mixture at 100° C. and maintained for 8 hours. Work-up was done by evaporating the phosphorus oxy chloride under vacuum and basified the residue to pH 8 with saturated Sodium carbonate solution, followed by extraction with methylene dichloride (50 ml×2). Combined extracts were washed with water and brine. Dried over anhydrous sodium sulphate and concentrated to give 3.2 gm of (4-Chloro-2,6-dimethyl-pyridin-3-yl)-(4-chloro-phenyl)-methanone

### STEP 18: Synthesis of 3-(4-Chloro-phenyl)-4,6-dimethyl-1H-pyrazolo[4,3-c]pyridine

(4-Chloro-2,6-dimethyl-pyridin-3-yl)-(4-chloro-phenyl)-methanone (3.4 gm, 0.012 mol) was taken in ethanol (10 ml) and hydrazine hydrate (5.8 ml, 0.185 mol) and two drops of acetic acid was added to the reaction mixture. Slowly raised the temperature and heated to reflux, maintained reflux for 6 hours. Reaction mixture was completely evaporated under vacuum. The crude mass was dumped in to ice, solid obtained was filtered off and suck dried to provide 700 mg of 3-(4-Chloro-phenyl)-4,6-dimethyl-1H-pyrazolo[4,3-c]pyridine.

Molecular Formula: C₃₂H₃₀CLN₅O₃S₂

Molecular Weight: 631.5

¹HNMR (DMSOd₆): 1.45 (s, 3H), 1.93 (s, 3H), 2.11 (s, 3H), 2.47 (s, 3H), 2.49 (s, 3H), 2.54 (s, 3H), 5.63 (s, 2H), 6.68 (s, 1H), 6.90-6.99 (m, 2H), 7.17 (s, 1H), 7.51 (s, 1H), 7.58-7.60 (d, J=8.4 Hz, 2H), 7.68-7.70 (d, J=8.4 Hz, 2H), 10.64 (br, 1H).

Mass Spectrum: (m⁻¹) 630.1

### Example 38

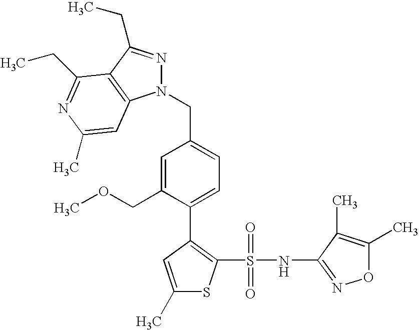

### 3-[4-(3,4-Diethyl-6-methyl-pyrazolo[4,3-c]pyridin-1-ylmethyl)-2-methoxymethyl-phenyl]-5-methyl-thiophene-2-sulfonic acid (4,5-dimethyl-isoxazol-3-yl)-amide

### Step 01: Synthesis of sodium salt of 3-Hydroxy-2-methyl-but-2-enenitrile.(sodium salt of 3-cyano-2-butanone)

Synthesis of sodium salt of 3-Hydroxy-2-methyl-but-2-enenitrile (sodium salt of 3-cyano-2-butanone) was carried out as per STEP 01 of Example 28

### Step 02: Synthesis of 2,2-(2-Methyl-[1,3] dioxolan-2-yl)-Propionitrile

Synthesis of 2,2-(2-Methyl-[1,3] dioxolan-2-yl)-Propionitrile was carried out as per STEP 02 of Example 28

### Step 03: Synthesis of N-Hydroxy-2-(2-methyl-[1,3]dioxolan-2-yl)-propionamidine OR 2-(β-ethylene dioxy aceto) propionamideoxime

Synthesis of N-Hydroxy-2-(2-methyl-[1,3] dioxolan-2-yl)-propionamidine OR 2-(β-ethylene dioxy aceto) propionamideoxime was carried out as per STEP 03 of Example 28

### Step 04: Synthesis of 3-Amino-4,5 dimethyl isoxazole

Synthesis of 3-Amino-4,5 dimethyl isoxazole was carried out as per STEP 04 of Example 28

### STEP 05: Synthesis of 4-bromo-3-bromomethyl-benzoic acid ethyl ester. Synthesis of 4-bromo-3-bromomethyl-benzoic acid ethyl ester was carried out as per STEP 02 of Example 01

### STEP 06: Synthesis of 4-Bromo-3-methoxymethyl-benzoic acid ethyl ester

Synthesis of 4-Bromo-3-methoxymethyl-benzoic acid ethyl ester was carried out as per STEP 04 of Example 20

### Step 07: Synthesis of 5-methyl thiophene-2-sulphonyl chloride

Synthesis of 5-methyl thiophene-2-sulphonyl chloride was carried out as per STEP 07 of Example 01

### Step 08: Synthesis of 5-methyl-thiophene-2-sulfonic acid (4,5-dimethyl-isoxazol-3yl) amide

Synthesis of 5-methyl-thiophene-2-sulfonic acid (4,5-dimethyl-isoxazol-3yl) amide was carried out as per STEP 08 of Example 01

### Step 09: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 10: Synthesis of 3-borono-N-(4,5-dimethyl-3-isoxazolyl)-N-[(2-methoxy-ethoxy)methyl]-5-methyl-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### STEP 11: Synthesis of (4-{2-[(4,5-Dimethyl-isoxazol-3-yl)-(2-methoxy-ethoxymethyl)-sulfamoyl]-5-methyl-thiophene-3-yl}-3-methoxymethyl-benzoic acid ethyl ester

Synthesis of (4-{2-[(4,5-Dimethyl-isoxazol-3-yl)-(2-methoxy-ethoxymethyl)-sulfamoyl]-5-methyl-thiophene-3-yl}-3-methoxymethyl-benzoic acid ethyl ester was carried out as per STEP 15 of Example 21

### STEP 12: Synthesis of 3-(2-ethoxymethyl-4-hydroxy methyl-phenyl)-5-methyl-thiophene-2-sulfonic acid-(4,5-dimethyl-isoxazol-3-yl)-2-methoxy-methyl) amide

Synthesis of 3-(2-ethoxymethyl-4-hydroxy methyl-phenyl)-5-methyl-thiophene-2-sulfonic acid-(4,5-dimethyl-isoxazol-3-yl)-2-methoxy-methyl) amide was carried out as per STEP 16 of Example 21

### STEP 13: Synthesis of methane sulfonic acid 4-{2-[(4,5-dimethyl-isoxazol-3-yl)-(2-methoxy-ethoxy methyl)-sulfamoyl]-5-methylthiophene-3-yl}-3-methoxy methyl-benzyl ester

Synthesis of methane sulfonic acid 4-{2-[(4,5-dimethyl-isoxazol-3-yl)-(2-methoxy-ethoxy methyl)-sulfamoyl]-5-methylthiophene-3-yl}-3-methoxy methyl-benzyl ester was carried out as per STEP 16 of Example 21

### Step 14: Synthesis of 3-[4-(3,4-Diethyl-6-methyl-pyrazolo[4,3-c]pyridin-1-ylmethyl)-2-methoxymethyl-phenyl]-5-methyl-thiophene-2-sulfonic acid (4,5-dimethyl-isoxazol-3-yl)-(2-methoxy-ethoxymethyl)-amide

To the stirred solution of 3,4-Diethyl-6-methyl-1H-pyrazolo[4,3-c]pyridine (536 mg, 2.8 mmol) in dimethyl formamide (10 ml) at 0° C. under nitrogen was added portion wise sodium hydride (60% in mineral oil) (204 mg, 4.2 mmol). After the addition, the reaction mixture was warmed to ambient temperature and maintained for 30 min. Reaction mixture was cooled to 0° C. and a solution of methane sulfonic acid 4-{2-[(4,5-dimethyl-isoxazol-3-yl)-(2-methoxy-ethoxy methyl)-sulfamoyl]-5-methylthiophene-3-yl}-3-methoxymethyl-benzyl ester (1.5 gm, 2.8 mmol) in (10)ml dimethyl formamide was added drop wise to the reaction mixture and stirred at room temperature for 16 hrs. The mixture was then diluted with ethyl acetate (40 ml), followed by 10 ml of cold water, Organic layer washed with water and brine then dried over sodium sulphate and evaporated under vacuum to afford crude 1.5 gms. Crude compound was purified by column chromatography on a silica gel ti yield 1.0 gm of 3-[4-(3,4-Diethyl-6-methyl-pyrazolo[4,3-c]pyridin-1-ylmethyl)-2-methoxymethyl-phenyl]-5-methyl-thiophene-2-sulfonic acid (4,5-dimethyl-isoxazol-3-yl)-(2-methoxy-ethoxymethyl)-amide as a viscous oily mass.

### Step 15: Synthesis of 3-[4-(3,4-diethyl-6-methyl-pyrazolo[4,3-c]pyridin-1-ylmethyl)-2-methoxymethyl-phenyl]-5-methyl-thiophene-2-sulfonic acid (4,5-dimethyl-isoxazol-3-yl)-amide

To, 1.5 gm of 3-[4-(3,4-Diethyl-6-methyl-pyrazolo[4,3-c]pyridin-1-ylmethyl)-2-methoxymethyl-phenyl]-5-methyl-thiophene-2-sulfonic acid (4,5-dimethyl-isoxazol-3-yl)-(2-methoxy-ethoxymethyl)-amide was added 95% ethanol (10 ml) and 6N aqueous hydrochloric acid (8 ml) at room temperature. Reaction mixture was refluxed for 3 hrs. The reaction mixture was concentrated under vacuum and pH of the solution was adjusted to 8 using a saturated solution of sodium bicarbonate, and the mixture was extracted with dichloromethane (25 ml×3). The combined organic extract were washed with water and brine then dried over sodium sulphate and concentrated under vacuum. The residue was purified over Silica Gel chromatography using hexane:ethyl acetate to afford 90 mg of 3-[4-(3,4-Diethyl-6-methyl-pyrazolo[4,3-c]pyridin-1-ylmethyl)-2-methoxymethyl-phenyl]-5-methyl-thiophene-2-sulfonic acid (4,5-dimethyl-isoxazol-3-yl)-amide.

### STEP 16: Synthesis of 5-Amino-hept-4-en-3-one

A mixture of Heptane-3,5-dione (8 gm, 0.062 mol) and ammonium acetate (14.43 gm, 0.187 mol) in dry methanol (50 ml) was stirred at room temperature for 24 hrs. The reaction mixture was concentrated completely under vacuum and chilled water was added to the residue. The reaction mixture was basified to pH8 using saturated sodium bicarbonate solution, followed by extraction with ethyl acetate (100 ml×2). Combine extracts were washed with water and brine. Dried over sodium sulphate and evaporated to give 6 gm of 5-Amino-hept-4-en-3-one.

### STEP 17: Synthesis of 2-Ethyl-6-methyl-3-propionyl-1H-pyridin-4-one

A mixture of 2,2,6-Trimethyl-[1,3] dioxin-4-one (13.4 gm, 0.094 mol) and 5-Amino-hept-4-en-3-one. (6 gm, 0.047 mol) was heated to reflux at 120° C. for 6 hrs. Reaction mixture was purified by column chromatography over silica gel column, eluting the desired product with 10% methanol and ethyl acetate to give 3.9 gm of 2-Ethyl-6-methyl-3-propionyl-1H-pyridin-4-one

### STEP 18: Synthesis of 1-(4-Chloro-2-ethyl-6-methyl-pyridin-3-yl)-propan-1-one

2-Ethyl-6-methyl-3-propionyl-1H-pyridin-4-one (3.9 gm) was added to 20 ml phosphorous oxychloride at 0° C. Stirred and heated the reaction mixture at 100° C. and maintained for 6 hours. Work-up was done by evaporating the phosphorus oxy chloride under vacuum and basified the residue to pH 8 with saturated Sodium carbonate solution, followed by extraction with methylene dichloride (50 ml×2). Combined extracts were washed with water and brine. Dried over anhydrous sodium sulphate and concentrated to give 2.95 gm of 1-(4-Chloro-2-ethyl-6-methyl-pyridin-3-yl)-propan-1-one.

### STEP 19: Synthesis of 3,4-Diethyl-6-methyl-1H-pyrazolo[4,3-c]pyridine

1-(4-Chloro-2-ethyl-6-methyl-pyridin-3-yl)-propan-1-one (2.95 gm) was taken in ethanol (20 ml) and hydrazine hydrate (6 ml) and two drops of acetic acid was added to the reaction mixture. Slowly raised the temperature and heated to reflux, maintained reflux for 6 hours. Reaction mixture was completely evaporated under vacuum. The crude mass was dumped on to the ice, solid obtained was filtered off and suck dried to provide 1.68 gm of 3,4-Diethyl-6-methyl-1H-pyrazolo[4,3-c]pyridine.

Molecular Formula: C₃₀H₃₅N₅O₄S₂

Molecular Weight: 593

¹HNMR (DMSOd₆): 1.29-1.36 (m, 6H), 1.49 (s, 3H), 2.14 (s, 3H), 2.48 (s, 3H), 2.54 (s, 3H), 3.07-3.13 (m, 5H), 4.03 (s, 2H), 5.59 (s, 2H), 6.70 (s, 1H), 6.92-7.01 (m, 2H), 7.34 (s, 1H), 7.48 (s, 1H), 10.75 (br, 1H).

Mass Spectrum: (m⁻¹) 592.2

### Example 39

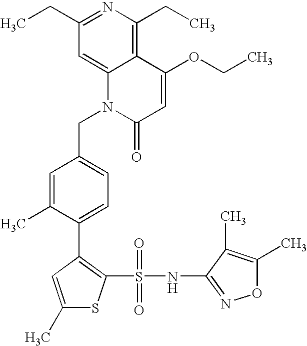

### 3-[4-(4-Ethoxy-5,7-diethyl-2-oxo-2H-[1,6]naphthyridin-1-ylmethyl)-2-methyl-phenyl]-5-methyl-thiophene-2-sulfonic acid (4,5-dimethyl-isoxazol-3-yl)-amide

### Step 01: Synthesis of sodium salt of 3-Hydroxy-2-methyl-but-2-enenitrile.(sodium salt of 3-cyano-2-butanone)

Synthesis of sodium salt of 3-Hydroxy-2-methyl-but-2-enenitrile (sodium salt of 3-cyano-2-butanone) was carried out as per STEP 01 of Example 28

### Step 02: Synthesis of 2,2-(2-Methyl-[1,3] dioxolan-2-yl)-Propionitrile

Synthesis of 2,2-(2-Methyl-[1,3] dioxolan-2-yl)-Propionitrile was carried out as per STEP 02 of Example 28

### Step 03: Synthesis of N-Hydroxy-2-(2-methyl-[1,3]dioxolan-2-yl)-propionamidine OR 2-(β-ethylene dioxy aceto) propionamideoxime

Synthesis of N-Hydroxy-2-(2-methyl-[1,3] dioxolan-2-yl)-propionamidine OR 2-(β-ethylene dioxy aceto) propionamideoxime was carried out as per STEP 03 of Example 28

### Step 04: Synthesis of 3-Amino-4,5 dimethyl isoxazole

Synthesis of 3-Amino-4,5 dimethyl isoxazole was carried out as per STEP 04 of Example 28

### Step 07: Synthesis of 5-methyl thiophene-2-sulphonyl chloride

Synthesis of 5-methyl thiophene-2-sulphonyl chloride was carried out as per STEP 07 of Example 01

### Step 08: Synthesis of 5-methyl-thiophene-2-sulfonic acid (4,5-dimethyl-isoxazol-3yl) amide

Synthesis of 5-methyl-thiophene-2-sulfonic acid (4,5-dimethyl-isoxazol-3yl) amide was carried out as per STEP 08 of Example 01

### Step 09: Synthesis of 5-methyl-thiophene-2-sulphonic acid (4,5 dimethyl-isoxazol-3-yl)-(2-methoxy-ethoxymethyl)-amide

Synthesis of 5-methyl-thiophene-2-sulphonic acid (4,5 dimethyl-isoxazol-3-yl)-(2-methoxy-ethoxymethyl)-amide was carried out as per STEP 09 of Example 01

### Step 10: Synthesis of 3-borono-N-(4,5-dimethyl-3-isoxazolyl)-N-[(2-methoxy-ethoxy)methyl]-5-methyl-thiophene sulphonamide

Synthesis of 3-borono-N-(4,5-dimethyl-3-isoxazolyl)-N-[(2-methoxy-ethoxy)methyl]-5-methyl-thiophene sulphonamide was carried out as per STEP 10 of Example 01

### STEP 13: Synthesis of (4-{2-[(4,5-Dimethyl-isoxazol-3-yl)-(2-methoxy-ethoxymethyl)-sulfamoyl]-5-methyl-thiophene-3-yl}-3-methyl-benzoic acid methyl ester

Synthesis of (4-{2-[(4,5-dimethyl-isoxazol-3-yl)-(2-methoxy-ethoxymethyl)-sulfamoyl]-5-methyl-thiophene-3-yl}-3-methyl-benzoic acid methyl ester was carried out as per STEP 08 of Example 19

### STEP 14: Synthesis of 3-(4-hydroxy methyl-2-methyl-phenyl)-5-methyl-thiophene-2-sulfonic acid-(4,5-Dimethyl-isoxazol-3-yl)-(2-methoxy-ethoxymethyl) amide

Synthesis of 3-(4-hydroxy methyl-2-methyl-phenyl)-5-methyl-thiophene-2-sulfonic acid-(4,5-dimethyl-isoxazol-3-yl)-(2-methoxy-ethoxymethyl) amide was carried out as per STEP 09 of Example 19

### STEP 15: Synthesis of Methane sulfonic acid 4-{2-[(4,5-dimethyl-isoxazol-3yl)-(2-methoxy-ethoxy methyl)-sulfamoyl]-5-methylthiophene-3-yl}-3-methyl-benzyl ester

Synthesis of Methane sulfonic acid 4-{2-[(4,5-dimethyl-isoxazol-3-yl)-(2-methoxy-ethoxy methyl)-sulfamoyl]-5-methylthiophene-3-yl}-3-methyl-benzyl ester was carried out as per STEP 10 of Example 19

### Step 12: Synthesis of 3-[4-(4-Chloro-5,7-diethyl-2-oxo-2H-[1,6]naphthyridin-1-ylmethyl)-2-methyl-phenyl]-5-methyl-thiophene-2-sulfonic acid (4,5-dimethyl-isoxazol-3-yl)-(2-methoxy-ethoxymethyl)-amide

To the stirred solution of 4-Chloro-5,7-diethyl-1H-[1,6]naphthyridin-2-one (0.5 gm, 2.2 mmol) in dimethyl formamide (10 ml) at ambient temperature under nitrogen was added potassium carbonate (437 mg, 3.2 mmol), and a solution of Methane sulfonic acid 4-{2-[(4,5-dimethyl-isoxazol-3-yl)-(2-methoxy-ethoxy methyl)-sulfamoyl]-5-methyl-thiophen-3-yl}-3-methyl-benzyl ester. (1.17 gm, 2 mmol) in (10 ml) dimethyl formamide was added drop wise to the reaction mixture and stirred at room temperature for 16 hrs. The reaction mixture was then filtered off and residue was washed with ethyl acetate (40 ml), combined Organic layer washed with water and brine then dried over sodium sulphate and evaporated under vacuum to afford 1.2 gm of crude material which was purified by column Chromatography over Silica Gel column using hexane:ethyl acetate to afford 0.8 gm of 3-[4-(4-Chloro-5,7-diethyl-2-oxo-2H-[1,6]naphthyridin-1-ylmethyl)-2-methyl-phenyl]-5-methyl-thiophene-2-sulfonic acid (4,5-dimethyl-isoxazol-3-yl)-(2-methoxy-ethoxymethyl)-amide as a viscous oily mass.

### Step 13: Synthesis of 3-[4-(4-Ethoxy-5,7-diethyl-2-oxo-2H-[1,6]naphthyridin-1-ylmethyl)-2-methyl-phenyl]-5-methyl-thiophene-2-sulfonic acid (4,5-dimethyl-isoxazol-3-yl)-(2-methoxy-ethoxymethyl)-amide

To a stirred solution of (800 mg, 1.15 mmol) 3-[4-(4-Chloro-5,7-diethyl-2-oxo-2H-[1,6]naphthyridin-1-ylmethyl)-2-methyl-phenyl]-5-methyl-thiophene-2-sulfonic acid (4,5-dimethyl-isoxazol-3-yl)-(2-methoxy-ethoxymethyl)-amide in (8 ml) ethanol, sodium ethoxide (120 mg, 1.7 mmol) was added at room temp. After completion of the addition reaction mixture was stirred and heated at 40° C. for 1 hr. Then reaction mixture was cooled to room temp. and evaporated under vacuum. To the residue water was added and pH was adjusted to 2 with aq. hydrochloric acid followed by extraction with ethyl acetate (50 ml×2) Organic layer was washed with water and brine finally dried over sodium sulphate and evaporated under vacuum to give 700 mg of 3-[4-(4-Ethoxy-5,7-diethyl-2-oxo-2H-[1,6]naphthyridin-1-ylmethyl)-2-methyl-phenyl]-5-methyl-thiophene-2-sulfonic acid (4,5-dimethyl-isoxazol-3-yl)-(2-methoxy-ethoxymethyl)-amide as oil.

### Step 14: Synthesis of 3-[4-(4-Ethoxy-5,7-diethyl-2-oxo-2H-[1,6]naphthyridin-1-ylmethyl)-2-methyl-phenyl]-5-methyl-thiophene-2-sulfonic acid (4,5-dimethyl-isoxazol-3-yl)-amide

To, 3-[4-(4-Ethoxy-5,7-diethyl-2-oxo-2H-[1,6]naphthyridin-1-ylmethyl)-2-methyl-phenyl]-5-methyl-thiophene-2-sulfonic acid (4,5-dimethyl-isoxazol-3-yl)-(2-methoxy-ethoxymethyl)-amide (0.3 gm) was added ethanol (5 ml) and 6N aqueous hydrochloric acid (3 ml) at room temperature. Reaction mixture was refluxed for 2 hrs. The reaction mixture was concentrated under vacuum and pH of the solution was adjusted to 8 using a saturated solution of sodium bicarbonate and the mixture was extracted with ethyl acetate (25 ml×2). The combined organic extract were washed with water and brine then dried over sodium sulphate and concentrated under vacuum. The residue was purified by column Chromatography over Silica Gel column using hexane:ethyl acetate to afford 80 mg of 3-[4-(4-Ethoxy-5,7-diethyl-2-oxo-2H-[1,6]naphthyridin-1-ylmethyl)-2-methyl-phenyl]-5-methyl-thiophene-2-sulfonic acid (4,5-dimethyl-isoxazol-3-yl)-amide as a brown solid.

### STEP 11: Synthesis of methyl 3-amino pentenoate

Synthesis of Methyl 3-amino pentenoate was carried out as per STEP 11 of Example 28

### STEP 12: Synthesis of 2,6-Diethyl-4-oxo-1,4-dihydro-pyridine-3-carboxylic acid methyl ester

Synthesis of 2,6-diethyl-4-oxo-1,4-dihydro-pyridine-3-carboxylic acid methyl ester was carried out as per STEP 12 of Example 28

### STEP 13: Synthesis of 2,6-diethyl-4-(toluene-4-sulfonylamino) nicotinic acid methyl ester

Synthesis of 2,6-Diethyl-4-(toluene-4-sulfonylamino) nicotinic acid methyl ester was carried out as per STEP 13 of Example 28

### STEP 14: Synthesis of 4-amino-2,6-diethyl-nicotinic acid methyl ester

Synthesis of 4-amino-2,6-diethyl-nicotinic acid methyl ester was carried out as per STEP 14 of Example 28

### STEP 19: Synthesis of 5,7-diethyl-4-hydroxy-2-oxo-1,2-dihydro-[1,6] naphthyridine-3-carboxylic acid ethyl ester

Diethylmalonate (15 ml, 0.093 mol) and methyl-4-amino-2,6-diethylpyridine-3-carboxylate (19.0 gm, 0.09 mol) were added to a solution of sodium ethoxide (7 gm, 0.10 mol) in ethanol (60 ml) and this reaction mixture was heated at 150° C. and 100 psi pressure for 20 hours in an autoclave. The reaction mixture was allowed to cool and the volatile material was removed by evaporation and the resulting semi solid was triturated with ether to give a white solid, which was collected by filtration and dissolved in water. This solution was then acidified with 1 N hydrochloric acid to give a white solid which was filtered and suction dried to yield 11 gm of ethyl 5,7-diethyl-4-hydroxy-2-oxo-1,2-dihydro-1,6-naphthyridine-3-carboxylate as off white solid.

### STEP 20: Synthesis of 5,7-diethyl-4-hydroxy-1H-[1,6] naphthyridin-2-one

Ethyl 5,7-diethyl-4-hydroxyl-2-oxo-1,2-dihydro-1,6-naphthyridine-3-carboxylate (11 gm) was dissolved in a mixture of water (11 ml), 1,4-dioxane (22 ml) and concentrated hydrochloric acid (11 ml) and the reaction mixture was heated to reflux for 3 hours. The reaction mixture was then cooled and the suspended solid was filtered off, washed with ethanol and ether and suction dried to give 4.3 gm of 5,7-diethyl-4-hydroxy-1,6-naphthyridin-2(1H)-one as an off white solid.

### STEP 21: Synthesis of 4-chloro-5,7-diethyl-1,6-naphthyridin-2(1H)-one

(4.3 gm, 0.019 mol) of 5,7-diethyl-4-hydroxy-1,6-naphthyridin-2(1H)-one was dissolved in (22 ml) phosphorous oxy chloride and the reaction mixture was refluxed for 24 hours. The reaction mixture was concentrated and the residue was dissolved in concentrated hydrochloric acid (16 ml) and 22 ml of water and refluxed for 4 hours. The reaction mixture was diluted with water and basified with solid sodium bicarbonate. The resulting solid was collected by filtration, washed with water and suction dried to give 3.0 gm of 4-chloro-5,7-diethyl-1,6-naphthyridin-2(1H)-one as an orange colored solid.

Molecular Formula: C₃₂H₃₆N₄O₅S₂

Molecular Weight: 620

¹HNMR (DMSOd₆): 1.16-1.27 (m, 6H), 1.47-1.50 (m, 6H), 1.92 (s, 3H), 2.12 (s, 3H), 2.48 (s, 3H), 2.67-2.72 (q, J=7.2 Hz, 2H), 3.20-3.26 (q, J=7.2 Hz, 2H), 4.21-4.26 (q, J=6.8 Hz, 2H), 5.46 (s, 2H), 6.10 (s, 1H), 6.70 (s, 1H), 6.84-6.91 (m, 2H), 7.11-7.16 (m, 2H), 10.26 (s, 1H).

Mass Spectrum: (m⁻¹) 619.2

Following compounds can also be prepared using the procedure mentioned in reaction scheme I, II & III as depicted above:
3-[4-(5,7-Dimethyl-2-oxo-2H-1,6-naphthyridin-1-ylmethyl)-phenyl]-5-methyl-furan-2-sulfonic acid (4,5-dimethyl-isoxazol-3-yl)-amide (Compound 40),
5-Methyl-3-[4-(7-oxo-2-propyl-4,5,6,7-tetrahydro-benzimidazol-1-ylmethyl)-phenyl]-thiophene-2-sulfonic acid (4-ethyl-5-methyl-isoxazol-3-yl)-amide (Compound 41),
3-{4-[2-(4,5-Dimethyl-isoxazol-3-yl sulfamoyl)-5-methyl-thiophen-3-yl]-benzyl}-4-oxo-2-propyl-3,4-dihydro-quinazoline-5-carboxylic acid (Compound 42),
5-Methyl-3-[4-(3,4,5,7-tetramethyl-2-oxo-2H-1,6-naphthyridin-1-ylmethyl)-phenyl]-thiophene-2-sulfonic acid (4,5-dimethyl-isoxazol-3-yl)-amide (Compound 43),
2-[4-(4,5-Diethyl-3,7-dimethyl-2-oxo-2H-1,6-naphthyridin-1-ylmethyl)-phenyl]-pyridine-3-sulfonic acid (4,5-dimethyl-isoxazol-3-yl)-amide (Compound 44),
2-Butyl-5-chloro-3-{4-[5-(4,5-dimethyl-isoxazol-3-ylsulfamoyl)-3-methyl-isoxazol-4-yl]-benzyl}-3H-imidazole-4-carboxylic acid (Compound 45),
3-[4-(2-Methyl-5,6,7,8-tetrahydro-quinolin-4-yloxymethyl)-phenyl]-benzo[b]thiophene-2-sulfonic acid (4,5-dimethyl-isoxazol-3-yl)-amide (Compound 46),
3-[4-(6-Ethyl-4-methyl-3-phenyl-pyrazolo[4,3-c]pyridin-1-ylmethyl)-phenyl]-benzo[b]thiophene-2-sulfonic acid (4,5-diethyl-isoxazol-3-yl)-amide (Compound 47),
3-[4-(5,7-Dimethyl-2-oxo-2H-1,6-naphthyridin-1-ylmethyl)-phenyl]-4,5,6,7-tetrahydro-enzo[b]thiophene-2-sulfonic acid (4,5-dimethyl-isoxazol-3-yl)-amide (Compound 48),
3-[2-Chloro-4-(5,7-dimethyl-2-oxo-2H-1,6-naphthyridin-1-ylmethyl)-phenyl]-benzofuran-2-sulfonic acid (4,5-dimethyl-isoxazol-3-yl)-amide (Compound 49),
3-[2-Chloro-4-(3,5-dipropyl-1,2,4-triazol-1-ylmethyl)-phenyl]-5-methyl-thiophene-2-sulfonic acid (4,5-dimethyl-isoxazol-3-yl)-amide (Compound 50),
3-{2-Chloro-4-[5,7-diethyl-3-(5-methyl-thiophen-2-yl)-2-oxo-2H-1,6-naphthyridin-1-ylmethyl]-phenyl}-5-methyl-thiophene-2-sulfonic acid (5-methylpropyl-isoxazol-3-yl)-amide (Compound 51),
4-[4-(2-Ethyl-5,7-dimethyl-imidazo[4,5-b]pyridin-3-ylmethyl)-phenyl]-3-methyl-isoxazole-5-sulfonic acid (4,5-dimethyl-isoxazol-3-yl)-amide (Compound 52),
3-[2-Chloro-4-(3-isobutyl-6-methoxy-2-methyl-quinolin yloxymethyl)-phenyl]-5-methyl-thiophene-2-sulfonic acid (4,5-dimethyl-isoxazol-3-yl)-amide (Compound 53),
3-[2-Chloro-4-(4-oxo-2-propyl-1,3-diaza-spiro[4.5]dec-1-en-3-ylmethyl)-phenyl]-5-methyl-thiophene-2-sulfonic acid (4-butyl-5-methyl-isoxazol-3-yl)-amide (Compound 54),
3-[2-Chloro-4-(5-phenyl-2-propyl-2H-1,2,4-triazol-3-ylmethyl)-phenyl]-5-methyl-thiophene-2-sulfonic acid (4,5-dimethyl-isoxazol-3-yl)-amide (Compound 55),
4-Methyl-2-[4-(7-oxo-2-propyl-4,5,6,7-tetrahydro-benzimidazol-1-ylmethyl)-phenyl]-pyridine-3-sulfonic acid (4,5-dimethyl-isoxazol-3-yl)-amide (Compound 56),
3-[4-(5,7-Dimethyl-2-oxo-2H-1,6-naphthyridin-1-ylmethyl)-phenyl]-thiophene-2-sulfonic acid (5-methoxy-thiazolo[5,4-b]pyridin-2-yl)-amide (Compound 57),
2-[4-(5,7-Dimethyl-2-oxo-2H-1,6-naphthyridin-1-ylmethyl)-phenyl]-pyridine-3-sulfonic acid (2,4-dimethoxy-pyrimidin-5-yl)-amide (Compound 58),
3-(4-[4,6-Dimethyl-3-(5-methyl-thiophen-2-yl)-pyrazolo[4,3-c]pyridin-1-ylmethyl]-phenyl)-thiophene-2-sulfonic acid (2,4-dimethoxy-pyrimidin-5-yl)-amide (Compound 59),
3-{4-[4,6-Dimethyl-3-(5-methyl-thiophen-2-yl)-pyrazolo[4,3-c]pyridin-1-ylmethyl]-phenyl}-5-methyl-thiophene-2-sulfonic acid isoxazol-3-ylamide (Compound 60),
3-[4-(6-Methoxy-2,3-dimethyl-quinolin-4-yloxymethyl)-phenyl]-thiophene-2-sulfonic acid (5-ethyl-1,3,4-thiadiazol-2-yl)-amide (Compound 61),
3-{4-[3-(3-Chloro-phenyl)-5,7-diethyl-2-oxo-2H-1,6-naphthyridin-1-ylmethyl]-phenyl}-thiophene-2-sulfonic acid (1H-tetrazol-5-yl)-amide (Compound 62),
3-{4-[4,6-Dimethyl-3-(3-trifluoromethyl-phenyl)-pyrazolo[4,3-c]pyridin-1-ylmethyl]-2-hydroxy-phenyl}-5-methyl-thiophene-2-sulfonic acid (4,5-dimethyl-isoxazol-3-yl)-amide (Compound 63),
3-[4-(4,6-Dimethyl-pyrazolo[4,3-c]pyridin-1-ylmethyl)-2-(2-hydroxy-ethyl)-phenyl]-5-methyl-thiophene-2-sulfonic acid (4,5-dimethyl-isoxazol-3-yl)-amide (Compound 64),
3-[4-(3-Chloro-4,6-dimethyl-pyrazolo[4,3-c]pyridin-1-ylmethyl)-2-methoxy-phenyl]-5-methyl-thiophene-2-sulfonic acid (4,5-dimethyl-isoxazol-3-yl)-amide (Compound 65),
3-[4-(3-1,3-Benzodioxol-5-yl-4,6-dimethyl-pyrazolo[4,3-c]pyridin-1-ylmethyl)-2-(2-methoxy-ethoxy)-phenyl]-5-methyl-thiophene-2-sulfonic acid (4,5-dimethyl-isoxazol-3-yl)-amide (Compound 66),
3-[4-[4,6-Dimethyl-3-(5-methyl-thiophen-2-yl)-pyrazolo[4,3-c]pyridin-1-ylmethyl]-2-(2-oxo-pyrrolidin-1-ylmethyl)-phenyl]-5-methyl-thiophene-2-sulfonic acid (4,5-dimethyl-isoxazol-3-yl)-amide (Compound 67),
3-[4-(4,6-Dimethyl-3-phenyl-pyrazolo[4,3-c]pyridin-1-ylmethyl)-2-(3,5-dimethyl-pyrazol-1-ylmethyl)-phenyl]-5-methyl-thiophene-2-sulfonic acid (4,5-dimethyl-isoxazol-3-yl)-amide (Compound 68),
3-[(4-[3-(3-Isobutoxy-phenyl)-4,6-dimethyl-pyrazolo[4,3-c]pyridin-1-ylmethyl]-2-methoxy-phenyl]-4,5-dimethyl-thiophene-2-sulfonic acid (4,5-dimethyl-isoxazol-3-yl)-amide (Compound 69),
3-{(4-[3-(3-Chloro-phenyl)-4,6-dimethyl-pyrazolo[4,3-c]pyridin-1-ylmethyl]-2-ethoxy-phenyl}-5-ethyl-thiophene-2-sulfonic acid (4,5-dimethyl-isoxazol-3-yl)-amide (Compound 70),
3-{4-[4-(4-Methoxy-phenyl)-5,7-dimethyl-2-oxo-2H-1,6-naphthyridin-1-ylmethyl]-2-propoxy-phenyl}-5-methyl-thiophene-2-sulfonic acid (4-chloro-5-methyl-isoxazol-3-yl)-amide (Compound 71),
3-{4-[5,7-Dimethyl-4-(4-methyl-piperazin-1-yl)-2-oxo-2H-1,6-naphthyridin-1-ylmethyl]-2-methoxy-phenyl}-5-methyl-thiophene-2-sulfonic acid (4,5-dimethyl-isoxazol-3-yl)-amide (Compound 72),
3-[4-(3-Methoxy-4,6-dimethyl-pyrazolo[4,3-c]pyridin-1-ylmethyl)-2-(2-methoxy-ethoxy)-phenyl]-5-methyl-thiophene-2-sulfonic acid (4,5-dimethyl-isoxazol-3-yl)-amide (Compound 73),
4-{4-[2-(4,5-Dimethyl-isoxazol-3-ylsulfamoyl)-5-methyl-thiophen-3-yl]-benzyloxy}-6-methyl-2-propyl-nicotinic acid ethyl ester (Compound 74),
4-{4-[2-(4,5-Dimethyl-isoxazol-3-ylsulfamoyl)-5-methyl-thiophen-3-yl]-benzyloxy}-6-methyl-2-propyl-nicotinic acid (Compound 75),
3-{4-[2-(4,5-Dimethyl-isoxazol-3-ylsulfamoyl)-5-methyl-thiophen-3-yl]-benzyl}-2-oxo-2,3-dihydro-1H-benzimidazole-4-carboxylic acid methyl ester (Compound 76),
3-[2-Ethoxymethyl-4-(6-oxo-4-phenyl-2-propyl-6H-pyrimidin-1-ylmethyl)-phenyl]-5-methyl-thiophene-2-sulfonic acid (4,5-dimethyl-isoxazol-3-yl)-amide (Compound 77),
3-[4-(2,4-Dimethyl-7-oxo-6,7-dihydro-5H-pyrido[2,3-d]pyrimidin-8-ylmethyl)-phenyl]-5-methyl-thiophene-2-sulfonic acid (4,5-dimethyl-isoxazol-3-yl)-amide (Compound 78),
3-[2-Ethoxymethyl-4-(4-ethyl-6-oxo-2-propyl-6H-pyrimidin-1-ylmethyl)-phenyl]-5-methyl-thiophene-2-sulfonic acid (4,5-dimethyl-isoxazol-3-yl)-amide (Compound 79),
3-{4-[(3-Cyano-5,7-dimethyl-1,6-naphthyridin-2-ylamino)-methyl]-phenyl}-thiophene-2-sulfonic acid (4,5-dimethyl-isoxazol-3-yl)-amide (Compound 80),
3-{4-[4,6-Dimethyl-3-(5-methyl-thiophen-2-yl)-pyrazolo[4,3-c]pyridin-1-ylmethyl]-2-ethoxymethyl-phenyl}-5-methyl-thiophene-2-sulfonic acid (4,5-dimethyl-isoxazol-3-yl)-amide (Compound 81),
3-{2-Chloro-4-[5,7-diethyl-3-(5-methyl-thiophen-2-yl)-2-oxo-2H-1,6-naphthyridin-1-ylmethyl]-phenyl}-5-methyl-thiophene-2-sulfonic acid (4-isobutyl-5-methyl-isoxazol-3-yl)-amide (Compound 82),
4-{4-[2-(5-Ethyl-4-methyl-isoxazol-3-ylsulfamoyl)-5-methyl-thiophen-3-yl]-benzyloxy}-6-methyl-2-propyl-nicotinic acid ethyl ester (Compound 83),
3-{2,6-Dichloro-4-[4,6-dimethyl-3-(5-methyl-thiophen-2-yl)-pyrazolo[4,3-c]pyridin-1-ylmethyl]-phenyl}-5-methyl-thiophene-2-sulfonic acid (4,5-dimethyl-isoxazol-3-yl)-amide (Compound 84),
3-{2-Chloro-4-[3-methyl-6-thiophen-2-ylmethyl-4-(3-trifluoromethyl-phenyl)-pyrazolo[4,3-c]pyridin-1-ylmethyl]-phenyl}-5-methyl-thiophene-2-sulfonic acid (4-ethyl-5-methyl-isoxazol-3-yl)-amide (Compound 85),
3-[4-(6-Cyclopropylmethyl-3,4-dimethyl-pyrazolo[4,3-c]pyridin-1-ylmethyl)-2-propyl-phenyl]-5-methyl-thiophene-2-sulfonic acid (5-ethyl-4-methyl-isoxazol-3-yl)-amide (Compound 86),
3-(4-{6-[2-(3-Chloro-phenyl)-ethyl]-3,4-dimethyl-pyrazolo[4,3-c]pyridin-1-ylmethyl}-2-hydroxy-phenyl)-5-methyl-thiophene-2-sulfonic acid (4,5-dimethyl-isoxazol-3-yl)-amide (Compound 87),
3-{2-(2-Hydroxy-ethyl)[3-methyl-6-(4-methyl-benzyl)-4-(5-methyl-thiophen-2-yl)-pyrazolo[4,3-c]pyridin-1-ylmethyl]-phenyl}-5-methyl-thiophene-2-sulfonic acid (4,5-dimethyl-isoxazol-3-yl)-amide (Compound 88),
3-[4-(4-Cyclopropylmethyl-6-isobutyl-pyrazolo[4,3-c]pyridin-1-ylmethyl)-2-methoxy-phenyl]-5-methyl-thiophene-2-sulfonic acid (4,5-dimethyl-isoxazol-3-yl)-amide (Compound 89),
3-[4-(3-Cyclopropyl-4,6-dimethyl-pyrazolo[4,3-c]pyridin-1-ylmethyl)-2-fluoro-phenyl]-5-methyl-thiophene-2-sulfonic acid (4,5-dimethyl-isoxazol-3-yl)-amide (Compound 90),
3-[4-[4-(3-Chloro-phenyl)-6-methyl-3-trifluoromethyl-pyrazolo[4,3-c]pyridin-1-ylmethyl]-2-(2-methoxy-ethoxy)-phenyl]-5-methyl-thiophene-2-sulfonic acid (4,5-dimethyl-isoxazol-3-yl)-amide (Compound 91),
3-[4-(3-Chloro-6-methyl-4-propyl-pyrazolo[4,3-c]pyridin-1-ylmethyl)-2-methyl-phenyl]-4,5-dimethyl-thiophene-2-sulfonic acid (4,5-dimethyl-isoxazol-3-yl)-amide (Compound 92),
3-{4-[3-(4-Butoxy-phenyl)-6-methyl-4-trifluoromethyl-pyrazolo[4,3-c]pyridin-1-ylmethyl]-2-chloro-phenyl}-5-ethyl-thiophene-2-sulfonic acid (5-ethyl-4-methyl-isoxazol-3-yl)-amide (Compound 93),
3-[4-(7-Isobutyl-2-oxo-5-phenyl-2H-1,6-naphthyridin-1-ylmethyl)-phenyl]-5-methyl-thiophene-2-sulfonic acid (4,5-dimethyl-isoxazol-3-yl)-amide (Compound 94),
3-[4-(7-Benzyl-2-oxo-5-propyl-2H-1,6-naphthyridin-1-ylmethyl)-2-chloro-phenyl]-5-ethyl-thiophene-2-sulfonic acid (4,5-dimethyl-isoxazol-3-yl)-amide (Compound 95),
3-{2,6-Dichloro-4-[7-cyclopropylmethyl-5-(5-methyl-thiophen-2-yl)-2-oxo-2H-1,6-naphthyridin-1-ylmethyl]-phenyl}-5-methyl-thiophene-2-sulfonic acid (4,5-dimethyl-isoxazol-3-yl)-amide (Compound 96),
3-[4-(4,5-Dimethyl-2-oxo-7-thiophen-2-ylmethyl-2H-1,6-naphthyridin-1-ylmethyl)-phenyl]-5-methyl-thiophene-2-sulfonic acid (4,5-dimethyl-isoxazol-3-yl)-amide (Compound 97),
3-[2-Chloro-4-(7-ethyl-2-oxo-5-thiophen-2-ylmethyl-2H-1,6-naphthyridin-1-ylmethyl)-phenyl]-5-methyl-thiophene-2-sulfonic acid (4,5-dimethyl-isoxazol-3-yl)-amide (Compound 98),
3-[4-(5-Cyclopropylmethyl-7-ethyl-2-oxo-2H-1,6-naphthyridin-1-ylmethyl)-2-methoxymethyl-phenyl]-5-methyl-thiophene-2-sulfonic acid (4,5-dimethyl-isoxazol-3-yl)-amide (Compound 99),
3-[2-Chloro-4-(7-cyclopropylmethyl-2-oxo-4-o-tolyl-5-trifluoromethyl-2H-1,6-naphthyridin-1-ylmethyl)-phenyl]-5-methyl-thiophene-2-sulfonic acid (4,5-dimethyl-isoxazol-3-yl)-amide (Compound 100),
3-{2-Cyclopropylmethoxy-4-[5,7-dimethyl-2-oxo-4-(pyridin-4-yloxy)-2H-1,6-naphthyridin-1-ylmethyl]-phenyl}-5-methyl-thiophene-2-sulfonic acid (4,5-dimethyl-isoxazol-3-yl)-amide (Compound 101),
3-[2-Chloro-4-(5,7-dimethyl-2-oxo-3-pyridin-2-yl-2H-1,6-naphthyridin-1-ylmethyl)phenyl]-5-methyl-thiophene-2-sulfonic acid (4,5-dimethyl-isoxazol-3-yl)-amide (Compound 102),
3-[2-Chloro-4-(5,7-diethyl-4-methyl-2-oxo-3-phenyl-2H-1,6-naphthyridin-1-ylmethyl)-phenyl]-5-methyl-thiophene-2-sulfonic acid (4,5-dimethyl-isoxazol-3-yl)-amide (Compound 103),
(S)-2-({4-[2-(4,5-Dimethyl-isoxazol-3-ylsulfamoyl)-5-methyl-thiophen-3-yl]-3-ethoxymethyl-benzyl}-pentanoyl-amino)-3-methyl-butyric acid, (Compound 104)

### Screen Model for Potency Determination In Vivo

### Animals:

Male SD rats (300-350 g) were anaesthetized with ketamine/xylazine (or ketamine/diazepam) After tracheal intubation; rats were ventilated with room air at a volume of 10 ml/kg b.wt. and respiration rate of 70 strokes/min. Animals were kept warm by means of homeothermic blanket system. The left jugular vein was cannulated for i.v. drug administration and right femoral artery for measurement of systemic blood pressure and heart rate. After stabilization of blood pressure (about 15 min), atropine (0.4 mg/kg i.v.) and mecamylamine (3 mg/kg i.v.) were injected to inhibit autonomic nervous reflexes.

### Potency on AT1 Receptors in Anaesthetized Rats

Blood pressure was elevated some 45 mm Hg by a constant infusion of human Ang II (50 ng/kg/min) during 2.5-3 h. To block all ETA receptors the selective ETA antagonist (ZD1611, 1.5 mg/kg) was given prior to start of the Ang II infusion. When blood pressure 10 had stabilised the compound under study was infused for 30 min at three increasing infusion rates. Blood samples were taken in the beginning and end of each infusion rate for determination of plasma concentration of drug. Likewise, blood samples were taken at intervals during 2 h subsequent to the end of drug infusion. The number of animals per compound were 6-8. Blood pressure was recorded although the experiment. PK/PD-modelling was then used for characterization of the pharmacodynamics (i.e. in vivo potency, efficacy), using the unbound plasma concentration and effect data (blood pressure).

### Potency on ETA Receptors in Anaesthetized Rats

Blood pressure was elevated by a constant infusion of human big ET-1 (0.05 ng/kg/min) during 2.5-3 h. To block all AT1 receptors, Losartan was given prior to start of the bid ET-1 infusion. When blood pressure had stabilised the compound under study was given at increasing infusion rates for 30 min. Blood samples were taken during the beginning and end of each infusion rate for determination of plasma concentration. Likewise, blood samples were taken at intervals during 2 h subsequent to the end of drug infusion. The number of animals per compound were 6-8. Blood pressure was recorded although the experiment. PK/PD-modelling was then used for characterization of the pharmacodynamics (i.e. in vivo potency, efficacy), using the unbound plasma concentration and effect data (blood pressure).

### Screen Model for Potency Determination In Vitro

### AT1 Functional Assay

CHO cell stably over-expressing the full length human AT1 receptor was cultured in DMEM (Gibco) with 10% FCS (Hyclone) and selection maintained using 0.5 mg/ml G418 (Gibco). Cell at 70% confluence were trypsnised, resuspended in media and counted. 20,000 cells/well were plated into black polystyrene 384-well plates with transparent bottoms and allowed 16 hours to adhere. The cells were loaded with Flou-4 (TEFLABS, USA) dye in HBSS (Gibco) for 1 hour and then washed in HBSS and 0.6 μl of diluted compounds in DMSO added to the cells in 30 μd of HBSS in Multimek liquid handling system (Beckman, USA). The cells were then placed in the Fluorometric Imaging Plate Readers (FLIPR, Molecular Devices, USA) and 20 μl of Angiotensin-II peptide as agonist (at EC80) in HBSS was added in the instrument.

The Ca efflux was followed for 2 mins and the maximum height of the peak was measured at various compound concentrations and plotted according to the equation

y=A+((B−A)/1+((C/x)̂D)))

and IC₅₀ estimated wherein
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor. x is the original known x values. y is the original known y values.

### ETA Functional Assay

CHO cell stably over-expressing the full length human ETA receptor was cultured in DMEM (Gibco) with 10% FCS (Hyclone) and selection maintained using 0.5 mg/ml G418 (Gibco). Cell at 70% confluence were trypsnised, resuspended in media and counted. 20,000 cells/well were plated into black polystyrene 384-well plates with transparent bottoms and allowed 16 hours to adhere. The cells were loaded with Flou-4 (TEFLABS, USA) dye in BSS (Gibco) for 1 hour and then washed in HBSS and 0.6 μl of diluted compounds in DMSO added to the cells in 30 μl of HBSS in Multimek liquid handling system (Beckman, USA). The cells were then placed in the Fluorometric Imaging Plate Readers (FLIPR, Molecular Devices, USA) and 20 μl of Endothelin-1 peptide as agonist in HBSS (at EC80) was added in the instrument.

The Ca efflux was followed for 2 mins and the maximum height of the peak was measured at various compound concentrations and plotted according to the equation

y=A+((B−A)/1+((C/x)̂D)))

and IC₅₀ estimated wherein
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor. x is the original known x values. y is the original known y values.

Generally, the potency of the compounds of the present invention ranges from 1 nM to 10 μM for AT1 and 10 nM to 50 μM for ETA: Examples of individual IC₅₀ values are:

| Example | IC₅₀ (AT1) (nM) | IC₅₀ (ETA) (μM) |
|---|---|---|
| 3 | 125 | 2.92 |
| 4 | 29 | 6.2 |
| 6 | 22.9 | 0.562 |
| 7 | 24.6 | 0.814 |
| 8 | 20.8 | 2.87 |
| 15 | 11.4 | 0.503 |
| 18 | 7.98 | 1.43 |
| 21 | 7.47 | 0.464 |
| 22 | 25.2 | 0.454 |
| 23 | 22.1 | 0.248 |
| 24 | 27.4 | 1.89 |
| 26 | 117 | 18.9 |
| 27 | 15.6 | 0.646 |
| 29 | 15.9 | 0.292 |
| 30 | 18.9 | 3.32 |
| 34 | 189 | 7.04 |
| 35 | 27.6 | 2.57 |
| 36 | 58.3 | 0.758 |
| 37 | 153 | 0.407 |
| 38 | 17.2 | 1.04 |
| 39 | 25.1 | 0.781 |

These values show that the selectivity balance between AT1 vs. ETA is good for compounds of the present invention, which is unexpected in relation to prior art.

1: A compound of formula 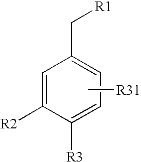
wherein R3 has any of the formulas 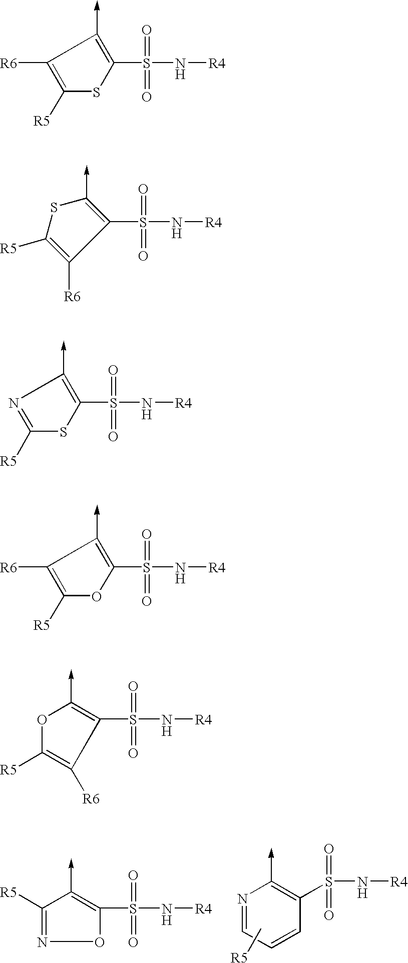
wherein R1 is selected from 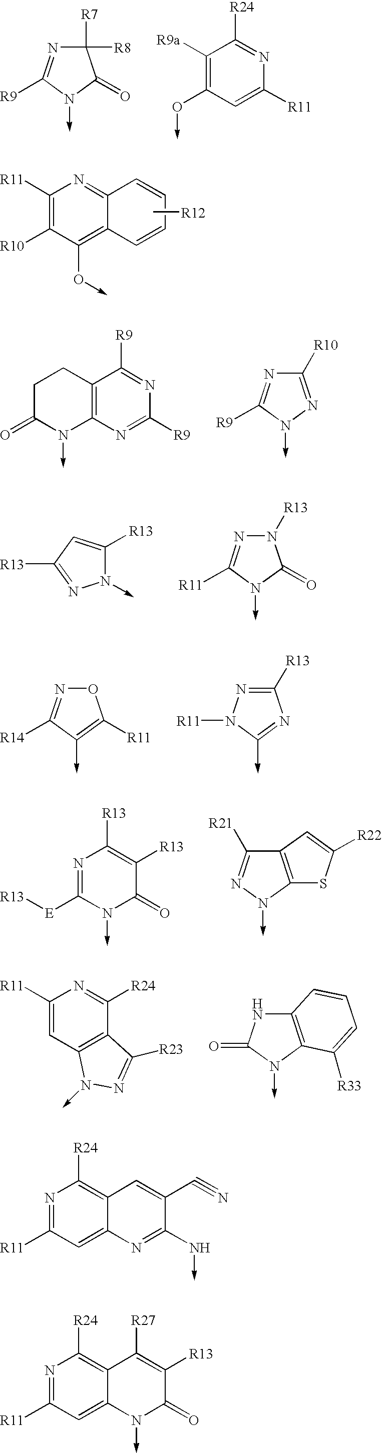 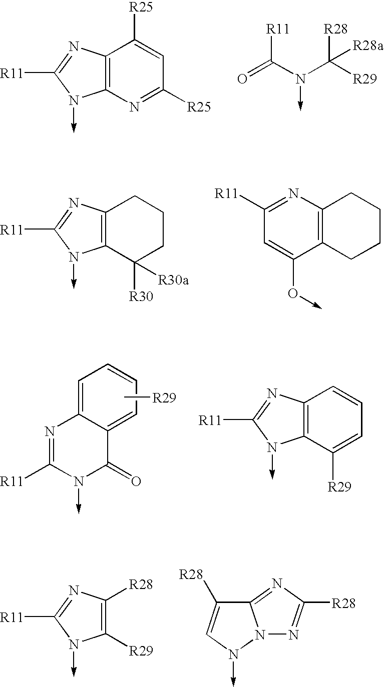
wherein
R2 is each independently hydrogen, halogen, C₁-C₈ alkyl, halo-C₁-C₈ alkyl, C₃-C₈ cycloalkyl, C₂-C₈ alkenyl, C₂-C₈ alkynyl, C₁-C₈ alkyl, C₁-C₈ alkoxy, aryloxy, C₁-C₈ alkoxy-C₁-C₈ alkoxy, cyano, hydroxyl, hydroxy-C₁-C₈ alkyl, nitro, —(CH2)_{W}NR18R19 wherein w is 0, 1, 2, or 3 and R18 and R19 are independently hydrogen, C₁-C₈ alkyl, aryl, aryl-C₁-C₈ alkyl, heteroaryl, heteroaryl-C₁-C₈ alkyl or may together form a five or six membered saturated or unsaturated ring structure optionally containing one to two heteroatoms, selected from oxygen, sulphur or nitrogen and may be optionally substituted by C₁-C₈ alkyl, hydroxyl or oxo;
R4 is a five or six membered mono or bicyclic ring system having one to three heteroatoms, selected from O, N and S such as pyridyl, pyrimidyl, pyrazinyl, pyridazinyl, triazinyl, oxazolyl, isoxazolyl, thiazolyl, thiadiazolyl, isothiazolyl, oxadiazolyl, imidazolyl, triazolyl, tetrazolyl and pyridothiazolyl each of which may optionally be substituted, where appropriate by one or more of the following: hydrogen, halogen, cyano, C₁-C₈ alkyl, C₁-C₈ alkoxy, trifluoromethyl, and —COR32;
R5 and R6 are independently hydrogen, halogen, C₁-C₈ alkyl, —COOR13, —CO—NR18R19, cyano and —NR18R19, or R5 and R6 may together form a five or six membered cycloalkyl, aryl ring or heteroaryl ring structure having one to two heteroatoms, selected from O, N and S, which may be further substituted with C₁-C₈ alkyl, C₁-C₈ alkoxy or hydroxy; wherein R18 and R19 are independently selected from hydrogen, C₁-C₈ alkyl, aryl-C₁-C₈ alkyl, heteroaryl-C₁-C₈ alkyl, (C₃-C₈ cycloalkyl)-C₁-C₈ alkyl or may together form a five or six member saturated ring structure optionally containing one to two heteroatoms selected from O, N and S;
R7 and R8 are each independently C₁-C₈ alkyl, hydroxy-C₁-C₈ alkyl, C₃-C₈ cycloalkyl, hydroxy substituted C₃-C₈ cycloalkyl, C₁-C₈ alkoxy-C₁-C₈ alkyl, hydroxy substituted C₁-C₈ alkoxy-C₁-C₈ alkyl, or R7 and R8 together form a cyclobutyl, cyclopentyl, cyclohexyl, tetrahydrofuranyl or tetrahydropyranyl ring, which may be optionally substituted with one or more hydroxyl groups;
R9 is independently C₁-C₈ alkyl, hydroxy-C₁-C₈ alkyl, hydroxy substituted halo-C₁-C₈ alkyl, C₃-C₈ cycloalkyl, (C₃-C₈ cycloalkyl)-C₁-C₈ alkyl, aryl-C₁-C₈ alkyl, C₁-C₈ alkoxy, hydroxy substituted C₁-C₈ alkoxy, C₁-C₈ alkoxy-C₁-C₈ alkyl, hydroxy substituted C₁-C₈ alkoxy-C₁-C₈ alkyl, C₁-C₈ alkylcarbonyl, arylcarbonyl, carboxy, C₁-C₈ alkoxycarbonyl, and heteroaryl-C₁-C₈ alkyl;
R9a is independently C₁-C₈ alkyl, C₁-C₈ alkoxy-C₁-C₈ alkyl, C₁-C₈ alkylcarbonyl, arylcarbonyl, heteroarylcarbonyl, carboxy, C₁-C₈ alkoxy and —COOR13;
R10 is hydrogen, C₁-C₈ alkyl, (C₃-C₈ cycloalkyl)-C₁-C₈ alkyl, or aryl-C₁-C₈ alkyl;
R11 is independently C₁-C₈-alkyl, C₁-C₈ alkoxy, aryl-C₁-C₈ alkyl, heteroaryl-C₁-C₈ alkyl and (C₃-C₈ cycloalkyl)-C₁-C₈ alkyl;
R12 is hydrogen, halogen, C₁-C₈ alkyl, —COOR17, C₁-C₈ alkyl-C₁-C₈ thioalkyl, C₁-C₈ alkoxy or C₁-C₈ alkoxy-C₁-C₈ alkyl, nitro, NHR24;
R13 independently is hydrogen, C₁-C₈ alkyl, aryl and heteroaryl;
R14 is independently hydrogen, C₁-C₈ alkyl, aryl, NHCOR13 and NR18R19, wherein R18, R19 are independently selected from hydrogen, C₁-C₈ alkyl, aryl-C₁-C₈ alkyl, or may together optionally form a five or six membered saturated ring structure optionally containing one to two heteroatoms selected from O, N and S;
E is a single bond, —(CH₂)— or —S—;
R17 is hydrogen, C₁-C₄ alkyl optionally substituted with an aryl;
R21 is
(a) C₁-C₈ alkyl, halo-C₁-C₈ alkyl, aryl-C₁-C₈ alkyl, or heteroaryl-C₁-C₈ alkyl,
(b) —(CH₂)NR18R19, wherein R18 and R19 are independently hydrogen, C₁-C₈ alkyl, aryl, heteroaryl or may together form a five or six membered saturated or unsaturated ring structure optionally containing having one to two heteroatoms, selected from O, N and S,
(c) aryl, or
(d) heteroaryl;
R22 is
(a) —CO₂R13, —CO₂—C₁-C₈ alkyl, —CO—NR18R19, or
(b) —(CH₂)NR18R19, wherein R18 and R19 are independently hydrogen, C₁-C₈ alkyl, aryl, heteroaryl or may together form a five or six membered saturated or unsaturated ring structure optionally containing having one to two heteroatoms, selected from O, N and S;
R23 is
(a) hydrogen, C₁-C₈ alkyl, aryl, C₁-C₈ alkoxy, halogen, heteroaryl, heteroaryl-C₁-C₈ alkyl, C₃-C₆ cycloalkyl, (C₃-C₈ cycloalkyl)-C₁-C₈ alkyl, —CH₂COOR13, —CH₂CONHR13, or trifluoromethyl, wherein any aryl and heteroaryl residues are optionally substituted with hydrogen, halogen, C₁-C₈ alkyl, C₁-C₈ alkoxy, cyano, trifluoromethyl, nitro, amino, —NHSO₂—R13, —SO₂NHR13, —COOR13, —CONHR13,
or
(b) —(CH₂)NR18R19, wherein R18 and R19 are independently hydrogen, C₁-C₈ alkyl, aryl, heteroaryl or may together form a five or six membered saturated or unsaturated ring structure optionally containing one to two heteroatoms, selected from oxygen, sulphur and nitrogen, aryl and heteroaryl optionally substituted with hydrogen, halogen, C₁-C₈ alkyl, C₁-C₈ alkoxy, cyano, trifluoromethyl, nitro, amino, —NHSO₂—R14, —SO₂NHR24, COOH, —COOR17, or —CONHR14;
R24 is
C₁-C₈ alkyl, C₁-C₈ alkoxy, aryl, heteroaryl, aryl-C₁-C₈ alkyl, heteroaryl-C₁-C₈ alkyl, (C₃-C₈ cycloalkyl)-C₁-C₈ alkyl, and trifluoromethyl,
wherein any aryl and heteroaryl residues are optionally mono- or disubstituted with halogen, C₁-C₈ alkyl, C₁-C₈ alkoxy, cyano, trifluoromethyl, nitro, amino, —NHSO₂—R13, —SO₂NHR13, COOR13, —CONHR13, —(CH₂)NR18R19, wherein R18 and R19 are independently hydrogen, C₁-C₈ alkyl, or may together form a five or six membered saturated or unsaturated ring structure optionally having one to two heteroatoms, selected from O, N and S;
R25 is independently C₁-C₆ alkyl, (C₃-C₆ cycloalkyl)-C₁-C₈ alkyl;
R27 is H, aryl, heteroaryl, C₁-C₈ alkyl, O-aryl, O-heteroaryl, S-aryl, S-heteroaryl or NR18R19, wherein R18 and R19 are independently selected from H, C₁-C₈ alkyl, heteroaryl-C₁-C₈ alkyl, (C₃-C₈ cycloalkyl)-C₁-C₈ alkyl, or may together form a five or six membered saturated ring structure optionally containing one to two heteroatoms selected from O, N and S, wherein aryl and heteroaryl residues are optionally mono- or disubstituted with halogen, C₁-C₈ alkyl, C₁-C₈ alkoxy, trifluoromethyl;
R28 and R28a are each independently hydrogen, halogen, C₁-C₈ alkyl, hydroxy-C₁-C₈ alkyl, C₃-C₈ cycloalkyl, (C₃-C₈ cycloalkyl)-C₁-C₈ alkyl, aryl, heteroaryl, aryl-C₁-C₈ alkyl, C₁-C₈ alkyl-C₁-C₈ thioalkyl, C₁-C₈ alkoxy, C₁-C₈ alkoxy-C₁-C₈ alkyl or R28 and R28a together with the carbon atom to which they are bonded form a C₃-C₈ cycloalkyl ring;
R29 is
(a) —(CH₂)_{W}—COOR17,
(b) —(CH₂)_{W}—(C═O)NR18R19, wherein R18 and R19 are independently selected from H, C₁-C₈ alkyl, aryl, heteroaryl, or R18 and R19 may together form a five or six membered saturated ring structure containing one or two heteroatoms selected from O, N and S, wherein an aryl or heteroaryl residues may be mono- or disubstituted by halogen, C₁-C₈ alkyl, C₁-C₈ alkoxy, and trifluoromethyl,
or
(c) —(CH₂)_{W}—CH₂—OH,
wherein w is 0, 1 or 2;
R30 and R30a are each independently hydrogen, C₁-C₈ alkoxy or together form a carbonyl;
R31 is each independently hydrogen, halogen, C₁-C₈ alkyl, C₁-C₈ alkoxy-C₁-C₈ alkyl, cyano, hydroxy, hydroxy-C₁-C₈ alkyl, C₂-C₈ alkynyl and halo-C₁-C₈ alkyl;
R32 is C₁-C₆ alkyl, C₃-C₆ cycloalkyl, aryl and heteroaryl; and
R33 is C₁-C₈ alkoxycarbonyl;
including pharmaceutically acceptable salts, hydrates, solvates, atropisomers, enantiomers, diastereomers, tautomers, polymorphs and prodrug forms thereof.

2: A compound of formula 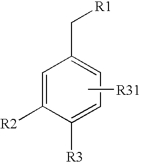
wherein R3 has any of the formulas 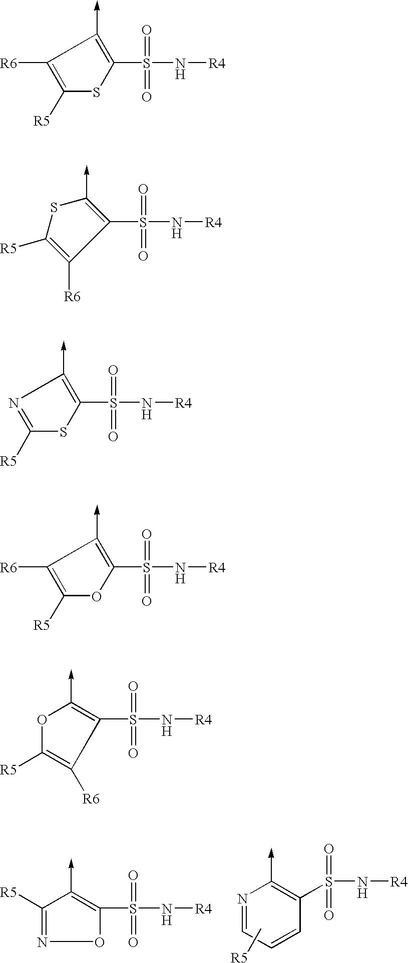
wherein R1 is selected from 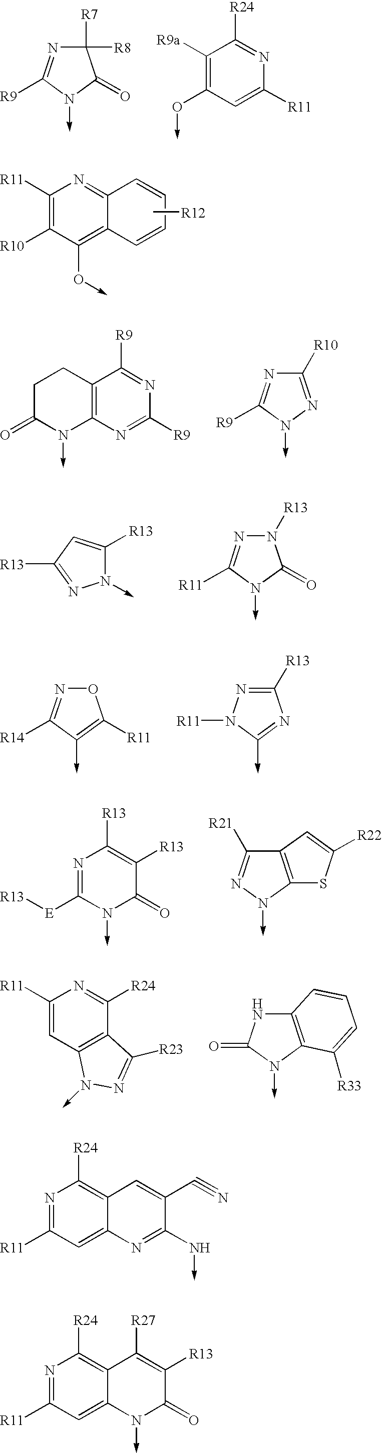 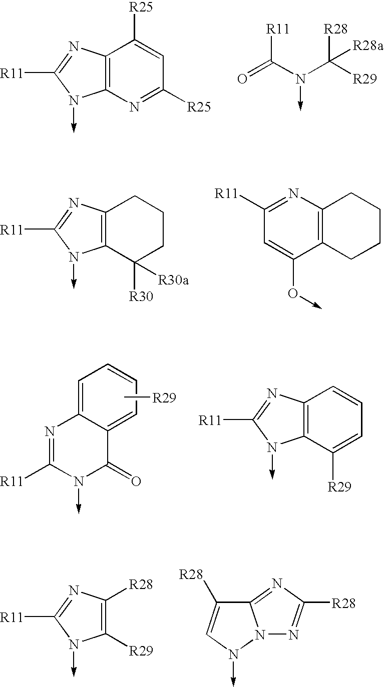
wherein
is each independently hydrogen, halogen, C₁-C₈ alkyl, halo-C₁-alkyl, C₃-C₈ cycloalkyl, C₂-C₈ alkenyl, C₂-C₈ alkynyl, C₁-C₈ alkoxy-C₁-C₈ alkyl, C₁-C₈ alkoxy, aryloxy, C₁-C₈ alkoxy-C₁-C₈ alkoxy, cyano, hydroxyl, hydroxy-C₁-C₈ alkyl, nitro, —(CH2)_{W}NR18R19 wherein w is 0, 1, 2, or 3 and R18 and R19 are independently hydrogen, C₁-C₈ alkyl, aryl, aryl-C₁-C₈ alkyl, heteroaryl, heteroaryl-C₁-C₈ alkyl or may together form a five or six membered saturated or unsaturated ring structure optionally containing one to two heteroatoms, selected from oxygen, sulphur or nitrogen and may be optionally substituted by C₁-C₈ alkyl, hydroxyl or oxo;
R4 is a five or six membered mono or bicyclic ring system having one to three heteroatoms, selected from O, N and S such as pyridyl, pyrimidyl, pyrazinyl, pyridazinyl, triazinyl, oxazolyl, isoxazolyl, thiazolyl, thiadiazolyl, isothiazolyl, oxadiazolyl, imidazolyl, triazolyl, tetrazolyl and pyridothiazolyl each of which may optionally be substituted, where appropriate by one or more of the following: hydrogen, halogen, cyano, C₁-C₈ alkyl, C₁-C₈ alkoxy, trifluoromethyl, and —COR32;
R5 and R6 are independently hydrogen, halogen, C₁-C₈ alkyl, —COOR13, —CO—NR18R19, cyano and —NR18R19, or R5 and R6 may together form a five or six membered cycloalkyl, aryl ring or heteroaryl ring structure having one to two heteroatoms, selected from O, N and S, which may be further substituted with C₁-C₈ alkyl, C₁-C₈ alkoxy or hydroxy; wherein R18 and R19 are independently selected from hydrogen, C₁-C₈ alkyl, aryl-C₁-C₈ alkyl, heteroaryl-C₁-C₈ alkyl, (C₃-C₈ cycloalkyl)-C₁-C₈ alkyl or may together form a five or six member saturated ring structure optionally containing one to two heteroatoms selected from O, N and S;
R7 and R8 are each independently C₁-C₈ alkyl, hydroxy-C₁-C₈ alkyl, C₃-C₈ cycloalkyl, hydroxy substituted C₃-C₈ cycloalkyl, C₁-C₈ alkoxy-C₁-C₈ alkyl, hydroxy substituted C₁-C₈ alkoxy-C₁-C₈ alkyl, or R7 and R8 together form a cyclobutyl, cyclopentyl, cyclohexyl, tetrahydrofuranyl or tetrahydropyranyl ring, which may be optionally substituted with one or more hydroxyl groups;
R9 is independently C₁-C₈ alkyl, hydroxy-C₁-C₈ alkyl, hydroxy substituted halo-C₁-C₈ alkyl, C₃-C₈ cycloalkyl, (C₃-C₈ cycloalkyl)-C₁-C₈ alkyl, aryl-C₁-C₈ alkyl, C₁-C₈ alkoxy, hydroxy substituted C₁-C₈ alkoxy, C₁-C₈ alkoxy-C₁-C₈ alkyl, hydroxy substituted C₁-C₈ alkoxy-C₁-C₈ alkyl, C₁-C₈ alkylcarbonyl, arylcarbonyl, carboxy, C₁-C₈ alkoxycarbonyl, and heteroaryl-C₁-C₈ alkyl;
R9a is independently C₁-C₈ alkyl, C₁-C₈ alkoxy-C₁-C₈ alkyl, C₁-C₈ alkylcarbonyl, arylcarbonyl, heteroarylcarbonyl, carboxy, C₁-C₈ alkoxy and —COOR13;
R10 is hydrogen, C₁-C₈ alkyl, (C₃-C₈ cycloalkyl)-C₁-C₈ alkyl, or aryl-C₁-C₈ alkyl;
R11 is independently C₁-C₈-alkyl, C₁-C₈ alkoxy, aryl-C₁-C₈ alkyl, heteroaryl-C₁-C₈ alkyl and (C₃-C₈ cycloalkyl)-C₁-C₈ alkyl;
R12 is hydrogen, halogen, C₁-C₈ alkyl, —COOR17, C₁-C₈ alkyl-C₁-C₈ thioalkyl, C₁-C₈ alkoxy or C₁-C₈ alkoxy-C₁-C₈ alkyl, nitro, NHR24;
R13 independently is hydrogen, C₁-C₈ alkyl, aryl and heteroaryl;
R14 is independently hydrogen, C₁-C₈ alkyl, aryl, NHCOR13 and NR18R19, wherein R18, R19 are independently selected from hydrogen, C₁-C₈ alkyl, aryl-C₁-C₈ alkyl, or may together optionally form a five or six membered saturated ring structure optionally containing one to two heteroatoms selected from O, N and S;
E is a single bond, —(CH₂)— or —S—;
R17 is hydrogen, C₁-C₄ alkyl optionally substituted with an aryl;
R21 is
(e) C₁-C₈ alkyl, halo-C₁-C₈ alkyl, aryl-C₁-C₈ alkyl, or heteroaryl-C₁-C₈ alkyl,
(f) —(CH₂)NR18R19, wherein R18 and R19 are independently hydrogen, C₁-C₈ alkyl, aryl, heteroaryl or may together form a five or six membered saturated or unsaturated ring structure optionally containing having one to two heteroatoms, selected from O, N and S,
(g) Aryl or
(h) heteroaryl;
R22 is
(a) —CO₂R13, —CO₂—C₁-C₈ alkyl, —CO—NR18R19 or
(b) —(CH₂)NR18R19, wherein R18 and R19 are independently hydrogen, C₁-C₈ alkyl, aryl, heteroaryl or may together form a five or six membered saturated or unsaturated ring structure optionally containing having one to two heteroatoms, selected from O, N and S;
R23 is
(a) hydrogen, C₁-C₈ alkyl, aryl, C₁-C₈ alkoxy, halogen, heteroaryl, heteroaryl-C₁-C₈ alkyl, C₃-C₆ cycloalkyl, (C₃-C₈ cycloalkyl)-C₁-C₈ alkyl, —CH₂COOR13, —CH₂CONHR13 or trifluoromethyl, wherein any aryl and heteroaryl residues are optionally substituted with hydrogen, halogen, C₁-C₈ alkyl, C₁-C₈ alkoxy, cyano, trifluoromethyl, nitro, amino, —NHSO₂—R13, —SO₂NHR13, —COOR13, —CONHR13,
or
(b) —(CH₂)NR18R19, wherein R18 and R19 are independently hydrogen, C₁-C₈ alkyl, aryl, heteroaryl or may together form a five or six membered saturated or unsaturated ring structure optionally containing one to two heteroatoms, selected from oxygen, sulphur and nitrogen, aryl and heteroaryl optionally substituted with hydrogen, halogen, C₁-C₈ alkyl, C₁-C₈ alkoxy, cyano, trifluoromethyl, nitro, amino, —NHSO₂—R14, —SO₂NHR24, COOH, —COOR17 or —CONHR14;
R24 is
C₁-C₈ alkyl, C₁-C₈ alkoxy, aryl, heteroaryl, aryl-C₁-C₈ alkyl, heteroaryl-C₁-C₈ alkyl, (C₃-C₈ cycloalkyl)-C₁-C₈ alkyl, and trifluoromethyl,
wherein any aryl and heteroaryl residues are optionally mono- or disubstituted with halogen, C₁-C₈ alkyl, C₁-C₈ alkoxy, cyano, trifluoromethyl, nitro, amino, —NHSO₂—R13, —SO₂NHR13, COOR13, —CONHR13, —(CH₂)NR18R19, wherein R18 and R19 are independently hydrogen, C₁-C₈ alkyl, or may together form a five or six membered saturated or unsaturated ring structure optionally having one to two heteroatoms, selected from O, N and S;
R25 is independently C₁-C₆ alkyl, (C₃-C₆ cycloalkyl)-C₁-C₈ alkyl;
R27 is H, aryl, heteroaryl, C₁-C₈ alkyl, C₁-C₈ alkoxy, O-aryl, O-heteroaryl, S-aryl, S-heteroaryl or NR18R19, wherein R18 and R19 are independently selected from H, C₁-C₈ alkyl, heteroaryl-C₁-C₈ alkyl, (C₃-C₈ cycloalkyl)-C₁-C₈ alkyl, or may together form a five or six membered saturated ring structure optionally containing one to two heteroatoms selected from O, N and S, wherein aryl and heteroaryl residues are optionally mono- or disubstituted with halogen, C₁-C₈ alkyl, C₁-C₈ alkoxy, trifluoromethyl;
R28 and R28a are each independently hydrogen, halogen, C₁-C₈ alkyl, hydroxy-C₁-C₈ alkyl, C₃-C₈ cycloalkyl, (C₃-C₈ cycloalkyl)-C₁-C₈ alkyl, aryl, heteroaryl, aryl-C₁-C₈ alkyl, C₁-C₈ alkyl-C₁-C₈ thioalkyl, C₁-C₈ alkoxy, C₁-C₈ alkoxy-C₁-C₈ alkyl or R28 and R28a together with the carbon atom to which they are bonded form a C₃-C₈ cycloalkyl ring;
R29 is
(d) —(CH₂)_{W}—COOR17,
(e) —(CH₂)_{W}—(C═O)NR18R19, wherein R18 and R19 are independently selected from H, C₁-C₈ alkyl, aryl, heteroaryl, or R18 and R19 may together form a five or six membered saturated ring structure containing one or two heteroatoms selected from O, N and S, wherein an aryl or heteroaryl residues may be mono- or disubstituted by halogen, C₁-C₈ alkyl, C₁-C₈ alkoxy, and trifluoromethyl
or
(f) —(CH₂)_{W}—CH₂—OH,
wherein w is 0, 1 or 2;
R30 and R30a are each independently hydrogen, C₁-C₈ alkoxy or together form a carbonyl;
R31 is each independently hydrogen, halogen, C₁-C₈ alkyl, C₁-C₈ alkoxy-C₁-C₈ alkyl, cyano, hydroxy, hydroxy-C₁-C₈ alkyl, C₂-C₈ alkynyl and halo-C₁-C₈ alkyl;
R32 is C₁-C₆ alkyl, C₃-C₆ cycloalkyl, aryl and heteroaryl; and
R33 is C₁-C₈ alkoxycarbonyl;
including pharmaceutically acceptable salts thereof.

3: The compound according to any one of claims 1 or 2, wherein
R3 has any of the formulas 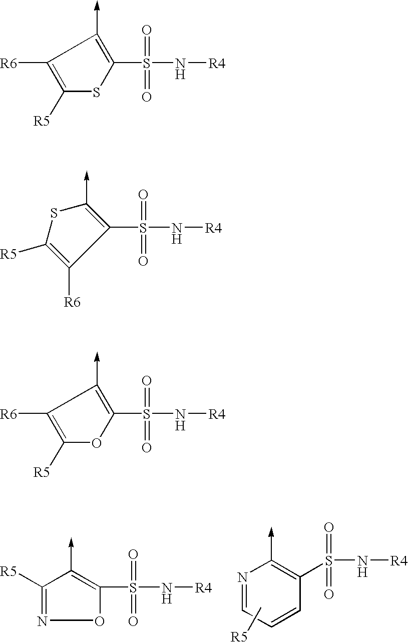
wherein R1 is selected from 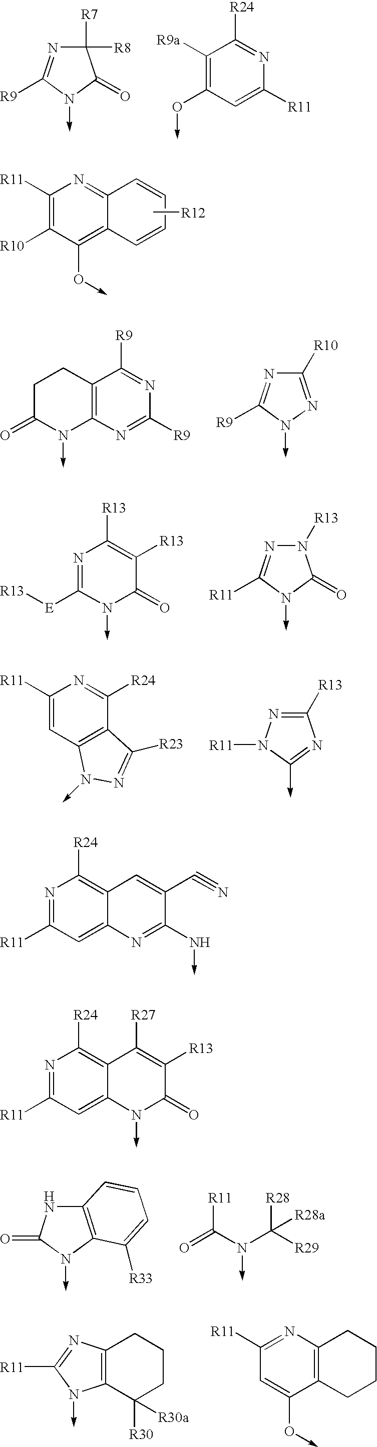 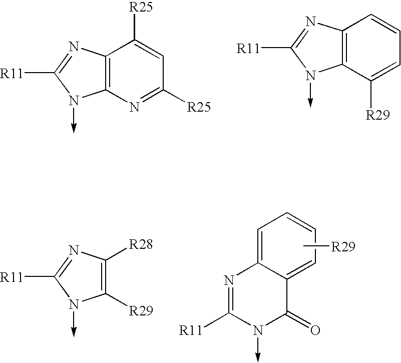
wherein
R2 is each independently hydrogen, halogen, C₁-C₈ alkyl, C₁-C₈ alkoxy-C₁-C₈ alkyl, C₁-C₈ alkoxy, C₁-C₈ alkoxy-C₁-C₈ alkoxy, hydroxyl, hydroxy-C₁-C₈ alkyl, —(CH2)_{W}NR18R19 wherein w is 1 and R18 and R19 form a five or six membered saturated or unsaturated ring structure optionally containing one to two heteroatoms, selected from oxygen, sulphur or nitrogen and may be optionally substituted by C₁-C₈ alkyl or oxo;
R4 is a five or six membered mono or bicyclic ring system having one to three heteroatoms, selected from O, N and S such as pyridyl, pyrimidyl, pyrazinyl, pyridazinyl, isoxazolyl, thiazolyl, thiadiazolyl, tetrazolyl and pyridothiazolyl each of which may optionally be substituted, where appropriate by one or more of the following: hydrogen, halogen, C₁-C₈ alkyl, C₁-C₈ alkoxy, R5 and R6 are independently hydrogen, C₁-C₈ alkyl, or R5 and R6 may together form a five or six membered cycloalkyl or aryl ring, which may be further substituted with C₁-C₈ alkyl;
R7 and R8 form together cyclobutyl, cyclopentyl, or cyclohexyl;
R9 is C₁-C₈ alkyl;
R9a is independently C₁-C₈ alkyl, C₁-C₈ alkoxy-C₁-C₈ alkyl, C₁-C₈ alkylcarbonyl, arylcarbonyl, heteroarylcarbonyl, carboxy and —COOR13;
R10 is hydrogen, C₁-C₈ alkyl or (C₃-C₈ cycloalkyl)-C₁-C₈ alkyl;
R11 is independently C₁-C₈-alkyl, C₁-C₈ alkoxy, aryl-C₁-C₈ alkyl, heteroaryl-C₁-C₈ alkyl and (C₃-C₈ cycloalkyl)-C₁-C₈ alkyl;
R12 is hydrogen, C₁-C₈ alkoxy or —COOR17;
R13 is hydrogen, C₁-C₈ alkyl, aryl or heteroaryl;
E is a single bond;
R17 is hydrogen;
R23 is hydrogen, C₁-C₈ alkyl, aryl, C₁-C₈ alkoxy, halogen, heteroaryl, heteroaryl-C₁-C₈ alkyl, C₃-C₆ cycloalkyl, or trifluoromethyl, wherein any aryl and heteroaryl residues are optionally substituted with hydrogen, halogen, C₁-C₈ alkyl, C₁-C₈ alkoxy, trifluoromethyl;
R24 is C₁-C₈ alkyl, aryl, heteroaryl, aryl-C₁-C₈ alkyl, heteroaryl-C₁-C₈ alkyl, (C₃-C₈ cycloalkyl)-C₁-C₈ alkyl, and trifluoromethyl, wherein any aryl and heteroaryl residues are optionally mono- or disubstituted with halogen, C₁-C₈ alkyl, C₁-C₈ alkoxy or trifluoromethyl;
R25 is C₁-C₆ alkyl and
R27 is H, aryl, heteroaryl, C₁-C₈ alkyl, C₁-C₈ alkoxy, O-aryl, O-heteroaryl, S-aryl, or NR18R19, wherein R18 and R19 form a five or six membered saturated ring structure optionally containing one to two heteroatoms selected from O, N and S, which may be further substituted with C₁-C₈ alkyl;
R28 and R28a are each independently hydrogen, halogen or C₁-C₈ alkyl;
R29 is —COOH;
R30 and R30a together form a carbonyl;
R31 is halogen and
R33 is C₁-C₈ alkoxycarbonyl.

4: The compound according to claim 1, which is selected from:
3-[4-(2-Butyl-4-oxo-1,3-diaza-spiro[4.4]non-1-en-3-ylmethyl)-2-ethoxymethyl-phenyl]-5-methyl-thiophene-2-sulphonic acid (4,5-dimethyl-isoxazol-3-yl)-amide (Compound 1),
3-[4-(6-Ethyl-4-methyl-3-phenyl-pyrazolo[4,3-c]pyridin-1-ylmethyl)-phenyl]-thiophene-2-sulphonic acid(4,5-dimethyl-isoxazol-3-yl)amide (Compound 2),
3-[4-(5,7-diethyl-2-oxo-4-phenylsulphanyl-2H-[1,6]naphthyridin-1-ylmethyl)-phenyl]-thiophene-2-sulphonic acid(4,5-dimethyl-isoxazol-3-yl)-amide (Compound 3),
3-[4-(3-Benzoyl-6-ethyl-2-methyl-pyridin-4-yloxymethyl)-phenyl]-thiophene-2-sulphonic acid (4,5-dimethyl-isoxazol-3-yl)-amide (Compound 4),
3-[4-(5,7-Diethyl-2-oxo-2H-[1,6]naphthyridin-1-ylmethyl)-phenyl]-thiophene-2-sulphonic acid(4,5-dimethyl-isoxazol-3-yl)-amide (Compound 5),
3-[2-Ethoxymethyl-4-(6-ethyl-3,4-dimethyl-pyrazolo[4,3-c]pyridine-1-ylmethyl)-phenyl]-5-methyl-thiophene-2-sulphonic acid(4,5-dimethyl-isoxazol-3-yl)-amide (Compound 6),
3-[4-(5,7-Diethyl-2-oxo-2H-[1,6]naphthyridin-1ylmethyl)-phenyl]-5-methyl-thiophene-2-sulphonic acid(4,5 dimethyl-isoxazol-3-yl)-amide (Compound 7),
3-[4-(5,7-diethyl-2-oxo-4-phenoxy-2H-[1,6]naphthyridin-1ylmethyl)-phenyl]-thiophene-2-sulphonic acid(4,5-dimethyl-isoxazol-3-yl)-amide (Compound 8),
3-[2-Ethoxymethyl-4-(6-ethyl-4-methyl-3-phenyl-pyrazolo[4,3-c]pyridine-1-ylmethyl)-phenyl]-5-methyl-thiophene-2-sulphonic acid(4,5-dimethyl-isoxazol-3-yl)-(2-methoxy-ethoxymethyl)-amide (Compound 9),
3-[4-(2-Methyl-quinolin-4-yloxymethyl)-phenyl]-thiophene-2-sulphonic acid(4,5-dimethyl-isoxazol-3-yl)-amide (Compound 10),
3-[4-(5,7-Diethyl-2-oxo-2H-[1,6]naphthyridin-1-ylmethyl)-2-ethoxymethyl-phenyl]-5-methyl-thiophene-2-sulphonic acid-(4,5-dimethyl-isoxazol-3-yl)-amide (Compound 11),
3-[4-(3-Acetyl-2,6-dimethyl-pyridine-4-yloxymethyl)-2-ethoxymethyl-phenyl]-5-methyl-thiophene-2-sulphonic acid(4,5-dimethyl-isoxazol-3-yl)-amide (Compound 12),
3-[4-(4,6-Dimethyl-3-para-tolyl-pyrazolo[4,3-c] pyridin-1-ylmethyl)-2-ethoxymethyl-phenyl]-5-methyl-thiophene-2-sulphonic acid (4,5-dimethyl-isoxazol-3-yl)-amide (Compound 13),
3-[4-(4,6-Dimethyl-3-thiophene-2-yl-pyrazolo[4,3-c] pyridin-1-ylmethyl)-2-ethoxymethyl-phenyl]-5-methyl-thiophene-2-sulphonic acid (4,5-dimethyl-isoxazol-3-yl)-amide (Compound 14),
3-{4-[3-(3,5-Dimethyl-pyrazol-1-ylmethyl)-4,6-dimethyl-pyrazolo[4,3-c]pyridin-1-ylmethyl]2-ethoxy methyl-phenyl}-5-methyl-thiophene-2-sulphonic acid (4,5-dimethyl-isoxazol-3-yl)-amide (Compound 15),
3-[2-Methoxymethyl-4-(4,5,7-trimethyl-2-oxo-2H-[1,6]naphthyridin-1-ylmethyl)-phenyl]-5-methyl-thiophene-2-sulphonic acid (4,5-dimethoxy-isoxazol-3-yl)-amide (Compound 16),
3-[4-(6-Ethyl-3,4-dimethyl-pyrazolo[4,3-c]pyridin-1-ylmethyl)-2-methyl-phenyl]-5-methyl-thiophene-2-sulphonic acid (4,5-dimethyl-isoxazol-3-yl)-amide (Compound 17),
3-(4,6-Dimethyl-3-phenyl-pyrazolo[4,3-c]pyridin-1-ylmethyl)-2-methyl-phenyl]-5-methyl-thiophene-2-sulphonic acid (4,5-dimethyl-isoxazol-3-yl)-amide (Compound 18),
3-[4-(5,7-Diethyl-2-oxo-2H-[1,6]naphthyridin-1-ylmethyl)-2-methyl-phenyl]-5-methyl-thiophene-2-sulphonic acid(4,5-dimethyl-isoxazol-3yl)-(2-methoxy-ethoxymethyl)amide (Compound 19),
4-{4-[2-(4,5-dimethyl-isoxazol-3-yl sulfamoyl)-5-methyl-thiophen-3-yl]-3-methoxymethyl-benzyloxy}-2-ethyl-quinoline-6-carboxylic acid (Compound 20),
3-[4-(4,6-dimethyl-3-thiophen-2-yl-pyrazolo[4,3-c]pyridin-1-ylmethyl)-2-methoxymethyl-phenyl]-5-methyl-thiophene-2-sulfonic acid (4,5-dimethyl-isoxazol-3-yl)-amide (Compound 21),
3-[2-Ethoxymethyl-4-(6-ethyl-4-methyl-3-thiophen-2-yl-pyrazolo [4,3-c] pyridin-1-ylmethyl)-phenyl]-5-methyl-thiophene-2-sulfonic acid (4,5-dimethyl-isoxazol-3-yl)-amide (Compound 22),
3-{2-Ethoxymethyl-4-[6-ethyl-3-(4-methoxy-phenyl)-4-methyl-pyrazolo[4,3-c] pyridin-1-ylmethyl]-phenyl}-5-methyl-thiophene-2-sulfonic acid (4,5-dimethyl-isoxazol-3-yl)-amide (Compound 23),
3-{4-[6-Ethyl-3-(4-methoxy-phenyl)-4-methyl-pyrazolo [4,3-c] pyridin-1-ylmethyl]-2-methyl-phenyl}-5-methyl-thiophene-2-sulfonic acid (4,5-dimethyl-isoxazol-3-yl)-amide (Compound 24),
2-[4-(6-Ethyl-4-methyl-3-phenyl-pyrazolo [4,3-c] pyridin-1-ylmethyl)-phenyl]-5-methyl-thiophene-3-sulfonic acid (5-methyl-isoxazol-3-yl)-amide (Compound 25),
3-{4-[2-(3,4-dimethyl-isoxazol-5-ylsulfamoyl)-5-methyl-thiophen-3-yl]-benzyl}-2-ethoxy-3H-benzoimidazole-4-carboxylic acid (Compound 26),
3-[2-Ethoxymethyl-4-(6-ethyl-4-methyl-3-thiophen-2-yl-pyrazolo [4,3-c] pyridin-1-ylmethyl)-phenyl]-5-methyl-thiophene-2-sulfonic acid (3,4-dimethyl-isoxazol-5-yl)-amide (Compound 27),
3-[4-(5,7-diethyl-2-oxo-2H-[1,6] naphthyridin-1-ylmethyl)-2-methyl-phenyl]-5-Propyl-thiophene-2-sulfonic acid (4,5-dimethyl-isoxazol-3-yl)-amide (Compound 28),
3-[4-(5,7-dimethyl-2-oxo-3-phenyl-2H-[1,6] naphthyridin-1-ylmethyl)-2-methyl-phenyl]-5-methyl-thiophene-2-sulfonic acid (4,5-dimethyl-isoxazol-3-yl)-amide (Compound 29),
3-[4-(5,7-diethyl-2-oxo-2H-[1,6] naphthyridin-1-ylmethyl)-2-methyl-phenyl]-5-methyl-thiophene-2-sulfonic acid (3,4-dimethyl-isoxazol-5-yl)-amide (Compound 30),
5-Methyl-3-[2-methyl-4-(3-methyl-5-oxo-1-phenyl-1,5-dihydro-[1, 2, 4] triazol-4-ylmethyl)-phenyl]-thiophene-2-sulfonic acid (4,5-dimethyl-isoxazol-3-yl)-amide (Compound 31),
5-Methyl-3-[2-methyl-4-(5-oxo-3-propyl-1-pyridin-2-yl-1,5-dihydro-[1, 2, 4] triazol-4-ylmethyl)-phenyl]-thiophene-2-sulfonic acid (4,5-dimethyl-isoxazol-3-yl)-amide (Compound 32),
3-[4-(5-Oxo-3-propyl-1-pyridin-2-yl-1,5-dihydro-[1, 2, 4] triazol-4-ylmethyl)-phenyl]-thiophene-2-sulfonic acid (4,5-dimethyl-thiazol-2-yl)-amide (Compound 33),
3-[4-(6-Ethyl-4-methyl-3-phenyl-pyrazolo[4,3-c]pyridin-1-ylmethyl)-phenyl]-thiophene-2-sulfonic acid isobutoxycarbamoyl-(3-methoxy-5-methyl-pyrazin-2-yl)-amide (Compound 34),
3-[4-(5,7-Diethyl-2-oxo-2H-[1,6]naphthyridin-1-ylmethyl)-2-fluoro-phenyl]-5-methyl-thiophene-2-sulfonic acid (4,5-dimethyl-isoxazol-3-yl)-amide (Compound 35),
3-[4-(5,7-Dimethyl-2-oxo-2H-[1,6]naphthyridin-1-ylmethyl)-2-isobutoxy-phenyl]-5-methyl-thiophene-2-sulfonic acid (4,5-dimethyl-isoxazol-3-yl)-amide (Compound 36),
3-{4-[3-(4-Chloro-phenyl)-4,6-dimethyl-pyrazolo [4,3-c] pyridin-1-ylmethyl]-2-methyl-phenyl}-5-methyl-thiophene-2-sulfonic acid (4,5-dimethyl-isoxazol-3-yl)-amide (Compound 37),
3-[4-(3,4-Diethyl-6-methyl-pyrazolo[4,3-c]pyridin-1-ylmethyl)-2-methoxymethyl-phenyl]-5-methyl-thiophene-2-sulfonic acid (4,5-dimethyl-isoxazol-3-yl)-amide (Compound 38),
3-[4-(4-Ethoxy-5,7-diethyl-2-oxo-2H-[1,6]naphthyridin-1-ylmethyl)-2-methyl-phenyl]-5-methyl-thiophene-2-sulfonic acid (4,5-dimethyl-isoxazol-3-yl)-amide (Compound 39),
3-[4-(5,7-Dimethyl-2-oxo-2H-1,6-naphthyridin-1-ylmethyl)-phenyl]-5-methyl-furan-2-sulfonic acid (4,5-dimethyl-isoxazol-3-yl)-amide (Compound 40),
5-Methyl-3-[4-(7-oxo-2-propyl-4,5,6,7-tetrahydro-benzimidazol-1-ylmethyl)-phenyl]-thiophene-2-sulfonic acid (4-ethyl-5-methyl-isoxazol-3-yl)-amide (Compound 41),
3-{4-[2-(4,5-Dimethyl-isoxazol-3-yl sulfamoyl)-5-methyl-thiophen-3-yl]-benzyl}-4-oxo-2-propyl-3,4-dihydro-quinazoline-5-carboxylic acid (Compound 42),
5-Methyl-3-[4-(3,4,5,7-tetramethyl-2-oxo-2H-1,6-naphthyridin-1-ylmethyl)-phenyl]-thiophene-2-sulfonic acid (4,5-dimethyl-isoxazol-3-yl)-amide (Compound 43),
2-[4-(4,5-Diethyl-3,7-dimethyl-2-oxo-2H-1,6-naphthyridin-1-ylmethyl)-phenyl]-pyridine-3-sulfonic acid (4,5-dimethyl-isoxazol-3-yl)-amide (Compound 44),
2-Butyl-5-chloro-3-{4-[5-(4,5-dimethyl-isoxazol-3-ylsulfamoyl)-3-methyl-isoxazol-4-yl]-benzyl}-3H-imidazole-4-carboxylic acid (Compound 45),
3-[4-(2-Methyl-5,6,7,8-tetrahydro-quinolin-4-yloxymethyl)-phenyl]-benzo[b]thiophene-2-sulfonic acid (4,5-dimethyl-isoxazol-3-yl)-amide (Compound 46),
3-[4-(6-Ethyl-4-methyl-3-phenyl-pyrazolo[4,3-c]pyridin-1-ylmethyl)-phenyl]-benzo[b]thiophene-2-sulfonic acid (4,5-dimethyl-isoxazol-3-yl)-amide (Compound 47),
3-[4-(5,7-Dimethyl-2-oxo-2H-1,6-naphthyridin-1-ylmethyl)-phenyl]-4,5,6,7-tetrahydro-enzo[b]thiophene-2-sulfonic acid (4,5-dimethyl-isoxazol-3-yl)-amide (Compound 48),
3-[2-Chloro-4-(5,7-dimethyl-2-oxo-2H-1,6-naphthyridin-1-ylmethyl)-phenyl]-benzofuran-2-sulfonic acid (4,5-dimethyl-isoxazol-3-yl)-amide (Compound 49),
3-[2-Chloro-4-(3,5-dipropyl-1,2,4-triazol-1-ylmethyl)-phenyl]-5-methyl-thiophene-2-sulfonic acid (4,5-dimethyl-isoxazol-3-yl)-amide (Compound 50),
3-{2-Chloro-4-[5,7-diethyl-3-(5-methyl-thiophen-2-yl)-2-oxo-2H-1,6-naphthyridin-1-ylmethyl]-phenyl}-5-methyl-thiophene-2-sulfonic acid
(5-methyl-4-propyl-isoxazol-3-yl)-amide (Compound 51), 4-[4-(2-Ethyl-5,7-dimethyl-imidazo[4,5-b]pyridin-3-ylmethyl)-phenyl]-3-methyl-isoxazole-5-sulfonic acid (4,5-dimethyl-isoxazol-3-yl)-amide (Compound 52),
3-[2-Chloro-4-(3-isobutyl-6-methoxy-2-methyl-quinolin-4-yloxymethyl)-phenyl]-5-methyl-thiophene-2-sulfonic acid (4,5-dimethyl-isoxazol-3-yl)-amide (Compound 53),
3-[2-Chloro-4-(4-oxo-2-propyl-1,3-diaza-spiro[4.5]dec-1-en-3-ylmethyl)-phenyl]-5-methyl-thiophene-2-sulfonic acid (4-butyl-5-methyl-isoxazol-3-yl)-amide (Compound 54),
3-[2-Chloro-4-(5-phenyl-2-propyl-2H-1,2,4-triazol-3-ylmethyl)-phenyl]-5-methyl-thiophene-2-sulfonic acid (4,5-dimethyl-isoxazol-3-yl)-amide (Compound 55),
4-Methyl-2-[4-(7-oxo-2-propyl-4,5,6,7-tetrahydro-benzimidazol-1-ylmethyl)-phenyl]-pyridine-3-sulfonic acid (4,5-dimethyl-isoxazol-3-yl)-amide (Compound 56),
3-[4-(5,7-Dimethyl-2-oxo-2H-1,6-naphthyridin-1-ylmethyl)-phenyl]-thiophene-2-sulfonic acid (5-methoxy-thiazolo[5,4-b]pyridin-2-yl)-amide (Compound 57),
2-[4-(5,7-Dimethyl-2-oxo-2H-1,6-naphthyridin-1-ylmethyl)-phenyl]-pyridine-3-sulfonic acid (2,4-dimethoxy-pyrimidin-5-yl)-amide (Compound 58),
3-{4-[4,6-Dimethyl-3-(5-methyl-thiophen-2-yl)-pyrazolo[4,3-c]pyridin-1-ylmethyl]-phenyl}-thiophene-2-sulfonic acid (2,4-dimethoxy-pyrimidin-5-yl)-amide (Compound 59),
3-{4-[4,6-Dimethyl-3-(5-methyl-thiophen-2-yl)-pyrazolo[4,3-c]pyridin-1-ylmethyl]-phenyl}-5-methyl-thiophene-2-sulfonic acid isoxazol-3-ylamide (Compound 60),
3-[4-(6-Methoxy-2,3-dimethyl-quinolin-4-yloxymethyl)-phenyl]-thiophene-2-sulfonic acid (5-ethyl-1,3,4-thiadiazol-2-yl)-amide (Compound 61),
3-{4-[3-(3-Chloro-phenyl)-5,7-diethyl-2-oxo-2H-1,6-naphthyridin-1-ylmethyl]-phenyl}-thiophene-2-sulfonic acid (1H-tetrazol-5-yl)-amide (Compound 62),
3-{4-[4,6-Dimethyl-3-(3-trifluoromethyl-phenyl)-pyrazolo[4,3-c]pyridin-1-ylmethyl]-2-hydroxy-phenyl}-5-methyl-thiophene-2-sulfonic acid (4,5-dimethyl-isoxazol-3-yl)-amide (Compound 63),
3-[4-(4,6-Dimethyl-pyrazolo[4,3-c]pyridin-1-ylmethyl)-2-(2-hydroxy-ethyl)-phenyl]-5-methyl-thiophene-2-sulfonic acid (4,5-dimethyl-isoxazol-3-yl)-amide (Compound 64),
3-[4-(3-Chloro-4,6-dimethyl-pyrazolo[4,3-c]pyridin-1-ylmethyl)-2-methoxy-phenyl]-5-methyl-thiophene-2-sulfonic acid (4,5-dimethyl-isoxazol-3-yl)-amide (Compound 65),
3-[4-(3-1,3-Benzodioxol-5-yl-4,6-dimethyl-pyrazolo[4,3-c]pyridin-1-ylmethyl)-2-(2-methoxy-ethoxy)-phenyl]-5-methyl-thiophene-2-sulfonic acid (4,5-dimethyl-isoxazol-3-yl)-amide (Compound 66),
3-[4-[4,6-Dimethyl-3-(5-methyl-thiophen-2-yl)-pyrazolo[4,3-c]pyridin-1-ylmethyl]-2-(2-oxo-pyrrolidin-1-ylmethyl)-phenyl]-5-methyl-thiophene-2-sulfonic acid (4,5-dimethyl-isoxazol-3-yl)-amide (Compound 67),
3-[4-(4,6-Dimethyl-3-phenyl-pyrazolo[4,3-c]pyridin-1-ylmethyl)-2-(3,5-dimethyl-pyrazol-1-ylmethyl)-phenyl]-5-methyl-thiophene-2-sulfonic acid (4,5-dimethyl-isoxazol-3-yl)-amide (Compound 68),
3-{4-[3-(3-Isobutoxy-phenyl)-4,6-dimethyl-pyrazolo[4,3-c]pyridin-1-ylmethyl]-2-methoxy-phenyl}-4,5-dimethyl-thiophene-2-sulfonic acid (4,5-dimethyl-isoxazol-3-yl)-amide (Compound 69),
3-{4-[3-(3-Chloro-phenyl)-4,6-dimethyl-pyrazolo[4,3-c]pyridin-1-ylmethyl]-2-ethoxy-phenyl}-5-ethyl-thiophene-2-sulfonic acid (4,5-dimethyl-isoxazol-3-yl)-amide (Compound 70),
3-{4-[4-(4-Methoxy-phenyl)-5,7-dimethyl-2-oxo-2H-1,6-naphthyridin-1-ylmethyl]-2-propoxy-phenyl}-5-methyl-thiophene-2-sulfonic acid (4-chloro-5-methyl-isoxazol-3-yl)-amide (Compound 71),
3-{4-[5,7-Dimethyl-4-(4-methyl-piperazin-1-yl)-2-oxo-2H-1,6-naphthyridin-1-ylmethyl]-2-methoxy-phenyl}-5-methyl-thiophene-2-sulfonic acid (4,5-dimethyl-isoxazol-3-yl)-amide (Compound 72),
3-[4-(3-Methoxy-4,6-dimethyl-pyrazolo[4,3-c]pyridin-1-ylmethyl)-2-(2-methoxy-ethoxy)-phenyl]-5-methyl-thiophene-2-sulfonic acid (4,5-dimethyl-isoxazol-3-yl)-amide (Compound 73),
4-{4-[2-(4,5-Dimethyl-isoxazol-3-ylsulfamoyl)-5-methyl-thiophen-3-yl]-benzyloxy}-6-methyl-2-propyl-nicotinic acid ethyl ester (Compound 74),
4-{4-[2-(4,5-Dimethyl-isoxazol-3-ylsulfamoyl)-5-methyl-thiophen-3-yl]-benzyloxy}-6-methyl-2-propyl-nicotinic acid (Compound 75),
3-{4-[2-(4,5-Dimethyl-isoxazol-3-ylsulfamoyl)-5-methyl-thiophen-3-yl]-benzyl}-2-oxo-2,3-dihydro-1H-benzimidazole-4-carboxylic acid methyl ester (Compound 76),
3-[2-Ethoxymethyl-4-(6-oxo-4-phenyl-2-propyl-6H-pyrimidin-1-ylmethyl)-phenyl]-5-methyl-thiophene-2-sulfonic acid (4,5-dimethyl-isoxazol-3-yl)-amide (Compound 77),
3-[4-(2,4-Dimethyl-7-oxo-6,7-dihydro-5H-pyrido[2,3-d]pyrimidin-8-ylmethyl)-phenyl]-5-methyl-thiophene-2-sulfonic acid (4,5-dimethyl-isoxazol-3-yl)-amide (Compound 78),
3-[2-Ethoxymethyl-4-(4-ethyl-6-oxo-2-propyl-6H-pyrimidin-1-ylmethyl)-phenyl]-5-methyl-thiophene-2-sulfonic acid (4,5-dimethyl-isoxazol-3-yl)-amide (Compound 79),
3-{4-[(3-Cyano-5,7-dimethyl-1,6-naphthyridin-2-ylamino)-methyl]-phenyl}-thiophene-2-sulfonic acid (4,5-dimethyl-isoxazol-3-yl)-amide (Compound 80),
3-{4-[4,6-Dimethyl-3-(5-methyl-thiophen-2-yl)-pyrazolo[4,3-c]pyridin-1-ylmethyl]-2-ethoxymethyl-phenyl}-5-methyl-thiophene-2-sulfonic acid (4,5-dimethyl-isoxazol-3-yl)-amide (Compound 81),
3-{2-Chloro-4-[5,7-diethyl-3-(5-methyl-thiophen-2-yl)-2-oxo-2H-1,6-naphthyridin-1-ylmethyl]-phenyl}-5-methyl-thiophene-2-sulfonic acid (4-isobutyl-5-methyl-isoxazol-3-yl)-amide (Compound 82),
4-{4-[2-(5-Ethyl-4-methyl-isoxazol-3-ylsulfamoyl)-5-methyl-thiophen-3-yl]-benzyloxy}-6-methyl-2-propyl-nicotinic acid ethyl ester (Compound 83),
3-{2,6-Dichloro-4-[4,6-dimethyl-3-(5-methyl-thiophen-2-yl)-pyrazolo[4,3-c]pyridin-1-ylmethyl]-phenyl}-5-methyl-thiophene-2-sulfonic acid (4,5-dimethyl-isoxazol-3-yl)-amide (Compound 84),
3-{2-Chloro-4-[3-methyl-6-thiophen-2-ylmethyl-4-(3-trifluoromethyl-phenyl)-pyrazolo[4,3-c]pyridin-1-ylmethyl]-phenyl}-5-methyl-thiophene-2-sulfonic acid (4-ethyl-5-methyl-isoxazol-3-yl)-amide (Compound 85),
3-[4-(6-Cyclopropylmethyl-3,4-dimethyl-pyrazolo[4,3-c]pyridin-1-ylmethyl)-2-propyl-phenyl]-5-methyl-thiophene-2-sulfonic acid (5-ethyl-4-methyl-isoxazol-3-yl)-amide (Compound 86),
3-(4-{6-[2-(3-Chloro-phenyl)-ethyl]-3,4-dimethyl-pyrazolo[4,3-c]pyridin-1-ylmethyl}-2-hydroxy-phenyl)-5-methyl-thiophene-2-sulfonic acid (4,5-dimethyl-isoxazol-3-yl)-amide (Compound 87),
3-{2-(2-Hydroxy-ethyl)-4-[3-methyl-6-(4-methyl-benzyl)-4-(5-methyl-thiophen-2-yl)-pyrazolo[4,3-c]pyridin-1-ylmethyl]-phenyl}-5-methyl-thiophene-2-sulfonic acid (4,5-dimethyl-isoxazol-3-yl)-amide (Compound 88),
3-[4-(4-Cyclopropylmethyl-6-isobutyl-pyrazolo[4,3-c]pyridin-1-ylmethyl)-2-methoxy-phenyl]-5-methyl-thiophene-2-sulfonic acid (4,5-dimethyl-isoxazol-3-yl)-amide (Compound 89),
3-[4-(3-Cyclopropyl-4,6-dimethyl-pyrazolo[4,3-c]pyridin-1-ylmethyl)-2-fluoro-phenyl]-5-methyl-thiophene-2-sulfonic acid (4,5-dimethyl-isoxazol-3-yl)-amide (Compound 90),
3-[4-[4-(3-Chloro-phenyl)-6-methyl-3-trifluoromethyl-pyrazolo[4,3-c]pyridin-1-ylmethyl]-2-(2-methoxy-ethoxy)-phenyl]-5-methyl-thiophene-2-sulfonic acid (4,5-dimethyl-isoxazol-3-yl)-amide (Compound 91),
3-[4-(3-Chloro-6-methyl-4-propyl-pyrazolo[4,3-c]pyridin-1-ylmethyl)-2-methyl-phenyl]-4,5-dimethyl-thiophene-2-sulfonic acid (4,5-dimethyl-isoxazol-3-yl)-amide (Compound 92),
3-{4-[3-(4-Butoxy-phenyl)-6-methyl-4-trifluoromethyl-pyrazolo[4,3-c]pyridin-1-ylmethyl]-2-chloro-phenyl}-5-ethyl-thiophene-2-sulfonic acid (5-ethyl-4-methyl-isoxazol-3-yl)-amide (Compound 93),
3-[4-(7-Isobutyl-2-oxo-5-phenyl-2H-1,6-naphthyridin-1-ylmethyl)-phenyl]-5-methyl-thiophene-2-sulfonic acid (4,5-dimethyl-isoxazol-3-yl)-amide (Compound 94),
3-[4-(7-Benzyl-2-oxo-5-propyl-2H-1,6-naphthyridin-1-ylmethyl)-2-chloro-phenyl]-5-ethyl-thiophene-2-sulfonic acid (4,5-dimethyl-isoxazol-3-yl)-amide (Compound 95),
3-{2,6-Dichloro-4-[7-cyclopropylmethyl-5-(5-methyl-thiophen-2-yl)-2-oxo-2H-1,6-naphthyridin-1-ylmethyl]-phenyl}-5-methyl-thiophene-2-sulfonic acid (4,5-dimethyl-isoxazol-3-yl)-amide (Compound 96),
3-[4-(4,5-Dimethyl-2-oxo-7-thiophen-2-ylmethyl-2H-1,6-naphthyridin-1-ylmethyl)-phenyl]-5-methyl-thiophene-2-sulfonic acid (4,5-dimethyl-isoxazol-3-yl)-amide (Compound 97),
3-[2-Chloro-4-(7-ethyl-2-oxo-5-thiophen-2-ylmethyl-2H-1,6-naphthyridin-1-ylmethyl)-phenyl]-5-methyl-thiophene-2-sulfonic acid (4,5-dimethyl-isoxazol-3-yl)-amide (Compound 98),
3-[4-(5-Cyclopropylmethyl-7-ethyl-2-oxo-2H-1,6-naphthyridin-1-ylmethyl)-2-methoxymethyl-phenyl]-5-methyl-thiophene-2-sulfonic acid (4,5-dimethyl-isoxazol-3-yl)-amide (Compound 99),
3-[2-Chloro-4-(7-cyclopropylmethyl-2-oxo-4-o-tolyl-5-trifluoromethyl-2H-1,6-naphthyridin-1-ylmethyl)-phenyl]-5-methyl-thiophene-2-sulfonic acid (4,5-dimethyl-isoxazol-3-yl)-amide (Compound 100),
3-{2-Cyclopropylmethoxy-4-[5,7-dimethyl-2-oxo-4-(pyridin-4-yloxy)-2H-1,6-naphthyridin-1-ylmethyl]-phenyl}-5-methyl-thiophene-2-sulfonic acid (4,5-dimethyl-isoxazol-3-yl)-amide (Compound 101),
3-[2-Chloro-4-(5,7-dimethyl-2-oxo-3-pyridin-2-yl-2H-1,6-naphthyridin-1-ylmethyl)-phenyl]-5-methyl-thiophene-2-sulfonic acid (4,5-dimethyl-isoxazol-3-yl)-amide (Compound 102),
3-[2-Chloro-4-(5,7-diethyl-4-methyl-2-oxo-3-phenyl-2H-1,6-naphthyridin-1-ylmethyl)-phenyl]-5-methyl-thiophene-2-sulfonic acid (4,5-dimethyl-isoxazol-3-yl)-amide (Compound 103),
(S)-2-({4-[2-(4,5-Dimethyl-isoxazol-3-ylsulfamoyl)-5-methyl-thiophen-3-yl]-3-ethoxymethyl-benzyl}-pentanoyl-amino)-3-methyl-butyric acid, (Compound 104)

5: The method for preparation of a compound according to claim 1, comprising at least one of the following steps:
a) reaction of a thiophene with a sulphuryl halide to give a thienylsulphuryl halide,
b) reaction of a thienylsulphurylhalide with a primary amine to give a sulphonamide,
c) N-protection of a sulphonamide to give an N-protected sulphonamide,
d) lithiation of a halogenated thiophene to give a lithiated thiophene,
e) coupling of a lithiated thiophene with a halogen substituted alkyl ester of an aromatic carboxylic acid or an aromatic aldehyde to give an arylthienyl ester or aldehyde,
f) reduction of an arylthienyl ester or aldehyde to give an arylthienyl alcohol,
g) conversion of the hydroxy group of an arylthienyl alcohol to an arylthienyl derivative having a leaving group,
h) reaction of an arylthienyl derivative having a leaving group with nucleophile, and
i) deprotection of an N-protected sulphonamide.

6: The combination comprising a compound according to claim 1, with at least one of beta blockers, calcium antagonists, diuretics, ACE inhibitors, renin inhibitors, angiotensin II antagonists, vasopeptidase inhibitors, mineralocorticoid receptor antagonists, antihypertensive agents, and antidiabetic agents.

7: The combination comprising a compound according to claim 1, with at least one of beta blockers, calcium antagonists, diuretics, ACE inhibitors, renin inhibitors, angiotensin II antagonists, vasopeptidase inhibitors, mineralocorticoid receptor antagonists, antihypertensive agents, antidiabetic agents, fibrinolytic agents, antithrombotic agents, and lipid lowering agents.

8: The pharmaceutical composition comprising a compound according to claim 1, in admixture with a pharmaceutically adjuvant, diluent or carrier.

9: The pharmaceutical composition comprising a combination according to claim 7, in admixture with a pharmaceutically adjuvant, diluent or carrier.

10: The use of a compound according to claim 1 for the preparation of a medicament for treating hypertension of different kinds, alleviating organ damage of different kinds, treating or preventing cardiomyopathies of different origins, diabetic vasculopathy and complications thereof, treating endothelin and angiotensin mediated disorders comprising but not limited to vascular inflammatory conditions including atherosclerosis, and treating prostate cancer.

11: The use of a combination according to claim 7 for the preparation of a medicament for treating hypertension of different kinds, alleviating organ damage of different kinds, treating or preventing cardiomyopathies of different origins, diabetic vasculopathy and complications thereof, treating endothelin and angiotensin mediated disorders comprising but not limited to vascular inflammatory conditions including atherosclerosis, and treating prostate cancer.

12: A dual action receptor antagonist at the AT1 and ETA receptors having higher affinity for AT1 than for ETA, for the preparation of a medicament for treating hypertension of different kinds, alleviating organ damage of different kinds, treating or preventing cardiomyopathies of different origins, diabetic vasculopathy and complications thereof, treating endothelin and angiotensin mediated disorders comprising but not limited to vascular inflammatory conditions including atherosclerosis, and treating prostate cancer.

13: The method for treating hypertension of different kinds, alleviating organ damage of different kinds, treating or preventing cardiomyopathies of different origins, diabetic vasculopathy and complications thereof, treating endothelin and angiotensin mediated disorders comprising but not limited to vascular inflammatory conditions including atherosclerosis, and treating prostate cancer, by administering a compound according to claim 1 to a mammal in need thereof.

14: The method for treating hypertension of different kinds, alleviating organ damage of different kinds, treating or preventing cardiomyopathies of different origins, diabetic vasculopathy and complications thereof, treating endothelin and angiotensin mediated disorders comprising but not limited to vascular inflammatory conditions including atherosclerosis, and treating prostate cancer, by administering a combination according to claim 7 to a mammal in need thereof.

15: A method for treating hypertension of different kinds, alleviating organ damage of different kinds, treating or preventing cardiomyopathies of different origins, diabetic vasculopathy and complications thereof, treating endothelin and angiotensin mediated disorders comprising but not limited to vascular inflammatory conditions including atherosclerosis, and treating prostate cancer, by administering a dual action receptor antagonist at the AT1 and ETA receptors having higher affinity for AT1 than for ETA, to a mammal in need thereof.

16. (canceled)
